(19) United States
(12) Patent Application Publication
(10) Pub. No.: US 2026/0109994 A1
(43) Pub. Date: Apr. 23, 2026

(54) HERBICIDE-RESISTANT GENES FOR MITOCHONDRIAL TRANSFORMATION

(71) Applicant: Napigen, Inc., Wilmington, DE (US)

(72) Inventors: Narendra Singh YADAV, Wilmington, DE (US); Mina HAMIDI, Elkton, MD (US); Hajime SAKAI, Newark, DE (US); Elmer HEPPARD, Wilmington, DE (US); Dilbag Singh MULTANI, Urbandale, IA (US); Sachie KIMURA, Wilmington, DE (US); Cheryl S. CASTER, Landenberg, PA (US); Colleen MCMICHAEL, Wilmington, DE (US); Emil Meyer OROZCO, Jr., Cochranville, PA (US); Max Gabriel SCHUBERT, Oakland, CA (US); Ganesh Murthy KISHORE, Creve Coeur, MO (US)

(21) Appl. No.: 19/317,310

(22) Filed: Sep. 3, 2025

Related U.S. Application Data
(63) Continuation of application No. PCT/US2024/019118, filed on Mar. 8, 2024.
(60) Provisional application No. 63/451,173, filed on Mar. 9, 2023.

(51) Int. Cl. C12N 15/82; C12N 9/10

(52) U.S. Cl. CPC C12N 15/8278; C12N 9/1022; C12N 15/8207; C12N 15/8209; C12Y 202/01006

(57) ABSTRACT

The present disclosure relates to genetically modified cells containing mitochondria that have been transformed with a polynucleotide encoding an herbicide-resistant enzyme, such that the cells can grow in the presence of an inhibitor of the enzyme.

### CROSS-REFERENCE

This application is a continuation of International Application No. PCT/US2024/019118, filed on Mar. 8, 2024, which claims the benefit of U.S. Provisional Application No. 63/451,173, filed on Mar. 9, 2023, which application is incorporated herein by reference in its entirety.

### STATEMENT AS TO FEDERALLY SPONSORED RESEARCH

This invention was made with government support under Agreement No. 58-5062-0-001 awarded by the United States Department of Agriculture (USDA) for a Cooperative Research and Development Agreement (CRADA). The government has certain rights in the invention.

### INCORPORATION BY REFERENCE OF SEQUENCE LISTING

The present application is being filed along with a Sequence Listing in electronic format. The Sequence Listing is provided as a file entitled 51090-707.301_SL.xml, created Dec. 8, 2025, which is 191,050 bytes in size. The information in the electronic format of the Sequence Listing is incorporated by reference in its entirety.

### BACKGROUND

Modification of mitochondrial genomes is of immense importance for basic and applied research. Transgenic plants with stably modified mitochondrial genomes can have new traits such as herbicide tolerance, insect resistance, and/or accumulation of valuable proteins such as pharmaceutical proteins and industrial enzymes.

### SUMMARY

Disclosed herein is a method for transforming a mitochondrion of a cell, the method comprising (a) introducing a first polynucleotide encoding a first polypeptide into the mitochondrion of the cell, wherein the cell is a plant cell or an algal cell, wherein the first polypeptide encoding the first polypeptide is a variant of a naturally occurring polypeptide, wherein the naturally occurring polypeptide comprises an enzyme activity that is inhibited by an herbicide, wherein the variant of the naturally occurring polypeptide comprises an enzyme activity that is resistant to the herbicide; (b) growing the cell under conditions wherein the first polypeptide is expressed; (c) growing the cell in a medium wherein the herbicide is present at an effective concentration; and (d) selecting a transformed cell comprising a transformed mitochondrion, wherein the transformed mitochondrion comprises the first polynucleotide. In some embodiments, disclosed herein is a cell produced by the method described herein, wherein the cell is the plant cell selected from the group consisting of a wheat cell, a maize cell, a rice cell, a barley cell, a sorghum cell, a rye cell, a canola cell, a broccoli cell, a cauliflower cell, a cotton cell, and a soybean cell. In some embodiments, disclosed herein is a plant, a cell, a tissue, a propagation material, a seed, a root, a leaf, a flower, a fruit, a pollen, a progeny, or a part thereof, or any combination thereof, produced from the plant cell described herein, wherein the cell, the tissue, the propagation material, the seed, the root, the leaf, the flower, the fruit, the pollen, the progeny, the part thereof, or the any combination thereof comprises the edited mitochondrial genome.

Disclosed herein is a method for transforming a mitochondrion of a cell, the method comprising: (a) introducing into the mitochondrion of the cell, wherein the cell is a plant cell or an algal cell: (i) a first polynucleotide encoding a first polypeptide, wherein the first polypeptide is a variant of a naturally occurring polypeptide, wherein the naturally occurring polypeptide comprises an enzyme activity that is inhibited by an herbicide, wherein the variant of the naturally occurring polypeptide comprises an enzyme activity that is resistant to the herbicide, and (ii) a second polynucleotide encoding a selectable marker, wherein the selectable marker enables the cell to grow in the presence of a selective agent, wherein the second polynucleotide does not encode the first polypeptide; (b) growing the cell under conditions wherein the selectable marker is expressed; (c) growing the cell in a medium comprising the selective agent of the selectable marker, wherein the selective agent is present at an effective concentration; and (d) selecting a transformed cell comprising a transformed mitochondrion, wherein the transformed mitochondrion comprises the first polynucleotide, and further wherein the transformed cell comprising the transformed mitochondrion can grow in a medium comprising the herbicide, wherein the herbicide is present at an effective concentration. In some embodiments, disclosed herein is a cell produced by the method described herein, wherein the cell is the plant cell selected from the group consisting of a wheat cell, a maize cell, a rice cell, a barley cell, a sorghum cell, a rye cell, a canola cell, a broccoli cell, a cauliflower cell, a cotton cell, and a soybean cell. In some embodiments, described herein is a plant, a cell, a tissue, a propagation material, a seed, a root, a leaf, a flower, a fruit, a pollen, a progeny, or a part thereof, or any combination thereof, produced from the plant cell described herein, wherein the cell, the tissue, the propagation material, the seed, the root, the leaf, the flower, the fruit, the pollen, the progeny, the part thereof, or the any combination thereof comprises the edited mitochondrial genome.

Described herein is a method of controlling weeds, the method comprising growing a plurality of plants in a presence of an herbicide that is an inhibitor of a plant enzyme, wherein at least one plant of the plurality of plants comprises a mitochondrion comprising a heterologous polynucleotide that encodes a variant of the plant enzyme, wherein the variant of the plant enzyme has an enzyme activity resistant to the herbicide, wherein the presence of the herbicide is sufficient to selectively promote growth of the at least one plant of the plurality of plants, resulting in an increased growth of the at least one plant of the plurality of plants relative to plants lacking the heterologous polynucleotide. In some embodiments, described herein is a transgenic plant or parts thereof comprising the cell described herein. In some embodiments, a field or a greenhouse can comprise the transgenic plant or parts thereof described herein. In some embodiments, a food product can comprise the cell described herein. In some embodiments, a field can comprise the cell described herein. In some embodiments, a kit can comprise the cell described herein or the transgenic plant or parts thereof described herein.

Described herein is a cell comprising an edited mitochondrial genome, wherein the cell is a plant cell or an algal cell, wherein the edited mitochondrial genome comprises a heterologous polynucleotide encoding a variant of a naturally occurring polypeptide having an enzyme activity, wherein the enzyme activity of the naturally occurring polypeptide is inhibited by an herbicide, wherein an enzyme activity of the variant of the naturally occurring polypeptide having the enzyme activity is resistant to the herbicide. In some embodiments, described herein is a transgenic plant or parts thereof comprising the cell described herein. In some embodiments, a field or a greenhouse can comprise the transgenic plant or parts thereof described herein. In some embodiments, a food product can comprise the cell described herein. In some embodiments, a field can comprise the cell described herein. In some embodiments, a kit can comprise the cell described herein or the transgenic plant or parts thereof described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 represents a map of plasmid pNAP170. The plasmid contains the following three nuclear expression cassettes: pUBI1::MTS-ALS(HR-LS)::OCS terminator; pACT1::MTS-ALS(SS)::NOS terminator; and p35S::HPT::CaMV terminator.

FIG. 2 represents growth of rice callus cells in a medium containing chlorsulfuron, in which the rice callus cells were transformed with pNAP170. The callus within the circle was subjected to further selection.

FIG. 3 represents a map of plasmid pNAP198. The plasmid contains the mALS(HR-LS) coding region operably linked to a hybrid T7+rice ATP1 promoter and a hybrid T7+rice ATP1 terminator. The plasmid also has a eGFP coding sequence operably linked to a rice COB1 promoter and 5′ UTR and a rice COB1 terminator. The plasmid also contains a B4 element that has been associated with autonomous replication in rice mitochondria.

FIG. 4 shows growth of rice callus cells in a medium containing chlorsulfuron, in which the rice callus cells were co-transformed with pNAP195 and pNAP198. The callus samples within the circle were subjected to further selection.

FIG. 5 shows a map of plasmid pNAP432. The plasmid contains a Donor DNA used to transform rice mitochondria. The Donor DNA has an mALS(HR-LS) coding region (with an nad4L RNA editing site) operably linked to a hybrid T7+rice ATP1 promoter and a truncated version of the hybrid T7+rice ATP1 terminator. The Donor DNA has rice mitochondrial DNA homologous regions at the 5′ and 3′ ends of 1.6 kb and 1.2 kb, respectively.

FIG. 6A and FIG. 6B show growth of transformed rice callus cells in a medium containing chlorsulfuron. FIG. 6A shows rice callus cells were co-transformed with plasmid pNAP195 and isolated Donor DNA from pNAP432. FIG. 6B shows rice callus cells were co-transformed with plasmid pNAP195 and isolated Donor DNA from pNAP433.

FIG. 7A and FIG. 7B show the effect of disulfiram on inhibition of wild-type rice callus growth by oligomycin. FIG. 7A show rice callus at day zero. FIG. 7B shows rice callus at day 14. Plates 1˜4 had ND2 medium as the base medium with the indicated additions. Plate 1:0.5% sucrose. Plate 2:0.5% sucrose and 100 μM disulfiram. Plate 3:0.5% sucrose and 1 mg/L oligomycin. Plate 4:0.5% sucrose and 100 μM disulfiram and 1 mg/L oligomycin. FIG. 7A and FIG. 7B show that disulfiram can increase the ability of oligomycin to inhibit growth of wild-type rice callus in the presence of 0.5% sucrose.

FIG. 8 shows the PCR analysis of the 5′ and 3′ integration sites in rice mitochondrial DNA of Donor DNA carrying the sulfonylurea resistance gene. The events analyzed here were initially co-transformed with the pNAP432 Donor DNA fragment and the pNAP195 plasmid DNA. The integration site was amplified with one primer complementary only to the rice mitochondrial DNA and the other primer complementary only to the Donor DNA (EXAMPLE 11). Samples of the second PCR round of nested PCR reactions were separated on a gel. Lanes 1-8: 5′ junction amplification. Lanes 9-15: 3′ junction amplification. Lanes 1-15 correspond to PCR analysis of the following samples: #1 & #9: HH43 event (regenerated leaf); #2 & #10: wild-type leaf; #3 & #11: HH43 event A callus; #4 & #12: HH43 event B callus; #5 & #13: no DNA control; #6 & #14: wild-type rice callus, #7 & #15: event T033 callus, #8: blank lane (no sample loaded). Arrows on the left and right sides of the gel show the expected locations of the 5′ junction fragment (1821 bp) and the 3′ junction fragment (1432 bp), respectively. Lanes M contain molecular size standards.

FIG. 9 shows the PCR analysis of the 5′ integration site in rice mitochondrial DNA of Donor DNA carrying the glyphosate resistance gene. The events analyzed here were initially transformed with the pNAP661 Donor DNA fragment. The integration site was amplified with one primer complementary only to the rice mitochondrial DNA and the other primer complementary only to the Donor DNA (EXAMPLE 12). Lanes 1-16 correspond to PCR analysis of callus tissue from events TT53-TT67 and wild type-callus, respectively. Lanes M contain molecular size standards. An arrow shows the location of the expected 5′ junction fragment. Events TT57 and TT58 produced PCR fragments of the expected size (1.8 kb) for 5′ integration in the targeted site.

FIG. 10 shows the PCR analysis of the 3′ integration site in rice mitochondrial DNA of Donor DNA carrying the glyphosate resistance gene. The events analyzed here were initially transformed with the pNAP661 Donor DNA fragment. The integration site was amplified with one primer complementary only to the rice mitochondrial DNA and the other primer complementary only to the Donor DNA (EXAMPLE 12). Lanes 1-15 correspond to PCR analysis of callus tissue from event TT51, TT57, TT58, TT13, TT14, TT17, TT25, TT31, TT33, TT41, TT42, TT46, TT61, TT64, and TT67, respectively. Lane 16: wild-type rice callus. Lane 17: no DNA control. Lanes M contain molecular size standards. Arrows show the location of a 1.4 kb 3′ junction fragment from events TT31 and TT46. This 3′ junction fragment was approximately 0.3 kb shorter than expected, as confirmed by subsequent DNA sequencing (EXAMPLE 12).

FIG. 11 shows the PCR analysis of the 5′ integration site in rice mitochondrial DNA of the Donor DNA carrying the glufosinate resistance gene. The events analyzed here were initially transformed with the truncated CsiI-BmtI Donor DNA fragment from pNAP643. The integration site was amplified with one primer complementary only to the rice mitochondrial DNA and the other primer complementary only to the truncated Donor DNA fragment (EXAMPLE 13). Lanes 1-18 correspond to PCR analysis of callus tissue from events S94-S111, respectively. Lanes M contain molecular size standards. Arrows show the location of the expected 5′ junction fragment. Events S96, S109, and S111 produced PCR fragments of the expected size (1.8 kb) for 5′ integration at the targeted site.

FIG. 12 shows the PCR analysis of the 3′ integration site in rice mitochondrial DNA of Donor DNA carrying the glufosinate resistance gene. The events analyzed here were initially transformed with the truncated CsiI-BmtI Donor DNA fragment from pNAP643. The integration site was amplified with one primer complementary only to the mitochondrial DNA and the other primer complementary only to the Donor DNA (EXAMPLE 13). Lanes 1-18 correspond to PCR analysis of callus tissue from events S94-S111, respectively. Lanes M contain molecular size standards. Arrows show the location of a 1.4 kb 3′ junction fragment from events S96 and S109. This 3′ junction fragment was approximately 0.3 kb shorter than expected, as confirmed by subsequent DNA sequencing (EXAMPLE 13).

### DETAILED DESCRIPTION

In some cases, mitochondrial genome editing can be more difficult than nuclear genome or plastid genome editing. In some cases, a new selectable marker gene can be used to generate and identify a cell comprising an edited mitochondrial genome. In some cases, a new selectable marker gene can be needed to edit a mitochondrial genome of a plant.

Disclosed herein in some embodiments, are methods and compositions for making and using organisms comprising a polynucleotide encoding a polypeptide, wherein the polypeptide is a variant of a naturally occurring polypeptide having an enzyme activity. In some embodiments, the enzyme activity of the naturally occurring polypeptide can play a critical role in plant growth, development, or survival. In some embodiments, the enzyme activity of the naturally occurring polypeptide is inhibited by one or more herbicides. In some embodiments, the enzyme activity of the naturally occurring polypeptide can comprise acetolactate synthase (ALS) activity, 5-enol-pyruvyl-shikimate-3-phosphate synthase (EPSPS) activity, glutamine synthetase (GS) activity. TABLE 1 provides non-limiting examples of herbicides and their corresponding targets. In some embodiments, an enzyme can be of bacterial or eukaryotic origin. In some embodiments, the variant of the naturally occurring enzyme (e.g., ALS, EPSPS, or GS) described herein can be resistant to the herbicide.

In some cases, the enzyme activity of the naturally occurring polypeptide is acetolactate synthase activity and the herbicide is an inhibitor of acetolactate synthase (e.g., sulfonylureas or imidazolinone). In some embodiments, a polynucleotide can encode an enzyme having acetolactate synthase (ALS) activity or a biologically active fragment thereof. In some embodiments, an enzyme can be an herbicide-resistant acetolactate synthase large subunit (ALS-LS) polypeptide or a biologically active fragment thereof of Oryza sativa. In some embodiments, the terms “ALS-LS”, “ALS (LS)”, “ALS large subunit” and “ALS catalytic subunit” may be used interchangeably. In some embodiments, an herbicide-resistant ALS-LS or a biologically active fragment thereof in a mitochondrion can enable growth in the presence of an inhibitor of ALS which can allow for its use as a selectable marker. In some embodiments, an herbicide-resistant ALS-LS polypeptide disclosed herein can comprise a sequence presented in SEQ ID NO: 7. In some embodiments, an herbicide-resistant ALS-LS polypeptide disclosed herein can comprise at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence presented in SEQ ID NO: 7.

In some cases, the enzyme activity of the naturally occurring polypeptide is 5-enolpyruvylshikimate-3-phosphate synthase (EPSPS) activity and the herbicide is an inhibitor of EPSPS (e.g., glyphosate). In some embodiments, a polynucleotide can encode an enzyme having EPSPS activity or a biologically active fragment thereof. In some embodiments, an enzyme can be an herbicide-resistant EPSPS activity or a biologically active fragment thereof. In some embodiments, an herbicide-resistant EPSPS or a biologically active fragment thereof in a mitochondrion can enable growth in the presence of an inhibitor of EPSPS which can allow for its use as a selectable marker.

In some cases, the enzyme activity of the naturally occurring polypeptide is glutamine synthetase (GS) activity and the herbicide is an inhibitor of GS (e.g., glufosinate). In some embodiments, a polynucleotide can encode an enzyme having GS activity or a biologically active fragment thereof. In some embodiments, an enzyme can be an herbicide-resistant glutamine synthetase polypeptide or a biologically active fragment thereof. In some embodiments, an herbicide-resistant GS or a biologically active fragment thereof in a mitochondrion can enable growth in the presence of an inhibitor of GS which can allow for its use as a selectable marker. In some embodiments, a transformed mitochondrion can comprise the polynucleotide. In some embodiments, a transformed mitochondrion can comprise the edited mitochondrial genome.

TABLE 1
Non-limiting examples of herbicides & targets

| Herbicide | Target | Biochemical Pathways |
|---|---|---|
| Glyphosate | 5-enolpyruvylshikimate-3- | Shikimate pathway (involved in |
| | phosphate synthase (EPSPS) | the synthesis of aromatic amino |
| | | acids) |
| Glufosinate | Glutamine synthetase (GS) | Nitrogen metabolism (synthesis |
| | | of glutamine) |
| Sulfonylureas | Acetolactate synthase (ALS) or | Synthesis of branched-chain |
| (e.g., Chlorsulfuron, | Acetohydroxyacid synthase | amino acids (valine, leucine, |
| Sulfometuron) | (AHAS) | isoleucine) |
| Imidazolinone (e.g., | Acetolactate synthase (ALS) or | Synthesis of branched-chain |
| Imazethapyr, Imazapyr) | Acetohydroxyacid synthase | amino acids |
| | (AHAS) | |
| Triazines (e.g., Atrazine, | Photosystem II | Photosynthesis (electron |
| Simazine) | | transport) |
| Phenylureas (e.g., Diuron, | Photosystem II | Photosynthesis (electron |
| Linuron) | | transport) |
| Bleachers (e.g., Isoxaben, | Various targets involved in cell | Cell growth and division |
| Trifluralin) | division and cell wall synthesis | |
| Dicamba | growth regulator pathways | Plant growth regulation |
| | mimicking auxins | |
| 2,4-D | growth regulator pathways | Plant growth regulation |
| | mimicking auxins | |
| Fenoxaprop | Acetyl-CoA carboxylase | Fatty acid synthesis |
| Pyridiazinones | Phytoene desaturase | Carotenoid biosynthesis |
| Isoxazoles | p-hydroxyphenyl pyruvate | Carotenoid biosynthesis |
| | dioxygenase (HPPD) | |
| Diphenylethers | Protoporphyrinogen oxidase | Chlorophyll and heme |
| | | biosynthesis |
| Asulam | 7,8-dihydropteroate synthase | Folic acid synthesis |

Further disclosed herein are methods for transforming a mitochondrion, the method comprising (a) introducing into the mitochondrion of a cell a first polynucleotide encoding a first polypeptide, wherein the first polypeptide is a variant of a naturally occurring polypeptide having an enzyme activity, wherein the enzyme activity of the naturally occurring polypeptide can be inhibited by an herbicides, wherein an enzyme activity of the variant of the naturally occurring polypeptide can be resistant to the herbicide, (b) growing the cell under conditions wherein the first polypeptide is expressed; and (c) growing the cell in a medium wherein the herbicide is present at an effective concentration. In some embodiments, the methods for selecting or screening for a genetic modification event, for example, a transformed mitochondrion comprising the edited mitochondrion genome expressing the polynucleotide encoding the polypeptide described herein, after introduction into an organelle (e.g., nucleus, mitochondria), a cell, or tissue of interest. Disclosed herein in some embodiments, are recombinant plant cells, recombinant plant tissues, transgenic plants, transgenic plant seeds, transgenic plant roots, transgenic plant flowers, transgenic plant fruits, transgenic plant pollens, and transgenic plant progenies comprising the edited mitochondrial genome as described herein. In some cases, the transgenic plants comprising the edited mitochondrial genome as described herein can be resistant to killing and/or growth inhibition by one or more herbicides. In some embodiments, transformed seeds and transgenic progeny plants of the parent transgenic plant comprising the edited mitochondrial genome as described herein can be used to produce food, feed, industrial products, oil, nutrients, and other valuable products. In some cases, the methods and compositions described herein can be used to control the growth of unwanted plants amongst crops or other plants comprising the edited mitochondrion as described herein, thereby enhancing growth and production of the crop or other plants of interest.

### Definitions

In some embodiments, the meaning of abbreviations can be as follows: “sec” can mean second(s), “min” can mean minute(s), “h” can mean hour(s), “d” can mean day(s), “μL” can mean microliter(s), “ml” can mean milliliter(s), “L” can mean liter(s), “μM” can mean micromolar, “mM” can mean millimolar, “M” can mean molar, “mmol” can mean millimole(s), “μmole” can mean micromole(s), “g” can mean gram(s), “μg” can mean microgram(s), “ng” can mean nanogram(s), “U” can mean unit(s), “nt” can mean nucleotide(s); “bp” can mean base pair(s), “kb” can mean kilobase(s) and “kbp” can mean kilobase pair(s).

In some embodiments, “transgenic” can refer to any cell, cell line, callus, tissue, organism part or whole organism (e.g., plant), the genome of which has been edited or altered by the presence of a heterologous or exogenous nucleic acid, such as a recombinant DNA construct. In some embodiments, transgenic events can include those created by sexual crosses or asexual propagation. In some embodiments, the term “transgenic” may not encompass an edited genome or alteration of a genome (e.g., chromosomal, or extra-chromosomal) by breeding methods or by naturally occurring events such as random cross-fertilization, non-recombinant viral infection, non-recombinant bacterial transformation, non-recombinant transposition, or spontaneous mutation. In some embodiments, the term “transgenic” may encompass an edited genome or alteration of a genome (e.g., chromosomal, or extra-chromosomal) by breeding methods or by naturally occurring events such as random cross-fertilization, non-recombinant viral infection, non-recombinant bacterial transformation, non-recombinant transposition, or spontaneous mutation.

In some embodiments, “genome”, for example, of a cell or whole organism can encompass chromosomal DNA found within a nucleus (nuclear DNA), and DNA found within a cytoplasmic organelle (e.g., mitochondrial DNA, plastid DNA). Methods and compositions of a disclosure can be used for editing the genome of a nucleus, a cytoplasmic organelle (e.g., mitochondrion, plastid), or any combination thereof.

In some embodiments, the terms “full complement” and “full-length complement” can be used interchangeably herein, and can refer to a complement of a given nucleotide sequence. In some aspects, a complement and a nucleotide sequence can comprise a same number of nucleotides. In some aspects, a complement and a nucleotide sequence can comprise 100% complementary. In some embodiments, a complement and a nucleotide sequence can differ in a number of nucleotides. In some embodiments, complementarity (e.g., between a complement and a nucleotide sequence) can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100%. In some embodiments, complementarity (e.g., between a complement and a nucleotide sequence) can be at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 91%, at most about 92%, at most about 93%, at most about 94%, at most about 95%, at most about 96%, at most about 97%, at most about 98%, at most about 99%, or 100%.

In some embodiments, “polynucleotide”, “nucleic acid”, “nucleic acid sequence”, “nucleotide sequence”, or “nucleic acid fragment”, which can be used interchangeably, can refer to a polymer of a nucleic acid (e.g., RNA, DNA, or both, and analogs thereof) that can be single-stranded or double-stranded (or both single-stranded and double-stranded), optionally containing synthetic, non-natural or altered nucleotide bases. In some embodiments, nucleotides (e.g., in their 5′-monophosphate form) can be referred to by a single letter designation as follows (for RNA or DNA, respectively): “A” for adenylate or deoxyadenylate, “C” for cytidylate or deoxycytidylate, “G” for guanylate or deoxyguanylate, “U” for uridylate, “T” for deoxythymidylate, “R” for purine-based nucleotides (A or G), “Y” for pyrimidine-based nucleotides (C or T), “K” for G or T, “H” for A or C or T, “I” for inosine, and “N” for any nucleotide. In some embodiments, a polynucleotide can be linear or circular.

In some embodiments, as used herein, a “nucleic acid” can refer to a polynucleotide sequence, or fragment thereof. In some embodiments, a nucleic acid can comprise nucleotides. In some embodiments, a nucleic acid can exist in a cell-free environment. In some embodiments, a nucleic acid can be a gene or fragment thereof. In some embodiments, a nucleic acid can be DNA. In some embodiments, a nucleic acid can be RNA. In some embodiments, a nucleic acid can comprise one or more analogs (e.g., altered backbone, sugar, or nucleobase). In some embodiments, non-limiting examples of analogs can include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In some embodiments, “polypeptide”, “peptide”, “amino acid sequence” and “protein”, which can be used interchangeably herein, can refer to a polymer of amino acid residues. In some embodiments, these terms can apply to amino acid polymers in which one or more amino acid residues can be, for example, an artificial chemical analogue of a corresponding naturally occurring amino acid and/or to naturally occurring amino acid polymers. In some embodiments, the terms “polypeptide”, “peptide”, “amino acid sequence”, and “protein” can be inclusive of modifications including, but not limited to, glycosylation, lipid attachment, sulfation, gamma-carboxylation of glutamic acid residues, hydroxylation and ADP-ribosylation. In some embodiments, the polypeptide having an enzymatic activity can comprise an active site that facilitate enzymatic activity. In some embodiments the active site of the polypeptide having an enzymatic activity can comprise a substrate binding domain and a catalytic domain that catalyze a reaction of the substrate.

In some embodiments, a “functional fragment” of a polynucleotide or polypeptide can refer to any subset of contiguous nucleotides or contiguous amino acids, respectively, in which an original (e.g., wild type) activity (or substantially similar activity) of a polynucleotide or polypeptide can be retained. In some embodiments, the terms “functional fragment”, “functional subfragment”, “fragment that is functionally equivalent”, “subfragment that is functionally equivalent”, “functionally equivalent fragment”, “a biologically active fragment” and “functionally equivalent subfragment” can be used interchangeably herein.

In some embodiments, the term an “effective concentration” of an herbicide or of a selective agent for a plant, callus, cell, or other plant tissue, can be an amount that will cause either a decreased growth rate, an arrest of growth, or death of the plant, callus, cell, or other plant tissue, as compared to a plant, callus, cell, or other plant tissue not exposed to the herbicide or selective agent. An effective concentration can allow one to distinguish between a sample that has resistance or tolerance to an herbicide or a selective agent versus one that does not.

In some embodiments, the terms “functional variant”, “variant that is functionally equivalent” and “functionally equivalent variant” can be used interchangeably herein. In some embodiments, in the context of a polynucleotide or a polypeptide, these terms can refer to a variant of the nucleic acid sequence or the amino acid sequence, respectively, in which the original activity (or substantially similar activity) of the polynucleotide or polypeptide can be retained. In some embodiments, fragments and variants can be obtained via methods such as site-directed mutagenesis and synthetic construction.

In some embodiments, an activity of a functional fragment or functional variant can be, for example, about: 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, or less than 10% of that of an original (e.g., wild type) activity.

In some embodiments, an “RNA transcript” can refer to a product resulting from an RNA polymerase-catalyzed transcription of a DNA sequence. In some embodiments, when an RNA transcript is a perfect complementary copy of a DNA sequence, it can be referred to as a primary transcript. In some embodiments, an RNA transcript can be referred to as a mature RNA, for example, when it is an RNA sequence derived from post-transcriptional processing of a primary transcript.

In some embodiments, a “messenger RNA” or “mRNA” can refer to an RNA that is without introns and that can be translated into protein by a cell.

In some embodiments, “sense” RNA can refer to an RNA transcript that includes an mRNA. In some embodiments, sense RNA can be translated into protein within a cell or in vitro.

In some embodiments, “antisense RNA” can refer to an RNA transcript that can be complementary to all or part of a target RNA (e.g., a primary transcript or mRNA). In some embodiments, antisense RNA can be used to block expression of a target gene. In some embodiments, a complementarity of an antisense RNA may be with any part of a specific gene transcript, i.e., at a 5′ non-coding sequence, 3′ non-coding sequence, introns, or a coding sequence. In some embodiments, “functional RNA” can refer to antisense RNA, ribozyme RNA, or other RNA that may not be translated but yet can have an effect on cellular processes. In some embodiments, the terms “complement” and “reverse complement” can be used interchangeably herein, for example, with respect to mRNA transcripts and can be used to define the antisense RNA of a message.

In some embodiments, “cDNA” can refer to a DNA that can be complementary to and synthesized from a mRNA template using a reverse transcriptase enzyme. In some embodiments, a cDNA can be single-stranded or converted into a double-stranded form using a Klenow fragment of DNA polymerase I.

In some embodiments, a “coding region” can refer to a portion of a messenger RNA (or a corresponding portion of another nucleic acid molecule such as a DNA molecule) which can encode a protein or polypeptide. In some embodiments, a “non-coding region” can refer to a portion of a messenger RNA or other nucleic acid molecule that is not a coding region, including but not limited to, for example, a promoter region, a 5′ untranslated region (“UTR”), a 3′ UTR, an intron, and a terminator. In some embodiments, the terms “coding region” and “coding sequence” can be used interchangeably herein. In some embodiments, the terms “non-coding region” and “non-coding sequence” can be used interchangeably herein.

In some embodiments, “coding sequence” can be abbreviated “CDS”. In some embodiments, “open reading frame” can be abbreviated “ORF”.

In some embodiments, “gene” can refer to a nucleic acid fragment that can express a functional molecule such as, but not limited to, a specific protein, including: introns, exons, regulatory sequences preceding (5′ non-coding sequences) and following (3′ non-coding sequences) a coding sequence. In some embodiments, “native gene” can refer to a gene as found in nature, for example, with its own regulatory sequences.

In some embodiments, a “mutated gene” can be a gene that has been altered relative to a corresponding naturally occurring gene; e.g., through human intervention. In some embodiments, such a “mutated gene” can have a sequence that differs from a sequence of a corresponding non-mutated gene by at least one nucleotide addition, deletion, or substitution. In some embodiments, a mutated gene can comprise an alteration that results from a polynucleotide guided polypeptide system as disclosed herein. In some embodiments, a mutated organism can be an organism comprising a mutated gene; e.g., a mutated plant with an organellar genome comprising a mutated gene. In some embodiments, the terms “mutated gene” and “mutant gene” can be used interchangeably herein.

In some embodiments, the term “SDN” can refer to “site-directed nuclease”. In some embodiments, an SDN-induced mutation can include; an induction of site-specific random mutations; an induction of mutations in a predefined sequence of a particular gene; a replacement or an insertion of an entire gene; or any combination thereof. In some embodiments, SDN-induced mutations can be referred to as SDN-1, SDN-2, and SDN-3, respectively.

In some embodiments, a “codon-modified gene” or “codon-preferred gene” or “codon-optimized gene” can be a gene having its frequency of codon usage designed to mimic a frequency of preferred codon usage of a host cell in a compartment of interest. In some embodiments, a compartment of interest can comprise a nucleus, a mitochondrion, a chloroplast, or any combination thereof.

In some embodiments, a “mature” protein can refer to a post-translationally processed polypeptide; for example, one from which any pre- or pro-peptides present in a primary translation product have been removed.

In some embodiments, a “precursor” protein can refer to a primary product of translation of an mRNA; for example, with pre- and pro-peptides still present. In some embodiments, pre- and pro-peptides may, for example, comprise intracellular localization signals.

In some embodiments, “isolated” can refer to materials, such as nucleic acid molecules, proteins, and cells that may be substantially free or otherwise removed from components that normally accompany or interact with materials in a naturally occurring environment. In some embodiments, isolated polynucleotides can be purified from a host cell in which they can naturally occur. In some embodiments, nucleic acid purification methods can be used to obtain isolated polynucleotides. In some embodiments, isolated polynucleotides can include, for example, recombinant polynucleotides and chemically synthesized polynucleotides.

In some embodiments, “heterologous”, for example, with respect to sequence, can mean a sequence that originates from a foreign species, or, if from the same species, is substantially modified from its native form in composition and/or genomic locus by deliberate human intervention. In some embodiments, the terms “heterologous nucleotide sequence”, “heterologous sequence”, “heterologous nucleic acid fragment”, and “heterologous nucleic acid sequence” can be used interchangeably herein. In some embodiments, “heterologous” can refer to a nucleic acid sequence which does not naturally occur in a genome. In some embodiments, “heterologous” can refer to a nucleic acid sequence which has been artificially introduced into a genome. In some embodiments, “heterologous” can refer to a nucleic acid sequence which is present in a genome as a result of genetic editing. In some embodiments, a heterologous nucleic acid can be exogenous or endogenous to a cell. In some embodiments, “heterologous” can refer to a nucleic acid sequence which does not naturally occur in one organelle of a cell, but which does naturally occur in another organelle of a same cell. In some embodiments, “heterologous” can refer to a nucleic acid sequence that has been introduced into a mitochondrion of a cell which does not naturally occur in the mitochondrion of that cell, but which does naturally occur in a nucleus of that cell. In some embodiments, methods disclosed herein can produce an organelle that comprises a heterologous nucleic acid sequence that has not been integrated into a genome of the organelle. In some embodiments, a heterologous nucleic acid sequence that has not been integrated into a genome of the organelle can comprise a sequence that is part of a plasmid.

In some embodiments, “recombinant” can refer to an artificial combination of two or more otherwise separated segments of sequence, e.g., by chemical synthesis or by a manipulation of isolated segments of nucleic acids by genetic engineering techniques. In some embodiments, “recombinant” can also include reference to a cell or vector, for example, that has been modified by an introduction of a heterologous nucleic acid or a cell derived from a cell so modified.

In some embodiments, a “recombinant DNA construct” can refer to a combination of nucleic acid fragments that may not normally be found together in nature. In some embodiments, a recombinant DNA construct may comprise, for example, regulatory sequences and coding sequences that are derived from different sources, or regulatory sequences and coding sequences derived from the same source. In some embodiments, sequences in a recombinant DNA construct can be arranged in a manner different than that normally found in nature. In some embodiments, the terms “recombinant DNA construct”, “recombinant DNA molecule”, “recombinant construct”, “DNA construct” and “construct” can be used interchangeably herein. In some embodiments, a recombinant DNA construct may be any of the following non-limiting examples: single-stranded, double-stranded, or both single-stranded and double-stranded; linear or circular; DNA, RNA, or a combination of DNA and RNA; a plasmid DNA, a viral DNA, a viral RNA, or a viroid RNA.

In some embodiments, “expression” can refer to a production of a functional product. For example, expression of a nucleic acid fragment may refer to transcription of the nucleic acid fragment (e.g., transcription resulting in mRNA or functional RNA) and/or translation of mRNA into a precursor or mature protein.

In some embodiments, an “expression cassette” can refer to a construct containing, for example, a polynucleotide, a regulatory element(s), and a polynucleotide that allow for expression of a polynucleotide in a host. In some embodiments, the terms “expression cassette” and “expression construct” can be used interchangeably herein.

In some embodiments, the terms “entry clone” and “entry vector” can be used interchangeably herein.

In some embodiments, “regulatory sequences” can refer to nucleotide sequences, for example, located upstream (e.g., 5′ non-coding sequences), within (e.g., in introns), or downstream (e.g., 3′ non-coding sequences) of a coding sequence. In some embodiments, regulatory sequences can influence, for example, the transcription, RNA processing or stability, or translation of the associated coding sequence. In some embodiments, regulatory sequences may include, but are not limited to, promoters, translation leader sequences, 5′ untranslated sequences, 3′ untranslated sequences, introns, polyadenylation target sequences, RNA processing sites, effector binding sites, and stem-loop structures. In some embodiments, a regulatory sequence may act in “cis” or “trans”. In some embodiments, the nucleic acid molecule regulated by a regulatory sequence may not necessarily have to encode a functional peptide or polypeptide, e.g., the regulatory sequence can modulate the expression of a short interfering RNA or an antisense RNA. In some embodiments, the terms “regulatory sequence” and “regulatory element” can be used interchangeably herein.

In some embodiments, “promoter” can refer to a nucleic acid fragment that can control transcription of another nucleic acid fragment. In some embodiments, a promoter can include a core promoter (also known as minimal promoter) sequence. In some embodiments, a core promoter can be a minimal sequence for direct transcription initiation. In some embodiments, a core promoter can optionally include enhancers or other regulatory elements. In some embodiments, promoters may be derived in their entirety from a native gene, or be composed of different elements derived from different promoters found in nature, or even comprise synthetic DNA segments. Different promoters may direct the expression of a gene in different tissues or cell types, or at different stages of development, or in response to different environmental conditions.

In some embodiments, a “promoter functional in a plant” can be a promoter that can control transcription in plant cells. In some embodiments, a promoter can be from any suitable origin, which can include plant cells and non-plant cells.

In some embodiments, a “tissue-specific promoter” and “tissue-preferred promoter” can be used interchangeably and can refer to a promoter that can be expressed predominantly in one tissue, one organ or one cell type. In some embodiments, a tissue-specific promoter may not be necessarily exclusive in one tissue, one organ or one cell type. In some embodiments, a root-preferred promoter can include, for example, the following: soybean root-specific glutamine synthetase gene; cytosolic glutamine synthetase (GS); root-specific control element in the GRP 1.8 gene of French bean; root-specific promoter of A. tumefaciens mannopine synthase (MAS); root-specific promoters isolated from Parasponia andersonii and Trema tomentosa; A. rhizogenes rolC and rolD root-inducing genes; Agrobacterium wound-induced TR1′ and TR2′ genes; VfENOD-GRP3 gene promoter; and rolB promoter. In some embodiments, a seed-preferred promoter can include a seed-specific promoter active during seed development, a seed-germinating promoter active during seed germination, or any combination thereof. In some embodiments, a seed-preferred promoter can include Cim1 (cytokinin-induced message); cZ19B1 (maize 19 kDa zein); mi1ps (myo-inositol-1-phosphate synthase); END1; and END2, or any combination thereof. In some embodiments, for a dicot, a seed-preferred promoter can include; bean β-phaseolin; napin; β-conglycinin; soybean lectin; cruciferin; and any combination thereof. In some embodiments, for monocots, a seed-preferred promoter can include maize 15 kDa zein; 22 kDa zein; 27 kDa gamma zein; waxy; shrunken 1; shrunken 2; globulin 1; oleosin; nud; Zea mays-Rootmet2 promoter, or any combination thereof. In some embodiments, a leaf-preferred promoter can include a plant rbcS promoter, such as a soybean rbcS promoter, a maize rbcS promoter; a Zea mays PEPC1 promoter, or any combination thereof.

In some embodiments, a “developmentally regulated promoter” can refer to a promoter whose activity can be determined by developmental events.

In some embodiments, an “inducible promoter” can refer to a promoter that selectively expresses an operably linked DNA sequence in response to a presence of an endogenous or exogenous stimulus, for example by a chemical compound (e.g., a chemical inducer) or in response to an environmental, hormonal, chemical, and/or developmental signal. In some embodiments, an inducible or regulated promoter can include, for example, promoters regulated by light, heat, stress, flooding, or drought, phytohormones, wounding, or chemicals such as ethanol, jasmonate, salicylic acid, or safeners. In some embodiments, a pathogen-inducible promoter that can be induced following infection by a pathogen can include, those regulating expression of PR proteins, SAR proteins, beta-1,3-glucanase, chitinase, or any combination thereof. In some embodiments, a stress-inducible promoter can include a plant RAB17 promoter, such as a maize RAB17 promoter. In some embodiments, a chemical-inducible promoter can include a maize ln2-2 promoter; a maize GST promoter; a tobacco PR-1a promoter, or any combination thereof. In some embodiments, a maize ln2-2 promoter can be activated by benzene sulfonamide herbicide safeners. In some embodiments, a maize GST promoter can be activated by a hydrophobic electrophilic compound. In some embodiments, a maize GST promoter can be used as a pre-emergent herbicide. In some embodiments, a tobacco PR-1a promoter can be activated by salicylic acid. In some embodiments, a chemical-regulated promoter can include a steroid-responsive promoter, for example, a glucocorticoid-inducible promoter, a tetracycline-inducible and a tetracycline-repressible promoter.

In some embodiments, a “constitutive promoter” can refer to promoters active in all or most tissues or cell types of an organism at all or most developing stages. In some embodiments, a promoter classified as “constitutive” (e.g., ubiquitin), some variation in absolute levels of expression can exist among different tissues or stages. In some embodiments, the term “constitutive promoter” or “tissue-independent promoter” can be used interchangeably herein. In some embodiments, constitutive promoters include the following: the core promoter of the Rsyn7 promoter; the core CaMV 35S promoter; plant actin promoter, such as a rice actin promoter and a maize actin promoter; plant ubiquitin promoter, such as a maize ubiquitin promoter and a soybean ubiquitin promoter; pEMU; MAS promoter; ALS promoter; plant GOS2 promoter, such as a maize GOS2 promoter; soybean GM-EF1 A2 promoter; plant U6 polymerase III promoter, such as a maize U6 polymerase III promoter and a soybean U6 polymerase III promoter (GM-U6-9.1 and GM-U6-13.1); and any combination thereof.

In some embodiments, an enhancer element can be any nucleic acid molecule that increases transcription of a nucleic acid molecule when functionally linked to a promoter regardless of its relative position. In some embodiments, an enhancer may be an innate element of the promoter, or a heterologous element inserted to enhance the level or tissue-specificity of a promoter.

In some embodiments, a repressor (also sometimes called herein silencer) can be defined as any nucleic acid molecule which inhibits the transcription when functionally linked to a promoter regardless of relative position.

In some embodiments, a “translation leader sequence” can refer to a polynucleotide sequence located between the promoter sequence of a gene and the coding sequence. In some embodiments, the translation leader sequence can be present in the fully processed mRNA upstream of the translation start sequence. In some embodiments, the translation leader sequence may affect processing of the primary transcript to mRNA, mRNA stability or translation efficiency.

In some embodiments, a “transcription terminator”, “termination sequence”, or “terminator” can refer to DNA sequences that, when operably linked to the 3′ end of a polynucleotide sequence that is to be expressed, can terminate transcription from the polynucleotide sequence. In some embodiments, a transcription termination can refer to the process by which RNA synthesis by RNA polymerase can be stopped and both the RNA and the enzyme are released from the DNA template.

In some embodiments, “operably linked” can refer to the association of fragments in a single fragment (e.g., a polynucleotide or polypeptide), or in a single complex, so that the function of one can be regulated by the other. In some embodiments, a linkage may be covalent or non-covalent. In some embodiments, with respect to nucleic acid fragments, a promoter can be operably linked with a nucleic acid fragment if the promoter can regulate the transcription of that nucleic acid fragment. In some embodiments, with respect to a polypeptide, an organelle targeting peptide can be operably linked with a polypeptide if the organelle targeting peptide can transport that polypeptide into the relevant organelle. In some embodiments, with respect to a complex, a guide RNA can be operably linked to a Cas polypeptide if the guide RNA/Cas polypeptide complex can cleave a target sequence as directed by the guide RNA.

In some embodiments, a “phenotype” can refer to the detectable characteristics of a cell or organism.

In some embodiments, the term “introduced” can mean providing a polynucleic acid (e.g., expression construct) or protein into a cell. In some embodiments, “introduced” can include reference to the incorporation of a nucleic acid into a eukaryotic or prokaryotic cell, for example, where the nucleic acid may be incorporated into the genome of the cell. In some embodiments, “introduced” can include reference to the transient provision of a nucleic acid or protein to the cell. In some embodiments, “introduced” can include reference to stable or transient gene editing method. In some embodiments, “introduced” can include reference to stable or transient transformation methods. Introduced can include sexually crossing. In some embodiments, “introduced”, for example, in the context of inserting a nucleic acid fragment (e.g., a recombinant DNA construct) into a cell, can include “transfection” or “transformation” or “transduction”. In some embodiments, “introduced” can include reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

In some embodiments, an “edited mitochondrial genome” may comprise introduction of (i) a substitution of at least one nucleotide, (ii) a deletion of at least one nucleotide (iii) an insertion of at least one nucleotide or (iv) any combination of (i)-(iii). In some embodiments, substitution and replacement are both used herein to mean the interchange of an existing nucleotide with an alternative nucleotide. In some embodiments, a cell may comprise an edited mitochondrial genome with at least one nucleotide substitution, deletion, or insertion. In some embodiments, a cell may comprise a transformed mitochondrion, wherein the transformed mitochondrial comprises the edited mitochondrial genome.

In some embodiments, a “transformed cell” can be any cell in which a nucleic acid fragment (e.g., a recombinant DNA construct) has been introduced or edited.

In some embodiments, “transformation” as used herein can refer to a stable transformation. In some embodiments, a transformation can refer to transient transformation.

In some embodiments, “stable transformation” can refer to an introduction of a nucleic acid fragment into a genome (e.g., of the nucleus, mitochondrion, plastid) of a host organism resulting in genetically stable inheritance. In some embodiments, once stably transformed, the nucleic acid fragment can be stably integrated in the genome of the host organism and any subsequent generation.

In some embodiments, a “transient transformation” can refer to the introduction of a nucleic acid fragment into the nucleus, or DNA-containing cytoplasmic organelle (e.g., mitochondrion, plastid), thereby editing or modifying a host organism nucleus or organelle genomes resulting in gene expression without genetically stable inheritance.

In some embodiments, host organisms containing the transformed nucleic acid fragments can be referred to as “transgenic” organisms.

In some embodiments, a “transformation cassette” can refer to a construct having elements that facilitates transformation of a particular host cell. In some embodiments, the terms “transformation cassette” and “transformation construct” can be used interchangeably herein.

In some embodiments, “homoplasmic”, when used with respect to mitochondria, can refer to a eukaryotic cell in which the copies of mitochondrial DNA are all identical. In some embodiments, “heteroplasmic” can refer to a eukaryotic cell in which the copies of mitochondrial DNA are not all identical.

In some embodiments, “homoplasmic”, when used with respect to plastids, can refer to a eukaryotic cell in which the copies of plastid DNA are all identical. In some embodiments, “heteroplasmic” can refer to a eukaryotic cell in which the copies of plastid DNA are not all identical.

In some embodiments, an “allele” can be one of several alternative forms of a gene occupying a given locus on a chromosome. In some embodiments, when the alleles present at a given locus on a pair of homologous chromosomes in a diploid plant are the same that plant can be homozygous at that locus. In some embodiments, if the alleles present at a given locus on a pair of homologous chromosomes in a diploid plant differ, that plant can be heterozygous at that locus. In some embodiments, if a transgene is present on one of a pair of homologous chromosomes in a diploid plant that plant can be hemizygous at that locus.

In some embodiments, an “organelle” can be a DNA-containing organelle of the cell. In some embodiments, an organelle can be a nucleus, a mitochondrion, a plastid (e.g., a chloroplast), or any combination thereof. In some embodiments, an organelle can be a DNA-containing cytoplasmic organelle. In some embodiments, an organelle can be a mitochondrion, a plastid (e.g., a chloroplast), or any combination thereof. In some embodiments, a plastid can be a proplastid, an etioplast, a leucoplast, an amyloplast, an elaioplast, a proteinoplast, a chromoplast, a chloroplast, a geronoplast, or any combination thereof.

In some embodiments, the terms “organelle-specific” and “organelle-preferred” can be used interchangeably, and when used to describe a regulatory element (e.g., an organelle-specific promoter), refer to a regulatory element that is functional within a given cell (e.g., a plant cell) predominantly but not necessarily exclusively in an organelle (e.g., a mitochondrion, a plastid).

In some embodiments, an organelle-specific regulatory domain may be derived from an organellar polynucleotide of interest (e.g., a mitochondrial polynucleotide, a plastid polynucleotide). In some embodiments, an organelle-specific regulatory domain may comprise all or part of the nucleic acid sequence of an organellar polynucleotide of interest. In some embodiments, the organelle-specific regulatory domain may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the organellar polynucleotide of interest.

In some embodiments, the terms “mitochondrial-specific” and “mitochondrial-preferred” can be used interchangeably, and when used to describe a regulatory element (e.g., a mitochondrial-specific promoter), refer to a regulatory element that is functional within a given cell (e.g., a plant cell) predominantly but not necessarily exclusively in mitochondria.

In some embodiments, the terms “plastid-specific” and “plastid-preferred” can be used interchangeably, and when used to describe a regulatory element (e.g., a plastid-specific promoter), refer to a regulatory element that is functional within a given cell (e.g., a plant cell) predominantly but not necessarily exclusively in plastids.

In some embodiments, the terms “chloroplast-specific” and “chloroplast-preferred” can be used interchangeably, and when used to describe a regulatory element (e.g., a chloroplast-specific promoter), refer to a regulatory element that is functional within a given cell (e.g., a plant cell) predominantly but not necessarily exclusively in chloroplasts.

In some embodiments, the terms “mitochondrial genome” and “genome of a mitochondrion” can be used interchangeably and refer to the nucleic acid sequences present within endogenous mitochondrial genetic elements. In some embodiments, the mitochondrial genome may be edited by the addition of a sequence (e.g., a heterologous sequence) into an endogenous mitochondrial genetic element. In some embodiments, an autonomously replicating heterologous episomal element (e.g., a plasmid DNA) introduced into a mitochondrion is considered to be an independent genetic element and is not considered to be part of the mitochondrial genome.

In some embodiments, the terms “plastid genome”, “chloroplast genome”, “genome of a plastid” and “genome of a chloroplast” can be used interchangeably and refer to a nucleic acid sequence present within endogenous plastid genetic elements. In some embodiments, a plastid genome may be edited by the addition of a sequence (e.g., a heterologous sequence) into an endogenous plastid genetic element. In some embodiments, an autonomously replicating heterologous episomal element (e.g., a plasmid DNA) introduced into a plastid is considered to be an independent genetic element and is not considered to be part of the plastid genome.

In some embodiments, a “chloroplast transit peptide” can be an amino acid sequence that can direct a protein to the chloroplast or other plastid types present in the cell. In some embodiments, a chloroplast transit peptide can be translated in conjunction with the protein in the cell in which the protein can be made. In some embodiments, the terms “chloroplast transit peptide”, “plastid transit peptide”, “chloroplast targeting peptide” and “plastid targeting peptide” can be used interchangeably herein. “Chloroplast transit sequence” can refer to a nucleotide sequence that can encode a chloroplast transit peptide.

In some embodiments, a “signal peptide” can be an amino acid sequence that can direct a protein to the secretory system. The signal peptide can be translated in conjunction with a protein. For example, if the protein is to be directed to a vacuole, a vacuolar targeting signal (supra) can further be added, or if to an endoplasmic reticulum, an endoplasmic reticulum retention signal (supra) may be added. If a protein is to be directed to the nucleus, any signal peptide present can be removed and a nuclear localization signal can be included.

In some embodiments, a “mitochondrial targeting peptide” can be an amino acid sequence which can direct a precursor protein into the mitochondria. In some embodiments, the terms “mitochondrial targeting peptide”, “mitochondrial signal peptide” and “mitochondrial transit peptide” can be used interchangeably herein.

In some embodiments, an “organelle targeting polynucleotide” can be a nucleotide sequence which can direct import of the polynucleotide into an organelle. In some embodiments, the terms “organelle targeting polynucleotide”, “organelle targeting nucleic acid” and “organelle targeting nucleic acid sequence” can be used interchangeably herein. In some embodiments, an organelle targeting polynucleotide may be directed to, for example, the plastid (“plastid targeting polynucleotide”) or the mitochondria (“mitochondria targeting polynucleotide”). In some embodiments, a polynucleotide can be RNA (“organelle targeting RNA”), DNA (“organelle targeting DNA) or a combination of RNA and DNA. In some embodiments, an organelle targeting RNA directed to the plastid can be termed a “plastid targeting RNA”. In some embodiments, the terms “plastid targeting RNA”, “chloroplast targeting RNA” and “transit RNA” are used interchangeably herein. In some embodiments, an organelle targeting RNA directed to the mitochondria can be termed a “mitochondria targeting RNA”.

In some embodiments, RNAs can be imported into mitochondria. In some embodiments, one such mitochondrial targeting RNA can be the yeast tRNALys. In some embodiments, yeast tRNALys and its variants can be imported into human mitochondria. In some embodiments, another RNA that can be imported into mitochondria can be 5S rRNA. In some embodiments, 5S rRNA can function as a vector for delivering heterologous RNA sequences into, for example, mitochondria (e.g., human). In some embodiments, RNAs can be used with the compositions and methods of the disclosure for example, for targeting an organelle (e.g., the mitochondria).

In some embodiments, RNAs can be imported into plastids. In some embodiments, plastid targeting RNAs that can mediate import of attached heterologous RNA can include vd-5′UTR (e.g., viroid-derived ncRNA sequence acting as 5′UTR) and eIF4E1 mRNA. In some embodiments, RNAs can be used with the compositions and methods of the disclosure for targeting to an organelle (e.g., the plastid).

In some embodiments, as used herein, “fusion” can refer to a protein and/or nucleic acid comprising one or more non-native sequences (e.g., moieties). In some embodiments, any of the molecules described herein (e.g., nucleic acids, proteins, polypeptides, polynucleic acid, Cas protein, guide polynucleotide) can be engineered as fusions. In some embodiments, a fusion can comprise one or more of the same non-native sequences. In some embodiments, a fusion can comprise one or more of different non-native sequences. In some embodiments, a fusion can be a chimera. In some embodiments, a fusion can comprise a nucleic acid affinity tag. In some embodiments, a fusion can comprise a barcode. In some embodiments, a fusion can comprise a peptide affinity tag. In some embodiments, a fusion can provide for subcellular localization of the site-directed polypeptide. In some embodiments, a fusion can provide a non-native sequence (e.g., affinity tag) that can be used to track or purify. In some embodiments, a fusion can be a small molecule such as biotin or a dye such as alexa fluor dyes, Cyanine3 dye, Cyanine5 dye, or any combination thereof.

In some embodiments, a fusion can refer to any protein with a functional effect. In some embodiments, a fusion protein can comprise deaminase activity, cytidine deaminase activity (US Patent Publication No. US20150166980, herein incorporated by reference), adenine deaminase activity (US Patent Publication No. US20180073012, herein incorporated by reference), uracil glycosylase inhibitor activity (US Patent Publication No. US20170121693, herein incorporated by reference), methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. In some embodiments, an effector protein can modify a genomic locus. In some embodiments, a fusion protein can be a fusion in a Cas protein. In some embodiments, a Cas protein can be a modified form that has nickase activity or that has no substantial nucleic acid-cleaving activity. In some embodiments, a fusion protein can be a non-native sequence in a Cas protein.

In some embodiments, “silencing”, as used herein with respect to the target gene, can refer to the suppression of levels of mRNA or protein/enzyme expressed by the target gene, and/or the level of the enzyme activity or protein functionality. In some embodiments, the terms “suppression”, “suppressing” and “silencing”, which can be used interchangeably herein, can include lowering, reducing, declining, decreasing, inhibiting, eliminating, or preventing. In some embodiments, “silencing” or “gene silencing” can occur by any suitable mechanism. In some embodiments, non-limiting examples of silencing can include antisense, co-suppression, viral-suppression, hairpin suppression, stem-loop suppression, RNAi-based approaches, small RNA-based approaches, and any combination thereof.

In some embodiments, suppression of gene expression can also be achieved by, for example, use of artificial miRNA precursors, ribozyme constructs and gene disruption. In some embodiments, a modified plant miRNA precursor may be used, wherein the precursor has been modified, for example, to replace the miRNA encoding region with a sequence designed to produce a miRNA directed to the nucleotide sequence of interest. In some embodiments, a gene disruption may be achieved by use of transposable elements or by use of chemical agents that cause site-specific mutations.

### Sequence Identity, Similarity, and Variation

In some embodiments, a sequence alignment and percent identity or similarity calculation may be determined using a variety of comparison methods designed to detect homologous sequences including, but not limited to, the MEGALIGN™ program of the LASERGENE™ bioinformatics computing suite (DNASTAR™ Inc., Madison, WI). In some embodiments, where sequence analysis software is used for analysis, results of an analysis can be based on “default values” of a program referenced. In some embodiments, as used herein “default values” can mean any set of values or parameters that originally load with the software when first initialized.

In some embodiments, “Clustal V method of alignment” can correspond to an alignment method labeled Clustal V and, for example, found in a MEGALIGN™ program of a LASERGENE™ bioinformatics computing suite (DNASTAR™ Inc., Madison, WI). In some embodiments, for multiple alignments, default values can correspond to GAP PENALTY=10 and GAP LENGTH PENALTY=10. In some embodiments, default parameters for pairwise alignments and calculation of percent identity of protein sequences using the Clustal method can be, for example, KTUPLE=1, GAP PENALTY=3, WINDOW=5 and DIAGONALS SAVED=5. In some embodiments, for nucleic acids these parameters can be for example KTUPLE=2, GAP PENALTY=5, WINDOW=4 and DIAGONALS SAVED=4. In some embodiments, after alignment of sequences using the Clustal V program, “percent identity” and “divergence” values can be obtained by viewing the “sequence distances” table in the same program.

In some embodiments, the “Clustal W method of alignment” can correspond to the alignment method labeled Clustal W and, for example, found in the MEGALIGN™ v6.1 program of the LASERGENE™ bioinformatics computing suite (DNASTAR™ Inc., Madison, Wl). In some embodiments, default parameters for multiple alignment can correspond to for example: GAP PENALTY=10, GAP LENGTH PENALTY=0.2, Delay Divergence Sequences=30%, DNA Transition Weight-0.5, Protein Weight Matrix-Gonnet Series, DNA Weight Matrix=IUB. In some embodiments, after alignment of the sequences using the Clustal W program, “percent identity” values can be obtained by viewing the “sequence distances” table in the same program.

In some embodiments, sequence identity/similarity values can also be obtained using GAP Version 10 (GCG, ACCELRYS™, San Diego, CA) using for example the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix. In some embodiments, GAP can use an algorithm to find an alignment of two complete sequences that can maximize the number of matches and minimize the number of gaps. In some embodiments, GAP can consider all possible alignments and gap positions. In some embodiments, GAP can create the alignment with the largest number of matched bases and the fewest gaps, using, for example, a gap creation penalty and a gap extension penalty in units of matched bases.

In some embodiments, “BLAST” can be a searching algorithm provided by the National Center for Biotechnology Information (NCBI) that can be used to find regions of similarity between biological sequences. In some embodiments, BLAST can compare nucleotide or protein sequences to sequence databases. In some embodiments, BLAST can calculate the statistical significance of matches to identify sequences having sufficient similarity to a query sequence such that the similarity may not be predicted to have occurred randomly. In some embodiments, BLAST can report the identified sequences and their local alignment to the query sequence.

In some embodiments, the term “conserved domain” or “motif” can mean a set of amino acids conserved at specific positions along an aligned sequence of evolutionarily related proteins. In some embodiments, while amino acids at other positions can vary between homologous proteins, amino acids that are highly conserved at specific positions can indicate, for example, amino acids that are essential to the structure, the stability, or the activity of a protein.

In some embodiments, conserved domains or motifs can be identified by their high degree of conservation in aligned sequences of a family of protein homologues. In some embodiments, conserved domains can be used as identifiers, or “signatures”, for example, to determine if a protein with a newly determined sequence belongs to a previously identified protein family.

In some embodiments, polynucleotide and polypeptide sequences, variants thereof, and the structural relationships of these sequences can be described by the terms “homology”, “homologous”, “substantially identical”, “substantially similar” and “corresponding substantially” which are used interchangeably herein. In some embodiments, these can refer to polypeptide or nucleic acid fragments wherein changes in one or more amino acids or nucleotide bases may not affect the function of the molecule, such as the ability to mediate gene expression or to produce a certain phenotype. In some embodiments, these terms can also refer to modification(s) of nucleic acid fragments that may not substantially alter the functional properties of the resulting nucleic acid fragment relative to the initial, unmodified fragment. In some embodiments, these modifications can include deletion, substitution, insertion, or any combination thereof, of one or more nucleotides in the nucleic acid fragment.

In some embodiments, substantially similar nucleic acid sequences encompassed may be defined by their ability to hybridize (for example, under moderately stringent conditions, e.g., 0.5×SSC, 0.1% SDS, 60° C.) with the sequences exemplified herein, or to any portion of the nucleotide sequences disclosed herein. In some embodiments, substantially similar nucleic acid sequences can be functionally equivalent to any of the nucleic acid sequences disclosed herein. In some embodiments, stringency conditions can be adjusted to screen for moderately similar fragments, such as homologous sequences from distantly related organisms to highly similar fragments, such as genes that duplicate functional enzymes from closely related organisms. In some embodiments, post-hybridization washes can determine stringency conditions.

In some embodiments, the term “selectively hybridizes” can include reference to hybridization, for example under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. In some embodiments, selectively hybridizing sequences can have, for example, about at least 80% sequence identity, or 90% sequence identity, up to and including 100% sequence identity (i.e., fully complementary) with each other.

In some embodiments, the term “stringent conditions” or “stringent hybridization conditions” can include reference to conditions under which a probe can selectively hybridize to its target sequence in an in vitro hybridization assay. In some embodiments, stringent conditions can be sequence-dependent. In some embodiments, stringent conditions can be different in different circumstances. In some embodiments, by controlling the stringency of the hybridization and/or washing conditions, target sequences can be identified which are 100% complementary to the probe (homologous probing).

In some embodiments, stringency conditions can be adjusted to allow some mismatching in sequences so that lower degrees of similarity are detected (heterologous probing). In some embodiments, a probe can be less than about 1000 nucleotides in length, optionally less than 500 nucleotides in length.

In some embodiments, stringent conditions can comprise those in which a salt concentration is less than about 1.5 M Na ion. In some embodiments, stringent conditions can comprise those in which a salt concentration is less than about 0.01 to 1.0 M Na ion concentration (or other salt(s)) at pH 7.0 to 8.3. In some embodiments, stringent conditions can comprise a temperature of about 30° C. for short probes (e.g., 10 to 50 nucleotides). In some embodiments, stringent conditions can comprise a temperature of at least about 60° C. for long probes (e.g., greater than 50 nucleotides). In some embodiments, stringent conditions can also be achieved with the addition of destabilizing agents such as formamide. In some embodiments, exemplary low stringency conditions can include hybridization with a buffer solution of, for example, 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. In some embodiments, exemplary moderate stringency conditions can include hybridization in 40 to 45% formamide, 1 M NaCl, 1% SDS at 37° C., and a wash in 0.5× to 1×SSC at 55 to 60° C. In some embodiments, exemplary high stringency conditions can include hybridization in, for example, 50% formamide, 1 M NaCl, 1% SDS at 37° C., and a wash in 0.1×SSC at 60 to 65° C.

In some embodiments, “sequence identity” or “identity” in the context of nucleic acid or polypeptide sequences can refer to the nucleic acid bases or amino acid residues in two sequences that are the same when aligned for maximum correspondence over a specified comparison window.

In some embodiments, the term “percentage of sequence identity” can refer to a value determined by comparing two optimally aligned sequences over a comparison window. In some embodiments, a portion of the polynucleotide or polypeptide sequence in the comparison window may comprise additions or deletions (i.e., gaps) as compared to the reference sequence (which may or may not comprise additions or deletions) for optimal alignment of the two sequences. In some embodiments, a percentage can be calculated by, for example, determining a number of positions at which an identical nucleic acid base or amino acid residue occurs in both sequences to yield a number of matched positions, dividing a number of matched positions by a total number of positions in a window of comparison and multiplying the results by 100 to yield the percentage of sequence identity. In some embodiments, percent sequence identities can include, but are not limited to, 50%, 55%, 60%, 65%, 70%, 75%, 80%, 85%, 90% or 95%, or any percentage from 50% to 100%. In some embodiments, sequence identity can include an integer percentage from 50% to 100%. In some embodiments, these identities can be determined using any of the programs described herein.

In some embodiments, sequence identity can be useful in identifying polypeptides from other species or modified naturally or synthetically wherein such polypeptides have the same or similar function or activity. In some embodiments, percent identities can include, but are not limited to, 50%, 55%, 60%, 65%, 70%, 75%, 80%, 85%, 90% or 95%. In some embodiments, sequence identity (e.g., amino acid sequence identity) can include an integer percentage from 50% to 100%. In some embodiments, sequence (e.g., amino acid) identity can include, for example, about: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Definitions, Traits, and Processes Relevant to Plants

In some embodiments, “plant” can include reference to whole plants, plant organs, plant tissues, plant propagules, seeds and plant cells and progeny of the same. In some embodiments, plant cells include, without limitation, cells from seeds, suspension cultures, embryos, meristematic regions, callus tissue, leaves, roots, shoots, gametophytes, sporophytes, pollen, and microspores.

In some embodiments, a “propagule” can include products of meiosis and/or mitosis able to propagate a new plant. In some embodiments, a propagule can include seeds, spores and parts of a plant that can serve as a means of vegetative reproduction, such as corms, tubers, offsets, or runners. In some embodiments, a propagule can include grafts where one portion of a plant can be grafted to another portion of a different plant (even one of a different species) to create a living organism. In some embodiments, a propagule can include plants and seeds produced by cloning or by bringing together meiotic products, or allowing meiotic products to come together to form an embryo or fertilized egg (naturally or with human intervention).

In some embodiments, a “progeny” can comprise any subsequent generation of a plant.

In some embodiments, the terms “monocot” and “monocotyledonous plant” can be used interchangeably herein. In some embodiments, a monocot can include the Gramineae.

In some embodiments, the terms “dicot” and “dicotyledonous plant” can be used interchangeably herein. In some embodiments, a dicot can include, for example, the following families: Brassicaceae, Leguminosae, and Solanaceae.

In some embodiments, “transgenic plant” can include reference to a plant which can comprise within its genome a heterologous polynucleotide. In some embodiments, a heterologous polynucleotide can be stably integrated within a genome (e.g., nuclear, plastid, mitochondrial) such that a polynucleotide can be passed on to successive generations. In some embodiments, a heterologous polynucleotide can be integrated into a genome alone or as part of a recombinant DNA construct.

In some embodiments, a “transgenic plant” can include reference to plants which can comprise more than one heterologous polynucleotide within their genome. In some embodiments, each heterologous polynucleotide can confer a different trait to a transgenic plant.

In some embodiments, multiple traits can be introduced into crop plants, and can be referred to as a gene stacking approach. In some embodiments, gene stacking can be used, for example, for development of genetically improved germplasm. In some embodiments, multiple genes conferring different characteristics of interest can be introduced into a plant. In some embodiments, gene stacking can be accomplished by many means including but not limited to co-transformation, retransformation, and crossing lines with different transgenes. In some embodiments, as used herein, the term “stacked” can include having multiple traits present in the same plant (e.g., both traits are incorporated into the nuclear genome, one trait is incorporated into the nuclear genome and one trait is incorporated into the genome of an organelle, or both traits are incorporated into the genome of an organelle).

In some embodiments, the term “crossed” or “cross” or “crossing” in the context of the disclosure can mean the fusion of gametes (e.g., via pollination) to produce progeny (e.g., cells, seeds, or plants). In some embodiments, the term can encompass both sexual crosses (e.g., the pollination of one plant by another) and selfing (e.g., self-pollination; when the pollen and ovule are from the same plant or genetically identical plants).

In some embodiments, the term “maternal inheritance” can refer to the transmission of traits that can be solely dependent on properties of the genome of the female gamete.

In some embodiments, the term “paternal inheritance” can refer to the transmission of traits that are solely dependent on properties of the genome of the male gamete.

In some embodiments, the term “introgression” can refer to the transmission of a desired allele of a genetic locus from one genetic background to another. In some embodiments, introgression of a desired allele at a specified locus can be transmitted to at least one progeny plant via a sexual cross between two parent plants, where at least one of the parent plants has the desired allele within its genome. In some embodiments, transmission of an allele can occur by recombination between two donor genomes, e.g., in a fused protoplast, where at least one of the donor protoplasts has the desired allele in its genome. In some embodiments, a desired allele can be, e.g., a transgene or a selected allele of a marker or QTL.

In some embodiments, “a plant-optimized nucleotide sequence” can be a nucleotide sequence that has been optimized for increased expression in plants, particularly for increased expression in a given plant or in one or more plants of interest. In some embodiments, a plant-optimized nucleotide sequence can be synthesized by modifying a nucleotide sequence encoding a protein by using plant-preferred codons for improved expression. In some embodiments, a host-preferred codon usage can be utilized for codon optimization. In some embodiments, a frequency of codon usage can be designed to mimic the frequency of preferred codon usage of a host cell in a compartment of interest, e.g., a nucleus, a mitochondrion, or a chloroplast.

In some embodiments, plant-preferred genes can be synthesized. In some embodiments, additional sequence modifications can enhance gene expression in a plant host. In some embodiments, these can include, for example, elimination of any of the following: one or more sequences encoding spurious polyadenylation signals, one or more exon-intron splice site signals, one or more transposon-like repeats, and sequences that can be deleterious to gene expression. In some embodiments, a G-C content of a sequence may be adjusted, for example, to levels average for a given plant host, as calculated by reference to genes expressed in a host plant cell. In some embodiments, when possible, a sequence can be modified to avoid one or more predicted hairpin secondary mRNA structures. In some embodiments, “a plant-optimized nucleotide sequence” of a present disclosure can comprise one or more of such sequence modifications.

In some embodiments, a “trait” can refer to, for example, a physiological, morphological, biochemical, or physical characteristic of a plant or particular plant material or cell. In some instances, a characteristic can be visible to a human eye, such as seed or plant size, or can be measured by biochemical techniques, such as detecting a protein, starch, or oil content of seed or leaves, or by observation of a metabolic or physiological process, e.g. by measuring tolerance to water deprivation or particular salt or sugar concentrations, or by an observation of an expression level of a gene or genes, or by agricultural observations such as osmotic stress tolerance or yield.

In some embodiments, an “agronomic characteristic” can be a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

### Herbicide Resistance in Plants

In some embodiments, an “herbicide resistance protein” or a protein resulting from expression of an “herbicide resistance-encoding nucleic acid molecule” can include proteins that can confer upon a cell the ability to tolerate a higher concentration of an herbicide, for example, compared with cells that do not express the protein. In some embodiments, an herbicide resistance protein can have enzymatic activity. In some embodiments, an herbicide resistance protein can have enzymatic activity in the presence of an herbicide that targets said enzymatic activity. In some embodiments, an herbicide resistance protein can have enzymatic activity that results in degradation and/or inactivation of an herbicide. In some embodiments, an herbicide resistance protein can be monomeric or multimeric. In some embodiments, an herbicide resistance protein can comprise a single polypeptide. In some embodiments, an herbicide resistance protein can comprise two or more distinct polypeptides. In some embodiments, the terms “herbicide resistance protein”, “herbicide-resistant protein”, “herbicide tolerance protein” and “herbicide tolerant protein” may be used interchangeably herein when used to describe a molecule (e.g., a protein, an enzyme, a subunit, or a nucleic acid) that can confer upon a cell (or plant) the ability to tolerate a higher concentration of an herbicide compared with a cell (or plant) that does not express the molecule.

In some embodiments, an herbicide resistance protein or a protein resulting from expression of a herbicide resistance-encoding nucleic acid molecule can include proteins that can confer upon a cell an ability to tolerate a concentration of a herbicide for a longer period of time than cells that do not express a protein. In some embodiments, herbicide resistance traits may be introduced into plants by, for example, genes coding for resistance to herbicides. In some embodiments, genes coding for resistance to herbicides include, for example, the following: genes that act to convey tolerance to inhibitors of acetolactate synthase (ALS), such as the sulfonylurea-type herbicides; genes (e.g., the bar gene, the pat gene) that act to convey tolerance to inhibitors of glutamine synthetase, such as phosphinothricin or basta; genes that act to convey tolerance to inhibitors of the EPSP synthase gene, such as glyphosate; genes that act to convey tolerance to inhibitors of HPPD; genes that act to convey tolerance to inhibitors of an acetyl coenzyme A carboxylase (ACCase); and genes that act to convey tolerance to inhibitors of protoporphyrinogen oxidase (PPO or PROTOX).

In some embodiments, genes useful for conferring herbicide resistance in plants can include genes that encode herbicide resistance proteins. In some embodiments, herbicide resistance proteins can include herbicide tolerant versions of: an acetyl coenzyme A carboxylase (ACCase); a 4-hydroxyphenylpyruvate dioxygenase (HPPD); a sulfonylurea-tolerant acetolactate synthase; an imidazolinone-tolerant acetolactate synthase; a glyphosate-tolerant 5-enolpyruvylshikimate-3-phosphate synthase (EPSPS); a glyphosate-tolerant glyphosate oxidoreductase (GOX); a glyphosate N-acetyltransferase (GAT); a phosphinothricin acetyl transferase (PAT); a protoporphyrinogen oxidase (PPO or PROTOX); an auxin enzyme or receptor; a P450 polypeptide, or any combination thereof.

In some embodiments, as used herein, “Hydroxyphenylpyruvate dioxygenase” and “HPPD”, “4-hydroxy phenyl pyruvate (or pyruvic acid) dioxygenase (4-HPPD)” and “p-hydroxy phenyl pyruvate (or pyruvic acid) dioxygenase (p-OHPP)” can be synonymous and can refer to a non-heme iron-dependent oxygenase that catalyzes the conversion of 4-hydroxyphenylpyruvate to homogentisate. In some embodiments, in organisms that degrade tyrosine, a reaction catalyzed by HPPD can be a second step in a pathway. In some embodiments, in plants, formation of homogentisate can be necessary for the synthesis of plastoquinone, which can serve as a redox cofactor, and tocopherol. In some embodiments, a polynucleotide molecule encoding a herbicide tolerant hydroxyphenylpyruvate dioxygenase (HPPD) can provide tolerance to HPPD inhibitors.

In some embodiments, as used herein, an “HPPD inhibitor” can comprise any compound or combinations of compounds which can decrease an ability of HPPD to catalyze a conversion of 4-hydroxyphenylpyruvate to homogentisate. In specific embodiments, an HPPD inhibitor can comprise an herbicidal inhibitor of HPPD. In some embodiments, non-limiting examples of HPPD inhibitors include, triketones (such as, mesotrione, sulcotrione, topramezone, and tembotrione); isoxazoles (such as, pyrasulfotole and isoxaflutole); pyrazoles (such as, benzofenap, pyrazoxyfen, and pyrazolynate); and benzobicyclon. In some embodiments, agriculturally acceptable salts of various inhibitors can include salts (e.g., cations or anions) for a formation of salts for agricultural or horticultural use.

In some embodiments, an “herbicide-resistant ALS polypeptide”, an “herbicide-tolerant ALS polypeptide” and an “ALS inhibitor-tolerant polypeptide” can be used interchangeably and can comprise any polypeptide which when expressed in a plant can confer tolerance to at least one acetolactate synthase (ALS) inhibitor. In some embodiments, ALS inhibitors can include, for example, sulfonylurea, imidazolinone, triazolopyrimidines, pryimidinyoxy(thio)benzoates, and/or sulfonylaminocarbonyltriazolinone herbicides. In some embodiments, ALS mutations can fall into different classes with regard to tolerance to, for example, sulfonylureas, imidazolinones, triazolopyrimidines, pyrimidinyl(thio)benzoates, and sulfonylaminocarbonyltriazolinone. In some embodiments, ALS mutations can include mutations having one or more of the following characteristics: (1) broad tolerance to all five of these groups (i.e., sulfonylureas, imidazolinones, triazolopyrimidines, pyrimidinyl(thio)benzoates, and sulfonylaminocarbonyltriazolinone); (2) tolerance to four of these groups (e.g., sulfonylureas, triazolopyrimidines, pyrimidinyl(thio)benzoates, and sulfonylaminocarbonyltriazolinone); (3) tolerance to imidazolinones and pyrimidinyl(thio)benzoates; (4) tolerance to sulfonylureas and triazolopyrimidines; and (5) tolerance to sulfonylureas and imidazolinones.

In some embodiments, the imidazolinone can include an imazapyr, an imazapic, an imazethapyr, an imazamox, an imazamethabenz, an imazaquin, a salt of any of these, a stereoisomer of any of these, or any combination thereofs. In some embodiments, the triazolopyrimidine can include a penoxsulam, a cloransulam-methyl, a diclosulam, a florasulam, a flumetsulam, a metosulam, a pyroxsulam, a salt of any of these, a stereoisomer of any of these, or any combination thereof. In some embodiments, the pyrimidinyl benzoate can include a bispyribac-sodium, a pyribenzoxim, a pyrithiobac-sodium, a salt of any of these, a stereoisomer of any of these, or any combination thereof. In some embodiments, the sulfonanilide can include a pyrimisulfan, a triafamone, a salt of any of these, a stereoisomer of any of these, or any combination thereof. In some embodiments, the sulfonylaminocarbonyltriazolinone can include a Flucarbazone-Na, a propoxycarbazone-Na, a thiencarbazone-methyl, a salt of any of these, a stereoisomer of any of these, or any combination thereof. In some embodiments, the sulfonylurea can include an amidosulfuron, an azimsulfuron, a bensulfuron-methyl, a chlorimuron-ethyl, a chlorsulfuron, a cinosulfuron, a cyclosulfamuron, an ethametsulfuron-methyl, an ethoxysulfuron, a flazasulfuron, a flucetosulfuron, a flupyrsulfuron-methyl-na, a foramsulfuron, a halosulfuron-methyl, an imazosulfuron, an iodosulfuron-methyl-na, a mesosulfuron-methyl, a metazosulfuron, a metsulfuron-methyl, a nicosulfuron, an orthosulfamuron, an oxasulfuron, a primisulfuron-methyl, a propyrisulfuron, a prosulfuron, a pyrazosulfuron-ethyl, a rimsulfuron, a sulfometuron-methyl, a sulfosulfuron, a thifensulfuron-methyl, a triasulfuron, a tribenuron-methyl, a trifloxysulfuron-na, a triflusulfuron-methyl, a tritosulfuron, a salt of any of these, a stereoisomer of any of these, or any combination thereof.

In some embodiments, polynucleotide molecules encoding proteins involved in herbicide resistance can include a polynucleotide molecule encoding a herbicide tolerant 5-enolpymvylshikimate-3-phosphate synthase (EPSPS) for example, for imparting glyphosate tolerance.

In some embodiments, glyphosate tolerance can also be obtained by expression of polynucleotide molecules encoding a glyphosate oxidoreductase (GOX) or a glyphosate-N-acetyl transferase (GAT).

In some embodiments, polynucleotides encoding a heterologous phosphinothricin acetyltransferase can be used for herbicide resistance. In some embodiments, plants containing a heterologous phosphinothricin acetyltransferase can exhibit improved tolerance to glufosinate herbicides, which can inhibit, for example, the enzyme glutamine synthetase.

In some embodiments, polynucleotides encoding proteins with altered protoporphyrinogen oxidase (PPO or PROTOX) activity can be used for herbicide resistance. In some embodiments, plants containing such polynucleotides can exhibit improved tolerance to any of a variety of herbicides which can target, for example, the PPO enzyme (also referred to as “PPO inhibitors” or “PROTOX inhibitors”).

In some embodiments, dicamba monooxygenase can be used for providing dicamba tolerance.

In some embodiments, a polynucleotide molecule encoding AAD12 or encoding AAD1 can be used for providing resistance to, for example, auxin herbicides.

In some embodiments, a P450-encoding polynucleotide can be used for conferring herbicide resistance. In some embodiments, a P450-encoding sequence can provide tolerance to HPPD inhibitors by, for example, metabolism of the herbicide. Such sequences include, but are not limited to, the NSF1 gene.

### Resistance to Plant Pests

In some embodiments, a “plant pest” can mean any living stage of an entity that can directly or indirectly injure, cause damage to, or cause disease in any plant or plant product. In some embodiments, a plant pest can include a protozoan, a nonhuman animal, a parasitic plant, a bacterium, a fungus, a virus, a viroid, an infectious agent, a pathogen, or any article similar to or allied thereof.

In some embodiments, a plant pest invertebrate can comprise a pest nematode, a pest mollusk, a pest insect, or any combination thereof. In some embodiments, a pest mollusk can comprise a slug, a snail, or a combination thereof. In some embodiments, a plant pathogen can comprise a fungi, a nematode, or a combination thereof.

In some embodiments, a plant pathogen can be a eukaryotic plant pathogen. In some embodiments, a plant pathogen can include, for example, a fungal pathogen, such as a phytopathogenic fungus.

In some embodiments, a target gene of interest (e.g., for gene silencing) can include any coding or non-coding sequence from any species (including, but not limited to, eukaryotes such as fungi; plants, including monocots and dicots, such as crop plants, ornamental plants, and non-domesticated or wild plants; invertebrates such as arthropods, annelids, nematodes, and mollusks; and vertebrates such as amphibians, fish, birds, and mammals). In some embodiments, non-limiting examples of a non-coding sequence (e.g., that can be expressed by a gene expression element such as a regulatory sequence) can include, 5′ untranslated regions, promoters, enhancers, or other non-coding transcriptional regions, 3′ untranslated regions, terminators, introns, microRNAs, microRNA precursor DNA sequences, small interfering RNAs, RNA components of ribosomes or ribozymes, small nucleolar RNAs, and other non-coding RNAs, or any combination thereof. In some embodiments, a gene of interest can include, translatable (coding) sequence, such as genes encoding transcription factors and genes encoding enzymes involved in a biosynthesis or catabolism of molecules of interest (such as amino acids, fatty acids and other lipids, sugars and other carbohydrates, biological polymers, and secondary metabolites including alkaloids, terpenoids, polyketides, non-ribosomal peptides, and secondary metabolites of mixed biosynthetic origin).

In some embodiments, a target gene (e.g., for gene silencing) can be an essential gene of a plant pest or plant pathogen. In some embodiments, essential genes can include genes that can be required for development of a pest or pathogen to a fertile reproductive adult. In some embodiments, essential genes can include genes that, when silenced or suppressed, can result in a death of an organism (e.g., as an adult or at any developmental stage, including gametes) or in an organism's inability to successfully reproduce (e. g., sterility in a male or female parent or lethality to a zygote, embryo, or larva).

In some embodiments, a plant can be transformed (e.g., in a nucleus, a cytoplasmic organelle, or both) with an expression cassette encoding, for example, a dsRNA, a siRNA or a miRNA. The dsRNA, siRNA, or miRNA can suppress (e.g., expression of) at least one (e.g., at least 1, at least 2, at least 3, at least 4, at least 5, at least 6, at least 7, at least 8, at least 9, or at least 10) target genes present in a plant pest. In some embodiments, a dsRNA, siRNA, or miRNA can suppress, for example, 1, 2, 3, 4, 5, 6, 7, 8, 9, 10, 15, 20 or more target genes of a plant pest. In some embodiments, suppression of a target gene present in a plant pest can provide complete or nearly complete protection from a plant pest. In some embodiments, “complete protection” can mean that no (e.g., substantial) damage can be caused to a plant by a plant pest.

In some embodiments, resistance to pests in plants can be achieved by, for example, transgenic control. In some embodiments, in-plant transgenic control of, for example, insect pests, can be achieved through, for example, plant expression of crystal (Cry) delta endotoxin genes and/or Vegetative Insecticidal Proteins (VIP) such as from Bacillus thuringiensis. In some embodiments, non-limiting examples of Cry toxins include, for example, the 60 main groups of “Cry” toxins (e.g., Cry1-Cry59) and VIP toxins. In some embodiments, cry toxins can include subgroups of Cry toxins, for example, Cry 1a.

In some embodiments, an expression cassette for use in transformation (e.g., into an organelle) may be constructed using, for example, a Cry sequence. In some embodiments, a Cry sequence can include, for example, a wild-type (e.g., native) nucleic acid sequence encoding at least one protein selected from a group consisting of: Cry1Ac, Cyt1Aa, Cry1Ab, Cry2Aa, Cry1I, Cry1C, Cry1D, Cry1E, Cry1Be, Cry1Fa and Vip3A. In some embodiments, a Cry sequence can include, for example, a modified (e.g., truncated or fusion) nucleic acid sequence encoding at least one protein selected from a group consisting of: Cry1Ac, Cyt1Aa, Cry1Ab, Cry2Aa, Cry1I, Cry1C, Cry1D, Cry1E, Cry1Be, Cry1Fa and Vip3A. In some embodiments, a modified sequence can comprise a truncated nucleic acid sequence. In some embodiments, a modified sequence can encode a modified protein fragment. In some embodiments, a truncated protein fragment can retain insecticidal activity. In some embodiments, a nucleic acid sequence can encode a full-length, or modified (e.g., truncated) protein. In some embodiments, a modified protein can be codon-optimized for an organelle of interest.

### Genome Modification

Disclosed herein in some embodiments, are compositions and methods that can be used, for genome modification of a target sequence in a genome (e.g., a nucleus, a plastid, or a mitochondrial genome) of an organism or cell (e.g., a plant or plant cell), for selecting the modified organism or cell, for gene editing, and for inserting a donor polynucleotide into the genome (e.g., a nucleus, a plastid, or a mitochondrial genome) of an organism or cell. In some embodiments, methods disclosed herein can employ a polynucleotide guided polypeptide system; e.g., a guide polynucleotide/Cas protein system. In some embodiments, a Cas protein can be guided by a guide polynucleotide to recognize a target polynucleic acid. In some embodiments, a Cas protein can introduce a single strand or double strand break at a specific target site into a genome of a cell. In some embodiments, a guide polynucleotide/Cas polypeptide system can provide for an effective system for modifying target sites within a genome of a plant, plant cell or seed.

In some embodiments, a variety of methods can be employed to further modify a target site to introduce a donor polynucleotide of interest. In some embodiments, a nucleotide sequence to be edited (e.g., a nucleotide sequence of interest) can be located within or outside a target site that can be recognized by a polynucleotide guided polypeptide.

Also disclosed herein are methods and compositions employing a polynucleotide guided polypeptide system for modification of multiple target sites within a genome of an organelle. Modification of multiple target sites within a genome of an organelle can facilitate a creation of a homoplasmic transformation event.

### Polynucleotide Guided Polypeptide Systems

In some embodiments, a polynucleotide-guided polypeptide can be a polypeptide that can bind to a target nucleic acid. In some embodiments, a polynucleotide-guided polypeptide can be a nuclease (e.g., a CRISPR nuclease). In some embodiments, a polynucleotide-guided polypeptide can be an endonuclease, a modified version thereof, and a biologically active fragment thereof. In some embodiments, a polynucleotide-guided polypeptide can be a Cas protein, a modified version thereof, and a biologically active fragment thereof. In some embodiments, a polynucleotide-guided polypeptide can be a MAD protein, a modified version thereof, and a biologically active fragment thereof. In some embodiments, a polynucleotide-guided polypeptide can be an Argonaute protein, a modified version thereof, and a biologically active fragment thereof. In some embodiments, a polynucleotide guided polypeptide can form a complex with a guide polynucleotide. In some embodiments, a polynucleotide guided polypeptide can be directed to a target nucleic acid by a guide polynucleotide. In some embodiments, a polynucleotide guided polypeptide can complex with a guide polynucleotide to recognize a target nucleic acid. In some embodiments, a polynucleotide guided polypeptide can introduce a single strand or double strand break at a specific target site (e.g., the genome of a cell).

In some embodiments, a polynucleotide guided polypeptide can be a Cas protein of a CRISPR/Cas system. In some embodiments, a Cas protein can be a Class 1 or a Class 2 Cas protein. In some embodiments, a Cas protein can be a Type I, Type II, Type III, Type IV, Type V, or Type VI Cas protein.

In some embodiments, a non-limiting examples of Cas proteins include c2c1, C2c2, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

In some embodiments, a Cas protein may be from any suitable organism. In some embodiments, a suitable organism can comprise Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, an organism can comprise Streptococcus pyogenes (S. pyogenes).

In some embodiments, a Cas protein can comprise a Cas9 protein. In some embodiments, a Cas9 protein can comprise a Cas9 sequences listed in SEQ ID NOS: 462, 474, 489, 494, 499, 505, and 518 of WO2007/025097 and incorporated herein by reference. In some embodiments, a Cas9 protein can unwind a DNA duplex in close proximity to a genomic target site. In some embodiments, a Cas9 protein can cleave both DNA strands upon recognition of a target sequence by a guide polynucleic acid. In some embodiments, a Cas9 endonuclease can cleave only if a correct protospacer-adjacent motif (PAM) is approximately oriented at a 3′ end of a target sequence. In some embodiments, a mutagenesis of Streptococcus pyogenes Cas9 catalytic domains can produce “nicking” enzymes (Cas9n) that can induce single-strand nicks rather than double-strand breaks.

In some embodiments, a polynucleotide guided polypeptide can be a MAD polypeptide, e.g., a MAD2 or a MAD7 polypeptide, with amino acid sequence corresponding to SEQ ID NO: 2 and SEQ ID NO: 7 of U.S. Pat. No. 9,982,279, respectively (herein incorporated by reference). In some embodiments, a MAD7 can be a Class 2 Type V-A CRISPR-Cas system isolated from Eubacterium rectale and re-engineered by INSCRIPTA™ (Boulder, CO). In some embodiments, analogous to Cas9, MAD7 can be an RNA-guided nuclease with a diverse protein structure, mechanism of action, and a demonstrated gene editing activity in E. coli and yeast cells. In some embodiments, similar to Acidaminococcus sp. Cas12a, MAD7 does not require a tracrRNA and prefers T-rich PAMs (TTTV and CTTV). In some embodiments, a mutagenesis of MAD2 or MAD7 can produce a “nicking” enzyme that can induce single-strand nicks rather than double-strand breaks.

In some embodiments, a polynucleotide guided polypeptide may be an Argonaute protein such as Natronobacterium gregoryi Argonaute (“NgAgo”). In some embodiments, an Argonaute protein can be a DNA-guided endonuclease. In some embodiments, an Argonaute protein can bind a guide DNA such as a 5′-phosphorylated single-stranded guide DNA (gDNA) of, for example, 24 nucleotides. In some embodiments, an Argonaute protein can create a site-specific target nucleic acid (e.g., DNA) break (e.g., double-stranded breaks) when loaded with a gDNA. In some embodiments, an Argonaute protein/gDNA system may not require a protospacer-adjacent motif (PAM) for recognition of a target nucleic acid.

In some embodiments, a polynucleotide guided polypeptide as used herein can be a wildtype or a modified form of a polynucleotide guided polypeptide. In some embodiments, a polynucleotide guided polypeptide can be an active variant, an inactive variant, or a fragment of a wild type or modified polynucleotide guided polypeptide. In some embodiments, a polynucleotide guided polypeptide can comprise an amino acid change such as a deletion, replacement, insertion, substitution, variant, mutation, fusion, chimera, or any combination thereof relative to a wild-type version of a polynucleotide guided polypeptide. In some embodiments, a polynucleotide guided polypeptide can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary polynucleotide guided polypeptide (e.g., Cas9 from S. pyogenes). In some embodiments, a polynucleotide guided polypeptide can be a polypeptide with at most about 5%, 10%, 20%, 30%, 40%, 50%, 60%, 70%, 80%, 90%, 100% sequence identity and/or sequence similarity to a wild type exemplary polynucleotide guided polypeptide. In some embodiments, variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified polynucleotide guided polypeptide or a portion thereof. In some embodiments, variants or fragments can be targeted to a nucleic acid locus in complex with a guide nucleic acid while lacking nucleic acid cleavage activity.

In some embodiments, a polynucleotide guided endonuclease can be a fusion protein. In some embodiments, a polynucleotide guided endonuclease can be fused to a cleavage domain, an epigenetic modification domain, a transcriptional activation domain, or a transcriptional repressor domain. In some embodiments, a non-limiting example of a suitable fusion partner can include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity, or any combination thereof. In some embodiments, a polynucleotide guided endonuclease can also be fused to a heterologous polypeptide providing increased or decreased stability. In some embodiments, a fused domain or heterologous polypeptide can be located at an N-terminus, a C-terminus, or internally within a polynucleotide guided endonuclease.

In some embodiments, a nucleic acid encoding a polynucleotide guided endonuclease (e.g., Cas endonuclease, Cas9 endonuclease, MAD polypeptide, MAD7 polypeptide), can be codon optimized for efficient translation into protein in a particular cell, organelle (e.g., nucleus, plastid, or mitochondrion), or organism (e.g., wheat or rice).

In some embodiments, a nucleic acid encoding a polynucleotide guided endonuclease can be stably integrated in a genome (nuclear, mitochondrial, plastid) of a cell. In some embodiments, a nucleic acid encoding a polynucleotide guided polypeptide can be operably linked to a regulatory sequence active in a cell. In some embodiments, a nucleic acid encoding a polynucleotide guided polypeptide can be in an expression construct. In some embodiments, an expression construct can include any regulatory sequence that can direct expression of a nucleic acid sequence of interest (promoter, terminator, RNA-editing site). In some embodiments, an expression construct can include any nucleic acid sequence that encodes a peptide capable of targeting a protein into an organelle of interest (e.g., into a nucleus, mitochondrion, or plastid).

In some embodiments, a polynucleotide guided polypeptide coding sequence can be modified to use codons preferred by a target organism, e.g., a plant, maize, or soybean (nuclear, mitochondrial or plastid) codon-optimized sequence. In some embodiments, a sequence that encodes a polynucleotide guided polypeptide can be operably linked to one or more sequences encoding nuclear localization signals; e.g., to a SV40 nuclear targeting signal upstream of a polynucleotide guided polypeptide coding region and a bipartite VirD2 nuclear localization signal downstream of the polynucleotide guided polypeptide coding region. In some embodiments, a sequence that encodes a polynucleotide guided polypeptide can be operably linked to one or more sequences encoding chloroplast or mitochondrial localization signals, i.e., a chloroplast transit sequence or a mitochondrial targeting sequence.

In some embodiments, a polynucleotide guided polypeptide (e.g., Cas polypeptide, Cas9 polypeptide, MAD polypeptide, MAD7 polypeptide), can be provided in any form. In some embodiments, a polynucleotide guided polypeptide can be provided in a form of a protein, such as a polynucleotide guided polypeptide alone or complexed with a guide nucleic acid. In some embodiments, a polynucleotide guided polypeptide can be provided in a form of a nucleic acid encoding a polynucleotide guided polypeptide, such as an RNA (e.g., messenger RNA (mRNA)) or DNA.

In some embodiments, a polynucleotide guided polypeptide can be a polypeptide moiety (e.g., a chimeric polypeptide) that can form a programmable nucleoprotein molecular complex with a specificity conferring nucleic acid (SCNA). In some embodiments, a programmable nucleoprotein molecular complex can assemble in-vivo, in a target cell, or in an organelle. In some embodiments, a programmable nucleoprotein molecular complex can interact with a predetermined target nucleic acid sequence. In some embodiments, a programmable nucleoprotein molecular complex may comprise a polynucleotide molecule encoding a chimeric polypeptide. In some embodiments, a chimeric polypeptide can comprise a functional domain that can modify a target nucleic acid site. In some embodiments, a functional domain can be devoid of a specific nucleic acid binding site. In some embodiments, a chimeric polypeptide can comprise a linking domain that can interact with a SCNA. In some embodiments, a linking domain can be devoid of a specific target nucleic acid binding site. In some embodiments, a SCNA can comprise a nucleotide sequence complementary to a region of a target nucleic acid flanking a target site. In some embodiments, a SCNA can comprise a recognition region that can specifically attach to a linking domain of a chimeric polypeptide. In some embodiments, assembly of a chimeric polypeptide and an SCNA within a target cell can form a functional nucleoprotein complex. In some embodiments, a nucleoprotein complex can specifically modify a target nucleic acid at a target site.

In some embodiments, a polynucleotide guided endonuclease gene can be a full-length polynucleotide guided endonuclease (e.g., Cas endonuclease, Cas9 endonuclease, MAD polypeptide, MAD7 polypeptide), or any functional fragment or functional variant thereof.

Disclosed herein in some embodiments are compositions and methods comprising use of an endonuclease. In some embodiments, an endonuclease can be an enzyme that cleaves a phosphodiester bond within a polynucleotide chain. In some embodiments, an endonuclease can comprise restriction endonucleases that cleave DNA at specific sites without damaging bases. In some embodiments, restriction endonucleases can include Type I, Type II, Type III, and Type IV endonucleases, which can further include subtypes. In some embodiments, Type I and Type III systems, both a methylase and restriction activity can be contained in a single complex. In some embodiments, an endonuclease can also include meganucleases, also known as homing endonucleases (Heases). In some embodiments, a meganuclease can bind and cut at a specific recognition site, which can be about 18 bp or more. In some embodiments, a meganuclease can be classified into four families based on conserved sequence motifs. In some embodiments, a meganuclease family can comprise LAGLIDADG (SEQ ID NO: 1), GIY-YIG, H-N-H, and His-Cys box families. In some embodiments, motifs can participate in a coordination of metal ions and hydrolysis of phosphodiester bonds. In some embodiments, Heases can have long recognition sites and can tolerate sequence polymorphisms in their DNA substrates. In some embodiments, a naming convention for a meganuclease can be similar to a convention for other restriction endonuclease.

In some embodiments, a meganuclease can also be characterized by prefix F-, I-, or PI- for enzymes encoded by free-standing ORFs, introns, and inteins, respectively. In some embodiments, one step in a recombination process can involve polynucleotide cleavage at or near a recognition site. In some embodiments, a cleaving activity can be used to produce a double-strand break. In some embodiments, a recombinase can be from an Integrase or Resolvase family.

In some embodiments, compositions and methods of a disclosure can use Transcription activator-like effector nucleases (TALENs; TAL effector nucleases). In some embodiments, TALENs can be a class of sequence-specific nucleases. In some embodiments, TALENs can be used to cleave (e.g., double-strand breaks) at specific target sequences (e.g., in a genome of a plant or other organism). In some embodiments, TALENs can be created by fusing a native or engineered transcription activator-like (TAL) effector, or functional part thereof, to the catalytic domain of an endonuclease, such as, for example, FokI. In some embodiments, a unique, modular TAL effector DNA binding domain can allow for a design of proteins with potentially any given DNA recognition specificity.

Disclosed herein in some embodiments, are compositions and methods comprising use of zinc finger nucleases (ZFNs). In some embodiments, ZFNs can be engineered cleavage (e.g., double-strand break) inducing agents comprised of a zinc finger DNA binding domain and a double-strand-break-inducing agent domain. In some embodiments, recognition site specificity can be conferred by a zinc finger domain, which can comprise two, three, or four zinc fingers, for example having a C2H2 structure. In some embodiments, a Zinc finger domain can be amenable for designing polypeptides which specifically bind a selected polynucleotide recognition sequence. In some embodiments, a ZFN can consist of an engineered DNA-binding zinc finger domain linked to a non-specific endonuclease domain, for example, a nuclease domain from a Type IIS endonuclease such as FokI. In some embodiments, additional functionalities can be fused to a zinc-finger binding domain, including transcriptional activator domains, transcription repressor domains, and methylases. In some examples, a dimerization of the nuclease domain may be required for cleavage activity. In some embodiments, each zinc finger can recognize, for example, three consecutive base pairs in a target DNA. In some embodiments, a 3-finger domain can recognize a sequence of 9 contiguous nucleotides, with a dimerization requirement of a nuclease, two sets of zinc finger triplets can be used to bind an 18 nucleotide recognition sequence.

### Guide Polynucleic Acid

In some embodiments, bacteria and archaea can have evolved adaptive immune defenses termed clustered regularly interspaced short palindromic repeats (CRISPR)/CRISPR-associated (Cas) systems that can use short RNA to direct degradation of foreign nucleic acids. In some embodiments, a type II CRISPR/Cas system from bacteria can employ a crRNA and tracrRNA to guide a Cas polypeptide to a nucleic acid target. In some embodiments, a crRNA (CRISPR RNA) can contain a region complementary to one strand of a double strand DNA target. In some embodiments, a crRNA can base pair with a tracrRNA (trans-activating CRISPR RNA) to form a RNA duplex that can direct a Cas polypeptide to recognize and optionally cleave a DNA target.

In some embodiments, as used herein, the term “guide polynucleotide”, can refer to a polynucleotide sequence that can form a complex with a polynucleotide guided polypeptide (e.g., a Cas protein, a MAD protein). In some embodiments, a guide polynucleotide can direct a polynucleotide guided polypeptide to recognize and optionally cleave (or nick) a DNA target site. In some embodiments, the terms “guide polynucleotide” and “guide polynucleic acid” can be used interchangeably herein. In some embodiments, a guide polynucleotide can be comprised of a single molecule (unimolecular) or two molecules (bimolecular). In some embodiments, a guide polynucleotide sequence can be an RNA sequence, a DNA sequence, or a combination thereof (an RNA-DNA combination sequence). In some embodiments, a guide polynucleotide that solely can comprise ribonucleic acids can also be referred to as a “guide RNA” (gRNA). In some embodiments, a guide polynucleic acid can be a guide RNA.

In some embodiments, the term “single guide RNA” (sgRNA) can refer to a synthetic fusion of two RNA molecules, for example, a crRNA (CRISPR RNA) comprising a variable targeting domain, and a tracrRNA. In some embodiments, a guide RNA can comprise a variable targeting domain (or VT domain) of 12 to 30 nucleotide sequences and an RNA fragment that can interact with a Cas protein.

In some embodiments, a guide polynucleotide can be bimolecular (i.e., two molecules; also referred to as “double molecule”, “dual” or “duplex” guide polynucleotide) comprising, for example, a first molecule having a nucleotide sequence domain (referred to as Variable Targeting domain or VT domain) that is complementary to a nucleotide sequence in a target polynucleic acid (e.g., target DNA) and a second molecule having a nucleotide sequence domain (referred to as Cas endonuclease recognition domain or CER domain) that interacts with a Cas polypeptide.

In some embodiments, complementarity between a guide polynucleic acid (e.g., the VT domain, spacer region) and a target polynucleic acid (e.g., protospacer) can be perfect, substantial, or sufficient. In some embodiments, perfect complementarity between two nucleic acids can mean that two nucleic acids can form a duplex in which every base in a duplex can be bonded to a complementary base by Watson-Crick pairing. In some embodiments, substantial or sufficient complementarity can mean that a sequence in one strand may not be completely and/or perfectly complementary to a sequence in an opposing strand, but that sufficient bonding occurs between bases on the two strands to form a stable hybrid complex in a set of hybridization conditions (e.g., salt concentration and temperature).

In some embodiments, the term “variable targeting domain” or “VT domain” can be used interchangeably herein and can refer to a nucleotide sequence that can be present in a guide polynucleotide. In some embodiments, a VT domain can be complementary to one strand of a double stranded DNA target site. In some embodiments, a percent complementation between a first nucleotide sequence domain (VT domain) and a target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, a variable target domain can be 12, 13, 14, 15, 16, 17, 18, 19, 20, 21, 22, 23, 24, 25, 26, 27, 28, 29 or 30 nucleotides in length. In some embodiments, a variable target domain can comprise at least 17 nucleotides that are complementary to at least 17 nucleotides of a target polynucleic acid. In some embodiments, a variable targeting domain can comprise a contiguous stretch of nucleotides that are complementary to a target polynucleic acid. In some embodiments, nucleotides of a guide polynucleic acid that are complementary to a target polynucleic acid can be non-contiguous. In some embodiments, a variable targeting domain can comprise a contiguous stretch of 12 to 30 nucleotides. In some embodiments, a variable targeting domain can be composed of a DNA sequence, an RNA sequence, a modified DNA sequence, a modified RNA sequence, or any combination thereof.

In some embodiments, a nucleotide sequence linking a crNucleotide and the tracrNucleotide of a single guide polynucleotide can comprise an RNA sequence, a DNA sequence, or an RNA-DNA combination sequence. In some embodiments, a nucleotide sequence linking a crNucleotide and a tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In some embodiments, a nucleotide sequence linking a crNucleotide and a tracrNucleotide of a single guide polynucleotide can comprise a tetranucleotide loop sequence, such as, but not limiting to a GAAA tetranucleotide loop sequence.

In some embodiments, a guide polynucleic acid can be introduced into a plant cell via transformation of a recombinant DNA construct comprising a polynucleotide encoding a guide polynucleic acid operably linked to a promoter functional in a plant; e.g., a plant U6 polymerase III promoter, a CaMV 35S polymerase II promoter, a mitochondrial promoter, a plastid promoter.

In some embodiments, a plurality of guide polynucleic acids can be multiplexed to target multiple target nucleic acids. For example, 2, 3, 4, 5, 6, 7, 8, 9, 10, or more than 10 target nucleic acids can be targeted simultaneously or iteratively.

### Target Sites for Genome Modification

In some embodiments, the terms “target site”, “target sequence”, “target polynucleotide”, “target polynucleic acid”, “target locus”, “genomic target site”, “genomic target sequence”, and “genomic target locus” can be used interchangeably herein. In some embodiments, a target polynucleic acid can refer to a polynucleotide sequence in a genome (e.g., a plastid or a mitochondrial genome). In some embodiments, a genome can be part of a plant cell. In some embodiments, a target polynucleic acid can refer to a site (e.g., in a genome) recognized by a guide polynucleic acid. In some embodiments, a target polynucleic acid can refer to a site (e.g., in a genome) at which a single-strand or double-strand break can be induced (e.g., by a Cas polypeptide). In some embodiments, a target site can be an endogenous site in a genome. In some embodiments, a target site can be heterologous to an organism and thereby not be naturally occurring in a genome. In some embodiments, a target site can be found in a heterologous genomic location compared to where it occurs in nature. In some embodiments, as used herein, the terms “endogenous target sequence” and “native target sequence” can be used interchangeably herein and can refer to a target sequence that can be endogenous or native to a genome of an organism. In some embodiments, endogenous target sequence can occur at an endogenous or native position of a target sequence in a genome of an organism.

In some embodiments, a target polynucleic acid can be DNA, RNA, or both. In some embodiments, a target polynucleic acid can be DNA (e.g., target DNA). In some embodiments, a target polynucleic acid can be genomic DNA. In some embodiments, a target polynucleic acid can be nuclear DNA, mitochondrial DNA, plastid DNA, or any combination thereof.

In some embodiments, the terms “artificial target site” and “artificial target sequence” can be used interchangeably herein and can refer to a target sequence that has been introduced into a genome of a plant. In some embodiments, such an artificial target sequence can be identical in sequence to an endogenous or native target sequence in a genome of an organism but may be located in a different position (i.e., a non-endogenous or non-native position) in a genome of an organism.

In some embodiments, an “altered target site”, “altered target sequence”, “modified target site”, “modified target sequence” can be used interchangeably herein and can refer to a target sequence as disclosed herein that can comprise at least one alteration when compared to a non-altered target sequence. In some embodiments, such “alterations” can include, for example: (i) a substitution of at least one nucleotide, (ii) a deletion of at least one nucleotide, (iii) an insertion of at least one nucleotide, or (iv) any combination of (i)-(iii).

In some embodiments, a length of a target site can vary and can include, for example, target sites that are at least 12, 13, 14, 15, 16, 17, 18, 19, 20, 21, 22, 23, 24, 25, 26, 27, 28, 29, 30 or more nucleotides in length. In some embodiments, a target site can be palindromic. In some embodiments, a palindromic sequence can comprise a sequence that on one strand reads the same in the opposite direction on the complementary strand. In some embodiments, a nick/cleavage site can be within a target sequence. In some embodiments, a nick/cleavage site can be outside of a target sequence. In some embodiments, a cleavage could occur at nucleotide positions immediately opposite each other to produce a blunt end cut or, in other cases, incisions could be staggered to produce single-stranded overhangs, also called “sticky ends”, which can be either 5′ overhangs, or 3′ overhangs.

In some embodiments, a target nucleic acid sequence can be 5′ or 3′ of a PAM. In some embodiments, a target nucleic acid sequence can be, for example, 16, 17, 18, 19, 20, 21, 22, or 23 bases immediately 5′ of the first nucleotide of the PAM. In some embodiments, a target nucleic acid sequence can be, for example, 16, 17, 18, 19, 20, 21, 22, or 23 bases immediately 3′ of a last nucleotide of a PAM. In some embodiments, a target nucleic acid sequence can be 20 bases immediately 5′ of a first nucleotide of a PAM. In some embodiments, a target nucleic acid sequence can be 20 bases immediately 3′ of a last nucleotide of a PAM.

In some embodiments, a site-specific cleavage of a target nucleic acid by a polynucleotide guided polypeptide (e.g., Cas protein) can occur at locations determined by base-pairing complementarity between a guide nucleic acid and a target nucleic acid. In some embodiments, a site-specific cleavage of a target nucleic acid by a polynucleotide guided polypeptide (e.g., Cas protein) can occur at locations determined by a protospacer adjacent motif (PAM). In some embodiments, a cleavage site of Cas (e.g., Cas9) can be about 1 to about 25, or about 2 to about 5, or about 19 to about 23 base pairs (e.g., 3 base pairs) upstream or downstream of a PAM sequence. In some embodiments, a cleavage site of a Cas (e.g., Cas9) can be 3 base pairs upstream of a PAM sequence. In some embodiments, a cleavage site of a Cas (e.g., Cpf1) can be 19 bases on a (+) strand and 23 bases on a (−) strand, producing a 5′ overhang 5 nt in length. In some cases, a cleavage can produce blunt ends. In some cases, a cleavage can produce staggered or sticky ends with 5′ overhangs. In some cases, a cleavage can produce staggered or sticky ends with 3′ overhangs.

In some embodiments, different organisms can comprise different PAM sequences. In some embodiments, different Cas proteins can recognize different PAM sequences. In some embodiments, in S. pyogenes, a PAM can be a sequence in a target nucleic acid that can comprise a sequence 5′-NRR-3′, where R can be either A or G, where N can be any nucleotide and N can be immediately 3′ of a target nucleic acid sequence targeted by a spacer sequence. In some embodiments, a PAM sequence of S. pyogenes Cas9 (SpyCas9) can be 5′-NGG-3′, where N can be any DNA nucleotide and can be immediately 3′ of a CRISPR recognition sequence of a non-complementary strand of a target DNA. In some embodiments, a PAM of Cpf1 can be 5′-TTN-3′, where N can be any DNA nucleotide and can be immediately 5′ of the CRISPR recognition sequence.

In some embodiments, a consensus PAM sequence for various MAD polypeptides has been determined (U.S. Pat. No. 9,982,279). In some embodiments, a consensus PAM for MAD1-MAD8, and MAD10-MAD12 was determined to be TTTN. In some embodiments, a consensus PAM for MAD9 was determined to be NNG. In some embodiments, a consensus PAM for MAD13-MAD15 was determined to be TTN. In some embodiments, a consensus PAM for MAD16-MAD18 was determined to be TA. In some embodiments, a consensus PAM for MAD19-MAD20 was determined to be TTCN.

In some embodiments, active variants of genomic target sites can also be used. In some embodiments, active variants can comprise at least 65%, 70%, 75%, 80%, 85%, 90%, 91%, 92%, 93%, 94%, 95%, 96%, 97%, 98%, 99% or more sequence identity to a given target site. In some embodiments, active variants can retain biological activity. In some embodiments, active variants can be recognized by a polynucleotide guided polypeptide (e.g., Cas protein). In some embodiments, active variants can be cleaved by a polynucleotide guided polypeptide (e.g., Cas protein). In some embodiments, assays can be used to measure a double-strand break of a target site by an endonuclease. In some embodiments, assays can measure an overall activity and/or specificity of an endonuclease on DNA substrates containing recognition sites (e.g., target sites, active variants).

### Methods for Integrating a Donor Polynucleotide

In some embodiments, the disclosure provides methods to obtain an organelle (e.g., mitochondrion or plastid) comprising a donor polynucleotide. In some embodiments, a method can employ homologous recombination to provide integration of a polynucleotide at a target site. In some embodiments, a homologous recombination can be enhanced by introducing a double-strand break (DSBs) at selected endonuclease target sites. In some embodiments, described herein is a use of a polynucleotide guided polypeptide system which can provide flexible genome cleavage specificity and can result in a high frequency of double-strand breaks at an organellar DNA target site. In some embodiments, a specific cleavage can enable efficient gene editing of a nucleotide sequence of interest. In some embodiments, a nucleotide sequence of interest to be edited can be located within or outside a target site recognized and/or cleaved by a polynucleotide guided polypeptide (e.g., a Cas polypeptide, a MAD polypeptide).

In some embodiments, a polynucleotide of interest can be provided to an organelle in a donor polynucleotide. In some embodiments, a donor polynucleotide can be a nucleic acid sequence (e.g., DNA, RNA, or both) that can be integrated into a target nucleic acid, for example, a genome of a mitochondrion or a plastid. In some embodiments, the donor polynucleotide can comprise a polynucleotide encoding a variant of a naturally occurring polypeptide having an enzyme activity (e.g., a herbicide-resistant ALS, a herbicide-resistant EPSPS, or a herbicide-resistant GS). In some embodiments, the donor polynucleotide can comprise the polynucleotide encoding the variant of the naturally occurring polypeptide having the enzyme activity, and an additional polynucleotide encoding a gene of interest. In some embodiments, a gene of interest can be a cytoplasmic male sterility (CMS) coding region. In some embodiments, a donor polynucleotide can be inserted into a genome e.g., at a cleavage site of a polynucleotide guided polypeptide. In some embodiments, a donor polynucleotide can be inserted into a genome by homologous recombination. In some embodiments, the method further comprises removing the polynucleotide encoding the variant of the naturally occurring polypeptide having the enzyme activity after integration of the gene of interest. In some embodiments, a donor polynucleotide can comprise DNA and can be referred to as donor DNA.

In some embodiments, a donor polynucleotide of any suitable size can be integrated into a genome. In some embodiments, a donor polynucleotide integrated into a genome can be less than 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be at least about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, about 500 kb, or less than about 500 kilobases (kb) in length. In some embodiments, a donor polynucleotide integrated into a genome can be up to about 1 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 10.5 kb, about 11 kb, about 11.5 kb, about 12 kb, about 12.5 kb, about 13 kb, about 13.5 kb, about 14 kb, about 14.5 kb, about 15 kb, about 16 kb, about 17 kb, about 18 kb, about 19 kb, about 20 kb, about 25 kb, about 30 kb, about 35 kb, about 40 kb, about 45 kb, about 50 kb, about 100 kb, about 150 kb, about 200 kb, about 250 kb, about 300 kb, about 350 kb, about 400 kb, about 450 kb, or up to about 500 kb in length.

In some embodiments, a donor polynucleotide can comprise a polynucleotide of interest, a polynucleotide modification template, a heterologous expression cassette, or any combination thereof. In some embodiments, the term “polynucleotide modification template” can refer to a polynucleotide that can comprise at least one nucleotide modification when compared to a nucleotide sequence to be edited. In some embodiments, a nucleotide modification can be at least one nucleotide substitution, addition, deletion, or any combination thereof. In some embodiments, a minor genome modification created by use of a polynucleotide modification template can include creation of a mutant allele (e.g., antibiotic resistant rRNA gene) and removal of a target site for a polynucleotide guided polypeptide. In some embodiments, a donor polynucleotide (e.g., donor DNA) can comprise a first and a second region of homology. In some embodiments, a donor polynucleotide comprises a heterologous sequence that is flanked by a first and a second region of homology. In some embodiments, a first and second region of homology of a donor polynucleotide (e.g., donor DNA) can share homology to a first and a second genomic region, respectively, present in or flanking a target site (e.g., of an organellar genome).

In some embodiments, “homology” can mean DNA sequences that are similar. In some embodiments, homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology or identity. In some embodiments, a “region of homology to a genomic region” can be a region of DNA that has a similar sequence to a given “genomic region” in an organellar genome. In some embodiments, a region of homology can be of any length that can be sufficient to promote homologous recombination at a cleaved target site. In some embodiments, a region of homology can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that a region of homology can have sufficient homology to undergo homologous recombination with a corresponding genomic region. In some embodiments, a “sufficient homology” can indicate that two polynucleotide sequences can have sufficient structural similarity to act as substrates for a homologous recombination reaction. In some embodiments, a donor polynucleotide (e.g., donor DNA) may comprise an expression cassette (e.g., encoding a heterologous polynucleotide of interest). In some embodiments, a donor polynucleotide may comprise multiple expression cassettes. In some embodiments, an expression cassette may be a polycistronic expression cassette, e.g., where multiple protein-coding regions, functional RNAs, or a combination of both, are expressed under control of a single promoter.

In some embodiments, the method can further comprise introducing into a nucleus of the cell, a polynucleotide comprising a Rep coding region, and introducing into the mitochondrion of the cell a VOR-Donor-VOR polynucleotide. In some embodiments, a modified Rep protein comprising a Rep protein operably linked to a mitochondrial targeting peptide can be introduced into the nucleus of the cell. In some embodiments, the modified Rep protein can comprise an amino acid sequence having at least about 70%, 75%, 80%, 85%, 90%, 95%, 96%, 97%, 98%, 99%, or 100% sequence identity to SEQ ID NO: 111. In some embodiments, the polynucleotide encoding a Rep protein or a modified Rep protein (e.g., nMTS-Rep) can be operably linked to an inducible promoter. In some embodiments, the polynucleotide encoding a Rep protein, or a modified Rep protein can be operably linked to a constitutively active promoter.

In some embodiments, the VOR-Donor DNA-VOR polynucleotide can comprise geminivirus VOR sequences such that the 5′ and 3′ Donor DNA regions with sequence homologous to target sites is flanked by a VOR sequences to yield a VOR-Donor DNA-VOR configuration. In some embodiments, the VOR sequence can comprise a polynucleotide sequence having at least about 70%, 75%, 80%, 85%, 90%, 95%, 96%, 97%, 98%, 99%, or 100% sequence identity to SEQ ID NO: 71.

In some embodiments, the Rep protein can induce replication of the donor DNA flanked by the VOR sequences, target sites for the geminivirus Rep protein. In some embodiments, amplification of the donor DNA mediated by Rep-VOR interaction, after its transformation into organelle, results in gene expression of the donor DNA being enhanced by at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400% or 500% compared to gene expression of the donor DNA lacking VOR elements, Rep protein, or both.

In some embodiments, a “donor RNA” can be a corresponding RNA molecule that can comprise, for example, a same nucleic acid sequence as a donor DNA; i.e., with uridylate (“U”) in place of deoxythymidylate (“T”). In some embodiments, a “donor polynucleotide” may be either a donor DNA or a donor RNA, or a combination of DNA and RNA. In some embodiments, a donor polynucleotide may be either single-stranded or double-stranded.

In some embodiments, an alternative method for modification of an organellar genome can be a replacement of part or all of an organelle DNA with a “replacement DNA”. In some embodiments, an endogenous organellar DNA can be reduced or eliminated by use of site-specific endonucleases such as polynucleotide guided polypeptides (e.g., Cas polypeptide, Cas9 polypeptide, MAD polypeptide, MAD7 polypeptide). In some embodiments, at the same time or subsequently, a replacement DNA can be introduced. In some embodiments, the term “replacement DNA” can refer to fragments of organellar DNA or complete organellar DNA that can convey a new genotype and corresponding trait(s) when transformed into an organelle. In some embodiments, the terms “replacement DNA” and “replacement organellar DNA” can be used interchangeably herein. In some embodiments, in the case of organellar DNA fragments, they can be integrated into a remaining endogenous organellar DNA by homologous recombination. In a case of complete organellar DNA replacement, a replacement DNA can be isolated from cultivars, lines, sub species and other species which possess DNA compositions distinct from an endogenous organellar DNA of recipient cells. In some embodiments, a replacement DNA can also be partially and/or completely synthesized in vitro. In some embodiments, a replacement DNA can comprise both native and non-native sequences. In some embodiments, when replacement DNA is created in vitro, it can be a linear DNA with a repeat sequence at the ends. In some embodiments, a repeat sequence can be direct repeats or inverted repeats. In some embodiments, the ends can facilitate homologous recombination in vitro or in vivo to create circular DNA for replication of organellar DNA in cells. In some embodiments, a DNA created in vitro can also include heterologous DNA elements such as ones to allow selected amplification in bacterial cells. In some embodiments, a replacement DNA can comprise a DNA element functioning as a DNA replication origin in a recipient organelle. In some embodiments, a replacement DNA can comprise multiple DNA fragments that are capable of recombination within an organelle to result in a complete replacement DNA.

In some embodiments, a sequence functional as an origin of replication can be included with compositions (e.g., polynucleotides, constructs, cassettes) of the disclosure. Such sequences can include the origin of replication for an organelle. In some embodiments, an origin of replication sequence can be a plastid origin of replication (e.g., plastid rRNA intergenic region) sequence. In some embodiments, an origin of replication sequence can be a mitochondrial origin of replication sequence.

In some embodiments, as used herein, a “genomic region” can refer to a segment of DNA in a genome of, for example, an organelle (e.g., a mitochondrion or a plastid). In some embodiments, a genomic region can be present on either side of a target site. In some embodiments, a genomic region can comprise a portion of a target site. In some embodiments, a genomic region can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases. In some embodiments, a genomic region can comprise sufficient homology to undergo homologous recombination with a corresponding region of homology that is present on a donor DNA.

In some embodiments, a donor polynucleotide, a polynucleotide of interest and/or trait can be stacked together in a complex trait locus. In some embodiments, a guide polynucleotide/polypeptide system can be used to generate double strand breaks and for stacking traits in a complex trait locus.

In some embodiments, two or more polynucleotides encoding RNA and/or proteins can be included in a cassette as a polycistronic unit. In some embodiments, a polynucleotide encoding an RNA can be expressed from separate cassettes.

In some embodiments, a guide polynucleotide/polypeptide system can be used for introducing one or more donor polynucleotides or one or more traits of interest into one or more target sites by providing one or more guide polynucleotides, one or more polynucleotide guided polypeptides (e.g., Cas polypeptides, MAD polypeptides), and optionally one or more donor polynucleotides (e.g., donor DNA) to a plant cell. In some embodiments, an organism can be produced from a cell that can comprise an alteration at said one or more target sites of an organellar DNA (e.g., mitochondrial DNA or plastid DNA), wherein an alteration can be selected from a group consisting of (i) a substitution of at least one nucleotide, (ii) a deletion of at least one nucleotide, (iii) an insertion of at least one nucleotide, and (iv) any combination of (i)-(iii).

In some embodiments, a structural similarity between a given genomic region and a corresponding region of homology of a donor polynucleotide (e.g., donor DNA) can be any degree of sequence identity that allows for homologous recombination to occur. In some embodiments, an amount of homology or sequence identity shared by a “region of homology” of a donor polynucleotide (e.g., donor DNA) and a “genomic region” of a plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

In some embodiments, a region of homology of a donor polynucleotide (e.g., donor DNA) can have homology to any sequence flanking a target site. While in some embodiments, regions of homology can share significant sequence homology to a genomic sequence immediately flanking a target site, the regions of homology can be designed to have sufficient homology to regions that may be further 5′ or 3′ to a target site. In still other embodiments, regions of homology can also have homology with a fragment of a target site along with downstream genomic regions. In one embodiment, a first region of homology can further comprise a first fragment of a target site and a second region of homology can comprise a second fragment of a target site, wherein a first and second fragments are dissimilar.

In some embodiments, as used herein, “homologous recombination” can refer to an exchange of DNA fragments between two DNA molecules at sites of homology. In some embodiments, a frequency of homologous recombination can be influenced by a number of factors. In some embodiments, a length of a region of homology can affect a frequency of homologous recombination events, for example, a longer a region of homology, can have a greater frequency of homologous recombination. In some embodiments, a length of a homology region needed to observe homologous recombination may vary among species.

In some embodiments, an intermolecular recombination can occur in mitochondria and in plastids, for example, plants with transformed mitochondrial DNA or transformed plastid DNA can arise through site-specific integration of foreign sequences by homologous recombination with a flanking sequence on a transformation vector.

In some embodiments, an intramolecular recombination between repeated sequences can generate, for example, inversions when repeats are palindromic or deletions when direct.

In some embodiments, endogenous mitochondrial or plastid sequences can be used to target insertions to achieve efficient foreign sequence integration by homologous recombination. In some embodiments, a positive correlation can be present between a rate of recombination and a length and/or degree of sequence homology.

In some embodiments, a minimum flanking sequence length for homologous recombination with an organellar genome can be influenced by an introduction of single-stranded or double-stranded breaks (or both) in an organellar genome, e.g., by polynucleotide guided polypeptide(s).

In some embodiments, an efficiency of a disclosed methods for genome engineering or modification can be at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%.

In some embodiments, a method can comprise introducing into an organelle (e.g., a mitochondrion or a plastid) of a cell (e.g., a plant cell) a donor polynucleotide (e.g., a donor DNA), a guide polynucleic acid (or multiple guide polynucleic acids) and a polynucleotide guided polypeptide. In some embodiments, at least one single-strand or double-strand break can be introduced in a target site by a polynucleotide guided polypeptide, a first and second region of homology of a donor polynucleotide (e.g., donor DNA) can undergo homologous recombination with their corresponding genomic regions of homology resulting in exchange of DNA between the donor and the genome. In some embodiments, methods disclosed herein can result in an integration of all or part of a donor polynucleotide (e.g., donor DNA) into a single-strand or double-strand break(s) in a target site in an organellar genome, thereby altering an original target site and producing an altered genomic target site.

In some embodiments, a cell can be a eukaryotic cell. In some embodiments, a cell can comprise, a human cell, an animal cell, a non-human animal cell, a bacterial cell, a fungal cell, an insect cell, a plant cell, a protist cell, a yeast cell, an algal cell, or any combination thereof. In some embodiments, a cell can be a wheat cell, a maize cell, a rice cell, a barley cell, a sorghum cell, a rye cell, a canola cell, a broccoli cell, a cauliflower cell, and a soybean cell. In some embodiments, a cell can be part of an organism or a tissue. In some embodiments, an organism can comprise a plant, a transgenic plant, or parts thereof comprising a cell, a tissue, a propagation material, a seed, a pollen, a progeny, or any combination thereof produced by the methods described herein. In some embodiments, a cell can be an isolated and purified human cell.

In some embodiments, a nucleotide to be edited can be located within or outside a target site recognized and cleaved by a polynucleotide guided polypeptide. In some embodiments, at least one nucleotide modification may not be a modification at a target site recognized and cleaved by a polynucleotide guided polypeptide. In some embodiments, there can be at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the organellar DNA target site. In some embodiments, a nucleotide to be edited can be located both within and outside a target site (or multiple target sites) recognized and cleaved by a polynucleotide guided polypeptide.

In some embodiments, a donor polynucleotide can comprise a donor DNA. In some embodiments, a donor polynucleotide can be introduced by any suitable means. In some embodiments, a plant having a target site can be provided. In some embodiments, a donor polynucleotide (e.g., donor DNA) can be provided by any suitable transformation method including, for example, Agrobacterium-mediated transformation or biolistic particle bombardment. In some embodiments, a donor polynucleotide (e.g., donor DNA) may be present transiently in a cell or it can be introduced via a viral replicon. In some embodiments, in a presence of a guide polynucleotide (e.g., guide RNA), a polynucleotide guided polypeptide (e.g., Cas polypeptide, MAD polypeptide) and a target site, a donor polynucleotide (e.g., donor DNA) can be inserted into an organellar genome.

### Polynucleotides of Interest for Integration at a Target Site

In some embodiments, further provided are methods for identifying at least one plant cell comprising an organelle comprising a genome comprising a polynucleotide of interest integrated at a target site. In some embodiments, an organelle can comprise a mitochondrion, a plastid, or a combination thereof. In some embodiments, a donor polynucleotide can comprise a polynucleotide of interest. In some embodiments, a variety of methods can be used for identifying those plant cells with an insertion into a genome at or near to a target site without using a screenable marker phenotype. In some embodiments, a method can be viewed as directly analyzing a target sequence to detect any change in a target sequence, including but not limited to PCR methods, sequencing methods, nuclease digestion, Southern blots, and any combination thereof.

In some embodiments, a method can also comprise recovering a plant from a plant cell comprising a polynucleotide of interest integrated into its organellar genome. In some embodiments, a plant can be sterile or fertile.

In some embodiments, a polynucleotide or polypeptide of interest can comprise a herbicide-tolerance coding sequence, an insecticidal coding sequence, a nematocidal coding sequence, an antimicrobial coding sequence, an antifungal coding sequence, an antiviral coding sequence, an abiotic stress tolerance coding sequence, a biotic stress tolerance coding sequence, a sequence modifying a plant trait, or any combination thereof. In some embodiments, a plant trait can comprise yield, grain quality, nutrient content, starch quality and quantity, nitrogen fixation and/or utilization, and oil content and/or composition, or any combination thereof. In some embodiments, a polynucleotide of interest can include, a gene that improves crop yield, a polypeptide that improves a desirability of a crop, a gene encoding a protein conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. In some embodiments, genes of interest can include, for example, those genes involved in information, such as zinc fingers, those involved in communication, such as kinases, and those involved in housekeeping, such as heat shock proteins. In some embodiments, a polynucleotide of interest can include a gene encoding an important trait for agronomics, insect resistance, disease resistance, herbicide resistance, fertility or sterility, grain characteristics, commercial products, or any combination thereof. In some embodiments, a gene of interest can include those involved in; oil, starch, carbohydrate, or nutrient metabolism; those affecting photosynthesis, photorespiration, and ATP metabolism; or any combination thereof.

In some embodiments, commercial traits can also be obtained by expression of proteins encoded on a polynucleotide. In some embodiments, a commercial use of transformed plants can be a production of polymers and bioplastics. In some embodiments, polynucleotides of interest can include genes encoding proteins such as B-ketothiolase, PHBase (polyhydroxybutyrate synthase), and acetoacetyl-CoA reductase which can facilitate expression of polyhydroxyalkanoates (PHAs). In some embodiments, a commercial use can be expression of a gene or genes that can increase starch for ethanol production.

In some embodiments, a polynucleotide or polypeptide that can influence amino acid biosynthesis can include, for example, anthranilate synthase (AS; EC 4.1.3.27) which can catalyze a first reaction branching from an aromatic amino acid pathway to a biosynthesis of tryptophan in plants, fungi, and bacteria. In some embodiments, in plants, a chemical process for a biosynthesis of tryptophan can be compartmentalized in a chloroplast. In some embodiments, additional donor sequences of interest can include Chorismate Pyruvate Lyase (CPL) which can refer to a gene encoding an enzyme which can catalyze a conversion of chorismate to pyruvate and pHBA. In some embodiments, a CPL gene can be from E. coli. In some embodiments, a CPL gene can bear GenBank accession number M96268.

In some embodiments, a polynucleotide sequence of interest can encode proteins involved in providing disease or pest resistance. In some embodiments, “disease resistance” or “pest resistance” can cause a plant to at least in part avoid a harmful symptom or outcome from a plant-pathogen interaction. In some embodiments, a pest resistance gene can encode resistance to a pest that has great yield drag. In some embodiments, a pest that has great yield drag can comprise rootworm, cutworm, European Corn Borer, or any combination thereof. In some embodiments, a disease resistance or insect resistance gene can comprise a lysozyme, a cecropin, or a combination thereof. In some embodiments, a disease resistance or insect resistance gene can provide antibacterial protection, antifungal protection, nematode protection, insect protection, or any combination thereof. In some embodiments, an antifungal resistance gene or protein can comprise a defensin, a glucanase, a chitinase or any combination thereof. In some embodiments, a nematode or insect protection gene or protein can comprise a Bacillus thuringiensis endotoxin, a protease inhibitor, a collagenase, a lectin, a glycosidase, or any combination thereof. In some embodiments, a gene encoding a disease resistance trait can include a detoxification gene. In some embodiments, a detoxification gene can comprise a fumonisin gene; an avirulence (avr) gene, a disease resistance (R) gene, or any combination thereof. In some embodiments, an insect resistance gene can encode resistance to pests that have great yield drag such as rootworm, cutworm, European Corn Borer, or any combination thereof. In some embodiments, an insect resistance gene can comprise a Bacillus thuringiensis (Bt) toxic protein gene.

In some embodiments, transgenes, recombinant DNA molecules, DNA sequences of interest, or donor polynucleotides can comprise one or more DNA sequences for gene silencing of a target gene. In some embodiments, a target gene can comprise a plant pest gene or a plant pathogen gene. In some embodiments, a method for gene silencing can comprise expression of a DNA sequence in a plant. In some embodiments, a method for gene silencing can comprise cosuppression, antisense suppression, double-stranded RNA (dsRNA) interference, hairpin RNA (hpRNA) interference, intron-containing hairpin RNA (ihpRNA) interference, transcriptional gene silencing, and microRNA (miRNA) interference.

In some embodiments, a fertile plant can be a plant that can produce viable male and female gametes and can be self-fertile. In some embodiments, a self-fertile plant can produce a progeny plant without a contribution from any other plant of a gamete and a genetic material contained therein. Also disclosed herein in some embodiments, are methods comprising a use of a plant that may not be self-fertile. In some embodiments, a plant may not produce male gametes, or female gametes, or both, that are viable or otherwise capable of fertilization. In some embodiments, as used herein, a “male-sterile plant” can be a plant that does not produce male gametes that are viable or otherwise capable of fertilization. In some embodiments, as used herein, a “female-sterile plant” can be a plant that does not produce female gametes that are viable or otherwise capable of fertilization. In some embodiments, male-sterile and female-sterile plants can be female-fertile and male-fertile, respectively. In some embodiments, a male-fertile (but female-sterile) plant can produce viable progeny when crossed with a female-fertile plant. In some embodiments, a female-fertile (but male-sterile) plant can produce viable progeny when crossed with a male-fertile plant. In some embodiments, in some crop species a use of hybrid plants has been shown to dramatically increase crop yield. In some embodiments, a hybrid crop system can require a male sterile line that can serve as a female parent to produce hybrid seed through fertilization with pollen donor plants. In some embodiments, a method to convey male sterility without manual or mechanical intervention can comprise a use of a cytoplasmic male sterility (CMS) gene. In some embodiments, a CMS gene can comprise a nucleic acid. In some embodiments, a CMS gene can comprise a heterologous nucleic acid. In some embodiments, a nucleic acid can comprise DNA, RNA, or a combination thereof. In some embodiments, a coding region, an open reading frame, or a combination thereof. In some embodiments, a CMS gene can be a maternally inherited trait conferred by a mitochondrial genome that results in a failure to produce functional pollen and/or male reproductive organs except in a presence of restorer-of-fertility (RF) genes. In some embodiments, a chimeric mitochondrial ORF can be found to lead to male sterility, producing unisex-female plants. In some embodiments, a creation of a chimeric CMS gene can be a consequence of the highly recombinogenic, repetitive nature of plant mitochondrial genomes. In some embodiments, methods described herein could be used to introduce one or more naturally occurring or custom-designed CMS protein-coding sequences into mitochondria of various monocot species, dicot species, or a combination thereof. In some embodiments, a monocot species can comprise wheat, maize, rice, barley, sorghum, sugarcane, rye, or any combination thereof. In some embodiments, a dicot can comprise soybean, potato, tomato, canola, broccoli, cauliflower, or any combination thereof. In some embodiments, a CMS protein-coding sequence of a CMS gene can be operably linked to heterologous regulatory sequences.

In some embodiments, a CMS gene can comprise all or part of an orf79 gene (e.g., an orf79 protein-coding sequence) from rice. In some embodiments, the CMS gene can have an amino acid sequence having at least about 50% sequence identity (e.g., at least about 55%, 60%, 65%, 70%, 75%, 80%, 85%, 90%, 95%, 96%, 97%, 98%, 99%, or greater) to SEQ ID NO: 47. In some embodiments, a CMS gene can comprise all or part of an orf256 gene (e.g., an orf256 protein-coding sequence) from wheat. In some embodiments, the CMS gene may have an amino acid sequence having at least about 50% sequence identity (e.g., at least about 55%, 60%, 65%, 70%, 75%, 80%, 85%, 90%, 95%, 96%, 97%, 98%, 99%, or greater) to SEQ ID NO: 54. In some embodiments, a CMS gene can comprise all or part of an orf279 gene (e.g., an orf279 protein-coding sequence) from wheat. In some embodiments, the CMS gene may have an amino acid sequence having at least about 50% sequence identity (e.g., at least about 55%, 60%, 65%, 70%, 75%, 80%, 85%, 90%, 95%, 96%, 97%, 98%, 99%, or greater) to SEQ ID NO: 56. In some embodiments, a CMS gene can comprise all or part of a T-urf13 gene (e.g., a T-urf13 protein-coding sequence) from maize.

### Selection of Transformed Cells Using Inhibitors of Plant Enzymes

In some embodiments, an embryogenic callus culture of a plant can be initiated and maintained for a minimum of 4-8 weeks (e.g., 4-6 weeks) on a Chu-N6-based induction & maintenance medium supplemented with the plant growth regulator 2,4-D. In some embodiments, the plant may be selected from the group consisting of: rice, wheat, maize, sorghum, barley, rye, canola, broccoli, cauliflower, and soybean. In some embodiments, the plant is rice. In some embodiments, four days prior to transformation, calli can be prepared for transformation by plating tissue in the target zone on the same N6-based medium supplemented with mannitol and sorbitol for osmotic protection.

In some embodiments, a plant callus (e.g., a rice callus) can be transformed with one or with multiple plant enzyme expression constructs (e.g., herbicide-resistant plant enzyme; regulatory plant enzyme). In some embodiments, a plant callus can be transformed with one or more plant enzymes (e.g., a herbicide-resistant ALS, a herbicide-resistant EPSPS, and/or a herbicide-resistant GS expression constructs). For example, the herbicide-resistant GS gene or EPSPS gene can be co-transformed with an herbicide-resistant ALS gene. In some embodiments, the herbicide-resistant plant enzyme expression cassette is a mitochondrial expression cassette. In some embodiments, the expression cassette is a nuclear expression cassette that encodes a plant enzyme (or a variant of plant enzyme) fused with a mitochondrial targeting sequence.

In some embodiments, a plant callus (e.g., a rice callus) can be transformed with one or with multiple ALS expression constructs (e.g., herbicide-resistant ALS-LS; regulatory ALS-SS). In some embodiments, the herbicide-resistant ALS-LS expression cassette is a mitochondrial expression cassette. In some embodiments, the ALS-SS expression cassette is a mitochondrial expression cassette. In some embodiments, the ALS-SS (or the herbicide-resistant ALS-LS) expression cassette is a nuclear expression cassette that encodes an ALS-SS (or the herbicide-resistant ALS-LS) fused with a mitochondrial targeting sequence.

In some embodiments, the herbicide-resistant ALS can comprise an amino acid sequence having at least about 70%, 80%, 85%, 90%, 91%, 92%, 93%, 94%, 95%, 96%, 97%, 98%, or 99% sequence identity to SEQ ID NO: 24. In some embodiments, the polynucleotide encoding the herbicide resistant ALS can comprise a nucleic acid sequence having at least about 70%, 80%, 85%, 90%, 91%, 92%, 93%, 94%, 95%, 96%, 97%, 98%, or 99% sequence identity to SEQ ID NO: 25. In some embodiments, the modified regulatory subunit of the acetolactate synthase or the modified biologically active fragment thereof can comprise an amino acid sequence having at least about 70%, 80%, 85%, 90%, 91%, 92%, 93%, 94%, 95%, 96%, 97%, 98%, 99%, or 100% sequence identity to SEQ ID NO: 21.

In some embodiments, a plant callus (e.g., a rice callus) can be transformed with one or with multiple EPSPS expression constructs (e.g., herbicide-resistant EPSPS, regulatory EPSPS, ATP1 promoter driving coding regions for herbicide-resistant version of EPSPS). In some embodiments, the herbicide-resistant EPSPS expression cassette is a mitochondrial expression cassette. In some embodiments, the EPSPS expression cassette is a mitochondrial expression cassette. In some embodiments, the EPSPS (or the herbicide-resistant EPSPS) expression cassette is a nuclear expression cassette that encodes an EPSPS (or the herbicide-resistant EPSPS) fused with a mitochondrial targeting sequence.

In some embodiments, the herbicide-resistant EPSPS can comprise an amino acid sequence having at least about 70%, 80%, 85%, 90%, 91%, 92%, 93%, 94%, 95%, 96%, 97%, 98%, or 99% sequence identity to SEQ ID NO: 62. In some embodiments, the polynucleotide encoding herbicide resistant EPSPS can comprise a nucleic acid sequence having at least about 70%, 80%, 85%, 90%, 91%, 92%, 93%, 94%, 95%, 96%, 97%, 98%, or 99% sequence identity to SEQ ID NO: 63.

In some embodiments, a plant callus (e.g., a rice callus) can be transformed with one or with multiple GS expression constructs (e.g., herbicide-resistant GS regulatory GS, ATP1 promoter driving coding regions for herbicide-resistant version of GS). In some embodiments, the herbicide-resistant GS expression cassette is a mitochondrial expression cassette. In some embodiments, the GS expression cassette is a mitochondrial expression cassette. In some embodiments, the GS (or the herbicide-resistant GS) expression cassette is a nuclear expression cassette that encodes an GS (or the herbicide-resistant EPSPS) fused with a mitochondrial targeting sequence.

In some embodiments, the herbicide-resistant GS can comprise an amino acid sequence having at least about 70%, 80%, 85%, 90%, 91%, 92%, 93%, 94%, 95%, 96%, 97%, 98%, or 99% sequence identity to SEQ ID NO: 72. In some embodiments, the polynucleotide encoding herbicide resistant GS can comprise a nucleic acid sequence having at least about 70%, 80%, 85%, 90%, 91%, 92%, 93%, 94%, 95%, 96%, 97%, 98%, or 99% sequence identity to SEQ ID NO: 73.

In some embodiments, a sequence encoding a start codon of the variant of a naturally occurring polypeptide having an enzyme activity (e.g., a herbicide-resistant ALS, a herbicide-resistant EPSPS, or a herbicide-resistant GS) can be replaced with a sequence encoding a mitochondrial RNA editing site. For example, the mitochondrial RNA editing site can comprise a rice mitochondrial nad4L gene (e.g., SEQ ID NO: 41), a rice mitochondrial cox2 gene (e.g., SEQ ID NO: 42), a wheat mitochondrial cox2 gene (e.g., SEQ ID NO: 80), and any combination thereof.

In some embodiments, transformation is performed using a technique selected from the group consisting of: microinjection, meristem transformation, electroporation, Agrobacterium-mediated transformation, viral based gene transfer, transfection, vacuum infiltration, biolistic particle bombardment or any combination thereof. In some embodiments, transformation may be performed using biolistic particle bombardment. In some embodiments, a variation of a transformation condition can comprise varying particle size and amount. In some embodiments, a variation of a transformation condition can comprise varying the amount of DNA on the particle. In some embodiments, a variation in transformation condition can be the concentration of a selective agent in the first selection after bombardment, or in subsequent selections. In some embodiments, the following steps can be followed for culture, selection, and regeneration:

After bombardment, a callus can be incubated in darkness for 16-20 hours at 26° C., then clumps approximately 1-3 mm in size can be sub-cultured to selective media. In some embodiments, selective media are supplemented with an inhibitor of the plant enzyme (e.g., ALS).

In some embodiments, the plant enzyme can be ALS, and the inhibitor of ALS is a sulfonylurea. In some embodiments, the inhibitor of ALS is chlorsulfuron. In some embodiments, the selective media are supplemented with chlorsulfuron at a concentration of 20 nM-100 nM, 100 nM-1 μM, 1 μM-20 μM, or 20 μM-100 μM. In some embodiments, the plant enzyme can be EPSPS, and inhibitor of EPSPS is a glyphosate. In some embodiments, the selective media can be supplemented with glyphosate at a concentration of at least about 0.1 mM, 0.5 mM, 1.0 mM, 1.5 mM, 2 mM, 2.5 mM, 3 mM, 3.5 mM, 4 mM, 4.5 mM, 5 mM, 10 mM, 20 mM, 30 mM, 40 mM, 50 mM, 60 mM, 70 mM, 80 mM, 90 mM, 100 mM, or more. In some embodiments, the plant enzyme can be GS, and inhibitor of GS can be a glyphosate, a bialaphos, a phosalacine. In some embodiments, the selective media are supplemented with glufosinate at a concentration of at 1-1000 mg/L, 10-500 mg/L, 20-400 mg/L, 30-300 mg/L, 40-200 mg/L, or 50-100 mg/L.

Calli on selective media can then be returned to dark incubation for 2-3 weeks. After 2-3 weeks of dark incubation, small clumps approximately 1-3 mm in size can again be subcultured to fresh selective medium containing a plant enzyme inhibitor (e.g., chlorsulfuron) and incubated for approximately 2 weeks in a lighted plant growth chamber with a 16 hr light-8 hr dark photoperiod, at intensity of 60 μmoles per square meter per second, at 26° C. In some embodiments, additional rounds of subculturing to fresh selection medium after 2-week periods of maintenance in the light can be performed.

At the end of the second selection period or later, between 5-8 weeks after bombardment, vigorously growing calli (individual initial events) can be picked from the surrounding dying tissue and transferred to individual plates of fresh selective medium supplemented with the plant enzyme inhibitor (e.g., chlorsulfuron), maintaining their individual identity.

At the end of this last 2-week or more selection period of individual plates, calli which are sustaining growth (representing putative mitochondrial transformation events) can be transferred to an N6-based medium for embryo maturation, still containing the plant enzyme inhibitor (e.g., chlorsulfuron) as a selective agent, but omitting the growth regulator 2,4-D, and supplementing with 2.5 g/L Phytagel.

After a minimum of 10-14 days, mature somatic embryos showing signs of normal maturation can be transferred to an N6-based germination medium, still containing the plant enzyme inhibitor (e.g., chlorsulfuron) as a selective agent. In some embodiments, this medium can be supplemented with growth regulators 0.2 mg/L naphthaleneacetic acid and 2 mg/L 6-benzylamino purine, and 2.5 g/L Phytagel.

In some embodiments, these events can be grown in a continuous light growth environment at 26-28° C. for root and shoot formation. In some embodiments, these events can be grown in a 16h/8h light/dark growth chamber at 26-28° C. for root and shoot formation.

In some embodiments, plants showing both root and shoot development after the previous step may be transferred to pots containing an artificial potting medium and gently acclimatized to greenhouse conditions. The plants may be grown to maturity and seed production in a greenhouse.

In some embodiments, immature scutella of a plant can be used. Approximately twenty-four hours prior to transformation, immature scutella approximately 2 mm in length of wheat cultivars Fielder and/or Bobwhite can be prepared for transformation by excising them from immature seeds, removing the small embryo axis, and plating them in a circular target zone on a high-osmotic medium. In some embodiments, the medium can comprise an agar-solidified MS basal medium supplemented with amino acids, sucrose and 2,4-D, with or without the addition of cefotaxime antibiotic at the rate of 250 mg/L for contamination control.

In some embodiments, the precultured wheat scutella can be transformed with one or with multiple plant enzyme expression constructs (e.g., a herbicide-resistant plant enzyme; a regulatory plant enzyme) using the biolistics method (particle bombardment). In some embodiments, the scutella can be co-transformed with a mitochondrial oligomycin resistance gene (oliR) linked to the plant enzyme described herein.

In some embodiments, immediately after bombardment, or up to 2 days after bombardment, the scutella can be spread out across the bombarded plates or spaced out onto additional new plates of the same high osmotic medium and incubated in the dark for up to 7 days at 26° C. In some embodiments, cultured scutella can be transferred to a selective callus induction medium (e.g., the MS-based high osmotic medium supplemented, an inhibitor of the plant enzyme and/or cefotaxime). In some embodiments, the culture can be in dark incubation for at least up to 1, 2, 3, 4, 5 weeks.

In some embodiments, after incubation on selective callus induction medium containing the inhibitor of the plant enzyme, the scutella can be continuously maintained on the MS-based selective callus induction medium with the inhibitor of plant enzyme (e.g., chlorsulfuron). In some embodiments, after incubation on selective callus induction medium containing the inhibitor of the plant enzyme, the scutella can be transferred to a first stage agarose-solidified regeneration medium (RZ) supplemented with maltose, 2,4-D, zeatin and silver nitrate in presence of the inhibitor of the plant enzyme. In some embodiments, cefotaxime use can be discontinued after three to six weeks of culture on the callus induction medium.

In some embodiments, the scutella on callus induction medium can be cultured in the light (16/8 photoperiod) and transferred to fresh medium with the inhibitor of the plant enzyme (e.g., chlorsulfuron) approximately every three weeks for 32 weeks. In some embodiments, calli induced from individual bombarded scutella can be subdivided into smaller pieces and maintain their original identity.

In some embodiments, scutella on shoot induction medium can be sub-cultured to fresh first stage regeneration medium every three weeks and cultured in the light until shoot formation is visible. In some embodiments, selected green sectors of callus and small shoots can be transferred to a second stage regeneration medium (R0) which is the same as the first stage regeneration medium, but without growth regulators. Developing plants can be transferred to domed clear culture vessels and grown on to transplantable size. In some embodiments, the developing plants can be transplanted to soil and acclimatized in the greenhouse.

### Dual Selection Process

In some embodiments, the methods described herein can have multiple selection processes. In some embodiments, the gene expression cassette comprising a plant enzyme or a variant thereof (e.g., a herbicide-resistant ALS) can be co-transformed with a second selectable marker expression cassette, for example, a 35S:HPT nuclear expression cassette conferring hygromycin B resistance. In some embodiments, a selective medium can comprise one or more inhibitors of the plant enzyme and selective agents (e.g., hygromycin B). In some embodiments, a selective medium can comprise one or more inhibitors of the plant enzyme (e.g., chlorsulfuron) and at least about 1-100 mg/L, 10-75 mg/L, or 25-50 mg/L of hygromycin B.

In some embodiments, the gene expression cassette comprising a plant enzyme or a variant thereof (e.g., a herbicide-resistant ALS) can be also linked and co-transformed with an oliR expression cassette, conferring resistance to the antibiotic oligomycin. In some embodiments, oligomycin can be incorporated into the selective medium at about 0.5-100 mg/L, 1-50 mg/L, 1-50 mg/L, or 1-5 mg/L. In some embodiments, the carbon source (e.g., sucrose) can be reduced to about 0.1% (1 mg/L), 0.2% (2 mg/L), 0.3% (3 mg/L), 0.4% (4 mg/L), 0.5% (5 mg/L) 0.6% (6 mg/L), 0.7% (7 mg/L), 0.8% (8 mg/L), 0.9% (9 mg/L) or replaced with at least about 10 mL, 20 mL, 30 mL, 40 mL, 50 mL, 60 ml, 70 mL, 80 mL, 90 mL, or 100 mL of a sterile 50% glycerol solution per L of selective medium to enhance the effectiveness of oligomycin.

In some embodiments, the compound disulfiram can be also incorporated into the selective medium at about 20 μM, 40 μM, 60 μM, 800 μM, 100 μM, 150 μM, 200 μM, 300 μM, 400 μM, or 500 μM, to inhibit the ability of cells to utilize any alcohol produced by anaerobic respiration of treated cells. In some embodiments, the gene expression cassette comprising a plant enzyme or a variant thereof (e.g., a herbicide-resistant ALS gene) can comprise of geminivirus VOR sequences.

In some embodiments, the gene expression cassette comprising a plant enzyme or a variant thereof (e.g., a herbicide-resistant ALS) can be co-transformed with a second selectable marker expression cassette comprising a polynucleotide encoding a phosphite dehydrogenase enzyme or a biologically active fragment thereof. In some embodiments, the selective medium can comprise one or more inhibitors of the plant enzyme and selective agents (e.g., phosphite). In some embodiments, the selective medium can comprise one or more inhibitors of the plant enzyme (e.g., chlorsulfuron) and at least about 0.1-0.25 mM, 0.25-0.5 mM, 0.5-0.75 mM, 0.75-1.0 mM, 1.0-2.5 mM, 2.5-5.0 mM, 5.0-7.5 mM, 7.5-10 mM, 10-15 mM, 15-20 mM, 20-25 mM, 25-30 mM, 30-35 mM, 35-40 mM, 40-45 mM, and 45-50 mM.

In some embodiments, the gene expression cassette comprising the plant enzyme or a variant thereof (e.g., a herbicide-resistant ALS gene) can be co-transformed with at least one expression cassette conferring resistance to an additional selection marker (e.g., conferring hygromycin B resistance or oligomycin resistance), at least two expression cassettes (e.g., conferring hygromycin B resistance and oligomycin resistance), or at least three expression cassettes (e.g., conferring hygromycin B resistance, oligomycin resistance and an additional selection agent). In some embodiments, one or more selective agents (e.g., hygromycin B) can be added in place of, or in addition to, the inhibitor of the plant enzyme. In some embodiments, variations can be made in the timing of the inception of one or more selection agents in conjunction with an inhibitor of the plant enzyme (e.g., chlorsulfuron). For example, after an initial period of one or more cycles of subculture and selection, the use of the plant enzyme can be discontinued and one or more selection agents (e.g., oligomycin) can be utilized.

### Inducible Expression System

In some embodiments, the target tissue for transformation (e.g., biolistics transformation) of the plant (e.g., rice) can be from a different source. The different source of callus tissue can be derived from a previous Agrobacterium tumefaciens transformation. In some embodiments, a dexamethasone-inducible system can be used to produce a geminivirus Rep protein. In some embodiments, to supply enough tissue for bombardment, the event can be maintained on the first selective medium, which can be supplemented with 40 mg/L hygromycin, amino acids, proline, maltose and 2,4-D growth regulator prior to the transformation.

In some embodiments, the tissue to be bombarded can be derived from the inducible line, precultured for 4 days prior to bombardment on the first selective medium. In some embodiments, the preculture medium can be also supplemented with 1,000 μl of 10 μM dexamethasone (DEX). In some embodiments, DMSO can be used as a control.

In some embodiments, calli can be prepared for bombardment by plating tissue in the target zone on the N6-based callus induction medium supplemented with mannitol and sorbitol for osmotic protection, but without DEX. After bombardment, the calli can be incubated in the dark for 16-20 hours at 26° C., then clumps of callus tissue approximately 1-3 mm in size can be sub-cultured to the N6-based callus maintenance medium supplemented with growth regulator 2,4-D and the appropriate selective agents including appropriate an inhibitor of the plant enzyme (e.g., 20-100 nM chlorsulfuron) and one or more additional selection marker (e.g., 25-50 mg/L hygromycin and 1-5 mg/L oligomycin) with reduced or alternate carbon source. In some embodiments, chemical induction with DEX in the medium can begin with the first round of selection. Calli on selective media with DEX can be then returned to dark incubation for the first round of selection. In some embodiments, DEX was introduced at a later point in the selection process. After 2-3 weeks of dark incubation, small (1-3 mm) clumps can be again sub-cultured to a fresh selective medium and then incubated as described. Additional rounds of sub-culturing to fresh selection medium with DEX and/or DMSO after 2 to 3-week periods of maintenance in the light can be most performed. In some embodiments, the induction with DEX can be continued throughout the entire selection process. In some embodiments, the use of DEX and/or DMSO can be discontinued for one or more culture periods. In some experiments, DEX can be re-introduced at a later point in the selection process.

### Screenable and Selectable Markers

In some embodiments, a polynucleotide (e.g., a donor polynucleotide) can also encode a phenotypic marker. In some embodiments, a phenotypic marker can be a screenable or a selectable marker that can include a visual screenable marker, a selectable marker, or a combination thereof. In some embodiments, a selectable marker can comprise a positive or negative selectable marker. In some embodiments, any phenotypic marker can be used. In some embodiments, a selectable or screenable marker can comprise a DNA segment that can allow one to identify or select for or against a molecule or a cell that contains it, e.g., under particular conditions. In some embodiments, a marker can encode an activity, such as, but not limited to, production of RNA, peptide, or protein, or can provide a binding site for RNA, peptides, proteins, inorganic and organic compounds or compositions and the like.

In some embodiments, an example of a selectable or screenable marker can include, but are not limited to, DNA segments that comprise restriction enzyme sites; DNA segments that encode products which provide resistance against otherwise toxic compounds including antibiotics, such as, spectinomycin, ampicillin, kanamycin, tetracycline, hygromycin; DNA segments that encode products which are otherwise lacking in a recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan fluorescent protein (CFP), yellow fluorescent protein (YFP), red fluorescent protein (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification, or any combination thereof.

In some embodiments, additional selectable markers can include polynucleotides that encode proteins that can confer resistance/tolerance to herbicidal compounds, such as glyphosate, sulfonylureas, glufosinate ammonium, bromoxynil, imidazolinones, and 2,4-dichlorophenoxyacetate (2,4-D). In some embodiments, a herbicide resistance protein can include a herbicide tolerant version of the following: an acetyl coenzyme A carboxylase (ACCase); a 4-hydroxyphenylpyruvate dioxygenase (HPPD); a sulfonylurea-tolerant acetolactate synthase (ALS); an imidazolinone-tolerant acetolactate synthase (ALS); a glyphosate-tolerant 5-enolpyruvylshikimate-3-phosphate synthase (EPSPS); a glyphosate-tolerant glyphosate oxidoreductase (GOX); a glyphosate N-acetyltransferase (GAT); a phosphinothricin acetyl transferase (PAT); a protoporphyrinogen oxidase (PPO or PROTOX); an auxin enzyme or receptor; a P450 polypeptide, or any combination thereof. In some embodiments, non-limiting examples of genes useful for conferring herbicide resistance in plants can include genes that encode the above proteins. In some embodiments, a neomycin phosphotransferase II (nptII) gene can encode a protein to provide resistance to antibiotics kanamycin and geneticin and a hygromycin phosphotransferase (HPT) gene can encode a protein to provide resistance to hygromycin.

In some embodiments, a DNA transformation of organellar genomes can be performed, for example, in plastids and mitochondria. In some embodiments, a selectable marker gene can include, for example, photosynthesis (atpB, tscA, psaA/B, petB, petA, ycf3, rpoA, rbcL), antibiotic resistance (rrnS, rrnL, aadA, nptII, aphA-6), herbicide resistance (psbA, bar, AHAS (ALS), EPSPS, HPPD, sul) and metabolism (BADH, codA, ARG8, ASA2) genes. In some embodiments, a sul gene from bacteria can comprise herbicidal sulfonamide-insensitive dihydropteroate synthase activity and can be used as a selectable marker when a protein product is targeted to plant mitochondria.

In some embodiments, a sequence encoding a marker can be incorporated into a genome of an organelle. In some embodiments, an incorporated sequence encoding a marker can be subsequently removed from a transformed organellar genome. In some embodiments, a removal of a sequence encoding a marker may be facilitated by a presence of direct repeats before and after a region encoding a marker. In some embodiments, removal of a sequence encoding a marker can occur via an endogenous homologous recombination system of an organelle or by use of a site-specific recombinase system such as cre-lox or a site-directed recombination method. In some embodiments, a site-directed recombination method can comprise FLP-FRT recombination.

In some embodiments, Caspase Activatable-GFP (CA-GFP) is a modified version of GFP in which fluorescence is completely quenched by appendage of a hydrophobic quenching peptide that tetramerizes GFP and prevents maturation of a chromophore. In some embodiments, a sequence of a CA-GFP protein can correspond to a GFP with a fusion of DEVDFQGPCNDSSDPLVVAASIIGILHLILWILDRL (SEQ ID NO: 2) at the carboxy terminus. In some embodiments, a caspase recognition sequence comprising the amino acids DEVD (SEQ ID NO: 3) can be present in CA-GFP between the fluorescence and the quenching domains. In some embodiments, GFP fluorescence can be fully restored in vivo by catalytic removal of a quenching peptide by cleavage with caspase. In some embodiments, a nucleic acid sequence encoding CA-GFP can be modified by replacement of a caspase recognition sequence with a mitochondrial RNA editing sequence. In some embodiments, an RNA editing sequence can be selected such that a C-to-U conversion results in creation of a stop codon in an mRNA. In some embodiments, expression of a nucleic acid sequence encoding a modified CA-GFP would result in quenching in a cytoplasm or in plastids but would produce fluorescence in mitochondria, thus providing a screenable marker. In some embodiments, a candidate RNA editing sequence for this purpose is present in a wheat mitochondrial cox2 gene at positions 449, 587 and 620 of a gene. In some embodiments, a candidate RNA editing sequence for this purpose that is present in a wheat mitochondrial cox2 gene at positions 449, 587 and 620 of a gene can comprise SEQ ID NO: 4, SEQ ID NO: 5, and SEQ ID NO: 6, respectively.

Disclosed herein in some embodiments, are methods that can provide transformation efficiency into an organelle (e.g., mitochondria, plastids) of, for example, at least about: 1%, 2%, 5%, 10%, 20%, 30%, 40%, 50%, 60%, 70%, 80%, 90%, or 100% transformation efficiency.

Use of Multiple Selectable and/or Screenable Markers

The systems and methods described herein may utilize at least one, at least two, at least three, at least four, or at least five selectable or screenable markers. Commonly used selectable marker genes in plant may include, for example, those that confer resistance or resistance to antibiotics, such as kanamycin and paromomycin (nptII), hygromycin B (aph IV), streptomycin or spectinomycin. (aadA) and gentamicin (aac3 and aacC4), or those that impart resistance or resistance to herbicides such as glufosinate (bar or pat), dicamba (DMO) and glyphosate (aroA or EPSPS). In some cases, a screenable marker may provide an ability to visually screen transformants such as luciferase or green fluorescent protein (GFP), or genes expressing known uidA genes (GUS) or beta glucuronidase of various chromogenic substrates. In some embodiments, one or more selectable or screenable markers may be used at different growth stages of a cell, a tissue, a propagation material, a seed, a pollen, a progeny, or any combination thereof. For example, a cell may be co-transformed with a first selectable marker (e.g., a gene that confers resistance to the antibiotic hygromycin) and a second selectable maker (a herbicide-resistant ALS-LS), and may grow in a presence of a first selective agent (hygromycin) and then subsequently in a presence of a second selective agent (e.g., an inhibitor of ALS) at different growth stage. The transformation may also be performed in the absence of selection during one or more stages or steps of development or regeneration of the transformed cell, tissue, propagation material, seed, pollen, progeny, or any combination thereof. In some embodiments, one or more selectable or screenable markers may be incorporated in different organelles (e.g., nucleus and mitochondrial genomes). In some embodiments, one or more selectable or screenable markers may be removed upon successful transformation.

### Herbicide-Resistant ALS Large Subunit as a Selectable Marker

The acetolactate synthase large subunit (ALS-LS; EC: 2.2.1.6) catalyzes the first common step of the biosynthetic pathway for the synthesis of the branched-chain amino acids leucine, isoleucine, and valine. Acetolactate synthase activity comprises conversion of two molecules of pyruvate to one molecule of acetolactate and one molecule of carbon dioxide. Acetolactate synthase is also known as acetohydroxyacid synthase (AHAS). In some cases, acetolactate synthase also has a regulatory small subunit (ALS-SS). In some cases, the regulatory small subunit stimulates activity of the acetolactate synthase catalytic large subunit seven- to ten-fold and confers sensitivity to inhibition by valine and activation by ATP. In yeast, ALS is in the mitochondria. In yeast, the genes for the large and small subunits are encoded in the nucleus and the primary translation products have mitochondrial targeting sequences. In plants and algae, ALS is in the plastid. In plants and algae, the genes for the large and small subunits of ALS are encoded in the nucleus and the primary translation products have plastid targeting sequences.

In some cases, inhibitors of ALS are used as herbicides by inhibiting the production of branch-chain amino acids. These inhibitors are not a chemistry class but rather a mechanism class having diverse chemistries. The ALS inhibitor family includes sulfonylureas (SUs), imidazolinones (IMIs), triazolopyrimidines (TPs), pyrimidinyl benzoates (PYBs), sulfonanilide, and sulfonylamino carbonyl triazolinones (SCTs). ALS herbicides do not bind to the catalytic site but instead at a site specific to herbicidal action. Consequently, resistance mutations have shown widely varying effects on normal ALS catalysis activity, i.e., positive, negative, and neutral. For example, resistance in Hordeum murinum due to a proline-to-serine substitution at amino acid 197 was found to increase ALS activity by 2-to-3 fold. Herbicide-resistance to ALS has been identified in many weed species.

In some embodiments, an herbicide-resistant ALS-LS can have the following; altered Km, altered Vmax, altered cofactor affinity, altered cofactor specificity, altered thermostability, altered feedback regulation, or any combination thereof.

In some embodiments, an herbicide-resistant ALS-LS can be identified from an herbicide-resistant weed population. In some embodiments, an herbicide-resistant weed population can be from the following species: Xanthium strumarium, Kochia scoparia, Amaranthus hybridus, Apera spica-venti, Amaranthus powellii, and Oryza sativa var. sylvantica. In some embodiments, an herbicide-resistant ALS-LS can have at least one mutation at any of the following amino acids: Ala-122, Pro-197, Ala-205, Asp-376, Arg-377, Trp-574, Ser-653, Gly-654, and any combination thereof. In some cases, the amino acid number is standardized to the Arabidopsis thaliana sequence.

In some embodiments, an herbicide-resistant ALS-LS can be intentionally selected, for example, by laboratory selections. In some embodiments, an herbicide-resistant population can be from the following species: Oryza sativa, Zea mays, Arabidopsis thaliana, Camelina sativa, Sorghum bicolor. In some embodiments, an herbicide-resistant ALS-LS can be from Oryza sativa and can have at least one of the following amino acid mutations: Trp548Leu, Ser627Ile, Trp548Met, Ser627Asn, and any combination thereof. In some embodiments, an herbicide-resistant ALS-LS can be from Zea mays and can have at least one of the following amino acid mutations: Pro165Ser, Pro165Ala, Pro165Leu, Pro165Trp. In some embodiments, an herbicide-resistant ALS-LS can be from Arabidopsis thaliana and can have at least one of the following amino acid mutations: Ser653Asn, Ala122Thr, Ala122Val, Ala205Val, Trp574Ser, Trp574Leu, Ser653Asn, Pro197Ala, and any combination thereof. In some embodiments, an herbicide-resistant ALS-LS can be from Camelina sativa and can have at least one of the following amino acid mutations: Ala122Thr, Pro197Ser, Trp574Leu, and any combination thereof. In some embodiments, an herbicide-resistant ALS-LS can be from Sorghum bicolor and can have at least one of the following amino acid mutations: Val531Ile, Trp545Leu, and any combination thereof.

In some embodiments, an herbicide-resistant ALS-LS can be of plant origin. In some embodiments, an herbicide-resistant ALS-LS can be lacking a chloroplast transit sequence. In some embodiments, a polynucleotide encoding an herbicide-resistant ALS-LS lacking a chloroplast transit sequence can be introduced into the mitochondria. In some embodiments, an enzyme can comprise both an herbicide-resistant ALS-LS and a regulatory ALS-SS. In some embodiments, an ALS-SS can be of plant origin. In some embodiments, an ALS-SS can be lacking a chloroplast transit sequence. In some embodiments, an ALS-SS is fused to a mitochondrial targeting sequence. In some embodiments, a polynucleotide encoding an ALS-SS fused to a mitochondrial targeting sequence can be introduced into the nuclear genome. In some embodiments, a polynucleotide encoding an ALS-SS lacking a chloroplast transit sequence can be introduced into the mitochondria. In some embodiments, an herbicide-resistant ALS-LS can be from Oryza sativa. In some embodiments, an ALS-SS can be from Oryza sativa. In some embodiments, the presence of an herbicide-resistant ALS-LS in mitochondria can enable synthesis in the cell of branched-chain amino acids (valine, leucine, and isoleucine) in the presence of an inhibitor of acetolactate synthase which can allow for its use as a selectable marker.

### Benefits of Organisms Having Mitochondria Transformed to Express a Polypeptide Having Herbicide-Resistant Enzyme Activity

In some embodiments, introduction into a mitochondrion of a polynucleotide encoding a polypeptide having herbicide-resistant enzyme activity can allow for selection of a plant cell or an algal cell having stably transformed mitochondria. In some embodiments, transformation of mitochondria with a polynucleotide encoding a polypeptide having herbicide-resistant enzyme activity can allow for co-transformation with an additional polynucleotide of interest. In some embodiments, at least 50%, 60%, 70%, 80%, 90%, or 100% of the mitochondrial genomes in a cell can be transformed. In some embodiments, a cell can be homoplasmic for the transformed mitochondria.

In some embodiments, the method described herein may promote growth or cultivation of a plant of interest comprising the edited mitochondrial genome, while suppressing the growth of an undesired plant (e.g., weed) that does not comprise the edited mitochondrial genome. For example, a plurality of plants may be grown in a presence of an inhibitor of plant enzyme described herein (e.g., ALS, EPSPS, GS), wherein at least one desired plant of the plurality of plants comprises a mitochondrion having a heterologous polynucleotide that encodes a polypeptide having herbicide-resistant plant enzyme activity or a biologically active fragment thereof and at least one undesired plant (e.g., weed) of the plurality of plants lacking a mitochondrion having a heterologous polynucleotide that encodes a polypeptide having herbicide-resistant acetolactate synthase activity or a biologically active fragment thereof. In some embodiments, the presence of the inhibitor of the plant enzyme is sufficient to selectively promote growth of the at least one desired plant of the plurality of plants, resulting in an increased growth of the at least one desired plant of the plurality of plants relative to undesired plants (e.g., weed) lacking a polypeptide having herbicide-resistant acetolactate synthase activity or a biologically active fragment thereof. In some embodiments, the inhibitor of the plant enzyme may be applied to the plant, the plurality of plants, soil adjacent to the plants or any combination thereof. In some embodiments, the inhibitor of the plant enzyme is applied as a foliar amendment, a soil amendment, or any combination thereof. In some embodiments, the inhibitor of the plant enzyme may be dissolved in water and applied to the plant, the plurality of plants, soil adjacent to the plants or any combination thereof.

In some embodiments, a plant having mitochondria transformed with a polynucleotide encoding a polypeptide having herbicide-resistant plant enzyme activity can transmit the transformed mitochondria to progeny plants by maternal inheritance. In some embodiments, a plant having mitochondria transformed with a polynucleotide encoding a polypeptide having herbicide-resistant plant enzyme activity can have less horizontal gene transfer (e.g., to a weed species) than a plant having a nuclear genome transformed with a polynucleotide encoding a polypeptide having herbicide-resistant acetolactate synthase activity.

### Methods Utilizing a Two Component RNA Guide and Polynucleotide Guided Polypeptide System

In some embodiments, a polynucleotide guided polypeptide system described herein can be especially useful for genome engineering in circumstances where endonuclease off-target cutting can be toxic to a targeted cell. In some embodiments, a polynucleotide guided polypeptide system described herein, a constant component, a polynucleotide encoding an organelle targeted polynucleotide guided polypeptide, can be stably integrated into a nuclear genome of a cell. In some embodiments, a polynucleotide encoding an organelle targeted polynucleotide guided polypeptide can be transiently expressed in a nuclear genome of a cell. In some embodiments, a polynucleotide can encode a modified polynucleotide guided polypeptide comprising an enzymatically active polynucleotide guided polypeptide (e.g., Cas polypeptide, a MAD polypeptide) fused to an organellar transport sequence (e.g., a mitochondrial targeting peptide or a chloroplast targeting peptide). In some embodiments, an expression of a polynucleotide encoding a modified polynucleotide guided polypeptide can be under control of a promoter. In some embodiments, a promoter can be a constitutive promoter, a tissue-specific promoter, or an inducible promoter, e.g., a temperature-inducible, stress-inducible, developmental stage inducible, or chemically inducible promoter. In some cases, in the absence of a variable component (e.g., a guide RNA or crRNA), a polynucleotide guided polypeptide may not cut a target nucleic acid. In an absence of a variable component (e.g., a guide RNA or crRNA) a presence of a polynucleotide guided polypeptide in a cell (e.g., a plant cell) may have little or no consequence. In some embodiments, a polynucleotide guided polypeptide system can be used to create and/or maintain a cell line or transgenic organism capable of efficient expression of a polynucleotide guided polypeptide. Expression of a polynucleotide guided polypeptide in a cell line or transgenic organism may have little or no consequence to cell viability.

In some embodiments, in order to induce cutting at desired genomic sites to achieve targeted genetic modifications, guide polynucleotides (e.g., guide RNAs or crRNAs) can be introduced by a variety of methods into cells containing a stably-integrated and expressed expression cassette for a polynucleotide guided polypeptide. In some embodiments, a guide polynucleotide (e.g., guide RNAs or crRNAs) can be chemically or enzymatically synthesized and introduced into a polynucleotide guided polypeptide expressing cells via direct delivery methods such a particle bombardment or electroporation. In some embodiments, a guide polynucleic acid can be fused to an RNA molecule that allows for transport into an organelle. In some embodiments, a guide polynucleic acid can be fused to an RNA molecule that allows for binding to a protein that facilitates transport into an organelle. In some embodiments, a guide polynucleic acid can be transported into an organelle by association with a modified polynucleotide guided polypeptide comprising an enzymatically active polynucleotide guided polypeptide fused to an organellar transport sequence.

In some embodiments, a gene can efficiently express a guide polynucleotide in a target cell. In some embodiments a guide polynucleotide can comprise a guide RNAs, a crRNAs, or a combination thereof. In some embodiments a gene that can efficiently express a guide polynucleotide in a target cell can be synthesized chemically, enzymatically or in a biological system. In some embodiments, a gene that can efficiently express a guide polynucleotide in a target cell can be introduced into a polynucleotide guided polypeptide expressing cell, via direct delivery methods, biological delivery methods, or a combination thereof. In some embodiments, a direct delivery method can comprise a particle bombardment, an electroporation, a vacuum infiltration, or any combination thereof. In some embodiments, a biological delivery method can comprise an Agrobacterium-mediated DNA delivery method.

In some embodiments, a method for altering a genome of an organelle can comprise: introducing into an organelle a first polynucleotide encoding at least one guide polynucleic acid. In some embodiments, at least one guide polynucleic acid can direct a polynucleotide guided polypeptide to cleave at least one target sequence present in an organelle genome. In some embodiments, a guide polynucleic acid can comprise a guide RNA. In some embodiments, a polynucleotide guided polypeptide can comprise a Cas polypeptide, a Cas9 polypeptide or a combination thereof. In some embodiments, a method can further comprise introducing into an organelle a second polynucleotide. In some embodiments, a second polynucleotide can encode a polynucleotide guided polypeptide. In some embodiments, a polynucleotide guided polypeptide, when associated with a guide polynucleic acid can cleave at least one target sequence. In some embodiments, a method can further comprise introducing into an organelle a third polynucleotide encoding at least one homologous organelle DNA sequence. In some embodiments, at least one homologous organelle DNA can be of sufficient size for homologous recombination. In some embodiments, integration of at least one homologous organelle DNA sequence into an organelle genome can result in removal of at least one target sequence. In some embodiments, an organelle can comprise a mitochondrion, a plastid, or a combination thereof.

Disclosed herein in some embodiments, are methods for selecting a plant comprising an altered organellar genome. In some embodiments, a method can be used to identify those cells having an altered genome at or near a target site without using a screenable or selectable marker phenotype. In some embodiments, a method can comprise directly analyzing a target sequence to detect any change in a target sequence, including but not limited to PCR methods, sequencing methods, nuclease digestion, Southern blots, and any combination thereof.

In some embodiments, sufficient homology or sequence identity can indicate that two polynucleotide sequences can have sufficient structural similarity to act as substrates for a homologous recombination reaction. In some embodiments, a structural similarity can include an overall length of each polynucleotide fragment, a sequence similarity of each polynucleotide, or a combination thereof. In some embodiments, a sequence similarity can be described by a percent sequence identity over a whole length of multiple sequences, by conserved regions comprising localized similarities such as contiguous nucleotides having 100% sequence identity, by percent sequence identity over a portion of a length of multiple sequences, or any combination thereof.

In some embodiments, an amount of homology or sequence identity shared by a target and a donor polynucleotide can vary. For example, a length of sequence homology can be at least about 20 bp, at least about 50 bp, at least about 100 bp, at least about 150 bp, at least about 250 bp, at least about 300 bp, at least about 400 bp, at least about 500 bp, at least about 600 bp, at least about 700 bp, at least about 800 bp, at least about 900 bp, at least about 1000 bp, at least about 1250 bp, at least about 1500 bp, at least about 1750 bp, at least about 2000 bp, at least about 2.5 kb, at least about 3 kb, at least about 4 kb, at least about 5 kb, at least about 6 kb, at least about 7 kb, at least about 8 kb, at least about 9 kb, or at least about 10 kb. In some embodiments, an amount of homology can also be described by a percent sequence identity over a full aligned length of two polynucleotides which can include a percent sequence identity of at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In some embodiments, sufficient homology can include any combination of polynucleotide length, global percent sequence identity, conserved regions of contiguous nucleotides, local percent sequence identity, or any combination thereof. In some embodiments, a sufficient homology can be described as a region of 75-150 bp having at least 80% sequence identity to a region of a target locus. In some embodiments, a sufficient homology can also be described by a predicted ability of two polynucleotides to specifically hybridize under high stringency conditions.

In some embodiments, a plant cell having an introduced sequence can be grown or regenerated into a plant. In some embodiments, a plant can then be grown, and either pollinated with a same transformed strain or with a different transformed or untransformed strain, and a resulting progeny having a desired characteristic and/or comprising an introduced polynucleotide or polypeptide identified. In some embodiments, two or more generations can be grown to ensure that a polynucleotide can be stably maintained and inherited, and seeds harvested.

In some embodiments, any plant can be used. In some embodiments, a plant can comprise a monocot, or a dicot plant. In some embodiments, a monocot plant can comprise a corn (Zea mays), a rice (Oryza sativa), a rye (Secale cereale), a sorghum (Sorghum bicolor, Sorghum vulgare), a millet (e.g., pearl millet (Pennisetum glaucum), a proso millet (Panicum miliaceum), a foxtail millet (Setaria italica), a finger millet (Eleusine coracana)), a maize, a wheat (Triticum aestivum), a sugarcane (Saccharum spp.), an oat (Avena), a barley (Hordeum), a switchgrass (Panicum virgatum), a pineapple (Ananas comosus), a banana (Musa spp.), a palm, an ornamental, a turfgrass, another grass, or any combination thereof. In some embodiments, a dicot plant can comprise a soybean (Glycine max), a canola (Brassica napus and B. campestris), an alfalfa (Medicago sativa), a tobacco (Nicotiana tabacum), an Arabidopsis (Arabidopsis thaliana), a sunflower (Helianthus annuus), a cotton (Gossypium arboreum), a peanut (Arachis hypogaea), a tomato (Solanum lycopersicum), a potato (Solanum tuberosum), or any combination thereof.

In some embodiments, after creating a designed change in an organellar DNA, a next step can be to maintain an edited organellar DNA in a pool of unmodified organellar DNA and to shift a balance among organellar DNA to favor a maintenance of genome edited organellar DNA. In some embodiments, this can be achieved by reducing an amplification of unmodified organellar DNA. In some embodiments, guide polynucleic acids can be designed for multiple target sites in an unmodified organelle genome. In some embodiments, a donor polynucleotide can comprise a donor DNA. In some embodiments, a donor polynucleotide can be designed such that a target site has been altered to no longer be recognized by a relevant polynucleotide guided polypeptide system. In some embodiments, an expression of a polynucleotide guided polypeptide can result in an introduction of single-strand or double-strand breaks into an unmodified organellar DNA and can thereby increase a proportion of modified genomes. In some embodiments, a cell can be pretreated with relevant polynucleotide guided polypeptide systems to introduce cleavages in organellar DNA. In some embodiments, a pretreatment can reduce a number of organelle DNA molecules available for homologous recombination.

In some embodiments, a cell may be selected that is homoplasmic for an altered genome of an organelle. In some embodiments, a cell may be selected that comprises a plurality of mitochondrial genomes, wherein at least 10%-100% of the plurality of mitochondrial genomes comprise the edited mitochondrial genome. In some embodiments, the selected cell may comprise a plurality of mitochondrial genomes that is about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% of the plurality of mitochondrial genomes comprise the edited mitochondrial genome. In some embodiments, the selected cell may comprise a plurality of mitochondrial genomes that is about 10%, about 20%, about 30%, about 40%, about 50%, about 60%, about 70%, about 80%, about 90%, or about 100% of the plurality of mitochondrial genomes comprise the edited mitochondrial genome. In some embodiments, the selected cell may comprise a plurality of mitochondrial genomes that is at least about 10%, about 20%, about 30%, about 40%, about 50%, about 60%, about 70%, about 80%, or about 90% of the plurality of mitochondrial genomes comprise the edited mitochondrial genome. In some embodiments, the selected cell may comprise a plurality of mitochondrial genomes that is at most about 20%, about 30%, about 40%, about 50%, about 60%, about 70%, about 80%, about 90%, or about 100% of the plurality of mitochondrial genomes comprise the edited mitochondrial genome. In some embodiments, an organelle can comprise a nucleus, a mitochondrion, a plastid, or a combination thereof.

In some embodiments, a method can comprise use of a single guide RNA (sgRNA). In some embodiments, a variable targeting domain can be fused to a polynucleotide that contains a tracrRNA sequence. In some embodiments, a method can comprise use of a duplex guide RNA. In some embodiments, a variable targeting domain and a tracrRNA sequence can be present on separate RNA molecules. In some embodiments, the terms “duplex guide RNA” and “dual guide RNA” can be used interchangeably.

In some embodiments, an expression level of a protein, an RNA, or a combination thereof can be higher when transformed into a plastid or mitochondrion as compared with that in a nucleus. In some embodiments, a protein and/or an RNA expression level can be at least about: 1%, 5%, 10%, 20%, 30%, 40%, 50%, 60%, 70%, 80%, or 90% higher with transformation of plastid or mitochondrial DNA as compared with a nuclear DNA transformation. In some embodiments, an expression stability of a protein, a transcript, or a combination thereof can be higher with a plastid or a mitochondrial transformation as compared with a nuclear transformation.

### Methods for Delivery

In some embodiments, any suitable delivery method can be used for introducing a composition and molecule disclosure herein into a host cell or organelle. In some embodiments, an organelle can comprise a mitochondrion, a plastid, or a combination thereof. In some embodiments, a host cell can comprise a yeast cell, a plant cell, or a combination thereof. In some embodiments, a composition can comprise a Cas protein, a polynucleotide-guided polypeptide, a guide polynucleic acid, a donor polynucleotide, a nucleic acid encoding a composition, or any combination thereof. In some embodiments, a composition can be delivered simultaneously or temporally separated. In some embodiments, a choice of method of genetic modification can be dependent on a type of cell being transformed, a circumstance under which a transformation is taking place, or a combination thereof. In some embodiments, a circumstance under which a transformation is taking place can be in vitro, ex vivo, in vivo, in planta, or any combination thereof.

In some embodiments, a delivery method or transformation can include, a viral or bacteriophage infection, a transfection, a conjugation, a protoplast fusion, a lipofection, an electroporation, a calcium phosphate precipitation, a polyethyleneimine (PEI)-mediated transfection, a DEAE-dextran mediated transfection, a liposome-mediated transfection, a particle gun technology, a calcium phosphate precipitation, a direct micro injection, a nanoparticle-mediated nucleic acid delivery, a lipid nanoparticle, lipid-based vectors, polymeric vectors, polyethylenimine, poly(L-lysine), a vacuum infiltration, or any combination thereof.

In some embodiments, a DNA transformation can comprise a yeast nuclear genome transformation. In some embodiments, a DNA transformation can be facilitated by a development of shuttle vectors that can replicate in E. coli and yeast as autonomous plasmids. In some embodiments, a vector system can include low-copy-number plasmids and integrative DNA through homologous recombination.

In some embodiments, disclosed herein are methods comprising delivering a polynucleotide as described herein, a vector as described herein, a transcript thereof, a protein translated therefrom, or any combination thereof to a host cell or organelle. In some embodiments, disclosed herein is a cell produced by a method disclosed herein, an organism produced by a method disclosed herein, an organelle comprising or produced from a cell disclosed herein, or any combination thereof. In some embodiments, an organism can comprise an animal, a plant, a fungus, or a combination thereof. In some embodiments, a polynucleotide guided polypeptide in combination with, and optionally complexed with, a guide sequence can be delivered to a cell or an organelle.

In some embodiments, a method to introduce nucleic acids can comprise viral based gene transfer methods, non-viral based gene transfer methods, or a combination thereof. In some embodiments, a method can be used to administer a nucleic acid encoding a composition of a disclosure to a cell in culture, or in a host organism. In some embodiments, a non-viral vector delivery system can include a DNA plasmid, an RNA, a naked nucleic acid, a nucleic acid complexed with a delivery vehicle, or any combination thereof. In some embodiments, a delivery vehicle can comprise a liposome. In some embodiments, an RNA can comprise a transcript of a vector described herein. In some embodiments, a viral vector delivery system can include a DNA virus, an RNA virus, or a combination thereof. In some embodiments, a viral vector delivery system can have either episomal or integrated genomes after delivery to a cell. In some embodiments, a viral vector based system for gene transfer can comprise a retrovirus, a lentivirus, an adenovirus, an adeno-associated virus, a herpes simplex virus, or any combination thereof.

In some embodiments, an adenoviral-based system can be used. In some embodiments, an adenoviral-based system can lead to a transient expression of a transgene. In some embodiments, an adenoviral based vector can have a high transduction efficiency in cells and may not require cell division. In some embodiments, a high titer, high levels of expression, or a combination thereof can be obtained with an adenoviral based vector. In some embodiments, an adeno-associated virus (“AAV”) vector can be used to transduce a cell with a target nucleic acid. In some embodiments, a vector can be used to transduce a cell with a target nucleic acid for an in vitro production of nucleic acids and peptides, for in vivo and ex vivo gene therapy procedures, or any combination thereof.

In some embodiments, a cell transfected with one or more vectors described herein can be used to establish a new cell line comprising one or more vector-derived sequences. In some embodiments, a cell can be transiently transfected with a composition disclosed herein. In some embodiments, transient transfection can comprise transient transfection of one or more vectors, transfection with RNA, or a combination thereof. In some embodiments, a transiently transfected cell can be modified through an activity of a CRISPR complex. In some embodiments, a cell modified through an activity of a CRISPR complex can be used to establish a new cell line comprising cells containing a modification but lacking any other heterologous sequence.

In some embodiments, a composition disclosed herein can be provided as an RNA. In some embodiments, a composition disclosed herein can be produced by direct chemical synthesis or may be transcribed in vitro from a DNA. In some embodiments, a composition disclosed herein can be synthesized in vitro using an RNA polymerase enzyme. In some embodiments, an RNA polymerase enzyme can comprise a T7 polymerase, a T3 polymerase, an SP6 polymerase, or any combination thereof. In some embodiments, an RNA can directly contact a target polynucleic acid. In some embodiments, a target polynucleic acid can comprise a target DNA. In some embodiments, a target polynucleic acid can be introduced into a cell using any suitable technique for introducing nucleic acid into a cell. In some embodiments, a suitable technique for introducing a nucleic acid into a cell can comprise a microinjection, an electroporation, a transfection, or any combination thereof.

In some embodiments, a polynucleotide encoding a guide nucleic acid can comprise DNA or RNA. In some embodiments, a polynucleotide encoding a polynucleotide guided polypeptide can comprise DNA, RNA, or a combination thereof. In some embodiments, a polynucleotide encoding a guide nucleic acid and a polynucleotide guided polypeptide can be provided to a cell using a suitable transfection technique. In some embodiments, a nucleic acid encoding a composition of a disclosure can be provided on a vector or a cassette. In some embodiments, a vector or a cassette can comprise a DNA vector. In some embodiments, a vector can comprise a plasmid, a cosmid, a minicircle, a phage, a virus, or any combination thereof. In some embodiments, a vector can transfer a nucleic acid into a target cell. In some embodiments, a vector comprising a nucleic acid can be maintained episomally. In some embodiments, a vector comprising a nucleic acid can comprise a plasmid, a minicircle DNA, a virus, or any combination thereof. In some embodiments, a virus can comprise a cytomegalovirus, an adenovirus, or a combination thereof. In some embodiments, a vector comprising a nucleic acid can be integrated into a target cell genome, through homologous recombination or random integration, e.g., retrovirus-derived vectors such as MMLV, HIV-1, and ALV.

In some embodiments, a polynucleotide guided polypeptide can be provided to cells as a polypeptide. In some embodiments, a protein can be fused to a polypeptide domain that increases solubility of a product. In some embodiments, a domain can be linked to a polypeptide through a defined protease cleavage site, e.g., a TEV sequence, which can be cleaved by a TEV protease. In some embodiments, a linker can comprise a flexible sequence. In some embodiments, a flexible sequence can comprise from 1 to 10 glycine residues.

In some embodiments, a composition as disclosed herein can be operably linked (e.g., covalently, or non-covalently) to a polypeptide permeant domain to promote uptake by a cell or an organelle. In some embodiments, a polynucleotide composition can comprise a DNA, an RNA, or a combination thereof. In some embodiments, a disclosure can be associated with a peptide-based polynucleotide carrier that can comprise two functional units: a polynucleotide-binding domain (e.g., a polycationic KH repeat domain) and a polypeptide permeant domain.

In some embodiments, a number of polypeptide permeant domains can be used in a non-integrating polypeptide as disclosed herein, including a peptide, a peptidomimetic, a non-peptide carrier, and any combination thereof. In some embodiments, the terms “permeant peptide”, “cell penetrating peptide”, “CPP”, “protein transduction domain” and “PTD” can be used interchangeably herein. In some embodiments, a permeant peptide can be derived from a third alpha helix of Drosophila melanogaster transcription factor Antennapaedia, referred to as penetratin. In some embodiments, a CPP can comprise an amino acid sequence as described in SEQ ID NOS: 10, 11, 12, 13, 14, 15, 16, 17, 18, 19, 20, 21, 22, 23, 24, 25, 26, or 27 of International Patent Application PCT/US22/80942, herein incorporated by reference. In some embodiments, a permeant peptide can comprise an HIV-1 tat basic region amino acid sequence, which can include, for example, amino acids 49-57 of a naturally-occurring tat protein. In some embodiments, a permeant domain can include a poly-arginine motif. In some embodiments, a poly-arginine motif can comprise a region of amino acids 34-56 of an HIV-1 rev protein, a nona-arginine, an octa-arginine, or any combination thereof. In some embodiments, a nona-arginine (R9) sequence can be used. In some embodiments, other cell penetrating peptides can include: Pep-1, MPG, gamma-ZEIN, Transportan, MAP, Pept 1, Pept 2, IVV-14, Ig (v), Amphiphilic model peptide, pVEC, HRSV, Bp100 TAT2 or any combination thereof. In some embodiments, a composition as disclosed herein can be fused to a combination of a polypeptide permeant domain. In some embodiments, a site at which a fusion can be made can be selected in order to optimize a biological activity, secretion, or binding characteristics of a polypeptide.

In some embodiments, a polynucleotide composition can comprise a DNA, an RNA, or any combination thereof. In some embodiments, a polynucleotide composition disclosed herein can be associated with a peptide-based polynucleotide carrier that can comprise an organellar targeting signal. In some embodiments, for organelle-specific delivery, a peptide-based polynucleotide carrier can comprise two functional units: a polynucleotide-binding domain (e.g., a polycationic KH repeat domain) and an organelle-targeting peptide (e.g., a chloroplast transit peptide, a mitochondrial targeting peptide).

Disclosed herein are compositions that can be prepared by in vitro synthesis. In some embodiments, various commercial synthetic apparatuses can be used. In some embodiments, by using synthesizers, naturally occurring amino acids can be substituted with unnatural amino acids. In some embodiments, a particular sequence and a manner of preparation can be determined by convenience, economics, and purity required.

In some embodiments, where two or more different targeting complexes can be provided to a cell (e.g., two different guide nucleic acids that are complementary to different sequences within a same or different target DNA), a complex can be provided simultaneously (e.g., as two polypeptides and/or nucleic acids). In some embodiments, two or more different targeting complexes can be provided consecutively, e.g., a targeting complex being provided first, followed by a second targeting complex, or vice versa.

In some embodiments, in cases in which a targeting complex and a donor DNA can be provided to a cell, a targeting complex and donor DNA can be provided simultaneously. In some embodiments, a targeting complex and a donor DNA can be provided consecutively, e.g., a targeting complex(es) being provided first, followed by a donor DNA, or vice versa.

### Methods of Plant Growth

In some embodiments, a cell, a plant, a transgenic seed, a progeny plant, or a transgenic plant comprising one or more exogenous polynucleotides in edited mitochondria genome described herein may be grown in a temperature-controlled incubator, a bioreactor, a greenhouse, or a combination thereof. In some cases, the temperature-controlled incubator and/or greenhouse is further configured to control a light-dark cycle. In some embodiment, a cell, a plant, a transgenic seed, a progeny plant, or a transgenic plant can be grown in darkness for predetermined duration in predetermined temperature. In some embodiments, a cell, a plant, a transgenic seed, a progeny plant, or a transgenic plant can be grown in darkness for 16-20 hours at 26° C. In some embodiments, a plant, a transgenic seed, a progeny plant, or a transgenic plant can be grown in a continuous light growth environment at 26-28° C. for root and shoot formation. In some embodiments, a plant, a transgenic seed, a progeny plant, or a transgenic plant can be grown in a 16h/8h light/dark growth chamber at 26-28° C. for root and shoot formation. In some embodiments, a progeny plant or a transgenic plant showing both root and shoot development may be transferred to pots containing an artificial potting medium and gently acclimatized to greenhouse conditions. In some embodiments, a plant, a transgenic seed, a progeny plant, or a transgenic plant can be grown in a field. In some embodiments, a field may be treated with an inhibitor of acetolactate synthase.

### Compositions and Kits

Also provided herein are compositions that include any of the polynucleotides, polypeptides, vectors, or reagents (e.g., phosphite) described herein. Any of the compositions can include any of the polynucleotides, polypeptides, vectors, or reagents described herein and one or more (e.g., 1, 2, 3, 4, or 5) acceptable carriers or diluents. In some embodiments, the kit can include a cell, a tissue, a propagation material, a seed, a pollen, a progeny, or any combination thereof.

In some embodiments, any of the compositions described herein can include one or more buffers (e.g., a neutral-buffered saline, a phosphate-buffered saline (PBS)), one or more growth regulators (e.g., naphthaleneacetic acid, 6-benzylamino purine, phytagel), and one or more medium (e.g., germination medium, growth medium, maturation medium, phosphite medium).

In some embodiments, any of the compositions described herein can further include one or more (e.g., 1, 2, 3, 4, or 5) agents that promote the entry of any of the vectors or nucleic acids described herein into a cell (e.g., a plant cell).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers, and poly(lactic-co-glycolic acid) (PLGA) polymers.

Also provided are kits that include any of the compositions described herein that include any of the polynucleotides, any of the polypeptides, any of the reagents, or any of the vectors described herein.

In some embodiments, the kit can include instructions for performing any of the methods described herein.

### EXEMPLARY EMBODIMENTS

The following non-limiting embodiments provide illustrative examples of the invention, but do not limit the scope of the invention.

Embodiment 1. A method for transforming a mitochondrion, the method comprising:
a) introducing into the mitochondrion of a cell, wherein the cell is a plant cell or an algal cell, a first polynucleotide encoding a first polypeptide to generate a transformed mitochondrion, wherein the first polypeptide has herbicide-resistant acetolactate synthase activity;
b) growing the cell under conditions wherein the first polypeptide is expressed;
c) growing the cell in a medium wherein an inhibitor of acetolactate synthase is present; and
d) selecting a cell comprising the transformed mitochondrion, wherein the transformed mitochondrion comprises the first polynucleotide.

Embodiment 2. A method for transforming a mitochondrion, the method comprising:
a) introducing into the mitochondrion of a cell, wherein the cell is a plant cell or an algal cell:
   i) a first polynucleotide encoding a first polypeptide to generate a transformed mitochondrion, wherein the first polypeptide has herbicide-resistant acetolactate synthase activity, and
   ii) an additional polynucleotide encoding a selectable marker;
b) growing the cell under conditions wherein the selectable marker is expressed;
c) growing the cell in a medium wherein a selective agent of the selectable marker is present; and
d) selecting a cell comprising the transformed mitochondrion, wherein the transformed mitochondrion comprises the first polynucleotide.

Embodiment 3. The method of embodiment 2, wherein the selectable marker encodes a product which provides resistance against an otherwise toxic compound.

Embodiment 4. The method of embodiment 2, wherein the selectable marker is a phosphite dehydrogenase enzyme or a biologically active fragment thereof, and wherein the selective agent is a phosphite.

Embodiment 5. A method for transforming a mitochondrion, the method comprising introducing a heterologous first polynucleotide encoding a first polypeptide into the mitochondrion of a cell, wherein the cell comprises a plant cell or an algal cell, wherein the first polypeptide comprises an acetolactate synthase enzyme or a biologically active fragment thereof.

Embodiment 6. The method of embodiment 5, wherein the acetolactate synthase enzyme or the biologically active fragment thereof has herbicide-resistant activity.

Embodiment 7. The method of embodiment 5 or 6, further comprising growing the cell under conditions wherein the first polypeptide is expressed.

Embodiment 8. The method of any one of embodiments 1-7, wherein the transformed mitochondrion comprises an edited mitochondrial genome comprising the first polynucleotide.

Embodiment 9. The method of any one of embodiments 5-8, further comprising growing the cell in a medium comprising an inhibitor of acetolactate synthase.

Embodiment 10. The method of embodiment 2, wherein the selective agent comprises an inhibitor of acetolactate synthase.

Embodiment 11. The method of embodiment 1, 9, or 10, wherein the inhibitor of acetolactate synthase comprises a sulfonylurea, an imidazolinone, a triazolopyrimidine, a pyrimidinyl benzoate, a sulfonanilide, a sulfonylaminocarbonyltriazolinone, a salt of any of these, a stereoisomer of any of these, or any combination thereof.

Embodiment 12. The method of embodiment 11, wherein the sulfonylurea comprises a chlorsulfuron.

Embodiment 13. The method of any one of embodiments 1-4 or 9-12, wherein the medium comprises chlorsulfuron at a concentration of 20 nM-100 nM, 100 nM-1 μM, 1 μM-20 μM, or 20 μM-100 μM.

Embodiment 14. The method of embodiment 11, wherein the imidazolinone comprises an imazapyr, an imazapic, an imazethapyr, an imazamox, an imazamethabenz, an imazaquin, a salt of any of these, a stereoisomer of any of these, or any combination thereof.

Embodiment 15. The method of embodiment 11, wherein the triazolopyrimidine comprises a penoxsulam, a cloransulam-methyl, a diclosulam, a florasulam, a flumetsulam, a metosulam, a pyroxsulam, a salt of any of these, a stereoisomer of any of these, or any combination thereof.

Embodiment 16. The method of embodiment 11, wherein the pyrimidinyl benzoate comprises a bispyribac-sodium, a pyribenzoxim, a pyrithiobac-sodium, a salt of any of these, a stereoisomer of any of these, or any combination thereof.

Embodiment 17. The method of embodiment 11, wherein the sulfonanilide comprises a pyrimisulfan, a triafamone, a salt of any of these, a stereoisomer of any of these, or any combination thereof.

Embodiment 18. The method of embodiment 11, wherein the sulfonylaminocarbonyltriazolinone comprises a Flucarbazone-Na, a propoxycarbazone-Na, a thiencarbazone-methyl, a salt of any of these, a stereoisomer of any of these, or any combination thereof.

Embodiment 19. The method of embodiment 11, wherein the sulfonylurea comprises an amidosulfuron, an azimsulfuron, a bensulfuron-methyl, a chlorimuron-ethyl, a chlorsulfuron, a cinosulfuron, a cyclosulfamuron, an ethametsulfuron-methyl, an ethoxysulfuron, a flazasulfuron, a flucetosulfuron, a flupyrsulfuron-methyl-na, a foramsulfuron, a halosulfuron-methyl, an imazosulfuron, an iodosulfuron-methyl-na, a mesosulfuron-methyl, a metazosulfuron, a metsulfuron-methyl, a nicosulfuron, an orthosulfamuron, an oxasulfuron, a primisulfuron-methyl, a propyrisulfuron, a prosulfuron, a pyrazosulfuron-ethyl, a rimsulfuron, a sulfometuron-methyl, a sulfosulfuron, a thifensulfuron-methyl, a triasulfuron, a tribenuron-methyl, a trifloxysulfuron-na, a triflusulfuron-methyl, a tritosulfuron, a salt of any of these, a stereoisomer of any of these, or any combination thereof.

Embodiment 20. The method of any one of embodiments 1-19, wherein the method further comprises introducing into the mitochondrion a second polynucleotide encoding a regulatory subunit of an acetolactate synthase or a biologically active fragment thereof and growing the cell under conditions wherein the second polypeptide is expressed.

Embodiment 21. The method of any one of embodiments 1-19, wherein the method further comprises introducing into a nucleus of the cell a third polynucleotide encoding a modified regulatory subunit of an acetolactate synthase or a modified biologically active fragment thereof, wherein the modified regulatory subunit of the acetolactate synthase or the modified biologically active fragment thereof comprises a mitochondrial targeting peptide, and growing the cell under conditions wherein the third polypeptide is expressed.

Embodiment 22. The method of embodiment 21, wherein the third polynucleotide encodes a third polypeptide that comprises at least 70%, 80%, 85%, 90%, 91%, 92%, 93%, 94%, 95% or 99% sequence identity to SEQ ID NO: 21.

Embodiment 23. The method of embodiment 20, wherein the method further comprises selecting a cell wherein the transformed mitochondrion comprises the second polynucleotide.

Embodiment 24. The method of any one of embodiments 21-22, wherein the method further comprises selecting a cell comprising a transformed nucleus, wherein the transformed nucleus comprises the third polynucleotide.

Embodiment 25. The method of any one of embodiments 1-24, wherein the cell is a plant cell selected from the group consisting of: a wheat cell, a maize cell, a rice cell, a barley cell, a sorghum cell, a rye cell, a canola cell, a broccoli cell, a cauliflower cell, and a soybean cell.

Embodiment 26. The method of any one of embodiments 1-25, wherein the method further comprises
introducing into the mitochondrion of the cell a donor DNA, wherein the donor DNA comprises:
a) a fourth polynucleotide, wherein the fourth polynucleotide is heterologous to the mitochondrion;
b) a fifth polynucleotide at a first end; and
c) a sixth polynucleotide at a second end;
wherein the fifth polynucleotide and the sixth polynucleotide each comprise a sequence capable of homologous recombination with an endogenous mitochondrial DNA sequence, wherein homologous recombination of all or part of the donor DNA with the endogenous mitochondrial DNA sequence results in integration of the fourth polynucleotide into the endogenous mitochondrial DNA sequence; and
selecting a cell with an edited mitochondrial genome, wherein the edited mitochondrial genome comprises the fourth polynucleotide.

Embodiment 27. The method of embodiment 26, wherein the donor DNA comprises the first polynucleotide, or both the first polynucleotide and the second polynucleotide.

Embodiment 28. The method of embodiment 27, wherein the edited mitochondrial genome comprises the first polynucleotide.

Embodiment 29. The method of embodiment 28, wherein the edited mitochondrial genome comprises the second polynucleotide.

Embodiment 30. The method of embodiment 26, wherein the donor DNA does not comprise the first polynucleotide.

Embodiment 31. The method of embodiment 30, wherein the donor DNA does not comprise the second polynucleotide.

Embodiment 32. The method of any one of embodiments 26-31, wherein the fourth polynucleotide encodes a fourth polypeptide or a functional RNA, or both.

Embodiment 33. The method of any one of embodiments 26-32, wherein the fourth polynucleotide comprises a cytoplasmic male sterility (CMS) coding region.

Embodiment 34. The method of embodiment 33, wherein the CMS coding region comprises orf79.

Embodiment 35. The method of embodiment 33, wherein the CMS coding region encodes a polypeptide that comprises at least 70%, 80%, 85%, 90%, 91%, 92%, 93%, 94%, 95% or 99% sequence identity to SEQ ID NO: 47.

Embodiment 36. The method of embodiment 34 or 35, wherein the cell is a rice cell.

Embodiment 37. The method of embodiment 33, wherein the CMS coding region comprises orf256 or orf279.

Embodiment 38. The method of embodiment 33, wherein the CMS coding region encodes a polypeptide comprises at least 70%, 80%, 85%, 90%, 91%, 92%, 93%, 94%, 95% or 99% sequence identity to SEQ ID NO: 54.

Embodiment 39. The method of embodiment 33, wherein the CMS coding region encodes a polypeptide comprises at least 70%, 80%, 85%, 90%, 91%, 92%, 93%, 94%, 95% or 99% sequence identity to SEQ ID NO: 56.

Embodiment 40. The method of any one of embodiments 37-39, wherein the cell is a wheat cell.

Embodiment 41. The method of any one of embodiments 26-40, wherein the sequence capable of homologous recombination in the fifth polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

Embodiment 42. The method of any one of embodiments 26-41, wherein the sequence capable of homologous recombination in the sixth polynucleotide has a size of 25-75 nucleotides, 25-100 nucleotides, 25-150 nucleotides, 25-200 nucleotides, 25-300 nucleotides, 25-400 nucleotides, 25-500 nucleotides, 25-1000 nucleotides, 25-1500 nucleotides, or 25-2000 nucleotides.

Embodiment 43. The method of any one of embodiments 26-42, wherein at least one selected from the group consisting of: the first polynucleotide, the second polynucleotide, the third polynucleotide, the fourth polynucleotide, the fifth polynucleotide, the sixth polynucleotide, and any combination thereof, is introduced into the cell via microinjection, meristem transformation, electroporation, Agrobacterium-mediated transformation, viral based gene transfer, transfection, vacuum infiltration, biolistic particle bombardment or any combination thereof.

Embodiment 44. The method of any one of embodiments 26-43, wherein at least one selected from the group consisting of: the first polynucleotide, the second polynucleotide, the third polynucleotide, the fourth polynucleotide, the fifth polynucleotide, the sixth polynucleotide, and any combination thereof, is introduced into the cell as a peptide-polynucleotide complex, wherein the peptide-polynucleotide complex comprises at least one peptide.

Embodiment 45. The method of embodiment 44, wherein the at least one peptide of the peptide-polynucleotide complex comprises at least one selected from the group consisting of: a cell penetrating peptide (CPP), an organellar targeting peptide, a mitochondrial targeting peptide, a histidine-rich peptide, a lysine-rich peptide, and any combination thereof.

Embodiment 46. The method of any one of embodiments 1-45, wherein the method further comprises:
a. introducing into the mitochondrion of the cell a recombinant DNA construct comprising:
   i. a first additional polynucleotide encoding at least one guide polynucleotide, wherein the at least one guide polynucleotide directs a polynucleotide guided polypeptide to cleave at least one target sequence present in an organelle genome; and
      a second additional polynucleotide encoding the polynucleotide guided polypeptide, wherein the polynucleotide guided polypeptide, when associated with the guide polynucleotide, cleaves the at least one target sequence.

Embodiment 47. The method of any one of embodiments 1-45, wherein the method further comprises:
a. introducing into a nucleus of the cell:
   i. a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and
   ii. a second additional polynucleotide encoding at least one guide RNA, wherein the at least one guide RNA directs the polynucleotide guided polypeptide to cleave the at least one target sequence present in the mitochondrial genome.

Embodiment 48. The method of any one of embodiments 1-45, wherein the method further comprises:
a. introducing into a nucleus of the cell:
   i. a first additional polynucleotide encoding a modified polynucleotide guided polypeptide, wherein the modified polynucleotide guided polypeptide comprises a polynucleotide guided polypeptide operably linked to a mitochondrial targeting peptide, wherein the polynucleotide guided polypeptide when associated with a guide RNA, cleaves at least one target sequence present in the mitochondrial genome; and
b. introducing into the mitochondrion of the cell:
   i. a second additional polynucleotide encoding at least one guide RNA, wherein the at least one guide RNA directs the polynucleotide guided polypeptide to cleave the at least one target sequence present in the mitochondrial genome.

Embodiment 49. The method of any one of embodiments 46-48, wherein the polynucleotide guided polypeptide is at least one selected from the group consisting of: a Cas9 protein, a Cas3 protein, a MAD2 protein, a MAD7 protein, a CRISPR nuclease, a nuclease domain of a Cas protein, a Cpf1 protein, an Argonaute, modified versions thereof, a biologically active fragment thereof, and any combination thereof.

Embodiment 50. The method of any one of embodiments 46-49, wherein homologous recombination of all or part of the donor DNA with the endogenous mitochondrial DNA sequence results in an edited mitochondrial genome lacking the at least one target sequence.

Embodiment 51. The method of any one of embodiments 46-50, wherein the method further comprises introducing into a nucleus of the cell a third additional polynucleotide, wherein the third additional polynucleotide encodes a modified site-directed nuclease, wherein the modified site-directed nuclease comprises a site-directed nuclease operably linked to a mitochondrial targeting peptide, wherein the site-directed nuclease cleaves at least one target sequence present in the mitochondrial genome.

Embodiment 52. The method of embodiment 51, wherein the site-directed nuclease is at least one selected from the group consisting of: a TALEN, a Zinc-Finger Nuclease, a Meganuclease, a restriction enzyme, and any combination thereof.

Embodiment 53. The method of any one of embodiments 1-52, wherein the first polynucleotide encoding the first polypeptide further comprises a T7 RNA polymerase promoter, wherein expression of the first polypeptide is under control of the T7 RNA polymerase promoter.

Embodiment 54. The method of any one of embodiments 1-53, further comprising introducing into a nucleus of the cell a fourth additional polynucleotide encoding a modified T7 RNA polymerase, wherein the modified T7 RNA polymerase comprises a T7 RNA polymerase operably linked to a mitochondrial targeting peptide.

Embodiment 55. The method of any one of embodiments 1-54, wherein the first polypeptide comprises an amino acid sequence with at least 70%, 80%, 85%, 90%, 91%, 92%, 93%, 94%, 95% or 99% sequence identity to SEQ ID NO: 24.

Embodiment 56. The method of any one of embodiments 1-55, wherein the first polypeptide comprises SEQ ID NO:24.

Embodiment 57. The method of any one of embodiments 1-56, wherein the first polynucleotide encoding the first polypeptide comprises at least 70%, 80%, 85%, 90%, 91%, 92%, 93%, 94%, 95% or 99% sequence identity to SEQ ID NO: 25.

Embodiment 58. The method of any one of embodiments 1-57, wherein the first polynucleotide comprises SEQ ID NO:25.

Embodiment 59. The method of any one of embodiments 1-58, wherein a sequence encoding a start codon of the first polypeptide is replaced with a sequence encoding a mitochondrial RNA editing site.

Embodiment 60. The method of embodiment 59, wherein the mitochondrial RNA editing site is from a mitochondrial nad4L gene or a mitochondrial cox2 gene.

Embodiment 61. The method of embodiment 60, wherein the sequence encoding the mitochondrial RNA editing site comprises SEQ ID NO: 41 or SEQ ID NO: 42.

Embodiment 62. The method of any one of embodiments 1-61, wherein the method further comprises introducing into a nucleus of the cell a fifth additional polynucleotide encoding an additional selectable marker polypeptide, wherein the additional selectable marker polypeptide provides tolerance to an additional selective agent, and selecting a cell that grows in the presence of the additional selective agent.

Embodiment 63. The method of embodiment 62, wherein the cell is grown simultaneously in a presence of an additional selective agent and in a presence of an inhibitor of acetolactate synthase.

Embodiment 64. The method of embodiment 62, wherein the cell is grown sequentially first in a presence of an additional selective agent and subsequently in a presence of an inhibitor of acetolactate synthase.

Embodiment 65. The method of any one of embodiments 62-64, wherein the additional selectable marker polypeptide is a polypeptide with hygromycin phosphotransferase (HPT) activity and the additional selective agent is hygromycin.

Embodiment 66. The method of any one of embodiments 26-65, wherein the method further comprises removing the first polynucleotide encoding the first polypeptide from the transformed mitochondrion after integration of the fourth polynucleotide.

Embodiment 67. The method of any one of embodiments 8-66, wherein the method further comprises selecting a cell that comprises a plurality of mitochondrial genomes, wherein at least 50%, 60%, 70%, 80%, 90%, or 100% of the plurality of mitochondrial genomes comprise the edited mitochondrial genome.

Embodiment 68. The method of any one of embodiments 8-67, wherein the method further comprises selecting a cell that is homoplasmic for the edited mitochondrial genome.

Embodiment 69. The method of any one of embodiments 1-68, wherein the cell is a plant cell and further wherein a plant is grown from the plant cell.

Embodiment 70. The method of embodiment 69, further comprising selecting a plant wherein the plant comprises the first polypeptide.

Embodiment 71. A cell produced by the method of any one of embodiments 1-70, wherein the cell is a plant cell selected from the group consisting of: a wheat cell, a maize cell, a rice cell, a barley cell, a sorghum cell, a rye cell, a canola cell, a broccoli cell, a cauliflower cell, and a soybean cell.

Embodiment 72. A plant, a cell, a tissue, a propagation material, a seed, a root, a leaf, a flower, a fruit, a pollen, a progeny, or a part thereof, or any combination thereof, produced from the plant cell of embodiment 71, wherein the cell, the tissue, the propagation material, the seed, the root, the leaf, the flower, the fruit, the pollen, the progeny, the part thereof, or the any combination thereof comprises the edited mitochondrial genome.

Embodiment 73. A method of controlling weeds, the method comprising growing a plurality of plants in a presence of an inhibitor of acetolactate synthase, wherein at least one plant of the plurality of plants comprises a mitochondrion comprising a heterologous polynucleotide that encodes a polypeptide having herbicide-resistant acetolactate synthase activity; wherein the presence of the inhibitor of acetolactate synthase is sufficient to selectively promote growth of the at least one plant of the plurality of plants, resulting in an increased growth of the at least one plant of the plurality of plants relative to plants lacking the polynucleotide encoding the polypeptide having herbicide-resistant acetolactate synthase activity.

Embodiment 74. The method of embodiment 73, further comprising applying the inhibitor of acetolactate synthase to the plant, the plurality of plants, soil adjacent to the plant, or any combination thereof.

Embodiment 75. The method of embodiment 74, wherein the inhibitor of acetolactate synthase is applied as a foliar fertilizer.

Embodiment 76. The method of embodiment 74, wherein the inhibitor of acetolactate synthase is applied as a soil amendment.

Embodiment 77. The method of any one of embodiments 73-76, wherein the at least one plant of the plurality of plants is selected from the group consisting of: wheat, maize, rice, barley, sorghum, rye, sugarcane, potato, tomato, canola, broccoli, cauliflower, and soybean.

Embodiment 78. The method of any one of embodiments 73-77, wherein the plants lacking the polynucleotide encoding the polypeptide having herbicide-resistant acetolactate synthase activity are weeds.

Embodiment 79. The method of any one of embodiments 73-78, wherein the polypeptide having herbicide-resistant acetolactate synthase activity comprises an amino acid sequence with at least 80%, 85%, 90%, 91%, 92%, 93%, 94%, or 95% sequence identity to SEQ ID NO: 24.

Embodiment 80. The method of embodiment 78, wherein the polypeptide having acetolactate synthase activity comprises an amino acid sequence of SEQ ID NO: 24.

Embodiment 81. A cell comprising an edited mitochondrial genome, wherein the cell is a plant cell or an algal cell, wherein the edited mitochondrial genome comprises a heterologous polynucleotide encoding a polypeptide having herbicide-resistant acetolactate synthase activity.

Embodiment 82. The cell of embodiment 81, wherein the cell is a plant cell selected from the group consisting of: a wheat cell, a maize cell, a rice cell, a barley cell, a sorghum cell, a rye cell, a canola cell, a broccoli cell, a cauliflower cell, and a soybean cell.

Embodiment 83. The cell of embodiment 81 or embodiment 82, wherein the edited mitochondrial genome comprises at least one nucleotide substitution, deletion, or insertion.

Embodiment 84. The cell of any one of embodiments 81-83, wherein the cell comprises a transformed mitochondrion, wherein the transformed mitochondrion comprises the edited mitochondrial genome.

Embodiment 85. The cell of any one of embodiments 81-84, wherein an amino acid sequence of the polypeptide having herbicide-resistant acetolactate synthase activity encoded by the heterologous polynucleotide comprises at least 80%, 85%, 90%, 95%, 96%, 97%, 98% or 99% sequence identity to SEQ ID NO: 24.

Embodiment 86. The cell of embodiment 85, wherein the amino acid sequence of the polypeptide having herbicide-resistant acetolactate synthase activity comprises SEQ ID NO: 24.

Embodiment 87. The cell of embodiment 86, wherein the heterologous polynucleotide encoding the polypeptide having herbicide-resistant acetolactate synthase activity comprises SEQ ID NO: 25.

Embodiment 88. The cell of any one of embodiments 81-87, wherein a sequence encoding a start codon of the heterologous polynucleotide is replaced with a sequence encoding a mitochondrial RNA editing site.

Embodiment 89. The cell of embodiment 88, wherein the mitochondrial RNA editing site is from a mitochondrial nad4L gene or a mitochondrial cox2 gene.

Embodiment 90. The cell of embodiment 89, wherein a sequence encoding the mitochondrial RNA editing site comprises SEQ ID NO: 41 or SEQ ID NO: 42.

Embodiment 91. The cell of any one of embodiments 81-90, wherein the edited mitochondrial genome further comprises a second polynucleotide encoding a polypeptide or a functional RNA, or both, wherein the polypeptide and the functional RNA are heterologous to the mitochondria.

Embodiment 92. The cell of embodiment 91, wherein the second polynucleotide comprises a cytoplasmic male sterility (CMS) coding region.

Embodiment 93. The cell of embodiment 92, wherein the CMS coding region is orf79.

Embodiment 94. The cell of embodiment 91, wherein the second polynucleotide encodes a polypeptide that comprises at least 70%, 80%, 85%, 90%, 91%, 92%, 93%, 94%, 95% or 99% sequence identity to SEQ ID NO: 47.

Embodiment 95. The cell of embodiment 91, wherein the second polynucleotide encodes a polypeptide that comprises SEQ ID NO: 47.

Embodiment 96. The cell of any one of embodiments 92-95, wherein the cell is a rice cell.

Embodiment 97. The cell of embodiment 92, wherein the CMS coding region is orf256 or is orf279.

Embodiment 98. The cell of any embodiment 91, wherein the second polynucleotide encodes a polypeptide that comprises at least 70%, 80%, 85%, 90%, 91%, 92%, 93%, 94%, 95% or 99% sequence identity to SEQ ID NO: 54.

Embodiment 99. The cell of embodiment 91, wherein the second polynucleotide encodes a polypeptide that comprises SEQ ID NO: 54.

Embodiment 100. The cell of embodiment 91, wherein the second polynucleotide encodes a polypeptide that comprises at least 70%, 80%, 85%, 90%, 91%, 92%, 93%, 94%, 95% or 99% sequence identity to SEQ ID NO: 56.

Embodiment 101. The cell of embodiment 91, wherein the second polynucleotide encodes a polypeptide that comprises SEQ ID NO: 56.

Embodiment 102. The cell any one of embodiments 97-101, wherein the cell is a wheat cell.

Embodiment 103. The cell of any one of embodiments 81-102, wherein the cell further comprises a third heterologous polynucleotide in a nucleus of the cell, wherein the third heterologous polynucleotide encodes an additional selectable marker polypeptide that provides the cell with tolerance to an additional selective agent.

Embodiment 104. The cell of embodiment 103, wherein the additional selectable marker polypeptide has hygromycin phosphotransferase (HPT) activity.

Embodiment 105. The cell of embodiment 104, wherein the additional selective agent is hygromycin.

Embodiment 106. The cell of any one of embodiments 81-105, wherein the cell comprises a plurality of mitochondrial genomes wherein at least 50%, 60%, 70%, 80%, 90%, or 100% of the plurality of mitochondrial genomes comprise the edited mitochondrial genome.

Embodiment 107. The cell of any one of embodiments 81-106, wherein the cell is homoplasmic for the edited mitochondrial genome.

Embodiment 108. The cell of any one of embodiments 81-107, wherein the cell expresses the polypeptide having herbicide-resistant acetolactate synthase activity.

Embodiment 109. The cell of any one of embodiments 81-96, wherein the edited mitochondrial genome comprises a fourth heterologous polynucleotide encoding a regulatory subunit of an acetolactate synthase or a biologically active fragment thereof.

Embodiment 110. The cell of embodiment 109, wherein the cell expresses the regulatory subunit of an acetolactate synthase or the biologically active fragment thereof encoded by the fourth heterologous polynucleotide.

Embodiment 111. The cell of any one of embodiments 81-108, wherein a nucleus of the cell comprises a fifth heterologous polynucleotide encoding a modified regulatory subunit of an acetolactate synthase or a modified biologically active fragment thereof, wherein the modified regulatory subunit of the acetolactate synthase or the modified biologically active fragment thereof comprises a mitochondrial targeting peptide.

Embodiment 112. The cell of embodiment 111, wherein the cell expresses the modified regulatory subunit of the acetolactate synthase or the modified biologically active fragment thereof.

Embodiment 113. The cell of any one of embodiments 81-112, wherein the cell grows in a medium wherein an inhibitor of acetolactate synthase is present.

Embodiment 114. The cell of embodiment 113, wherein the polypeptide having herbicide-resistant acetolactate synthase activity is resistant to at least one herbicide selected from the group consisting of: sulfonylureas, imidazolinones, triazolopyrimidines, pyrimidinyl(thio)benzoates, sulfonanilides, sulfonylaminocarbonyltriazolinones, and any combination thereof.

Embodiment 115. The cell of embodiment 114, wherein the polypeptide having herbicide-resistant acetolactate synthase activity is resistant to a sulfonylurea.

Embodiment 116. The cell of embodiment 115, wherein the sulfonylurea is a chlorsulfuron.

Embodiment 117. The cell of embodiment 116, wherein the polypeptide having herbicide-resistant acetolactate synthase activity is resistant to chlorsulfuron at a concentration of at least 20 nM-100 nM, 100 nM-1 μM, 1 μM-20 μM, or 20 μM-100 μM.

Embodiment 118. A transgenic plant or parts thereof comprising the cell of any one of embodiments 81-117.

Embodiment 119. The transgenic plant or parts thereof of embodiment 118, further comprising a cell, a tissue, a propagation material, a seed, a pollen, a progeny, or any combination thereof.

Embodiment 120. The transgenic plant or parts thereof of embodiment 118 or embodiment 119, wherein the transgenic plant or parts thereof is grown in a temperature-controlled incubator.

Embodiment 121. The transgenic plant or parts thereof of embodiment 120, wherein the temperature-controlled incubator further comprises a light-dark cycle.

Embodiment 122. A field or a greenhouse comprising the transgenic plant or parts thereof of embodiment 118.

Embodiment 123. A food product comprising the cell of any one of embodiments 81-117.

Embodiment 124. A field comprising the cell of any one of embodiments 81-117.

Embodiment 125. A kit comprising the cell of any one of embodiments 81-117 or the transgenic plant or parts thereof of anyone of embodiments 118-121.

### EXAMPLES

The present disclosure is further defined in the following Examples, in which parts and percentages are by weight and degrees are Celsius, unless otherwise stated. It should be understood that these Examples, while indicating embodiments, are given by way of illustration only. From the above discussion and these Examples, the essential characteristics of this disclosure can be ascertained, and without departing from the spirit and scope thereof, various changes and modifications of the disclosure can be envisioned to adapt it to various usages and conditions. Such modifications are also intended to fall within the scope of the appended claims.

### Example 1

### Chlorsulfuron Selection of Transformed Rice Cells

Embryogenic callus cultures of a wild-type rice variety were initiated and maintained for a minimum of 4-6 weeks on a Chu-N6-based callus induction & maintenance medium supplemented with the plant growth regulator 2,4-D. Four days prior to transformation, callus cultures were subcultured to fresh N6-based callus maintenance medium. Approximately four hours prior to transformation, calli were prepared for transformation by plating tissue in the target zone on the same N6-based medium supplemented with mannitol and sorbitol for osmotic protection.

Rice calli were transformed with various ALS expression constructs (e.g., herbicide-resistant ALS large subunit; regulatory ALS small subunit) using the biolistics method (particle bombardment). The following steps were used for culture, selection, and regeneration.

1. After bombardment, the calli were incubated in the dark for 16-20 hours at 26° C., then clumps of callus tissue approximately 1-3 mm in size were subcultured to a selective medium, which was the callus maintenance medium supplemented with 20-100 nM chlorsulfuron and 25-50 mg/L hygromycin as appropriate for the genes bombarded. Calli on selective media were then returned to dark incubation for 2-3 weeks.

2. After 2-3 weeks of dark incubation, small (1-3 mm) clumps were again subcultured to fresh selective medium containing chlorsulfuron and incubated for approximately 2 weeks in a plant growth chamber with a 16 hr light-8 hr dark photoperiod, at light intensity of 60 μmoles per square meter per second, at 26° C. Additional rounds of subculturing to fresh selection medium after 2-week periods of maintenance in the light were most often performed.

3. At the end of the second selection period or later, between 5-8 weeks after bombardment, vigorously growing calli (individual initial events) were picked from the surrounding dying tissue and transferred to individual plates of fresh selective medium supplemented with chlorsulfuron, maintaining their individual identity. If there were many initial events, each event occupied from one eighth to one half of a plate, the identity of each event being kept separate. In some cases, calli with moderate or poor growth were also maintained for comparison.

### Multiple Selection Processes

When an ALS gene expression cassette was co-transformed with a 35S:HPT nuclear expression cassette conferring hygromycin B resistance, selection of events was facilitated by the use of 25-50 mg/L hygromycin B along with chlorsulfuron in the selective medium. Variations in the timing of introduction of the hygromycin selection in conjunction with chlorsulfuron selection were performed for highest recovery of events expressing the ALS gene(s).

In some experiments (with or without 35S:HPT co-expression), the herbicide-resistant ALS gene expression cassette was also linked and co-transformed with an oliR expression cassette conferring resistance to the antibiotic oligomycin. In some experiments, geminivirus VOR sequences (target sites for the geminivirus Rep protein) were also present in the construct. In some experiments, oligomycin was incorporated into the selective medium at a rate of 1-5 mg/L. In some experiments, when oligomycin selection was included, the carbon source sucrose was reduced to 0.5% (5 mg/L) or replaced with 60 ml of a sterile 50% glycerol solution per L of medium to enhance the effectiveness of oligomycin. In some experiments where oligomycin was a selective agent, the compound disulfiram was also incorporated into the medium at 100 μM to inhibit the ability of cells to utilize any alcohol produced by anaerobic respiration of treated cells.

In some experiments, variations were made in the timing of the inception of oligomycin selection in conjunction with chlorsulfuron selection. In some experiments, after an initial period of one or more cycles of subculture and selection, the use of chlorsulfuron was discontinued and oligomycin alone was utilized at the above concentration.

### Sampling for Molecular Analysis

After approximately 10 weeks or more of selection, samples of individual events were taken for PCR analysis. One or more small clumps of tissue ˜1 mm in diameter were supplied for each initial event to be analyzed. Maintenance and sampling of callus events continued for as long as 6-to-8 months.

### Embryo Maturation and Plant Regeneration

4. In some experiments, at the end of two or more weeks of proliferation of individual initial events, calli which were sustaining growth (representing putative mitochondrial transformation events) were transferred to an N6-based medium for embryo maturation, still containing chlorsulfuron as selective agent, but omitting the growth regulator 2,4-D, and supplementing with 2.5 g/L Phytagel.

5. After a minimum of 10-14 days, mature somatic embryos showing signs of normal maturation were transferred to an N6-based germination medium, still containing chlorsulfuron as selective agent. This medium was supplemented with growth regulators 0.2 mg/L naphthaleneacetic acid and 2 mg/L 6-benzylamino purine, and 2.5 g/L Phytagel. These events were grown in a 16 hr/8 hr light/dark growth chamber at 26° C. for root and shoot formation.

6. Plants showing both root and shoot development after step 5 were transferred to pots containing an artificial potting medium and acclimatized and grown in a greenhouse.

### Inducible Expression System

In some experiments, the target tissue for biolistics transformation of rice was from a different source. The different source of callus tissue was derived from a previous Agrobacterium tumefaciens transformation. That transformation had created an event with a dexamethasone-inducible system for production of a geminivirus Rep protein. Inducible transgenic lines were identified by prescreening using an RFP visual marker, as described in EXAMPLE 6. To supply enough tissue for bombardment, the event was maintained on the first selective medium, which was supplemented with 40 mg/L hygromycin, amino acids, proline, maltose and 2,4-D growth regulator, for approximately two months prior to biolistics transformation.

When the tissue to be bombarded was derived from the inducible line, it was precultured for 4 days prior to bombardment on the first selective medium. In some experiments, this preculture medium was also supplemented with either 1,000 μl of 10 μM dexamethasone (DEX) dissolved in DMSO or 1,000 μl DMSO alone as a negative control. DEX was included as the chemical inducer of the Rep protein.

Approximately four hours prior to transformation, calli were prepared for bombardment by plating tissue in the target zone on the N6-based callus induction medium supplemented with mannitol and sorbitol for osmotic protection, but without DEX or DMSO.

1. After bombardment, the calli were incubated in the dark for 16-20 hours at 26° C., then clumps of callus tissue approximately 1-3 mm in size were subcultured to the N6-based callus maintenance medium supplemented with growth regulator 2,4-D and the appropriate selective agents including 20-100 nM chlorsulfuron and 25-50 mg/L hygromycin and 1-5 mg/L oligomycin with reduced or alternate carbon source as described above and as appropriate for the genes bombarded. In some experiments, chemical induction with DEX in the medium was begun with the first round of selection. Calli on selective media with DEX and/or DMSO were then returned to dark incubation for the first round of selection. In some experiments, DEX and/or DMSO was introduced at a later point in the selection process.

2. After 2-3 weeks of dark incubation, small (1-3 mm) clumps were again subcultured to fresh selective medium and then incubated in the light as described above. Additional rounds of subculturing to fresh selection medium with DEX and/or DMSO after 2 to 3-week periods of maintenance in the light were most often performed.

3. In some experiments, the induction with DEX continued throughout this entire selection process. In some experiments, the use of DEX and/or DMSO was discontinued for one or more culture periods. In some experiments, DEX was re-introduced at a later point in the selection process.

### Sampling for Molecular Analysis

After approximately 10 or more weeks of selection, samples of individual events were taken for PCR analysis. One or more small clumps of tissue ˜1 mm in diameter were supplied for each initial event to be analyzed. Maintenance and sampling of callus events continued for as long as 6-to-8 months.

### Example 2

Sulfonylurea Selection of Transformed Cells by Importing Herbicide-Resistant ALS Large Subunit Protein into Rice Mitochondria

The use of herbicide-resistant acetolactate synthase (ALS) as a selectable marker for mitochondrial transformation was tested by importing the protein made in the cytoplasm into mitochondria of rice callus cells. For this experiment, we made a plasmid, pNAP170, that contained nuclear expression cassettes encoding the following three polypeptides: 1) MTS-ALS(HR-LS), the herbicide-resistant ALS catalytic large subunit, ALS(HR-LS), fused with the mitochondria-targeting sequence (MTS) of the Arabidopsis rsp10 protein, 2) MTS-ALS(SS), the regulatory small subunit of ALS, ALS(SS), fused with the mitochondria-targeting sequence (MTS) of the Arabidopsis At5g47030 gene, and 3) HPT, the hygromycin phosphotransferase protein for use as a selectable marker for nuclear transformation.

To produce the MTS-ALS(HR-LS) protein, a polynucleotide sequence encoding a rice herbicide-resistant ALS large subunit polypeptide (SEQ ID NO: 7; rice gene ID: AB049823) was used, which was shown to confer sulfonylurea (SU) resistance in rice (Kawai et al., Plant Biotech. 27:75). The encoded protein contains a chloroplast targeting sequence. For targeting it to mitochondria, the sequence encoding the first 18 aa residues (SEQ ID NO: 8), consisting of the chloroplast targeting sequence, were deleted and replaced with a sequence encoding the mitochondrial targeting sequence of the Arabidopsis mitochondrial ATP synthase subunit delta protein (SEQ ID NO: 9; gene ID: At5g47030). The corresponding DNA fragment, franked by BamHI and KpnI restriction sites, was synthesized by an external vendor, GENEWIZ®. The nucleotide sequence encoding the MTS-ALS(HR-LS) protein is presented as SEQ ID NO: 10.

The amino acid sequence of the MTS-ALS(HR-LS) protein is presented as SEQ ID NO: 11. The first 56 amino acids of SEQ ID NO: 11 correspond to the MTS of the Arabidopsis thaliana rsp10 protein.

The synthesized DNA containing the coding region for MTS-ALS(HR-LS) was cloned into pNAP148, to create an expression cassette having the MTS-ALS(HR-LS) coding region operably linked to the maize UBI1 promoter and intron (SEQ ID NO: 12) and the NOS terminator (SEQ ID NO: 13). The resulting plasmid, pNAP152, also contains an expression cassette with a nucleotide sequence encoding the hygromycin phosphotransferase (SEQ ID NO: 14) operably linked to the 35S promoter (SEQ ID NO: 15) and CaMV terminator (SEQ ID NO: 16).

The activity of the ALS catalytic large subunit protein has been shown to be enhanced by the presence of a small subunit protein, which we have designated as ALS(SS). For our experiments in rice, we identified a rice homolog of ALS(SS) (SEQ ID NO: 17; XP_015615160, Os11g14950). We used a prediction program for chloroplast targeting sequences, ChloroP1.1, to identify a putative chloroplast targeting sequence as the first 47 amino acid residues (SEQ ID NO: 18) of the rice ALS(SS) protein. To target the ALS(SS) protein to mitochondria, we designed a nucleotide sequence in which the sequence encoding the first 47 aa residues of the rice ALS(SS) protein were deleted and replaced with a sequence encoding the MTS of the Arabidopsis At5g47030 gene (SEQ ID NO: 19).

The resulting DNA sequence encoding the MTS-ALS(SS) is presented as SEQ ID NO: 20.

The amino acid sequence of the MTS-ALS(SS) encoded by SEQ ID NO: 20 is presented as SEQ ID NO: 21. The first 36 amino acids of SEQ ID NO: 21 correspond to the MTS from the Arabidopsis At5g47030 gene.

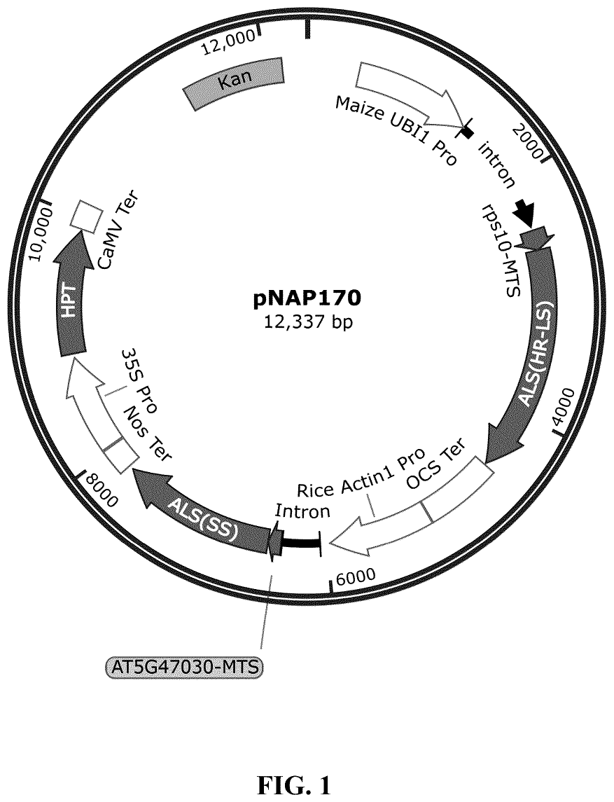

The DNA fragment encoding the MTS-ALS(SS) was synthesized and operably linked to the rice Actin1 promoter and intron (SEQ ID NO: 22) and the NOS terminator (SEQ ID NO: 13) in the construction of plasmid pNAP151. Plasmid pNAP151 has an OCS terminator (SEQ ID NO: 23) 5′ to the MTS-ALS(SS) expression cassette. The OCS terminator and the entire MTS-ALS(SS) expression cassette from pNAP151 were cloned into pNAP152 to create pNAP170. FIG. 1 shows a map of plasmid pNAP170. The plasmid pNAP170 contains the following three nuclear expression cassettes: pUBI1::MTS-ALS(HR-LS)::OCS terminator; pACT1::MTS-ALS(SS)::NOS terminator; and p35S::HPT::CaMV terminator.

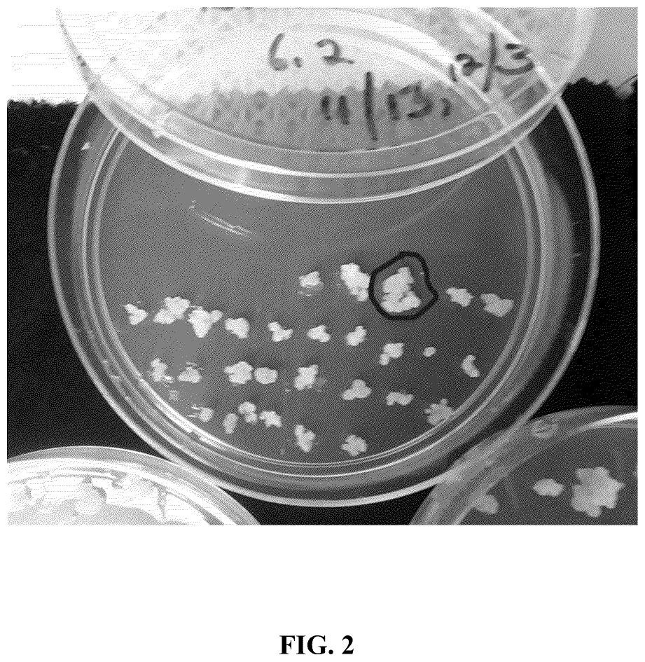

pNAP170 was transformed into rice callus cells essentially as described in EXAMPLE 1. After transformation of pNAP170 into rice callus cells using the biolistic method, we selected events that grew on media containing the sulfonylurea herbicide, chlorsulfuron, as the selective agent (FIG. 2). FIG. 2 shows growth of rice callus cells in a medium containing chlorsulfuron, in which the rice callus cells were transformed with pNAP170. The callus within the circle was subjected to further selection.

### Example 3

### Sulfonylurea Selection of Transformed Cells by Expression of Herbicide-Resistant ALS Large Subunit Protein in Rice Mitochondria

A polynucleotide encoding the ALS(HR-LS) protein was introduced into rice mitochondria to evaluate its efficacy as a selectable marker. For this purpose, the sequence encoding 13 of the 18 amino acids comprising the chloroplast targeting sequence of the ALS(HR-LS) protein (described in EXAMPLE 2) was deleted. The resulting protein, mALS(HR-LS), therefore has no functional organellar targeting sequence. The amino acid sequence of the mALS(HR-LS) protein is presented as SEQ ID NO: 24.

The sequence encoding the mALS(HR-LS) was optimized for expression in rice mitochondria by replacing rare codons with more frequently used codons and eliminating unwanted restriction sites. The nucleotide sequence of the optimized mALS(HR-LS) coding region is presented as SEQ ID NO: 25.

To express the mALS(HR-LS) gene in rice mitochondria, the sequence encoding mALS(HR-LS) was operably linked to the promoter and terminator of the ATP1 gene encoded in rice mitochondrial genome. The ATP1 gene sequence was identified in the GenBank database (NC_011033). To strengthen the transcription in mitochondria, we added the T7 promoter (SEQ ID NO: 26) upstream of the transcription start site. The nucleotide sequence of this hybrid ATP1+T7 promoter is presented as SEQ ID NO: 27.

Additionally, the T7 terminator (SEQ ID NO: 28) was fused to the 5′ end of the ATP1 terminator to produce a hybrid T7+ATP1 terminator (SEQ ID NO: 29) used in this experiment.

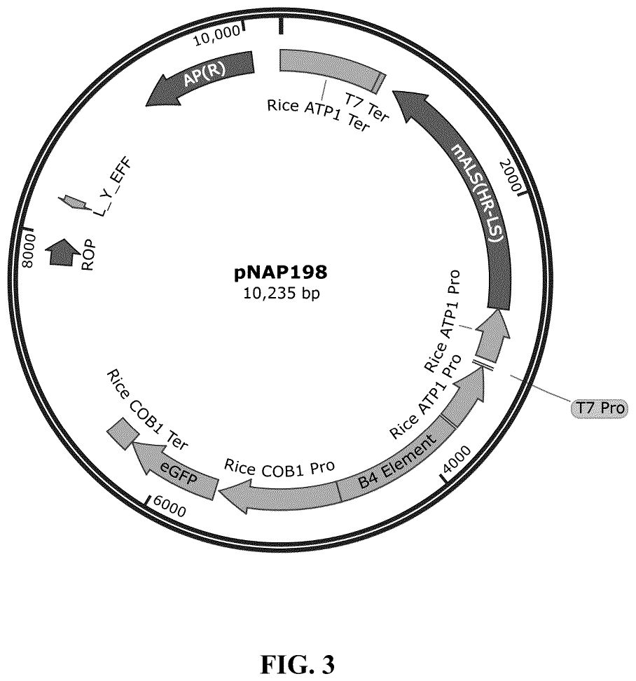

The mitochondrial expression cassette for mALS(HR-LS) was then cloned into pNAP76, which was a pBR322 based vector carrying the B4 element that was associated with autonomous replication in rice mitochondria. Plasmid pNAP76 also encodes a eGFP reporter with an RNA editing site derived from rice COX2 to create the translation initiation site by a natural RNA editing specific to rice mitochondria (SEQ ID NO: 30). The eGFP coding sequence was operably linked to the rice COB1 promoter and 5′ UTR (SEQ ID NO: 31) and the rice COB1 terminator (SEQ ID NO: 32). The resulting construct, pNAP198, contains the mitochondrial expression cassette for mALS(HR-LS). FIG. 3 shows a map of plasmid pNAP198. The plasmid contains the mALS(HR-LS) coding region operably linked to a hybrid T7+rice ATP1 promoter and a hybrid T7+rice ATP1 terminator. The plasmid also has a eGFP coding sequence operably linked to a rice COB1 promoter and 5′ UTR and a rice COB1 terminator. The plasmid also contains a B4 element that has been associated with autonomous replication in rice mitochondria.

To enhance sulfonylurea selection in mitochondria, we made a construct for nuclear expression of the MTS-ALS(SS) and the MTS-T7 RNA polymerase. The construct, pNAP195, carried the following three expression cassettes: pUBI1::MTS-T7 Pol::OCS terminator; pACT1::MTS-ALS(SS)::NOS terminator; and p35S::HPT::CaMV terminator. The nucleotide sequence encoding the MTS-T7 RNA polymerase is presented as SEQ ID NO: 33. The corresponding amino acid sequence of the MTS-T7 RNA polymerase is presented as SEQ ID NO: 34. The MTS (SEQ ID NO: 35) used for the MTS-T7 RNA polymerase was from the At5g47030 gene.

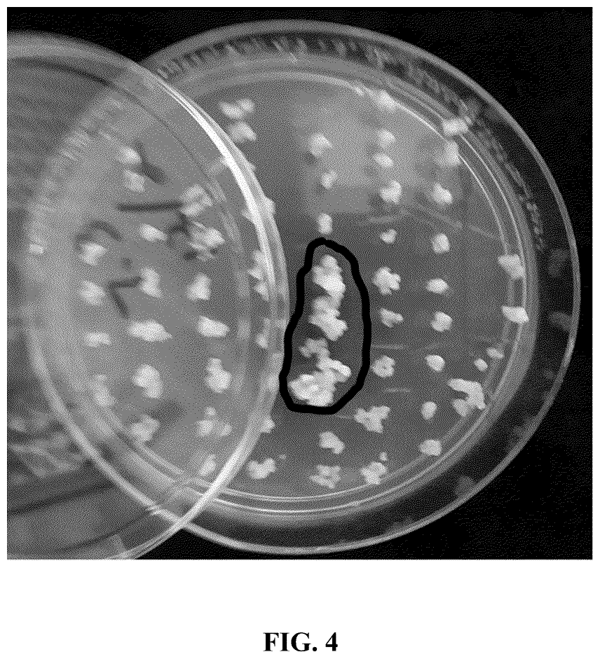

The two constructs, pNAP195 and pNAP198, were co-transformed into rice callus essentially as described in EXAMPLE 1. After co-transformation of pNAP195 and pNAP198 into rice callus cells using the biolistic method, we selected events that grew on media containing the sulfonylurea herbicide, chlorsulfuron, as the selective agent (FIG. 4). FIG. 4 shows growth of rice callus cells in a medium containing chlorsulfuron, in which the rice callus cells were co-transformed with pNAP195 and pNAP198. The callus samples within the circle were subjected to further selection.

### Example 4

Mitochondrial Gene Editing with Donor DNA Encoding mALS(HR-LS)

In this experiment, DNA fragments containing Donor DNA encoding mALS(HR-LS) was used to transform rice mitochondria. The Donor DNA fragments carried the regions at the ends that were homologous to the ATP6 gene in the rice mitochondrial genome. The length of homologous regions at the 5′ and 3′ ends were 1.6 kb and 1.2 kb, respectively. The 5′ homologous region of the Donor DNA is presented as SEQ ID NO: 36. Certain nucleotides were changed in the 5′ homologous region to prevent recognition by gRNA2 (SEQ ID NO: 37) and the MAD7 enzyme (SEQ ID NO: 38).

The 3′ homologous region of the Donor DNA is presented as SEQ ID NO: 39. Certain nucleotides were changed in the 3′ homologous region to prevent recognition by gRNA4 (SEQ ID NO: 40) and the MAD7 enzyme.

To further ensure the expression of the mALS gene in mitochondria, in two Donor DNA plasmids we replaced the region encoding the initiation codon of mALS(HR-LS) with either of the following two elements derived from RNA editing sites occurring naturally in rice mitochondria: a sequence encoding an RNA editing site of the rice nad4L transcript (SEQ ID NO: 41); or a sequence encoding an RNA editing site of the rice cox2 transcript (SEQ ID NO: 42).

Without RNA editing from one of these sites, which are specific to mitochondria, the resulting mRNA would have no in-frame AUG codons near the start of the protein-coding region. Hence, no functional mALS(HR-LS) protein would be expected to be produced, further ensuring the mitochondrial selection.

Each of the two mALS(HR-LS) coding regions (with alternate RNA editing sites) were operably linked with the hybrid ATP1+T7 promoter and a truncated version (SEQ ID NO: 43) of the hybrid T7+ATP1 terminator described in EXAMPLE 3.

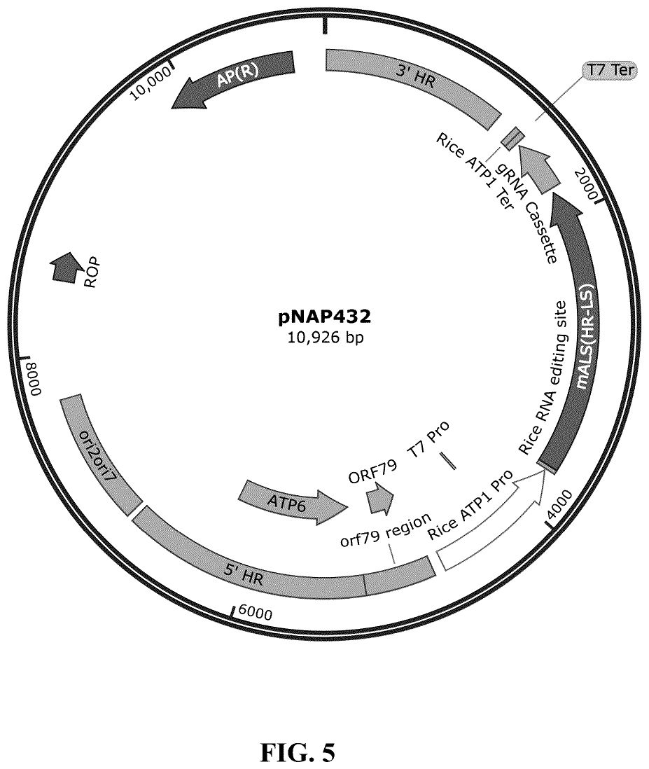

The nucleotide sequence of the Donor DNA fragments created with the nad4L and cox2 RNA editing sites (from plasmids pNAP432 and pNAP433, respectively) are presented in SEQ ID NO: 44 and SEQ ID NO: 45, respectively. A map of plasmid pNAP432 is presented in FIG. 5. The plasmid contains a Donor DNA used to transform rice mitochondria. The Donor DNA has an mALS(HR-LS) coding region (with an nad4L RNA editing site) operably linked to a hybrid T7+rice ATP1 promoter and a truncated version of the hybrid T7+rice ATP1 terminator. The Donor DNA has rice mitochondrial DNA homologous regions at the 5′ and 3′ ends of 1.6 kb and 1.2 kb, respectively. Plasmid pNAP433 is identical to plasmid pNAP432, except for having a cox2 RNA editing site instead of a nad4L RNA editing site.

Each Donor DNA fragment also contained a nucleotide sequence (SEQ ID NO: 46) which encodes the orf79 protein (SEQ ID NO: 47). Each Donor DNA sequence also encodes a gRNA cassette (SEQ ID NO: 48) for potential use with a MAD7 nuclease.

The two Donor DNA fragments each were transformed into rice callus cells either alone, or together with a nuclear expression construct, i.e., pNAP195 (for MTS-ALS(SS) expression) or pNAP159 (lacking MTS-ALS(SS) expression). pNAP195 was described in EXAMPLE 3, and has the following three nuclear expression cassettes: pUBI1::MTS-T7 Pol::OCS terminator; pACT1::MTS-ALS(SS)::NOS terminator; and p35S::HPT::CaMV terminator. pNAP159 was constructed without an MTS-ALS(SS) nuclear expression cassette, and has the following two nuclear expression cassettes: pUBI1::MTS-T7 Pol: NOS terminator; and p35S::HPT::CaMV terminator.

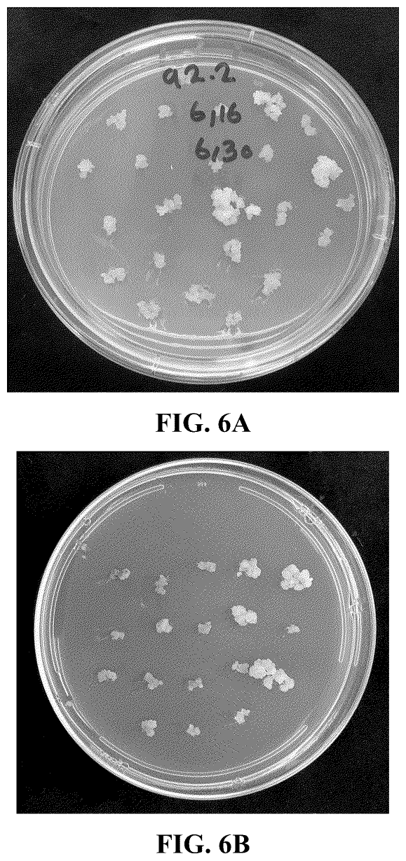

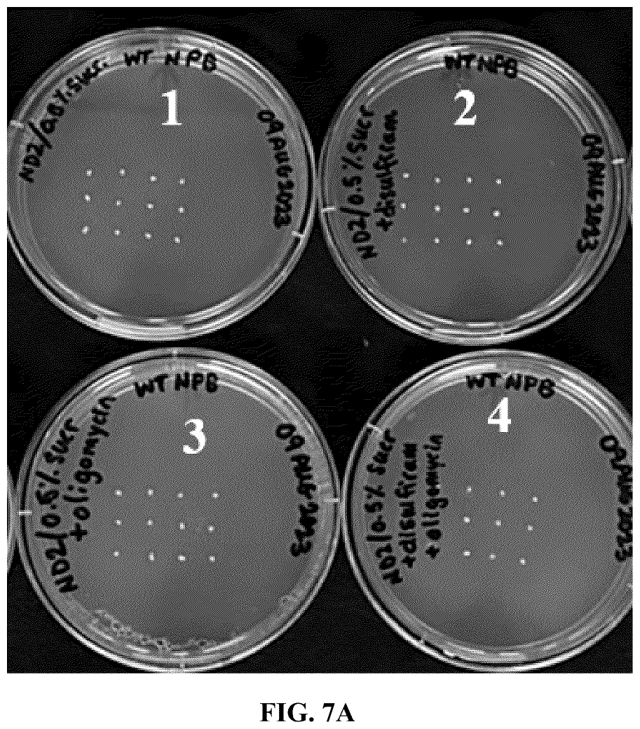

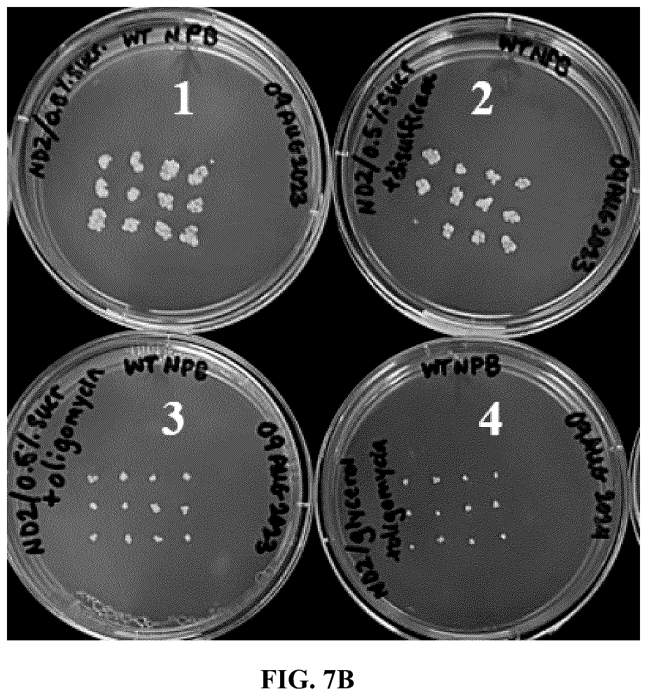

After transformation into rice callus cells with Donor DNA fragment alone, or co-transformation of Donor DNA fragment with either plasmid pNAP159 or plasmid pNAP195, we selected events that grew on media containing the sulfonylurea herbicide, chlorsulfuron, as the selective agent (FIG. 6A and FIG. 6B). FIG. 6A: Rice callus cells were co-transformed with plasmid pNAP195 and isolated Donor DNA from pNAP432. FIG. 6B: Rice callus cells were co-transformed with plasmid pNAP195 and isolated Donor DNA from pNAP433.

### Integration Analysis

To confirm the integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. The primer pair that we used for amplifying the 5′ junction region was: 5 HR Primer A (SEQ ID NO: 49) and ORF Primer B (SEQ ID NO: 50). The primer pair that we used for amplifying the 3′ junction region was: 3 HR Primer A (SEQ ID NO: 51) and 420 Primer A (SEQ ID NO: 52).

For PCR analyses, callus of each positive event (5-20 mg) was sampled in a tube. 300 μl of 0.02 N NaOH with 1 mM EDTA was added to each sample and heated for 20 min at 100° C. Then, the aqueous phase was extracted with phenol/chloroform and subsequently with chloroform. Total DNA was precipitated by the addition of NaOAc and ethanol. DNA was resuspended in 30 μl TE. The DNA yield was about 200 ng/μl on average. 1 μl of DNA was used for each PCR reaction. The PCR reaction was prepared as follows: 1 μl of total DNA, 10 pmol of each primer and 12.5 μl LongAmp Taq 2× Master Mix (New England Labs Inc.) in a 25 μl reaction mix. The PCR reaction for the 5′ junction amplification was performed by 30 seconds at 95° C., then 35 cycles of 15 seconds at 95° C. and 3 minutes at 65° C., followed by the final incubation for ten minutes at 65° C. The PCR reaction for the 3′ junction amplification was performed by 30 seconds at 95° C., then 35 cycles of 15 seconds at 95° C., 30 seconds at 63° C. and 2 minutes at 65° C., followed by the final incubation for 10 minutes at 65° C. The PCR samples were separated on 0.7% agarose gels. The junction DNA with the correct size (1,742 bp for the 5′ junction and 1,438 bp for the 3′ junction) was amplified from multiple samples. Selected DNA bands were isolated from gels and subjected to sequence analysis. All showed the correct integration of Donor DNA at the homologous regions. In terms of frequency of obtaining events under sulfonylurea selection, there was no significant difference between the two RNA editing sites we used in constructs pNAP432 and pNAP433. Comparing the results from 34 events from three different combinations of mitochondrial and nuclear expression constructs (TABLE 2), a higher frequency of Donor DNA integration when combined with concurrent T7 RNA Polymerase expression was observed; potentially due to stronger mALS(HR-LS) expression from the T7 promoter in mitochondria having T7 RNA polymerase. Furthermore, a higher frequency of integration when combined with concurrent ALS(SS) expression was observed.

TABLE 2
Results from different combinations of mitochondrial
and nuclear expression constructs

| Donor DNA | Nuclear | | | | Total |
|---|---|---|---|---|---|
| Fragment Source | Constructs | ALS(SS) | T7 Pol | PCR+ | Event # |
| pNAP432/433 | None | No | No | 0 | 8 |
| pNAP432/433 | pNAP159 | No | Yes | 2 | 8 |
| pNAP432/433 | pNAP195 | Yes | Yes | 5 | 18 |

### Example 5

### Selection of Transformed Rice Cells Using Glyphosate or Glufosinate

The induction, maintenance, and pre-culture of tissue in experiments utilizing glyphosate or glufosinate for selection of transformed rice cells were the same as those described in EXAMPLE 1 for calli of wild-type Nipponbare and the tissue of the callus event with the dexamethasone-inducible system.

Rice calli of both sources were transformed with various GS1-HR or EPSPS-HR expression constructs (e.g., ATP1 promoter driving coding regions for herbicide-resistant (HR) versions of GS1 or EPSPS) using the biolistics method (particle bombardment). In some experiments, the herbicide-resistant GS1-HR gene or the herbicide-resistant EPSPS-HR gene was the sole selectable marker gene, while in other experiments it was delivered along with an herbicide-resistant ALS gene and/or an oligomycin-resistant oliR gene. After bombardment, the following steps were used for culture and selection.

1. After bombardment, the calli were incubated in the dark for 16-20 hours at 26° C., then clumps of callus tissue approximately 1-3 mm in size were subcultured to a selective medium, which was the callus maintenance medium supplemented with either 1.5 mM glyphosate or 50-100 mg/L glufosinate ammonium (glufosinate). Tissue of the inducible line was cultured on the first selective medium, supplemented with glyphosate or glufosinate, and with DEX and/or DMSO as described in EXAMPLE 1. Calli on selective media+/−DEX were then returned to dark incubation for 2-3 weeks.

2. After 2-3 weeks of dark incubation, small (1-3 mm) clumps were again subcultured to fresh selective media containing glyphosate or glufosinate, +/−DEX, and incubated for approximately 2 weeks in a lighted plant growth chamber with a 16 hr light-8 hr dark photoperiod, at intensity of 60 μmoles per square meter per second, at 26° C. Additional rounds of subculturing to fresh selective medium+/−DEX after 2-to-3-week periods of maintenance in the light were performed for up to 6 months or more. In some experiments, the concentration of glufosinate was reduced by 50% at 7-12 weeks after bombardment. In some experiments, the incorporation of DEX in the selection medium was suspended for up to six or more culture periods. In some experiments, the DEX in the selective medium was reintroduced after the culture periods without it.

3. At the end of the second selection period or later, calli derived from individual bombarded callus clumps were divided into two or more smaller pieces as they were transferred to plates of the fresh selective media, marking on the plate all pieces which grew from the same original bombarded piece, to maintain their original identity. Sampling for PCR analysis of the clumps derived from original bombarded pieces was done three-to-five months or more after bombardment.

### Dual Selection Process

When a GS1-HR or EPSPS-HR gene expression cassette was co-transformed with a 35S:HPT nuclear expression cassette conferring hygromycin B resistance, or when a 35S:HPT nuclear expression cassette existed in the previously transformed source tissue, selection of events was facilitated by the addition to the medium of 25-50 mg/L hygromycin B.

### Example 6

### Nuclear Construct to Induce Expression of a Mitochondrially Targeted Rep Protein in Plant Cells

The Rep protein can induce the replication of DNA that is flanked by the VOR elements. The replication system is originally derived from geminivirus. To amplify Donor DNA after its transformation into mitochondria, the 5′ and 3′ Donor DNA regions with sequences homologous to target sites in the mitochondrial DNA were flanked by a VOR element in the direct repeat orientation to yield a VOR-Donor DNA-VOR configuration as described in other Examples. For the VOR-dependent DNA replication in mitochondria, a nuclear construct was made having an expression cassette for the Rep coding region under control of an inducible promoter. The induction of expression was achieved by the dexamethasone-inducible pOp6/LhGR system similar to that described in Samalova and Moore in BMC Plant Biology 21:461, 2021 (DOI: 10.1186/s12870-021-03241-w). The construct that was made, pNAP560, had the following expression cassettes cloned into the binary vector pCAMBIA0380:
1. Maize UBI1 promoter with intron—LhGR2 transcription activator ORF—ocs terminator
2. pOp promoter—MTS:Rep ORF—35S terminator
3. pOp promoter—TagRFP ORF—nos terminator
4. 35S promoter—Hygromycin phosphotransferase ORF—CaMV terminator

The LhGR2 expression cassette is presented in SEQ ID NO: 59 and has the following elements: Maize UBI1 promoter with intron—LhGR2 transcription activator ORF—ocs terminator. The expression of two genes, MTS:Rep and TagRFP, were driven by the pOp promoter which was a bidirectional promoter, i.e., it was capable of inducing genes that were linked to each end of the promoter. The dual expression cassette containing both the MTS-Rep coding region and the TagRFP coding region is presented in SEQ ID NO: 60. The MTS sequence in the MTS-Rep ORF was derived from the Arabidopsis gene At5G47030. The expression cassette for hygromycin selection in plant cells is presented in SEQ ID NO: 61 and has the following elements: 35S promoter—hygromycin phosphotransferase ORF—CaMV terminator.

The entire insert of 10 kbp with all expression cassettes (SEQ ID NOS: 59-61) was cloned into the restriction sites PvuI and PspOMI of the Agrobacterium binary vector, pCAMBIA0380. The resulting 17 kb-long construct, named pNAP560, was transformed into the Agrobacterium tumefaciens strain, AGL1.

After Agrobacterium transformation, eight transformed lines (lines #1-#8) were selected and treated with either 10 μM DEX for induction or 10 μM DMSO as a negative control.

After 24 h, 2 days, 5 days, 6 Days and 7 days, DEX-induced RFP signal was observed using an OLYMPUS® fluorescence stereomicroscope with the RFP filter (Light volume of light source was adjusted to 100). Images were obtained by use of the OLYMPUS® CELLSENS® software. Brightfield images were captured with 300 msec light exposure. RFP images were obtained with 3 sec light exposure. Line #2 with moderate RFP expression was selected for subsequent experiments with VOR-Donor-DNA-VOR polynucleotides.

### Example 7

### Glyphosate Selection by Expression of an Herbicide-Resistant Rice EPSPS Protein in Rice Mitochondria

A polynucleotide encoding an herbicide-resistant rice EPSPS protein (EPSPS-HR) was introduced into rice mitochondria to evaluate its efficacy as a selectable marker. For expression in mitochondria, the sequence encoding the chloroplast targeting sequence of the EPSPS-HR protein is not required. The herbicide-resistant protein lacking an organellar targeting sequence was designated as mEPSPS-HR. The amino acid sequence of the mEPSPS-HR protein is presented as SEQ ID NO: 62. Two amino acid residues that can confer resistance to glyphosate in the EPSPS-HR protein are isoleucine at position 103 and serine at position 107.

The sequence encoding the mEPSPS-HR protein was optimized for expression in rice mitochondria by replacing rare codons with more frequently used codons. Several restriction enzyme sites were eliminated to facilitate cloning into existing constructs. The sequence of the optimized mEPSPS-HR coding region is presented as SEQ ID NO: 63.

Instead of encoding an AUG start codon, the 5′ end of the mEPSPS-HR ORF was fused with the 41 bp sequence (SEQ ID NO: 41) encoding the natural RNA editing site of the rice mitochondrial nad4L gene. The resulting fusion of SEQ ID NO: 41 with SEQ ID NO: 63 is presented as SEQ ID NO: 64. The rice mitochondrial nad4L RNA editing site (SEQ ID NO: 41) was used to create a translation initiation codon by use of the natural C-to-U RNA editing mechanism present in rice mitochondria. Besides an initiation methionine, SEQ ID NO: 41 also encodes an additional three new amino acids at the amino-terminus of the mEPSPS-HR protein, to result in a 449 aa herbicide-resistant protein, mEPSPS-HR*. The amino acid sequence of mEPSPS-HR* is presented as SEQ ID NO: 65.

To express the mEPSPS-HR* coding region in rice mitochondria, the sequence encoding mEPSPS-HR* (SEQ ID NO: 64) was operably linked to the promoter of the rice mitochondrial ATP1 gene. The ATP1 promoter sequence was identified from the GenBank sequence (NC_011033) and is presented as SEQ ID NO: 66.

The terminator sequence for the mEPSPS-HR* coding region was a composite of the putative terminator regions of the rice ATP1 and orf79 genes and is presented as SEQ ID NO: 67.

The homologous regions of the Donor DNA were designed to target orf79 integration downstream of the ATP1 gene. This is the position of the male-sterility orf79 gene in some rice CMS cultivars but not in the Nipponbare cultivar used for mitochondrial transformation. The 5′ homologous region (5′-HR; 1168 bp) is presented as SEQ ID NO: 68 and was derived from the mitochondrial genomic region of the rice ATP6 gene. One codon was changed with respect to the wild-type sequence at position 1025-1027 to convey oligomycin resistance.

The 3′ homologous region (3′-HR; 1203 bp) was derived from the rice mitochondrial genomic region downstream of the ATP6 gene and is presented as SEQ ID NO: 69.

The orf79 region containing the orf79 CMS gene is presented as SEQ ID NO: 70 and was inserted at the 3′-end of the 5′-HR, downstream of the ATP1 gene.

Additionally, a geminivirus VOR element was present in the plasmid DNA construct at each end of the Donor DNA, to give a VOR-Donor DNA-VOR configuration. This configuration was designed to enable Donor DNA amplification when the corresponding geminivirus Rep protein is present in the transformed rice mitochondria. The VOR element sequence used is presented as SEQ ID NO: 71.

The MEPSPS-HR* expression cassette was inserted after the 5′-HR with the orf79 gene and before the 3′-HR in the plasmid DNA construction. The resulting construct, pNAP661, was made using the cloning vector pUC-GW and has sequence elements in the following configuration: 5′-ApaI restriction site-VOR-5′ homologous region (including the rice mitochondrial ATP6 gene)-orf79 ORF-orf79 terminator-ATP1 promoter-nad4L RNA editing site-mEPSPS-HR*-ATP1/orf79 terminators-3′ homologous region-VOR-AscI restriction site-3′.

The entire VOR-Donor DNA-VOR fragment (6307 bp) was released from the plasmid DNA by digestion with the two restriction enzymes ApaI and AscI. The VOR-Donor DNA-VOR fragment was used for rice mitochondrial transformation with glyphosate selection as described in EXAMPLE 5.

### Example 8

### Glufosinate Selection by Expression of an Herbicide-Resistant Rice GS1 Protein in Rice Mitochondria

A polynucleotide encoding an herbicide-resistant rice glutamine synthetase GS1 protein (GS1-HR) was introduced into rice mitochondria to evaluate its efficacy as a selectable marker. The rice GS1 glutamine synthetase is a cytosolic protein. The amino acid sequence of the GS1-HR protein that was expressed in mitochondria is presented as SEQ ID NO: 72. A change of serine-to-glycine at amino acid residue 61 can confer resistance to glufosinate.

The sequence encoding GS1-HR was optimized for expression in rice mitochondria by replacing rare codons with more frequently used codons as well as eliminating several restriction sites to facilitate cloning into existing constructs. The sequence of the optimized GS1-HR coding region minus the translation initiation codon is presented as SEQ ID NO: 73.

Instead of encoding an AUG start codon, the 5′-end of the GS1-HR ORF was fused with the 41 bp sequence (SEQ ID NO: 41) encoding the natural RNA editing site of the rice mitochondrial nad4L gene. The resulting fusion of SEQ ID NO: 41 with SEQ ID NO: 73 is presented as SEQ ID NO: 74. The rice mitochondrial nad4L RNA editing site (SEQ ID NO: 41) was used to create a translation initiation codon by use of the natural C-to-U RNA editing mechanism present in rice mitochondria. SEQ ID NO: 41 also encodes an additional three new amino acids at the amino-terminus of the GS1-HR protein, to result in a 373 aa herbicide-resistant protein, GS1-HR*. The amino acid sequence of GS1-HR* is presented as SEQ ID NO: 75.

To express the GS1-HR* coding region in rice mitochondria, the sequence encoding GS1-HR* (SEQ ID NO: 74) was operably linked to the promoter of the rice mitochondrial ATP1 gene (SEQ ID NO: 66).

The terminator sequence for the GS1-HR* coding region was a composite of the putative terminator regions of the rice ATP1 and orf79 genes and is presented as SEQ ID NO: 67.

The homologous regions of the Donor DNA were designed to target orf79 integration downstream of the ATP1 gene. This is the position of the male-sterility orf79 gene in some rice CMS cultivars but not in the Nipponbare cultivar used for mitochondrial transformation. The 5′ homologous region (5′-HR; 1168 bp) is presented as SEQ ID NO: 68 and was derived from the mitochondrial genomic region of the rice ATP6 gene. One codon was changed with respect to the wild-type sequence at position 1025-1027 to convey oligomycin resistance. A CsiI restriction site is present at nucleotides 570-576.

The 3′ homologous region (3′-HR; 1203 bp) was derived from the rice mitochondrial genomic region downstream of the ATP6 gene and is presented as SEQ ID NO: 69. A BmtI restriction site is present at nucleotides 1076-1081.

The orf79 region containing the orf79 CMS gene is presented as SEQ ID NO: 70 and was inserted at the 3′-end of the 5′-HR, downstream of the ATP1 gene.

The GS1-HR* expression cassette was inserted after the 5′-HR with the orf79 gene and before the 3′-HR in the plasmid DNA construction. The resulting construct, pNAP643, was made in the cloning vector pUC-GW and has sequence elements in the following configuration: 5′ homologous region (including the rice mitochondrial ATP6 gene)-orf79 (rice mitochondrial male-sterility gene)-ATP1 promoter-nad4L RNA editing site-GS1-HR*-ATP1/orf79 terminators-3′ homologous region-3′.

A truncated Donor DNA fragment of 4949 bp was released from the construct by digestion with two restriction enzymes, CsiI and BmtI, present in the 5′-HR and 3′-HR, respectively. The gel-purified 4949 bp truncated Donor DNA fragment was used for rice mitochondrial transformation as described in EXAMPLE 5.

### Example 9

### Chlorsulfuron Selection of Transformed Wheat Cells

Approximately twenty-four hours prior to transformation, immature scutella approximately 2 mm in length of wheat cultivars Fielder and/or Bobwhite were prepared for transformation by excising them from immature seeds, removing the small embryo axis, and plating them in a circular target zone on a high-osmotic medium. This medium was an agar-solidified MS basal medium supplemented with amino acids, 90 g/L sucrose and 2 mg/L 2,4-D, with or without the addition of cefotaxime antibiotic at the rate of 250 mg/L for contamination control.

The precultured wheat scutella were transformed with mitochondrial ALS-HR expression constructs (e.g., herbicide-resistant ALS large subunit) using the biolistics method (particle bombardment). In some examples, the scutella were co-transformed with a mitochondrial oligomycin resistance gene (oliR) linked to the ALS chlorsulfuron resistance gene. In some experiments, geminivirus VOR sequences (target sites for the geminivirus Rep protein) were also present in the construct. After bombardment, the following steps were used for culture, selection, and regeneration.

1. Immediately after bombardment, or up to 2 days after bombardment, the scutella were spread out across the bombarded plates or spaced out onto additional new plates of the same high osmotic medium and incubated in the dark for up to 7 days at 26° C. Next the cultured scutella were transferred to a selective callus induction medium, which was the MS-based high osmotic medium supplemented with 25-30 nM chlorsulfuron and 250 mg/L cefotaxime, then returned to dark incubation for 3 weeks.

After 3 weeks of incubation on selective callus induction medium containing chlorsulfuron, the scutella received one of two treatments. In one treatment, approximately half of the scutella were continuously maintained on the MS-based selective callus induction medium with chlorsulfuron, while in a second treatment the remaining scutella were transferred to a first stage agarose-solidified regeneration medium (RZ) supplemented with maltose, 2,4-D, zeatin and silver nitrate with continued chlorsulfuron selection. Cefotaxime use was discontinued after three to six weeks of culture on the callus induction medium.

The scutella on callus induction medium were cultured in the light (16/8 photoperiod) and transferred to fresh medium with chlorsulfuron approximately every three weeks for 32 weeks, then sampled for PCR analysis for donor DNA integration. Calli induced from individual bombarded scutella were sometimes subdivided into smaller pieces as they were transferred to plates of the fresh selective media, marking on the plate all pieces which came from each original scutellum, to maintain their original identity.

Scutella on shoot induction medium were subcultured to fresh first stage regeneration medium every three weeks and cultured in the light until shoot formation was visible. At that time, selected green sectors of callus and small shoots were transferred to a second stage regeneration medium (R0) which was the same as the first stage regeneration medium, but without growth regulators. Developing plantlets were transferred to domed clear culture vessels and grown on to transplantable size. They were then transplanted to soil and acclimatized in the greenhouse.

### Dual Selection Process

When an ALS-HR expression cassette was linked to an oligomycin resistance expression cassette, selection of transformation events was in some cases augmented by the addition of 1 mg/L oligomycin to the callus induction medium. In some experiments where oligomycin was a selective agent, the compound disulfiram was also incorporated into the medium at 100 μM concentration.

In other experiments (with or without 35S:HPT co-expression), the ALS-HR expression cassette was also linked and co-transformed with an oligomycin-resistant oliR expression cassette. In some experiments, geminivirus VOR sequences (target sites for the geminivirus Rep protein) were also present in the construct. In some experiments, oligomycin was incorporated into the selective medium at a rate of 1-5 mg/L.

### Example 10

Wheat Mitochondrial Gene Editing with Donor DNA Encoding Rice mALS(HR-LS)

DNA fragments containing Donor DNA encoding an herbicide-resistant ALS fusion protein were used to transform wheat mitochondria. A fusion protein was designed in which amino acids 6-631 of the herbicide-resistant rice mALS(HR-LS) protein (SEQ ID NO: 24) were fused with the eGFP protein (SEQ ID NO: 76) by use of a 4 aa PVAT linker (SEQ ID NO: 77). The resulting mALS(HR-LS)-eGFP fusion protein is presented as SEQ ID NO: 78.

A DNA sequence encoding the mALS(HR-LS)-eGFP fusion protein was designed with optimized codons for the expression in wheat mitochondria and is presented as SEQ ID NO: 79.

Instead of encoding an AUG start codon, the 5′ end of the mALS(HR-LS)-eGFP ORF was fused with a 75 bp sequence (SEQ ID NO: 80) encoding the natural RNA editing site of the wheat mitochondrial cox2 gene. The resulting fusion of SEQ ID NO: 80 with SEQ ID NO: 79 is presented as SEQ ID NO: 81. The wheat mitochondrial cox2 RNA editing site (SEQ ID NO: 80) was used to create a translation initiation codon by use of the natural C-to-U RNA editing mechanism present in wheat mitochondria. Besides an initiation methionine, SEQ ID NO: 80 also encodes an additional 11 new amino acids at the amino-terminus of the mALS(HR-LS)-eGFP protein, to result in an 881 aa herbicide-resistant fusion protein, mALS(HR-LS)-eGFP*. The amino acid sequence of mALS(HR-LS)-eGFP* is presented as SEQ ID NO: 82.

The DNA sequence encoding mALS(HR-LS)-eGFP* was fused with the rice mitochondrial ATP1 promoter sequence (SEQ ID NO: 66).

The DNA fragment containing the sequence of the ATP1 promoter-mALS(HR-LS)-eGFP* region was fused at its 3′ end with a DNA fragment (SEQ ID NO: 83) containing a gRNA expression cassette and terminators. The gRNA cassette was composed of the T7 promoter and four gRNAs with constant repeat regions required for MAD7 processing. All gRNAs were targeted to the mitochondrial genomic region that was replaced with Donor DNA to eliminate wild-type mitochondrial DNA if needed. The terminator region was composed of a T7 RNA polymerase terminator and a rice ATP1 terminator.

A DNA fragment (SEQ ID NO: 84) containing the wheat mitochondrial atp6-1 gene with promoter and terminator regions was added to the plasmid DNA construct. The wheat atp6-1 sequence in SEQ ID NO: 84 was altered from the wild-type sequence to encode a variant protein with oligomycin resistance.

A 222 bp-long DNA (SEQ ID NO: 85) was inserted at the 3′-end of the atp6-1 element in the Donor DNA region. SEQ ID NO: 85 has an I-SceI restriction site followed by the orf279 terminator region. This second copy of the orf279 terminator region could serve as a directly repeated sequence within the Donor DNA to facilitate deletion of the herbicide-resistant and oligomycin-resistant selectable marker genes upon cleavage by the I-SceI restriction enzyme.

For the target site of the Donor DNA, we chose the atp8-1 gene region of the wheat mitochondrial genome. This is the location of the cytoplasmic male-sterility gene, orf279, in the Triticum timopheevii background (Melonek et al. 2021 Nature Communication 12:1036, DOI: 10.1038/s41467-021-21225-0) and was published in GenBank (accession #NC_022714). The orf279 ORF from Triticum timopheevii is a fusion of mitochondrial atp8-1 sequence also present in Triticum aestivum and a sequence specific to Triticum timopheevii mitochondrial DNA. The 5′ homologous region of the Donor DNA contains 1201 bp of Triticum aestivum sequence that includes wild-type atp8-1 sequence present in the orf279 coding region. The DNA sequence comprising both the 5′ homologous region (5′-HR) from Triticum aestivum and the Triticum timopheevii specific part of the orf279 coding region is presented as SEQ ID NO: 86. The Triticum aestivum 5′-HR of the Donor DNA corresponds to nucleotides 1-1201 of SEQ ID NO: 86 and the Triticum timopheevii specific sequence corresponds to nucleotides 1202-1952.

The sequence of the 3′ homologous region (3′-HR) of the Donor DNA is from Triticum aestivum and is presented as SEQ ID NO: 87.

Additionally, a geminivirus VOR element was present in the plasmid DNA construct at each end of the Donor DNA, to give a VOR-Donor DNA-VOR configuration. This configuration was designed to enable Donor DNA amplification when the corresponding geminivirus Rep protein is present in the transformed rice mitochondria. The VOR element sequence used is presented as SEQ ID NO: 71.

The resulting construct, pNAP653, was made using the cloning vector pUC-GW and has sequence elements in the following configuration: VOR-5′ homologous region (atp8-1 sequence)-Triticum timopheevii specific part of orf279 ORF-orf279 terminator-I-SceI-ATP1 promoter-cox2 RNA editing site-mALS(HR-LS)-eGFP*-T7 promoter-gRNA cassette-T7/ATP1 terminators-atp6-1 with oligomycin resistance-I-SceI-orf279 terminator-3′ homologous region-VOR.

The pNAP653 plasmid DNA was digested with restriction enzymes, AscI and NotI to release a 10 kb Donor DNA fragment for mitochondrial transformation.

A related plasmid DNA, pNAP652, contains the rice orf79 coding region fused to the 3′-end of the wheat atp6-1 ORF. The sequence of the Donor DNA region from pNAP652 containing the wheat atp6-1 ORF and the rice orf79 ORF is presented as SEQ ID NO: 88.

The pNAP652 plasmid DNA has sequence elements in the following features: VOR-5′ homologous region (atp8-1 sequence)-Triticum timopheevii specific part of orf279 ORF-orf279 terminator-I-SceI-ATP1 promoter-cox2 RNA editing site-mALS(HR-LS)-eGFP*-T7 promoter-gRNA cassette-T7/ATP1 terminators-atp6-1 with oligomycin resistance-rice orf79-I-SceI-orf279 terminator-3′ homologous region-VOR.

Plasmid pNAP652 DNA was digested with restriction enzymes AscI and NotI to release an 11 kb Donor DNA fragment for mitochondrial transformation.

Wheat mitochondrial transformations were performed by bombardment essentially as described in EXAMPLE 9. After 2-3 months of selection on sulfonylurea, callus growth of putative transformed events was observed.

### Example 11

### PCR and Sequence Analysis of Sulfonylurea-Resistant Events

To confirm the integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. In this example, we used two primer pairs for amplifying each junction region. The first set of primers for the 5′ junction was: 5HRBst (SEQ ID NO: 89) and ORF79st (SEQ ID NO: 90). The second set of primers for nested PCR was: 5HRAst (SEQ ID NO: 91) and ORFBst (SEQ ID NO: 92). For the 3′ junction region, we used the following first set of primer pairs: 3HRBst (SEQ ID NO: 93) and 420Bst (SEQ ID NO: 94). The second set of primers for nested PRC was: 3HRAst (SEQ ID NO: 95) and 420Ast (SEQ ID NO: 96).

For PCR analyses, plant material (from either callus or leaf tissue) of each positive event (5-20 mg) was sampled in a tube. 300 μl of 0.02 N NaOH with 10 mM EDTA was added to each sample and heated for 20 min at 100° C. Then, the aqueous phase was extracted with phenol/chloroform and subsequently with chloroform. Total DNA was precipitated by the addition of NaOAc and ethanol. DNA was resuspended in 30-60 μl 0.1×TE. The DNA yield was about 100-200 ng/μl on average. The PCR reaction was prepared as follows: 0.8 μl of total DNA, 10 pmol of each primer and 7.5 μl LONGAMP® Hot Start Taq 2× Master Mix (New England Biolabs, Inc.) in a 15 μl reaction mix.

The PCR reaction with the first primer pair for the 5′ junction amplification was performed for 2 min at 94° C., then 22 cycles of 15 seconds at 94° C., 30 seconds at 63° C., and 2 min at 65° C. followed by the final incubation for ten minutes at 65° C. The nested PCR reaction with the second primer pair was performed with 0.3 μl of the first PCR reaction in 15 μl total volume under the amplification condition of 2 min at 94° C., then 22 cycles of 15 seconds at 94° C., 30 seconds 62° C., and 2 minutes at 65° C., followed by the final incubation for ten minutes at 65° C.

The first PCR reaction with the first primer pair for the 3′ junction amplification was performed by 2 min at 94° C., then 22 cycles of 15 seconds at 94° C., 30 seconds at 62° C., and 1 min 30 seconds at 65° C., followed by the final incubation for ten minutes at 65° C. The nested PCR reaction with the second primer pair was performed with 0.3 μl of the first PCR reaction in 15 μl total volume under the amplification condition of 2 min at 94° C., then 22 cycles of 15 seconds at 94° C., 30 seconds 62° C., and 1 minute 30 seconds at 65° C., followed by the final incubation for ten minutes at 65° C.

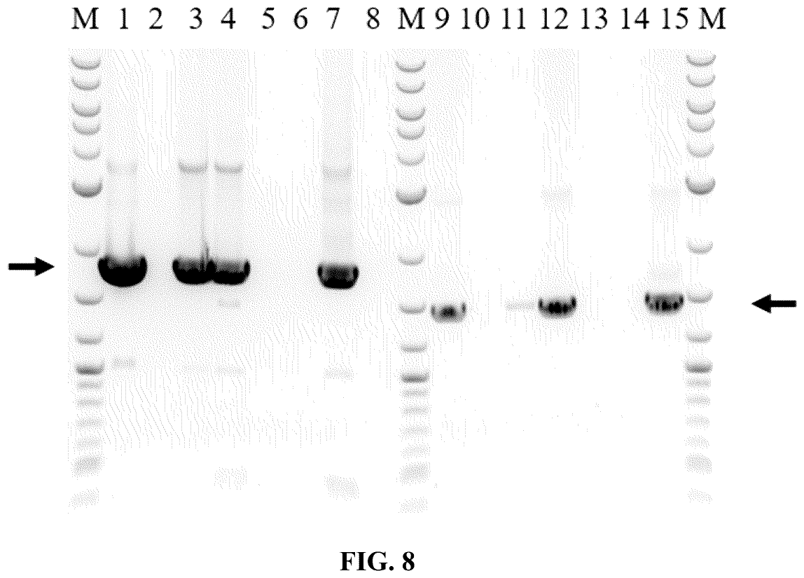

PCR products having the correct sizes for the 5′-junction DNA and the 3′-junction DNA were amplified from multiple samples that were selected on media containing sulfonylurea (FIG. 8).

PCR fragments corresponding to the 5′-junction were isolated from gels and subjected to sequence analysis. Sequencing of the 5′-junction fragments was performed with primers 5HRAst (SEQ ID NO: 91), 5HR_for_6 (SEQ ID NO: 97), 5HR_for_4 (SEQ ID NO: 98), and Invitro_for_1 (SEQ ID NO: 99).

PCR fragments corresponding to the 3′-junction were isolated from gels and subjected to sequence analysis. Sequencing of the 3′-junction fragments was performed with primers 3HR_rev_4 (SEQ ID NO: 100), 3HR_seq_for (SEQ ID NO: 101), 3HR_seq_rev (SEQ ID NO: 102).

Sequence data from the 5′-junction and the 3′-junction regions were obtained from leaf tissues of a regenerated positive event, HH43. This event was from rice callus that had been cotransformed with the Donor DNA fragment of pNAP432 and the plasmid pNAP195 (EXAMPLE 4), and had been selected under the continuous application of sulfonylurea herbicide. All junction fragments had sequences corresponding to the correct integration of Donor DNA at the homologous regions.

Sequence data from a 5′-junction PCR fragment from leaf tissue of the sulfonylurea-resistant HH43 plant is presented as SEQ ID NO: 103 (1739 nt). Nucleotides 1-31 correspond to wild-type mitochondrial sequence not present in the Donor DNA. Nucleotides 32-1646 correspond to sequence of the 5′-HR. Nucleotides 1647-1739 correspond to sequence specific to the Donor DNA and not present in the wild-type mitochondrial sequence.

Sequence data from a 3′-junction PCR fragment from leaf tissue of the sulfonylurea-resistant HH43 plant is presented as SEQ ID NO: 104 (1370 nt). Nucleotides 1-13 correspond to sequence specific to the Donor DNA and not present in the wild-type mitochondrial sequence. Nucleotides 14-1216 correspond to sequence of the 3′-HR. Nucleotides 1217-1370 correspond to wild-type mitochondrial sequence not present in the Donor DNA.

SEQ ID NO: 103 and SEQ ID NO: 104 demonstrate that in the HH43 event the Donor DNA was correctly integrated into the targeted 5′ homologous region and the targeted 3′ homologous region of the rice mitochondrial DNA.

### Example 12

### PCR and Sequence Analysis of Glyphosate-Resistant Events

To confirm the integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. In this example, we used two primer pairs for amplifying the 5′ junction region. The first set of primers was: 5HRBst (SEQ ID NO: 89) and ORF79st (SEQ ID NO: 90). The second set of primers for nested PCR was: 5HRAst (SEQ ID NO: 91) and ORFBst (SEQ ID NO: 92). For the 3′ junction region, we used one primer pair. The primer pair that we used for amplifying the 3′ junction region was: 3HRAst (SEQ ID NO: 95) and 420Ast (SEQ ID NO: 96).

For PCR analyses, callus of each positive event (5-20 mg) was sampled in a tube. 300 μl of 0.02 N NaOH with 1 mM EDTA were added to each sample and heated for 20 min at 100° C. Then, the aqueous phase was extracted with phenol/chloroform and subsequently with chloroform. Total DNA was precipitated by the addition of NaOAc and ethanol. DNA was resuspended in 30-60 μl TE. The DNA yield was about 100-200 ng/μl on average. The PCR reaction was prepared as follows: 0.6 μl, 0.8 μl, or 1.0 μl of total DNA; 10 pmol of each primer; and 7.5 μl, 10 μl, or 12.5 μl LONGAMP® Hot Start Taq 2× Master Mix (New England Biolabs Inc.); in a 15 μl, 20 μl, or 25 μl reaction mix, respectively.

The PCR reaction with the first primer pair for the 5′ junction amplification was performed for 1 min 30 seconds at 94° C., then 22 cycles of 15 seconds at 94° C., 30 seconds at 63° C., and 2 min 15 seconds at 65° C. followed by the final incubation for ten minutes at 65° C. The nested PCR reaction with the second primer pair was performed with 0.8 μl of the first PCR reaction under the amplification condition of 1 min 30 seconds at 94° C., then 22 cycles of 15 seconds at 94° C., 30 seconds 63° C., and 2 minutes at 65° C., followed by the final incubation for ten minutes at 65° C.

The PCR reaction for the 3′ junction amplification was performed for 1 min 30 seconds at 94° C., then 40 cycles of 15 seconds at 94° C., 30 seconds at 60° C. and 2 minutes at 65° C., followed by the final incubation for 10 minutes at 65° C. The PCR samples were separated on 0.7-0.8% agarose gels.

Selected DNA bands were isolated from gels and subjected to sequence analysis. Sequencing of PCR fragments was performed with primers used for PCR amplification. Some of the PCR fragments were sequenced with primers hybridizing in the 3′ HR to obtain the sequence over the 3′ HR- and Donor DNA-specific regions. Those additional primers were 3Hrrev3 (SEQ ID NO: 105) and 3HR_seq_Rev (SEQ ID NO: 102).

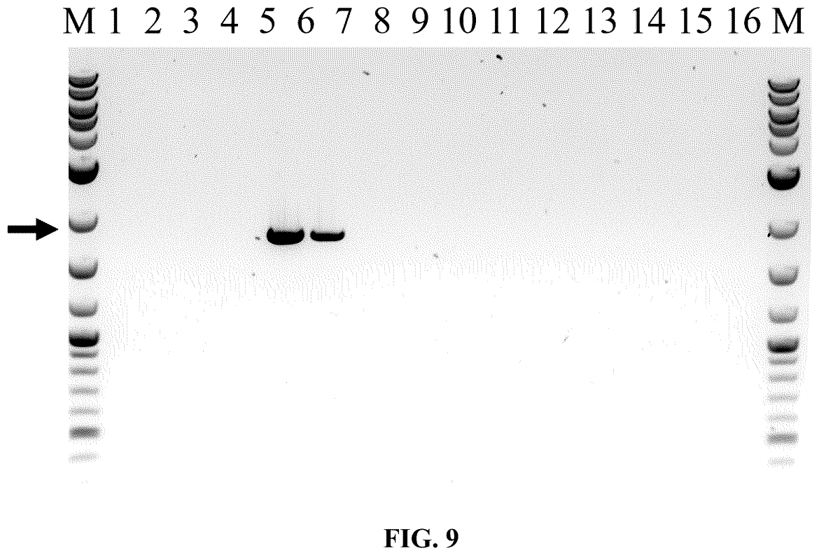

Rice callus tissue was transformed with a Donor DNA fragment from plasmid Pnap661, and glyphosate-resistant events were selected (EXAMPLE 7). One of the glyphosate-resistant events, TT57, was shown to have a 5′-junction fragment of 1.8 kb, which was the expected length (FIG. 9). The two ends of this PCR fragment were sequenced. The 5′-end is presented as SEQ ID NO: 106 and the 3′-end is presented as SEQ ID NO: 107. In SEQ ID NO: 106, nucleotides 1-425 correspond to wild-type mitochondrial sequence not present in the Donor DNA and nucleotides 426-796 correspond to sequences from the 5′-HR of the Donor DNA. In SEQ ID NO: 107, nucleotides 1-780 correspond to sequences from the 5′-HR of the Donor DNA and nucleotides 781-850 correspond to sequences specific to the Donor DNA and not present in the wild-type mitochondrial sequence. SEQ ID NO: 106 and SEQ ID NO: 107 demonstrate that the Donor DNA was correctly integrated at the targeted 5′-homologous region of the rice mitochondrial DNA.

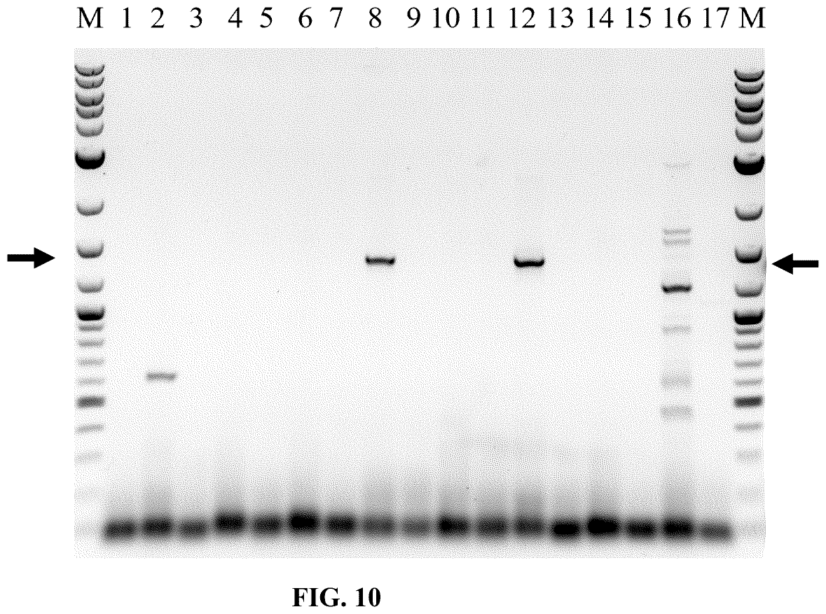

One of the glyphosate-resistant events, TT31, was shown to have a 3′-junction fragment of 1.4 kb, which was approximately 0.3 kb shorter than expected (FIG. 10). This PCR fragment was sequenced, and the sequence is presented in SEQ ID NO: 108. In SEQ ID NO: 108, nucleotides 1-38 correspond to sequences specific to the Donor DNA and not present in the wild-type mitochondrial sequence; however, a 297 nt sequence containing the second copy of the orf79 terminator in the Donor DNA appears to have been deleted. Nucleotides 39-42 correspond to a 4 nt novel sequence at the site of the deletion of the second orf79 terminator. Nucleotides 43-1241 correspond to sequences from the 3′-HR of the Donor DNA. Nucleotides 1242-1365 correspond to sequences from wild-type mitochondrial sequence not present in the Donor DNA. SEQ ID NO: 108 shows that the Donor DNA has integrated at the targeted 3′-homologous region of the rice mitochondrial DNA; however, the second copy of the orf79 terminator in the Donor DNA was deleted, presumably by an endogenous mitochondrial DNA recombination mechanism.

### Example 13

### PCR and Sequence Analysis of Glufosinate-Resistant Events

To confirm the integration of Donor DNA into the mitochondrial genome, PCR analyses of the junction regions were performed. Each junction region was amplified with a primer specific to the Donor DNA region and another primer specific to mitochondrial genomic sequence in the vicinity of the homologous region of Donor DNA. In this example, we used two primer pairs for amplifying the 5′ junction region. The first set of primers was: 5HRBst (SEQ ID NO: 89) and ORF79st (SEQ ID NO: 90). The second set of primers for nested PCR was: 5HRAst (SEQ ID NO: 91) and ORFBst (SEQ ID NO: 92). For the 3′ junction region, we used one primer pair. The primer pair that we used for amplifying the 3′ junction region was: 3HRAst (SEQ ID NO: 95) and 420Ast (SEQ ID NO: 96).

For PCR analyses, callus of each positive event (5-20 mg) was sampled in a tube. 300 μl of 0.02 N NaOH with 1 mM EDTA was added to each sample and heated for 20 min at 100° C. Then, the aqueous phase was extracted with phenol/chloroform and subsequently with chloroform. Total DNA was precipitated by the addition of NaOAc and ethanol. DNA was resuspended in 30-60 μl TE. The DNA yield was about 100-200 ng/μl on average. The PCR reaction was prepared as follows: 0.6 μl, 0.8 μl, or 1.0 μl of total DNA; 10 pmol of each primer; and 7.5 μl, 10 μl, or 12.5 μl LONGAMP® Hot Start Taq 2× Master Mix (New England Biolabs, Inc.); in a 15 μl, 20 μl, or 25 μl reaction mix, respectively.

The PCR reaction with the first primer pair for the 5′ junction amplification was performed for 1 min 30 seconds at 94° C., then 22 cycles of 15 seconds at 94° C., 30 seconds at 63° C., and 2 min 15 seconds at 65° C. followed by the final incubation for ten minutes at 65° C. The nested PCR reaction with the second primer pair was performed with 0.8 μl of the first PCR reaction under the amplification condition of 1 min 30 seconds at 94° C., then 22 cycles of 15 seconds at 94° C., 30 seconds 63° C., and 2 minutes at 65° C., followed by the final incubation for ten minutes at 65° C.

The PCR reaction for the 3′ junction amplification was performed for 1 min 30 seconds at 94° C., then 40 cycles of 15 seconds at 94° C., 30 seconds at 60° C. and 2 minutes at 65° C., followed by the final incubation for 10 minutes at 65° C. The PCR samples were separated on 0.7-0.8% agarose gels.

Selected DNA bands were isolated from gels and subjected to sequence analysis. Sequencing of PCR fragments was performed with primers used for PCR amplification. Some of the PCR fragments were sequenced with primers hybridizing in the 3′ HR to obtain the sequence over the 3′ HR- and Donor DNA-specific regions. Those additional primers were 3HRrev3 (SEQ ID NO: 105) and 3HR_seq_Rev (SEQ ID NO: 102).

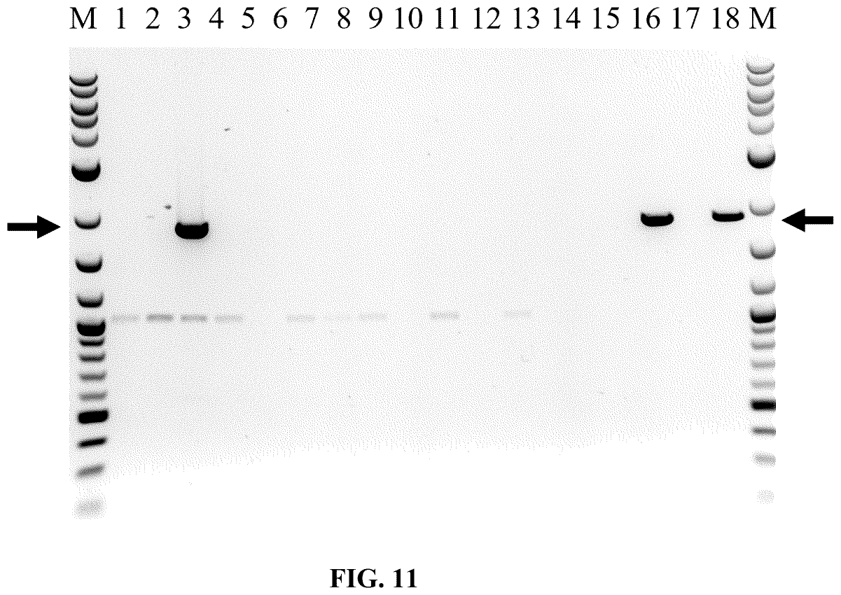

Rice callus tissue was transformed with a truncated CsiI-BmtI Donor DNA fragment from plasmid pNAP643, and glufosinate-resistant events were selected (EXAMPLE 8). Events S96, S109 and S111 produced PCR fragments of the expected size (1.8 kb) for the 5′ integration site (FIG. 11). SEQ ID NO: 109 corresponds to the nucleotide sequence (871 nt) of the 5′-junction PCR fragment from glufosinate-resistant line S96. Nucleotides 1-202 correspond to wild-type mitochondrial sequence not present in the truncated Donor DNA fragment. Nucleotides 203-800 correspond to sequences from the 5′-HR of the truncated Donor DNA fragment. Nucleotides 801-871 correspond to sequences specific to the truncated Donor DNA fragment and not present in the wild-type mitochondrial sequence. SEQ ID NO: 109 demonstrates that the Donor DNA was correctly integrated at the targeted 5′-homologous region of the rice mitochondrial DNA.

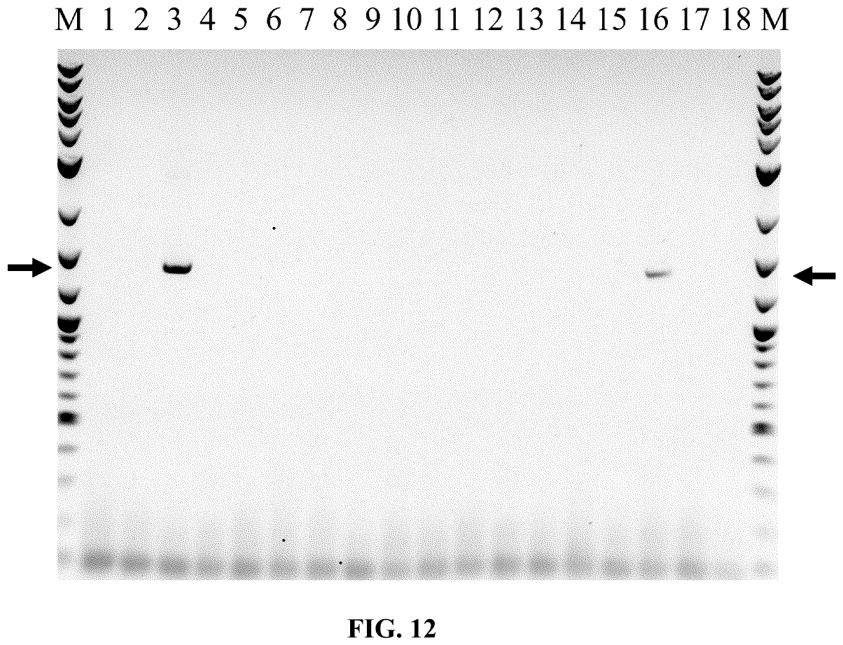

Two of the glufosinate-resistant events, S96 and S109, were shown to have a 3′-junction fragment of 1.4 kb, which was approximately 0.3 kb shorter than expected (FIG. 12). SEQ ID NO: 110 corresponds to the nucleotide sequence (1376 nt) from the 3′-junction PCR fragment from glufosinate-resistant line S96. Nucleotides 1-36 correspond to sequences specific to the truncated Donor DNA fragment and not present in the wild-type mitochondrial sequence; however, a 297 nt sequence containing the second copy of the orf79 terminator in the Donor DNA appears to have been deleted. Nucleotides 37-40 correspond to novel sequence at the site of the deletion of the second orf79 terminator. Nucleotides 41-1121 correspond to sequences from the 3′-HR of the truncated Donor DNA fragment. Nucleotides 1121-1376 correspond to sequences from wild-type mitochondrial sequence not present in the truncated Donor DNA fragment. SEQ ID NO: 110 shows that the Donor DNA has integrated at the targeted 3′-homologous region of the rice mitochondrial DNA; however, the second copy of the orf79 terminator in the Donor DNA was deleted, presumably by an endogenous mitochondrial DNA recombination mechanism.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### BRIEF DESCRIPTION OF THE SEQUENCES

A full list of the sequences disclosed in this application is provided in TABLE 3. Information regarding the sequences is provided below and throughout the description and examples of the specification:

SEQ ID NO: 1 (Artificial sequence) corresponds to a conserved sequence motif for one of the four Meganuclease families.

SEQ ID NO: 2 (Artificial sequence) corresponds to the amino acid sequence of a hydrophobic quenching peptide that tetramerizes GFP and prevents maturation of the chromophore.

SEQ ID NO: 3 (Artificial sequence) corresponds to the amino acid sequence of a caspase recognition sequence.

SEQ ID NO: 4 (Triticum aestivum) corresponds to the nucleotide sequence for a candidate RNA editing sequence present in the wheat mitochondrial cox2 gene at position 449 of the gene.

SEQ ID NO: 5 (Triticum aestivum) corresponds to the nucleotide sequence for a candidate RNA editing sequences present in the wheat mitochondrial cox2 gene at position 587 of the gene.

SEQ ID NO: 6 (Triticum aestivum) corresponds to the nucleotide sequence for a candidate RNA editing sequence present in the wheat mitochondrial cox2 gene at position 620 of the gene.

SEQ ID NO: 7 (Oryza sativa) corresponds to the amino acid sequence of an herbicide-resistant ALS large subunit (ALS(HR-LS)) polypeptide from Oryza sativa.

SEQ ID NO: 8 (Oryza sativa) corresponds to the nucleotide sequence encoding the first 18 amino acid residues (the chloroplast targeting sequence) of the herbicide-resistant ALS large subunit polypeptide from Oryza sativa encoded by SEQ ID NO: 7.

SEQ ID NO: 9 (Arabidopsis thaliana) corresponds to the nucleotide sequence encoding the mitochondrial targeting sequence (MTS) of the Arabidopsis thaliana mitochondrial ATP synthase subunit delta protein (gene ID: At5g47030).

SEQ ID NO: 10 (Artificial sequence) corresponds to the nucleotide sequence encoding the MTS-ALS(HR-LS) fusion protein.

SEQ ID NO: 11 (Artificial sequence) corresponds to the amino acid sequence of the MTS-ALS(HR-LS) fusion protein encoded by SEQ ID NO: 10. The first 56 amino acids correspond to the MTS of the Arabidopsis thaliana rsp10 protein.

SEQ ID NO: 12 (Artificial sequence) corresponds to the nucleotide sequence of a maize UBI1 promoter and intron.

SEQ ID NO: 13 (Agrobacterium tumefaciens) corresponds to the nucleotide sequence of a NOS terminator.

SEQ ID NO: 14 (Artificial sequence) corresponds to the nucleotide sequence encoding the hygromycin phosphotransferase protein.

SEQ ID NO: 15 (Artificial sequence) corresponds to the nucleotide sequence of a 35S promoter.

SEQ ID NO: 16 (Artificial sequence) corresponds to the nucleotide sequence of a CaMV terminator.

SEQ ID NO: 17 (Oryza sativa) corresponds to the amino acid sequence of a rice ALS(SS), an ALS small subunit polypeptide.

SEQ ID NO: 18 (Oryza sativa) corresponds to the amino acid sequence of the first 47 amino acids (i.e., the putative chloroplast targeting sequence) of the rice ALS(SS).

SEQ ID NO: 19 (Arabidopsis thaliana) corresponds to the nucleotide sequence encoding the MTS of the Arabidopsis At5g47030 gene.

SEQ ID NO: 20 (Artificial sequence) corresponds to the nucleotide sequence encoding the MTS-ALS(SS) fusion protein.

SEQ ID NO: 21 (Artificial sequence) corresponds to the amino acid sequence of the MTS-ALS(SS) fusion protein encoded by SEQ ID NO: 20. The first 36 amino acids correspond to the MTS of the Arabidopsis At5g47030 gene.

SEQ ID NO: 22 (Oryza sativa) corresponds to the nucleotide sequence of a rice Actin1 promoter and intron.

SEQ ID NO: 23 (Artificial sequence) corresponds to the nucleotide sequence of an OCS terminator.

SEQ ID NO: 24 (Artificial sequence) corresponds to the amino acid sequence of mALS(HR-LS), the herbicide-resistant ALS large subunit protein lacking a functional amino-terminal chloroplast transit sequence, for expression in mitochondria.

SEQ ID NO: 25 (Artificial sequence) corresponds to the nucleotide sequence encoding mALS(HR-LS) which was modified by removal of certain restriction sites and was optimized for expression in rice mitochondria by replacing rare codons for mitochondria with more frequently used codons.

SEQ ID NO: 26 (Escherichia phage T7) corresponds to the nucleotide sequence of a T7 promoter.

SEQ ID NO: 27 (Artificial sequence) corresponds to the nucleotide sequence of a hybrid ATP1+T7 promoter.

SEQ ID NO: 28 (Escherichia phage T7) corresponds to the nucleotide sequence of a T7 terminator.

SEQ ID NO: 29 (Artificial sequence) corresponds to the nucleotide sequence of a hybrid T7+ATP1 terminator.

SEQ ID NO: 30 (Artificial sequence) corresponds to the nucleotide sequence of a eGFP reporter in which the coding sequence has been modified to have an RNA editing element derived from rice COX2 to create the translation initiation codon. The first 27 nucleotides comprise the RNA editing element and the C residue at nucleotide 17 is the RNA editing site.

SEQ ID NO: 31 (Oryza sativa) corresponds to the nucleotide sequence of a rice COB1 promoter and 5′ UTR.

SEQ ID NO: 32 (Oryza sativa) corresponds to the nucleotide sequence of a rice COB1 terminator.

SEQ ID NO: 33 (Artificial sequence) corresponds to the nucleotide sequence encoding the MTS-T7 RNA polymerase. The first 108 nucleotides encode the MTS derived from At5g47030.

SEQ ID NO: 34 (Artificial sequence) corresponds to the amino acid sequence of the MTS-T7 RNA polymerase. The first 36 amino acids are the MTS derived from At5g47030.

SEQ ID NO: 35 (Arabidopsis thaliana) corresponds to the amino acid sequence of the MTS derived from At5g47030.

SEQ ID NO: 36 (Artificial sequence) corresponds to the nucleotide sequence of the 5′ homologous region in the Donor DNA of plasmids pNAP432 and pNAP433. Certain nucleotides were changed in the 5′ homologous region to prevent future recognition by gRNA2 and the MAD7 enzyme.

SEQ ID NO: 37 (Artificial sequence) corresponds to the nucleotide sequence encoding gRNA2.

SEQ ID NO: 38 (Artificial sequence) corresponds to the amino acid sequence of MAD7.

SEQ ID NO: 39 (Artificial sequence) corresponds to the nucleotide sequence of the 3′ homologous region of the Donor DNA of plasmids pNAP432 and pNAP433. Certain nucleotides were changed in the 3′ homologous region to prevent future recognition by gRNA4 and the MAD7 enzyme.

SEQ ID NO: 40 (Artificial sequence) corresponds to the nucleotide sequence encoding gRNA4.

SEQ ID NO: 41 (Oryza sativa) corresponds to the nucleotide sequence encoding an RNA editing site of the rice mitochondrial nad4L transcript.

SEQ ID NO: 42 (Oryza sativa) corresponds to the nucleotide sequence encoding an RNA editing site of the rice mitochondrial cox2 transcript.

SEQ ID NO: 43 (Artificial sequence) corresponds to the nucleotide sequence of a truncated version of the hybrid T7+ATP1 terminator presented as SEQ ID NO: 29.

SEQ ID NO: 44 (Artificial sequence) corresponds to the nucleotide sequence of the pNAP432 Donor DNA fragment having the rice mitochondrial nad4L RNA editing site. In TABLE 3, the homologous regions are underlined, the mALS(HR-LS) ORF is highlighted in bold, and the RNA editing site to create AUG in mitochondria is shown in lower case.

SEQ ID NO: 45 (Artificial sequence) corresponds to the nucleotide sequence of the pNAP433 Donor DNA fragment having the rice mitochondrial cox2 RNA editing site. In TABLE 3, the homologous regions are underlined, the mALS(HR-LS) ORF is highlighted in bold, and the RNA editing site to create AUG in mitochondria is shown in lower case.

SEQ ID NO: 46 (Oryza sativa) corresponds to the nucleotide sequence encoding an orf79 protein.

SEQ ID NO: 47 (Oryza sativa) corresponds to the amino acid sequence of the orf79 protein encoded by SEQ ID NO: 46.

SEQ ID NO: 48 (Artificial sequence) corresponds to the nucleotide sequence encoding a gRNA cassette for use with a MAD7 nuclease.

SEQ ID NO: 49 (Oryza sativa) corresponds to the nucleotide sequence for 5 HR Primer A.

SEQ ID NO: 50 (Oryza sativa) corresponds to the nucleotide sequence for ORF Primer B.

SEQ ID NO: 51 (Oryza sativa) corresponds to the nucleotide sequence for 3 HR Primer A.

SEQ ID NO: 52 (Artificial sequence) corresponds to the nucleotide sequence for 420 Primer A.

SEQ ID NO: 53 (Triticum aestivum) corresponds to the nucleotide sequence encoding an orf256 protein.

SEQ ID NO: 54 (Triticum aestivum) corresponds to the amino acid sequence of the orf256 protein encoded by SEQ ID NO: 53.

SEQ ID NO: 55 (Triticum aestivum) corresponds to the nucleotide sequence encoding an orf279 protein.

SEQ ID NO: 56 (Triticum aestivum) corresponds to the amino acid sequence of the orf279 protein encoded by SEQ ID NO: 55.

SEQ ID NO: 57 (Triticum timopheevii) corresponds to a 552-nucleotide sequence present in the mitochondrial genome of Triticum timopheevii that is also present in SEQ ID NO: 55.

SEQ ID NO: 58 (Triticum timopheevii) corresponds to the 184 amino acid sequence encoded by SEQ ID NO: 57.

SEQ ID NO: 59 (artificial sequence) corresponds to the nucleotide sequence (4945 nt) of the expression cassette having the following elements: Maize UBI1 promoter with intron-LhGR2 transcription activator gene (underlined in TABLE 3)-ocs terminator.

SEQ ID NO: 60 (artificial sequence) corresponds to the nucleotide sequence (3149 nt) of the dual expression cassette containing MTS-Rep coding region and the TagRFP coding region. The MTS sequence was derived from the Arabidopsis gene At5G47030 and is shown in italic letters in TABLE 3 and the Rep ORF is in bold font. The TagRFP coding region on the opposite strand is underlined.

SEQ ID NO: 61 (artificial sequence) corresponds to the nucleotide sequence (2066 nt) of the expression cassette for hygromycin selection in plant cells and has the following elements: 35S promoter-hygromycin phosphotransferase ORF-CaMV terminator. In TABLE 3, the hygromycin phosphotransferase ORF is presented in bold font.

SEQ ID NO: 62 (artificial sequence) corresponds to the amino acid sequence (445 aa) of the mEPSPS-HR protein that is lacking a chloroplast transit peptide. Two amino acids residues that can confer resistance to glyphosate in the EPSPS-HR protein are isoleucine at position 103 and serine at position 107 (shown in bold font in TABLE 3).

SEQ ID NO: 63 (artificial sequence) corresponds to the optimized nucleotide sequence (1338 nt) of the MEPSPS-HR coding region. In TABLE 3, lower case letters were the nucleotides modified from the corresponding rice EPSPS gene.

SEQ ID NO: 64 (artificial sequence) corresponds to the nucleotide sequence (1379 nt) produced by the fusion of SEQ ID NO: 41 with SEQ ID NO: 63. This fusion introduces an initiation codon by use of a naturally occurring mitochondrial RNA editing site.

SEQ ID NO: 65 (artificial sequence) corresponds to the amino acid sequence (449 aa) of mEPSPS-HR* that is encoded by SEQ ID NO: 64. The MEPSPS-HR* protein has an initiation methionine and three additional amino acids at the amino-terminus relative to SEQ ID NO: 62.

SEQ ID NO: 66 (Oryza sativa) corresponds to the nucleotide sequence (901 nt) of the promoter region corresponds to the nucleotide sequence of a rice ATP1 gene.

SEQ ID NO: 67 (artificial sequence) corresponds to the nucleotide sequence (468 nt) of the composite terminator containing sequences from the rice ATP1 and orf79 genes. In TABLE 3, italic letters correspond to ATP1 terminator sequence and bold letters correspond to orf79 terminator sequence.

SEQ ID NO: 68 (artificial sequence) corresponds to the nucleotide sequence of the 5′ homologous region (1168 bp) used in the donor DNA containing the EPSPS-HR* coding region. This sequence was derived from the mitochondrial genomic region of the rice ATP6 gene and one codon was changed at position 1025-1027 (underlined and in bold font in TABLE 3) to convey oligomycin resistance.

SEQ ID NO: 69 (Oryza sativa) corresponds to the nucleotide sequence for the 3′ homologous region (1203 bp) used in the donor DNA containing the EPSPS-HR* coding region. This sequence was derived from the mitochondrial genomic region downstream of the ATP6 gene.

SEQ ID NO: 70 (Oryza sativa) corresponds to the nucleotide sequence (699 nt) of the orf79 region containing the orf79 CMS gene used in the donor DNA containing the EPSPS-HR* coding region. This sequence was inserted at the 3′-end of the 5′ HR downstream of the ATP1 gene. In TABLE 3, the orf79 ORF is in bold font.

SEQ ID NO: 71 (Beet curly top virus) corresponds to the nucleotide sequence (201 nt) of a geminivirus VOR element that is present in the plasmid DNA construct at each end of the Donor DNA containing the EPSPS-HR* coding region, to give a VOR-Donor DNA-VOR configuration.

SEQ ID NO: 72 (artificial sequence) corresponds to the amino acid sequence (370 aa) of the herbicide-resistant GS1-HR protein. One amino acid residue at position 61 (underlined and in bold font in TABLE 3) can to confer resistance to glufosinate.

SEQ ID NO: 73 (artificial sequence) corresponds to the nucleotide sequence (1110 nt) of the optimized GS1-HR coding region minus the translation initiation codon. In TABLE 3, lower case letters indicate the nucleotides modified from the corresponding wild-type rice GS1 gene.

SEQ ID NO: 74 (artificial sequence) corresponds to the nucleotide sequence (1151 nt) produced by the fusion of SEQ ID NO: 41 with SEQ ID NO: 73. This fusion introduces an initiation codon by use of a naturally occurring mitochondrial RNA editing site.

SEQ ID NO: 75 (artificial sequence) corresponds to the amino acid sequence (373 aa) of GS1-HR* that is encoded by SEQ ID NO: 74. The GS1-HR* protein has three additional amino acids following the amino-terminal methionine relative to SEQ ID NO: 72.

SEQ ID NO: 76 (artificial sequence) corresponds to the amino acid sequence (239 aa) of the eGFP polypeptide present in the mALS(HR-LS)-eGFP fusion protein.

SEQ ID NO: 77 (artificial sequence) corresponds to the amino acid sequence of the linker present in the mALS(HR-LS)-eGFP fusion protein.

SEQ ID NO: 78 (artificial sequence) corresponds to the amino acid sequence (869 aa) of the mALS(HR-LS)-eGFP fusion protein. In TABLE 3, an initiation methionine is not present, the linker amino acids are underlined, and the eGFP region is shown in bold font.

SEQ ID NO: 79 (artificial sequence) corresponds to the nucleotide sequence (2610 nt) encoding the mALS(HR-LS)-eGFP fusion protein.

SEQ ID NO: 80 (artificial sequence) corresponds to the nucleotide sequence (75 nt) that encodes the wheat mitochondrial cox2 RNA editing site at the cox2 translation start site. In TABLE 3, the sequence derived from the wheat cox2 RNA editing site is underlined and the C nucleotide at position 41 that is edited in U in the corresponding RNA is in bold font.

SEQ ID NO: 81 (artificial sequence) corresponds to the nucleotide sequence (2685 nt) produced by the fusion of SEQ ID NO: 80 with SEQ ID NO: 79. This fusion introduces an initiation codon by use of a naturally occurring mitochondrial RNA editing site.

SEQ ID NO: 82 (artificial sequence) corresponds to the amino acid sequence (881 aa) of the mALS(HR-LS)-eGFP* fusion protein, where the initial methionine is the consequence of mitochondrial RNA editing.

SEQ ID NO: 83 (artificial sequence) corresponds to the nucleotide sequence (1162 nt) of a DNA fragment containing a T7 promoter, a gRNA polycistronic cassette, and both T7 and ATP1 terminators. In TABLE 3, the sequences encoding gRNAs (with constant repeat regions) are underlined; the T7 promoter and terminator sequences are in italics, and the rice ATP1 terminator sequence is in bold font.

SEQ ID NO: 84 (artificial sequence) corresponds to the nucleotide sequence of a DNA fragment containing the wheat mitochondrial atp6-1 gene with promoter and terminator. The wheat atp6-1 sequence in SEQ ID NO: 84 was altered from the wild-type sequence to encode a variant protein with oligomycin resistance. In TABLE 3, the atp6-1 ORF at nucleotides 714-1874 is underlined and the altered codon at nucleotides 1767-1769 providing oligomycin resistance is in bold font.

SEQ ID NO: 85 corresponds to the nucleotide sequence (222 nt) containing the I-SceI restriction site and the orf279 terminator. In TABLE 3, the orf279 terminator sequence (nucleotides 19-222) is underlined.

SEQ ID NO: 86 (artificial sequence) corresponds to the nucleotide sequence (1952 nt) from the Donor DNA comprising the Triticum aestivum 5′ homologous region (5′-HR) fused to the Triticum timopheevii specific region of the orf279 gene. In TABLE 3, the Triticum aestivum 5′-HR of the Donor DNA corresponds to nucleotides 1-1201 of SEQ ID NO: 86 and the Triticum timopheevii specific sequence corresponds to nucleotides 1202-1952 (underlined). The orf279 ORF (nucleotides 912-1751), which is a fusion of Triticum aestivum atp8-1 sequence and Triticum timopheevii specific sequence, is shown in italic. In the upstream region, one nucleotide (nucleotide 610 shown in lower case) was altered from wild-type sequence to eliminate a BamHI restriction site.

SEQ ID NO: 87 (Triticum aestivum) corresponds to the nucleotide sequence (1200 nt) of the 3′ homologous region (3′-HR) of the Donor DNA.

SEQ ID NO: 88 (artificial sequence) comprises the nucleotide sequence (1636 nt) of the Donor DNA region from pNAP652 containing the wheat atp6-1 ORF and the rice orf79 ORF. In TABLE 3, the atp6-1 ORF is underlined and the orf79 ORF is shown in bold font.

SEQ ID NO: 89 (artificial sequence) corresponds to the nucleotide sequence of the PCR primer 5HRBst.

SEQ ID NO: 90 (artificial sequence) corresponds to the nucleotide sequence of the PCR primer ORF79st.

SEQ ID NO: 91 (artificial sequence) corresponds to the nucleotide sequence of the PCR primer 5HRAst.

SEQ ID NO: 92 (artificial sequence) corresponds to the nucleotide sequence of the PCR primer ORFBst.

SEQ ID NO: 93 (artificial sequence) corresponds to the nucleotide sequence of the PCR primer 3HRBst.

SEQ ID NO: 94 (artificial sequence) corresponds to the nucleotide sequence of the PCR primer 420Bst.

SEQ ID NO: 95 (artificial sequence) corresponds to the nucleotide sequence of the PCR primer 3HRAst.

SEQ ID NO: 96 (artificial sequence) corresponds to the nucleotide sequence of the PCR primer 420Ast.

SEQ ID NO: 97 (artificial sequence) corresponds to the nucleotide sequence of the sequencing primer 5HR_for_6.

SEQ ID NO: 98 (artificial sequence) corresponds to the nucleotide sequence of the sequencing primer 5HR_for_4.

SEQ ID NO: 99 (artificial sequence) corresponds to the nucleotide sequence of the sequencing primer Invitro_for_1.

SEQ ID NO: 100 (artificial sequence) corresponds to the nucleotide sequence of the sequencing primer 3HR_rev_4.

SEQ ID NO: 101 (artificial sequence) corresponds to the nucleotide sequence of the sequencing primer 3HR_seq_for.

SEQ ID NO: 102 (artificial sequence) corresponds to the nucleotide sequence of the sequencing primer 3HR_seq_rev.

SEQ ID NO: 103 (artificial sequence) corresponds to the nucleotide sequence (1739 nt) from the 5′-junction PCR fragment amplified from leaf tissue of event HH43. In TABLE 3, nucleotides 1-31 (underlined) correspond to wild-type mitochondrial sequence not present in the Donor DNA. Nucleotides 32-1646 (bold font) correspond to sequence of the 5′-HR. Nucleotides 1647-1739 (italic font) correspond to sequence specific to the Donor DNA and not present in the wild-type mitochondrial sequence.

SEQ ID NO: 104 (artificial sequence) corresponds to the nucleotide sequence (1370 nt) from the 3′-junction PCR fragment amplified from leaf tissue of event HH43. In TABLE 3, Nucleotides 1-13 (italic font) correspond to sequence specific to the Donor DNA and not present in the wild-type mitochondrial sequence. Nucleotides 14-1216 (bold font) correspond to sequence of the 3′-HR. Nucleotides 1217-1370 (underlined) correspond to wild-type mitochondrial sequence not present in the Donor DNA.

SEQ ID NO: 105 (artificial sequence) corresponds to the nucleotide sequence of the sequencing primer 3HRrev3.

SEQ ID NO: 106 (artificial sequence) corresponds to the nucleotide sequence of a portion (796 nt) of the 5′-end of the 5′-junction PCR fragment from glyphosate-resistant line TT57. In TABLE 3, nucleotides 1-425 (underlined) correspond to wild-type mitochondrial sequence not present in the Donor DNA and nucleotides 426-796 (bold font) correspond to sequences from the 5′-HR of the Donor DNA.

SEQ ID NO: 107 (artificial sequence) corresponds to the nucleotide sequence of a portion (850 nt) of the 3′-end of the 5′-junction PCR fragment from glyphosate-resistant line TT57. In TABLE 3, nucleotides 1-780 (bold font) correspond to sequences from the 5′-HR of the Donor DNA and nucleotides 781-850 (italic font) correspond to sequences specific to the Donor DNA and not present in the wild-type mitochondrial sequence.

SEQ ID NO: 108 (artificial sequence) corresponds to the nucleotide sequence (1365 nt) from the 3′-junction PCR fragment from glyphosate-resistant line TT31. In TABLE 3, nucleotides 1-38 (italic font) correspond to sequences specific to the Donor DNA and not present in the wild-type mitochondrial sequence. Nucleotides 39-42 correspond to novel sequence at the site of the deletion of the orf79 terminator. Nucleotides 43-1241 (bold font) correspond to sequences from the 3′-HR of the Donor DNA. Nucleotides 1242-1365 correspond to sequences from wild-type mitochondrial sequence not present in the Donor DNA.

SEQ ID NO: 109 (artificial sequence) corresponds to the nucleotide sequence (871 nt) of the 5′-junction PCR fragment from glufosinate-resistant line S96. In TABLE 3, nucleotides 1-202 (underlined) correspond to wild-type mitochondrial sequence not present in the truncated Donor DNA fragment. Nucleotides 203-800 (bold font) correspond to sequences from the 5′-HR of the truncated Donor DNA fragment. Nucleotides 801-871 (italic font) correspond to sequences specific to the truncated Donor DNA fragment and not present in the wild-type mitochondrial sequence.

SEQ ID NO: 110 corresponds to the nucleotide sequence (1376 nt) from the 3′-junction PCR fragment from glufosinate-resistant line S96. In TABLE 3, nucleotides 1-36 (italic font) correspond to sequences specific to the truncated Donor DNA fragment and not present in the wild-type mitochondrial sequence. Nucleotides 37-40 correspond to novel sequence at the site of the deletion of the orf79 terminator. Nucleotides 41-1121 (bold font) correspond to sequences from the 3′-HR of the truncated Donor DNA fragment. Nucleotides 1121-1376 correspond to sequences from wild-type mitochondrial sequence not present in the truncated Donor DNA fragment.

SEQ ID NO: 111 corresponds to the amino acid sequence (393 aa) of the MTS-Rep polypeptide that is encoded by SEQ ID NO: 60. The amino-terminal 36-aa MTS was derived from the protein encoded by the Arabidopsis gene At5G47030.

TABLE 3
Sequence Listing

| SEQ | |
|---|---|
| ID NO | SEQUENCE |
| 1 | LAGLIDADG |
| 2 | DEVDFQGPCNDSSDPLVVAASIIGILHLILWILDRL |
| 3 | DEVD |
| 4 | ACUUUUGACAGUUAUACGAUUCCAGAA |
| 5 | UGGGCUGUACCUUCCUCAGGUGUCAAA |
| 6 | GCUGUACCUGGUCGUUCAAAUCUUACC |
| 7 | MATTAAAAAAALSAAATAKTGRKNHQRHHVLPARGRVGAAAVRCSAVSP |
| | VTPPSPAPPATPLRPWGPAEPRKGADILVEALERCGVSDVFAYPGGASMEIHQ |
| | ALTRSPVITNHLFRHEQGEAFAASGYARASGRVGVCVATSGPGATNLVSALA |
| | DALLDSVPMVAITGQVPRRMIGTDAFQETPIVEVTRSITKHNYLVLDVEDIPR |
| | VIQEAFFLASSGRPGPVLVDIPKDIQQQMAVPVWDTSMNLPGYIARLPKPPAT |
| | ELLEQVLRLVGESRRPILYVGGGCSASGDELRWFVELTGIPVTTTLMGLGNFP |
| | SDDPLSLRMLGMHGTVYANYAVDKADLLLAFGVRFDDRVTGKIEAFASRA |
| | KIVHIDIDPAEIGKNKQPHVSICADVKLALQGLNALLQQSTTKTSSDFSAWHN |
| | ELDQQKREFPLGYKTFGEEIPPQYAIQVLDELTKGEAIIATGVGQHQMWAAQ |
| | YYTYKRPRQWLSSAGLGAMGFGLPAAAGASVANPGVTVVDIDGDGSFLMNI |
| | QELALIRIENLPVKVMVLNNQHLGMVVQLEDRFYKANRAHTYLGNPECESEI |
| | YPDFVTIAKGFNIPAVRVTKKSEVRAAIKKMLETPGPYLLDIIVPHQEHVLPMI |
| | PIGGAFKDMILDGDGRTVY |
| 8 | ATGGCTACGACCGCCGCGGCCGCGGCCGCCGCCCTGTCCGCCGCCGCGAC |
| | GGCC |
| 9 | ATGGCCGCCAAGATcCGCATcGTGATGAAATCTTTTATGAGCCAAGCTAAC |
| | AAAGTTGAAGGGGTTATTCCATACGCGCAGAAGGTTGGATTGCCTGAATC |
| | ACGATCCTTGTATACCGTGCTACGATCGCCTCACATcGACAAGAAGTCGA |
| | GGGAGCAGTTCTCGATG |
| 10 | ATGGCCGCCAAGATcCGCATcGTGATGAAATCTTTTATGAGCCAAGCTAAC |
| | AAAGTTGAAGGGGTTATTCCATACGCGCAGAAGGTTGGATTGCCTGAATC |
| | ACGATCCTTGTATACCGTGCTACGATCGCCTCACATcGACAAGAAGTCGA |
| | GGGAGCAGTTCTCGATGAAGACCGGCCGTAAGAACCACCAGCGACACCA |
| | CGTCCTTCCCGCTCGAGGCCGGGTGGGGGCGGCGGCGGTCAGGTGCTCGG |
| | CGGTGTCCCCGGTCACCCCGCCGTCCCCGGCGCCGCCGGCCACGCCGCTC |
| | CGGCCGTGGGGGCCGGCCGAGCCCCGCAAGGGCGCGGACATCCTCGTGG |
| | AGGCGCTGGAGCGGTGCGGCGTCAGCGACGTGTTCGCCTACCCGGGCGG |
| | CGCGTCCATGGAGATCCACCAGGCGCTGACGCGCTCCCCGGTCATCACCA |
| | ACCACCTCTTCCGCCACGAGCAGGGCGAGGCGTTCGCGGCGTCCGGGTAC |
| | GCGCGCGCGTCCGGCCGCGTCGGGGTCTGCGTCGCCACCTCCGGCCCCGG |
| | GGCAACCAACCTCGTGTCCGCGCTCGCCGACGCGCTGCTCGACTCCGTCC |
| | CGATGGTCGCCATCACGGGCCAGGTCCCCCGCCGCATGATCGGCACCGAC |
| | GCCTTCCAGGAGACGCCCATAGTCGAGGTCACCCGCTCCATCACCAAGCA |
| | CAATTACCTTGTCCTTGATGTGGAGGACATCCCCCGCGTCATACAGGAAG |
| | CCTTCTTCCTCGCGTCCTCGGGCCGTCCTGGCCCGGTGCTGGTCGACATCC |
| | CCAAGGACATCCAGCAGCAGATGGCCGTGCCGGTCTGGGACACCTCGAT |
| | GAATCTACCAGGGTACATCGCACGCCTGCCCAAGCCACCCGCGACAGAA |
| | TTGCTTGAGCAGGTCTTGCGTCTGGTTGGCGAGTCACGGCGCCCGATTCT |
| | CTATGTCGGTGGTGGCTGCTCTGCATCTGGTGACGAATTGCGCTGGTTTGT |
| | TGAGCTGACTGGTATCCCAGTTACAACCACTCTGATGGGCCTCGGCAATT |
| | TCCCCAGTGACGACCCGTTGTCCCTGCGCATGCTTGGGATGCATGGCACG |
| | GTGTACGCAAATTATGCCGTGGATAAGGCTGACCTGTTGCTTGCGTTTGG |
| | TGTGCGGTTTGATGATCGTGTGACAGGGAAAATTGAGGCTTTTGCAAGCA |
| | GGGCCAAGATTGTGCACATTGACATTGATCCAGCAGAGATTGGAAAGAA |
| | CAAGCAACCACATGTGTCAATTTGCGCAGATGTTAAGCTTGCTTTACAGG |
| | GCTTGAATGCTCTGCTACAACAGAGCACAACAAAGACAAGTTCTGATTTT |
| | AGTGCATGGCACAATGAGTTGGACCAGCAGAAGAGGGAGTTTCCTCTGG |
| | GGTACAAAACTTTTGGTGAAGAGATCCCACCGCAATATGCCATTCAGGTG |
| | CTGGATGAGCTGACGAAAGGTGAGGCAATCATCGCTACTGGTGTTGGGC |
| | AGCACCAGATGTGGGCGGCACAATATTACACCTACAAGCGGCCACGGCA |
| | GTGGCTGTCTTCGGCTGGTCTGGGCGCAATGGGATTTGGGCTGCCTGCTG |
| | CAGCTGGTGCTTCTGTGGCTAACCCAGGTGTCACAGTTGTTGATATTGAT |
| | GGGGATGGTAGCTTCCTCATGAACATTCAGGAGCTGGCATTGATCCGCAT |
| | TGAGAACCTCCCTGTGAAGGTGATGGTGTTGAACAACCAACATTTGGGTA |
| | TGGTGGTGCAATTGGAGGATAGGTTTTACAAGGCGAATAGGGCGCATAC |
| | ATACTTGGGCAACCCGGAATGTGAGAGCGAGATATATCCAGATTTTGTGA |
| | CTATTGCTAAGGGGTTCAATATTCCTGCAGTCCGTGTAACAAAGAAGAGT |
| | GAAGTCCGTGCCGCCATCAAGAAGATGCTCGAGACTCCAGGGCCATACTT |
| | GTTGGATATCATCGTCCCGCACCAGGAGCATGTGCTGCCTATGATCCCAA |
| | TTGGGGGCGCATTCAAGGACATGATCCTGGATGGTGATGGCAGGACTGTG |
| | TATTAA |
| 11 | MAAKIRIVMKSFMSQANKVEGVIPYAQKVGLPESRSLYTVLRSPHIDKKSRE |
| | QFSMKTGRKNHQRHHVLPARGRVGAAAVRCSAVSPVTPPSPAPPATPLRPW |
| | GPAEPRKGADILVEALERCGVSDVFAYPGGASMEIHQALTRSPVITNHLFRHE |
| | QGEAFAASGYARASGRVGVCVATSGPGATNLVSALADALLDSVPMVAITGQ |
| | VPRRMIGTDAFQETPIVEVTRSITKHNYLVLDVEDIPRVIQEAFFLASSGRPGP |
| | VLVDIPKDIQQQMAVPVWDTSMNLPGYIARLPKPPATELLEQVLRLVGESRR |
| | PILYVGGGCSASGDELRWFVELTGIPVTTTLMGLGNFPSDDPLSLRMLGMHG |
| | TVYANYAVDKADLLLAFGVRFDDRVTGKIEAFASRAKIVHIDIDPAEIGKNK |
| | QPHVSICADVKLALQGLNALLQQSTTKTSSDFSAWHNELDQQKREFPLGYKT |
| | FGEEIPPQYAIQVLDELTKGEAIIATGVGQHQMWAAQYYTYKRPRQWLSSA |
| | GLGAMGFGLPAAAGASVANPGVTVVDIDGDGSFLMNIQELALIRIENLPVKV |
| | MVLNNQHLGMVVQLEDRFYKANRAHTYLGNPECESEIYPDFVTIAKGFNIPA |
| | VRVTKKSEVRAAIKKMLETPGPYLLDIIVPHQEHVLPMIPIGGAFKDMILDGD |
| | GRTVY |
| 12 | CTGCAGTGCAGCGTGACCCGGTCGTGCCCCTCTCTAGAGATAATGAGCAT |
| | TGCATGTCTAAGTTATAAAAAATTACCACATATTTTTTTTGTCACACTTGT |
| | TTGAAGTGCAGTTTATCTATCTTTATACATATATTTAAACTTTACTCTACG |
| | AATAATATAATCTATAGTACTACAATAATATCAGTGTTTTAGAGAATCAT |
| | ATAAATGAACAGTTAGACATGGTCTAAAGGACAATTGAGTATTTTGACAA |
| | CAGGACTCTACAGTTTTATCTTTTTAGTGTGCATGTGTTCTCCTTTTTTTTT |
| | GCAAATAGCTTCACCTATATAATACTTCATCCATTTTATTAGTACATCCAT |
| | TTAGGGTTTAGGGTTAATGGTTTTTATAGACTAATTTTTTTAGTACATCTA |
| | TTTTATTCTATTTTAGCCTCTAAATTAAGAAAACTAAAACTCTATTTTAGT |
| | TTTTTTATTTAATAATTTAGATATAAAATAGAATAAAATAAAGTGACTAA |
| | AAATTAAACAAATACCCTTTAAGAAATTAAAAAAACTAAGGAAACATTTT |
| | TCTTGTTTCGAGTAGATAATGCCAGCCTGTTAAACGCCGTCGACGAGTCT |
| | AACGGACACCAACCAGCGAACCAGCAGCGTCGCGTCGGGCCAAGCGAAG |
| | CAGACGGCACGGCATCTCTGTCGCTGCCTCTGGACCCCTCTCGAGAGTTC |
| | CGCTCCACCGTTGGACTTGCTCCGCTGTCGGCATCCAGAAATTGCGTGGC |
| | GGAGCGGCAGACGTGAGCCGGCACGGCAGGCGGCCTCCTCCTCCTCTCAC |
| | GGCACCGGCAGCTACGGGGGATTCCTTTCCCACCGCTCCTTCGCTTTCCCT |
| | TCCTCGCCCGCCGTAATAAATAGACACCCCCTCCACACCCTCTTTCCCCAA |
| | CCTCGTGTTGTTCGGAGCGCACACACACACAACCAGATCTCCCCCAAATC |
| | CACCCGTCGGCACCTCCGCTTCAAGGTACGCCGCTCGTCCTCCCCCCCCCC |
| | CCCTCTCTACCTTCTCTAGATCGGCGTTCCGGTCCATGGTTAGGGCCCGGT |
| | AGTTCTACTTCTGTTCATGTTTGTGTTAGATCCGTGTTTGTGTTAGATCCGT |
| | GCTGCTAGCGTTCGTACACGGATGCGACCTGTACGTCAGACACGTTCTGA |
| | TTGCTAACTTGCCAGTGTTTCTCTTTGGGGAATCCTGGGATGGCTCTAGCC |
| | GTTCCGCAGACGGGATCGATTTCATGATTTTTTTTGTTTCGTTGCATAGGG |
| | TTTGGTTTGCCCTTTTCCTTTATTTCAATATATGCCGTGCACTTGTTTGTCG |
| | GGTCATCTTTTCATGCTTTTTTTTGTCTTGGTTGTGATGATGTGGTCTGGTT |
| | GGGCGGTCGTTCTAGATCGGAGTAGAATTAATTCTGTTTCAAACTACCTG |
| | GTGGATTTATTAATTTTGGATCTGTATGTGTGTGCCATACATATTCATAGT |
| | TACGAATTGAAGATGATGGATGGAAATATCGATCTAGGATAGGTATACAT |
| | GTTGATGCGGGTTTTACTGATGCATATACAGAGATGCTTTTTGTTCGCTTG |
| | GTTGTGATGATGTGGTGTGGTTGGGCGGTCGTTCATTCGTTCTAGATCGG |
| | AGTAGAATACTGTTTCAAACTACCTGGTGTATTTATTAATTTTGGAACTGT |
| | ATGTGTGTGTCATACATCTTCATAGTTACGAGTTTAAGATGGATGGAAAT |
| | ATCGATCTAGGATAGGTATACATGTTGATGTGGGTTTTACTGATGCATAT |
| | ACATGATGGCATATGCAGCATCTATTCATATGCTCTAACCTTGAGTACCT |
| | ATCTATTATAATAAACAAGTATGTTTTATAATTATTTTGATCTTGATATAC |
| | TTGGATGATGGCATATGCAGCAGCTATATGTGGATTTTTTTAGCCCTGCCT |
| | TCATACGCTATTTATTTGCTTGGTACTGTTTCTTTTGTCGATGCTCACCCTG |
| | TTGTTTGGTGTTACTTCTGCAGGTCGACTCTAGAGGATCC |
| 13 | GATCGTTCAAACATTTGGCAATAAAGTTTCTTAAGATTGAATCCTGTTGCC |
| | GGTCTTGCGATGATTATCATATAATTTCTGTTGAATTACGTTAAGCATGTA |
| | ATAATTAACATGTAATGCATGACGTTATTTATGAGATGGGTTTTTATGATT |
| | AGAGTCCCGCAATTATACATTTAATACGCGATAGAAAACAAAATATAGC |
| | GCGCAAACTAGGATAAATTATCGCGCGCGGTGTCATCTATGTTACTAGAT |
| | C |
| 14 | ATGAAAAAGCCTGAACTCACCGCGACGTCTGTCGAGAAGTTTCTGATCGA |
| | AAAGTTCGACAGCGTCTCCGACCTGATGCAGCTCTCGGAGGGCGAAGAA |
| | TCTCGTGCTTTCAGCTTCGATGTAGGAGGGCGTGGATATGTCCTGCGGGT |
| | AAATAGCTGCGCCGATGGTTTCTACAAAGATCGTTATGTTTATCGGCACT |
| | TTGCATCGGCCGCGCTCCCGATTCCGGAAGTGCTTGACATTGGGGAGTTT |
| | AGCGAGAGCCTGACCTATTGCATCTCCCGCCGTGCACAGGGTGTCACGTT |
| | GCAAGACCTGCCTGAAACCGAACTGCCCGCTGTTCTACAACCGGTCGCGG |
| | AGGCTATGGATGCGATCGCTGCGGCCGATCTTAGCCAGACGAGCGGGTTC |
| | GGCCCATTCGGACCGCAAGGAATCGGTCAATACACTACATGGCGTGATTT |
| | CATATGCGCGATTGCTGATCCCCATGTGTATCACTGGCAAACTGTGATGG |
| | ACGACACCGTCAGTGCGTCCGTCGCGCAGGCTCTCGATGAGCTGATGCTT |
| | TGGGCCGAGGACTGCCCCGAAGTCCGGCACCTCGTGCACGCGGATTTCGG |
| | CTCCAACAATGTCCTGACGGACAATGGCCGCATAACAGCGGTCATTGACT |
| | GGAGCGAGGCGATGTTCGGGGATTCCCAATACGAGGTCGCCAACATCTTC |
| | TTCTGGAGGCCGTGGTTGGCTTGTATGGAGCAGCAGACGCGCTACTTCGA |
| | GCGGAGGCATCCGGAGCTTGCAGGATCGCCACGACTCCGGGCGTATATG |
| | CTCCGCATTGGTCTTGACCAACTCTATCAGAGCTTGGTTGACGGCAATTTC |
| | GATGATGCAGCTTGGGCGCAGGGTCGATGCGACGCAATCGTCCGATCCG |
| | GAGCCGGGACTGTCGGGCGTACACAAATCGCCCGCAGAAGCGCGGCCGT |
| | CTGGACCGATGGCTGTGTAGAAGTACTCGCCGATAGTGGAAACCGACGC |
| | CCCAGCACTCGTCCGAGGGCAAAGAAATAG |
| 15 | ATGGTGGAGCACGACACTCTCGTCTACTCCAAGAATATCAAAGATACAGT |
| | CTCAGAAGACCAAAGGGCTATTGAGACTTTTCAACAAAGGGTAATATCG |
| | GGAAACCTCCTCGGATTCCATTGCCCAGCTATCTGTCACTTCATCAAAAG |
| | GACAGTAGAAAAGGAAGGTGGCACCTACAAATGCCATCATTGCGATAAA |
| | GGAAAGGCTATCGTTCAAGATGCCTCTGCCGACAGTGGTCCCAAAGATGG |
| | ACCCCCACCCACGAGGAGCATCGTGGAAAAAGAAGACGTTCCAACCACG |
| | TCTTCAAAGCAAGTGGATTGATGTGATAACATGGTGGAGCACGACACTCT |
| | CGTCTACTCCAAGAATATCAAAGATACAGTCTCAGAAGACCAAAGGGCT |
| | ATTGAGACTTTTCAACAAAGGGTAATATCGGGAAACCTCCTCGGATTCCA |
| | TTGCCCAGCTATCTGTCACTTCATCAAAAGGACAGTAGAAAAGGAAGGTG |
| | GCACCTACAAATGCCATCATTGCGATAAAGGAAAGGCTATCGTTCAAGAT |
| | GCCTCTGCCGACAGTGGTCCCAAAGATGGACCCCCACCCACGAGGAGCA |
| | TCGTGGAAAAAGAAGACGTTCCAACCACGTCTTCAAAGCAAGTGGATTG |
| | ATGTGATATCTCCACTGACGTAAGGGATGACGCACAATCCCACTATCCTT |
| | CGCAAGACCTTCCTCTATATAAGGAAGTTCATTTCATTTGGAGAGGACAC |
| | GCTGAAATCACCAGTCTCTCTCTACAAATCTATCTCT |
| 16 | GATCTGTCGATCGACAAGCTCGAGTTTCTCCATAATAATGTGTGAGTAGT |
| | TCCCAGATAAGGGAATTAGGGTTCCTATAGGGTTTCGCTCATGTGTTGAG |
| | CATATAAGAAACCCTTAGTATGTATTTGTATTTGTAAAATACTTCTATCAA |
| | TAAAATTTCTAATTCCTAAAACCAAAATCCAGTACTAAAATCCAGATCCC |
| | CCGAATTA |
| 17 | MATATAASRLAVAGAEPARARRHRPTTVAVCGGARPRSRPAAVVAAAGAA |
| | APSPATGGVAPVPPSPRGSIIKRHTLSVFVGDESGMINRIAGVFARRGYNIESL |
| | AVGLNKDKALFTIVVSGTEKILKQVVEQLNKLVNVIQVDDLSKEPQVERELM |
| | LIKLNVEPDKRPEVMGLVDIFRAKVVDLSDHTLTIEVTGDPGKIVAVQRNLS |
| | KFGIKEIARTGKIALRREKMGESAPFWRFSAASYPDLEVAMPSKSHVNTAMK |
| | TANQNSEESSQGDVYPVESYENFTTNQILDAHWGVMADGDPTGLCSHSLSIL |
| | VNDFPGVLNVVTGVFSRRGYNIQSLAVGPAEKEGTSRITTVVPGTDESIAKLV |
| | HQLYKLIDVYEVQDLTHLPFAARELMIIKIAVNTTARRAILDIADIFRAKTVD |
| | VSDHTVTLQLTGDLDKMVALQRMLEPYGICEVARTGRVALRRESGVDSKYL |
| | RGFSLPL |
| 18 | MATATAASRLAVAGAEPARARRHRPTTVAVCGGARPRSRPAAVVAAA |
| 19 | ATGTTTAAACAAGCTTCTCGTCTCCTCTCCCGATCTGTCGCCGCCGCATCT |
| | TCCAAATCGGTGACGACTCGTGCCTTTTCAACGGAACTTCCATCGACGCT |
| | CGATTCC |
| 20 | ATGTTTAAACAAGCTTCTCGTCTCCTCTCCCGATCTGTCGCCGCCGCATCT |
| | TCCAAATCGGTGACGACTCGTGCCTTTTCAACGGAACTTCCATCGACGCT |
| | CGATTCCGGCGCCGCCGCTCCCTCTCCGGCCACCGGCGGCGTCGCGCCGG |
| | TTCCGCCAAGCCCCAGGGGCTCGATTATAAAGCGTCATACACTATCAGTT |
| | TTTGTTGGAGATGAAAGTGGGATGATTAATCGAATTGCTGGAGTTTTTGC |
| | TAGAAGAGGATACAACATTGAGTCATTGGCAGTTGGGCTGAACAAGGAC |
| | AAGGCATTATTTACAATAGTAGTATCTGGAACTGAGAAAATACTGAAACA |
| | GGTTGTAGAGCAACTAAACAAACTTGTTAATGTTATACAGGTTGACGATC |
| | TGTCGAAGGAACCACAAGTTGAAAGAGAGCTAATGCTTATAAAACTAAA |
| | TGTTGAGCCAGATAAGCGACCGGAGGTGATGGGTTTGGTTGACATCTTCA |
| | GAGCAAAGGTGGTTGACCTTTCTGACCACACACTAACTATTGAGGTAACT |
| | GGAGATCCTGGAAAAATTGTTGCTGTACAGAGGAACCTAAGCAAATTTG |
| | GAATCAAAGAAATTGCCAGAACTGGCAAGATAGCTTTGCGGCGTGAGAA |
| | AATGGGAGAAAGTGCTCCTTTTTGGCGGTTCTCTGCAGCTTCCTATCCTGA |
| | TCTTGAAGTGGCAATGCCATCAAAATCTCATGTCAACACTGCGATGAAAA |
| | CAGCTAACCAGAATTCTGAAGAATCTTCACAAGGTGATGTCTATCCAGTG |
| | GAGTCATATGAAAACTTCACAACAAATCAAATTCTTGATGCTCATTGGGG |
| | TGTCATGGCTGACGGTGATCCAACAGGGCTTTGTTCACATTCTTTGTCCAT |
| | TTTGGTGAATGATTTCCCTGGAGTTCTCAATGTTGTAACAGGTGTTTTCTC |
| | CCGAAGAGGCTACAATATTCAGAGTCTGGCTGTTGGCCCAGCTGAAAAA |
| | GAAGGCACTTCTCGGATCACTACTGTTGTCCCTGGAACTGATGAGTCTAT |
| | TGCCAAGCTAGTACACCAACTGTATAAGCTCATTGATGTTTATGAGGTCC |
| | AGGATCTTACTCATTTACCATTTGCTGCTAGAGAGTTAATGATCATAAAG |
| | ATTGCTGTAAACACCACAGCCCGCAGGGCTATCCTAGATATTGCTGATAT |
| | TTTCCGGGCCAAAACTGTGGATGTATCAGATCACACCGTAACTCTTCAGC |
| | TTACTGGAGACCTTGATAAAATGGTCGCATTACAAAGGATGCTCGAGCCC |
| | TATGGCATTTGTGAGGTTGCACGAACTGGCAGGGTTGCGTTGCGCCGTGA |
| | GTCAGGAGTCGATTCCAAATACCTCCGTGGGTTTTCCCTTCCTCTGTAG |
| 21 | MFKQASRLLSRSVAAASSKSVTTRAFSTELPSTLDSGAAAPSPATGGVAPVPP |
| | SPRGSIIKRHTLSVFVGDESGMINRIAGVFARRGYNIESLAVGLNKDKALFTIV |
| | VSGTEKILKQVVEQLNKLVNVIQVDDLSKEPQVERELMLIKLNVEPDKRPEV |
| | MGLVDIFRAKVVDLSDHTLTIEVTGDPGKIVAVQRNLSKFGIKEIARTGKIAL |
| | RREKMGESAPFWRFSAASYPDLEVAMPSKSHVNTAMKTANQNSEESSQGDV |
| | YPVESYENFTTNQILDAHWGVMADGDPTGLCSHSLSILVNDFPGVLNVVTG |
| | VFSRRGYNIQSLAVGPAEKEGTSRITTVVPGTDESIAKLVHQLYKLIDVYEVQ |
| | DLTHLPFAARELMIIKIAVNTTARRAILDIADIFRAKTVDVSDHTVTLQLTGDL |
| | DKMVALQRMLEPYGICEVARTGRVALRRESGVDSKYLRGFSLPL |
| 22 | TAGCTAGCATACTCGAGGTCATTCATATGCTTGAGAAGAGAGTCGGGATA |
| | GTCCAAAATAAAACAAAGGTAAGATTACCTGGTCAAAAGTGAAAACATC |
| | AGTTAAAAGGTGGTATAAGTAAAATATCGGTAATAAAAGGTGGCCCAAA |
| | GTGAAATTTACTCTTTTCTACTATTATAAAAATTGAGGATGTTTTGTCGGT |
| | ACTTTGATACGTCATTTTTGTATGAATTGGTTTTTAAGTTTATTCGCGATTT |
| | GGAAATGCATATCTGTATTTGAGTCGGTTTTTAAGTTCGTTGCTTTTGTAA |
| | ATACAGAGGGATTTGTATAAGAAATATCTTTAAAAAACCCATATGCTAAT |
| | TTGACATAATTTTTGAGAAAAATATATATTCAGGCGAATTCCACAATGAA |
| | CAATAATAAGATTAAAATAGCTTGCCCCCGTTGCAGCGATGGGTATTTTT |
| | TCTAGTAAAATAAAAGATAAACTTAGACTCAAAACATTTACAAAAACAA |
| | CCCCTAAAGTCCTAAAGCCCAAAGTGCTATGCACGATCCATAGCAAGCCC |
| | AGCCCAACCCAACCCAACCCAACCCACCCCAGTGCAGCCAACTGGCAAA |
| | TAGTCTCCACCCCCGGCACTATCACCGTGAGTTGTCCGCACCACCGCACG |
| | TCTCGCAGCCAAAAAAAAAAAAAGAAAGAAAAAAAAGAAAAAGAAAAA |
| | CAGCAGGTGGGTCCGGGTCGTGGGGGCCGGAAAAGCGAGGAGGATCGCG |
| | AGCAGCGACGAGGCCCGGCCCTCCCTCCGCTTCCAAAGAAACGCCCCCCA |
| | TCGCCACTATATACATACCCCCCCCTCTCCTCCCATCCCCCCAACCCTACC |
| | ACCACCACCACCACCACCTCCTCCCCCCTCGCTGCCGGACGACGAGCTCC |
| | TCCCCCCTCCCCCTCCGCCGCCGCCGGTAACCACCCCGCCCCTCTCCTCTT |
| | TCTTTCTCCGTTTTTTTTTTCGTCTCGGTCTCGATCTTTGGCCTTGGTAGTT |
| | TGGGTGGGCGAGAGCGGCTTCGTCGCCCAGATCGGTGCGCGGGAGGGGC |
| | GGGATCTCGCGGCTGGCGTCTCCGGGCGTGAGTCGGCCCGGATCCTCGCG |
| | GGGAATGGGGCTCTCGGATGTAGATCTTCTTTCTTTCTTCTTTTTGTGGTA |
| | GAATTTGAATCCCTCAGCATTGTTCATCGGTAGTTTTTCTTTTCATGATTT |
| | GTGACAAATGCAGCCTCGTGCGGAGCTTTTTTGTAGGTAGAAG |
| 23 | GAGTTTGAATCAAATCTTCACTTGTTTAATGAGATATGCGAGACGCCTAT |
| | GATCGCATGATATTTGCTTTCAATTCTGTTGTGCACGTTGTAAAAAACCTG |
| | AGCATGTGTAGCTCAGATCCTTACCGCCGGTTTCGGTTCATTCTAATGAAT |
| | ATATCACCCGTTACTATCGTATTTTTATGAATAATATTCTCCGTTCAATTT |
| | ACTGATTGTACCCTACTACTTATATGTACAATATTAAAATGAAAACAATA |
| | TATTGTGCTGAATAGGTTTATAGCGACATCTATGATAGAGCGCCACAATA |
| | ACAAACAATTGCGTTTTATTATTACAAATCCAATTTTAAAAAAAGCGGCA |
| | GAACCGGTCAAACCTAAAAGACTGATTACATAAATCTTATTCAAATTTCA |
| | AAAGTGCCCCAGGGGCTAGTATCTACGACACACCGAGCGGCGAATTCAG |
| | TACATTAAAAACGTCCGCAATGTGTTATTAAGTTGTCTAAGCGTCAATTT |
| | GTTTACACCACAATATATCCTGCCACCAGCCAGCCAACAGCTCCCCGACC |
| | GGCAGCTCGGCACAAAATCACCACTCGATACAGGCAGCCCATCAGTCCG |
| | GGACGGCGTCAGCGGGA |
| 24 | MAATAKTGRKNHQRHHVLPARGRVGAAAVRCSAVSPVTPPSPAPPATPLRP |
| | WGPAEPRKGADILVEALERCGVSDVFAYPGGASMEIHQALTRSPVITNHLFR |
| | HEQGEAFAASGYARASGRVGVCVATSGPGATNLVSALADALLDSVPMVAIT |
| | GQVPRRMIGTDAFQETPIVEVTRSITKHNYLVLDVEDIPRVIQEAFFLASSGRP |
| | GPVLVDIPKDIQQQMAVPVWDTSMNLPGYIARLPKPPATELLEQVLRLVGES |
| | RRPILYVGGGCSASGDELRWFVELTGIPVTTTLMGLGNFPSDDPLSLRMLGM |
| | HGTVYANYAVDKADLLLAFGVRFDDRVTGKIEAFASRAKIVHIDIDPAEIGK |
| | NKQPHVSICADVKLALQGLNALLQQSTTKTSSDFSAWHNELDQQKREFPLG |
| | YKTFGEEIPPQYAIQVLDELTKGEAIIATGVGQHQMWAAQYYTYKRPRQWL |
| | SSAGLGAMGFGLPAAAGASVANPGVTVVDIDGDGSFLMNIQELALIRIENLP |
| | VKVMVLNNQHLGMVVQLEDRFYKANRAHTYLGNPECESEIYPDFVTIAKGF |
| | NIPAVRVTKKSEVRAAIKKMLETPGPYLLDIIVPHQEHVLPMIPIGGAFKDMIL |
| | DGDGRTVY |
| 25 | ATGGCTGCGACtGCCAAGACCGGCCGTAAGAACCAtCAaCGACAtCAtGTCC |
| | TTCCCGCTCGAGGCCGaGTGGGGGCGGCGGCGGTCAGGTGCTCGGCGGTG |
| | TCCCCaGTCACCCCaCCaTCCCCaGCGCCaCCaGCCACtCCaCTCCGaCCaTGG |
| | GGGCCaGCCGAGCCCCGtAAGGGCGCGGACATCCTCGTGGAGGCGCTGGA |
| | GCGaTGCGGCGTCAGCGACGTGTTCGCCTAtCCaGGCGGCGCGTCCATGGA |
| | GATCCAtCAaGCGCTGACtCGtTCCCCaGTCATCACCAACCAtCTCTTCCGtCA |
| | tGAGCAaGGCGAGGCGTTCGCGGCGTCCGGGTAtGCGCGtGCGTCCGGCCGt |
| | GTCGGGGTCTGCGTCGCCACCTCCGGCCCCGGGGCAACCAACCTCGTGTC |
| | CGCGCTCGCCGACGCGCTGCTCGACTCCGTCCCaATGGTCGCCATCACtGG |
| | CCAaGTCCCCCGtCGtATGATCGGCACCGACGCCTTCCAaGAGACtCCCATA |
| | GTCGAGGTCACCCGtTCCATCACCAAGCAtAATTAtCTTGTCCTTGATGTGG |
| | AGGACATCCCCCGtGTCATACAaGAAGCCTTCTTCCTCGCGTCCTCGGGCC |
| | GTCCTGGCCCaGTGCTGGTCGACATCCCCAAGGACATCCAaCAaCAaATGG |
| | CCGTGCCaGTCTGGGACACCTCGATGAATCTACCAGGGTAtATCGCACGtC |
| | TGCCCAAGCCACCCGCGACAGAATTGCTTGAGCAaGTCTTGCGTCTGGTT |
| | GGCGAGTCACGaCGtCCaATTCTCTATGTCGGTGGTGGCTGCTCTGCATCTG |
| | GTGACGAATTGCGtTGGTTTGTTGAGCTGACTGGTATCCCAGTTACAACCA |
| | CTCTGATGGGCCTCGGCAATTTCCCCAGTGACGACCCaTTGTCCCTGCGtA |
| | TGCTTGGGATGCATGGCACtGTGTAtGCAAATTATGCCGTGGATAAGGCTG |
| | ACCTGTTGCTTGCGTTTGGTGTGCGaTTTGATGATCGTGTGACAGGGAAAA |
| | TTGAGGCTTTTGCAAGCAGGGCCAAGATTGTGCAtATTGACATTGATCCAG |
| | CAGAGATTGGAAAGAACAAGCAACCACATGTGTCAATTTGCGCAGATGT |
| | TAAGCTTGCTTTACAaGGCTTGAATGCTCTGCTACAACAaAGCACAACAAA |
| | GACAAGTTCTGATTTTAGTGCATGGCAtAATGAGTTGGACCAaCAaAAGAG |
| | GGAGTTTCCTCTGGGGTAtAAAACTTTTGGTGAAGAGATCCCACCaCAATA |
| | TGCCATTCAaGTGCTGGATGAGCTGACtAAAGGTGAGGCAATCATCGCTAC |
| | TGGTGTTGGGCAaCAtCAaATGTGGGCGGCACAATATTAtACCTAtAAGCGa |
| | CCACGaCAaTGGCTGTCTTCGGCTGGTCTGGGCGCAATGGGATTTGGGCTG |
| | CCTGCTGCAGCTGGTGCTTCTGTGGCTAACCCAGGTGTCACAGTTGTTGAT |
| | ATTGATGGGGATGGTAGCTTCCTCATGAACATTCAaGAGCTGGCATTGAT |
| | CCGtATTGAGAACCTCCCTGTGAAGGTGATGGTGTTGAACAACCAACATTT |
| | GGGTATGGTGGTGCAATTGGAGGATAGGTTTTAtAAGGCGAATAGGGCGC |
| | ATACATAtTTGGGCAACCCaGAATGTGAGAGCGAGATATATCCAGATTTTG |
| | TGACTATTGCTAAGGGGTTCAATATTCCTGCAGTCCGTGTAACAAAGAAG |
| | AGTGAAGTCCGTGCCGCCATCAAGAAGATGCTCGAGACTCCAGGGCCAT |
| | AtTTGTTGGATATCATCGTCCCaCAtCAaGAGCATGTGCTGCCTATGATCCC |
| | AATTGGGGGCGCATTCAAGGACATGATCCTGGATGGTGATGGCAGGACT |
| | GTGTATTAA |
| 26 | TAATACGACTCACTATAG |
| 27 | tctgcttgaaagcctgcagagtccaattttgagtattttcagttagaatctagagtcagcctattcagttcttagcccttaagggt |
| | aaggcagggggtaatatggatagtctctgtccctgtattcacattccaccttcaacaaagtgttgatttcccgtaaagctaact |
| | gtagtcctttaagtaagtagatatcttaggcaagttagcaatctcgttatattaccaaggccttcccttctattgtagaaagagtt |
| | ctcagccatctaattgcagtgccagttgccagctatccagtttcatttgaagttgctgggggtccaaacgagctagttgctttt |
| | attcgtcctataagtccttccacaagcgagtcaatagggtgctggctagttgtagttgttggcgtgcctttcctttcatcttgaat |
| | attaataaatatttggataaattactttagaataagaagttcatgtttTAATACGACTCACTATAGtaagtaata |
| | cgaatccatactaggaaaatgaaaatgtgagtcctaggcactggaattggttctcttctccctaatccctataagccagaaag |
| | ggtaataggcttcagtgtaagcatttccttcaagcaagtcatctcaagttttaaattctagagaatagctccgatcaacccatttt |
| | agtttggttctgcaattcattcgcataaatgaaaaaaaaagcgagatgtgcacgaaagaagatcatagttcagctttaaaatg |
| | gtggtgtccctgtgttagtaagtggttgaaatagctcatgggagtgtctgccccattcgataatggcatttatgatctagtgga |
| | gtgagtgattgtgtggtgttcagtctaaggctttttgaaaagcggatttctcccttctctcatccatcgtctttgttaaagt |
| 28 | AACCCCTTGGGGCCTCTAAACGGGTCTTGAGGGGTTTTTTG |
| 29 | AACCCCTTGGGGCCTCTAAACGGGTCTTGAGGGGTTTTTTGtagatagacctttttatt |
| | tttcgtcattcgatcacgaaaacagggattctggaacggccaagaatcccagcggttgttcgggtcgaaaaaccgagaac |
| | aagacatgccacaaagtggcagatgaaggcaggggggagagcctagtcctcaacctcttcttccccaaaaggtagttatg |
| | aacgtgccaaacttattggatttattcttggaatgctcataaccacctttactctttttttcattctttactcagaggaagccatg |
| | ccgtttggagaagagcaccaagtggggggagtgtggagtccccgaaagaggagctttctaaaggcaagagaaaagctccg |
| | atggagcccttggagctacagggaccaccaacccttcgcagcttggacgatttgattcttgtgccactcagccctgaggag |
| | ggcgcctgctcgacccagtcaggtactactccgccgccggccccgagtaactctgcgggggtcggtgcagccctttcttc |
| | tattccggagtgcataaacaaggatcctcaaaaagcgaaatcatttcgttaatggcatttcagaaatgagtcataggcgcct |
| | gtacaatgacagaatagagagtcctttttttccagaatgaatcattctattcaaatctcacaagttctctttacgcgtcttctagg |
| | ggcattgttgaacgcaatctgcaggaacaagaaatgattctttcttattttgaaacagaa |
| 30 | acttttgacagttatacgattccagaaGTGAGTAAGGGAGAGGAGCTGTTCACCGGGGTG |
| | GTGCCTATCCTGGTCGAGCTGGATGGTGATGTAAACGGTCATAAATTCAG |
| | TGTGTCCGGTGAAGGTGAAGGTGATGCCACCTATGGTAAGCTGACCCTTA |
| | AGTTCATCTGTACCACCGGAAAGCTGCCTGTGCCTTGGCCTACCCTCGTG |
| | ACCACCCTGACATATGGAGTGCAATGTTTCAGTCGTTATCCTGATCATAT |
| | GAAGCAACATGATTTCTTTAAATCCGCCATGCCTGAAGGTTATGTCCAAG |
| | AGCGTACCATATTCTTTAAAGATGATGGTAACTATAAGACCCGTGCCGAG |
| | GTGAAGTTCGAGGGTGATACCCTGGTGAACCGTATTGAGCTTAAGGGTAT |
| | CGATTTCAAGGAGGATGGAAACATCCTGGGGCATAAGCTGGAGTATAAC |
| | TATAACAGTCATAACGTCTATATCATGGCCGATAAGCAAAAGAACGGTAT |
| | CAAGGTGAACTTCAAGATCCGTCATAATATCGAAGATGGAAGTGTGCAA |
| | CTCGCCGATCATTATCAACAAAACACCCCTATCGGTGATGGTCCTGTGCT |
| | GCTGCCTGATAACCATTATCTGAGTACCCAATCCGCCCTGAGTAAAGATC |
| | CTAACGAGAAGCGTGATCAAATGGTACTGCTTGAGTTCGTTACCGCCGCC |
| | GGGATCACTCTCGGTATGGATGAGCTGTATAAGTAA |
| 31 | GGGGTCATCCCATTGGCCAGACTGAGCATCAAGCCAGCCAAGAAGTAAA |
| | AGCTGAGAAGGAGTGACTCGCATGAGTCAACACTTACTACTCAGGTCCGG |
| | TAGAGCAATCTCAAATTATCATATAGAAATGTTAATGTTATGATTTCGGT |
| | ATTGATCAAAAGGTGCTGGGACCTTAGGGCATACATTAGTGCCATGCCCT |
| | ATTGCGGAACGGTCGTATCCTGGTAACCTAGCCCCCGTAAGAGCTCTACC |
| | TAATCGTCGGGGTAGAAGGCTGTGCTTATTCTCGGCAAATAGCTAAGTCG |
| | ACACCCCGAGGGAGCAACTCAACTCTTCGTAGATCAAAACAAGTGTTCAC |
| | TGGAAAGTGGATCAAAGAAAAAAACTTCTTCGTTTCGTTGGAAAAACCG |
| | ACGCCAATATCATATTGACTCTCTCTCGTCCAATAAGAGTTTCCGAGAGTT |
| | ACTTTATTCAAATTCTCTCCTTTCCAAAGCTCCACAAGGCAGGCAAAAAG |
| | AGTAATAGGACAACAAGCAATCTTGTCTTTCATTTATTTGGAGTTCTTTCT |
| | TTGTTGAGATGGAAATCGACGTTCTTTTGAAAAGGGCTAGGTAGTTTGCA |
| | CGCAGGCAAAACTTCTTCATGAAAGGTAATAAATAGACTTTTTTTTCATG |
| | GGTTTCTTAATGACTAGTCGTTCGTTTGAAGCCTTAAGAAACCGGCAGTTT |
| | TTTTTCCGAATGACCTTATTTCGAGAATCAACTAACCGACAAATCCGTAG |
| | CCCAGGTGATTCGCTGCCTCCCTCTCGCCAAAATGGGATGAATCTTCTCAT |
| | GCAGCTTTTTTCTTGTTCAGGGCGCAGCGAAGCCAATTTCCATCAAGGCA |
| | AGGGGGTAAATAAGGGGGAAGAGGAGTTGTCACGATAGAAAAGAGAA |
| 32 | TAGACGGATGAGACTGATCACACCTGATCAGTGATCAATTCTGGCACAAT |
| | GAATTTACGAGTTATTTTACACAATGAATTTACAAGCAGATGAGTTTGCA |
| | ACGGTAGACCTATCTCCTGAAAAGAGTTCAGTAAACAAGGGAACGAAGC |
| | GACCGATAACGTCCCCTCGGGGAGGAGTGTTTT |
| 33 | ATGTTTAAACAAGCTTCTCGTCTCCTCTCCCGATCTGTCGCCGCCGCATCT |
| | TCCAAATCGGTGACGACTCGTGCCTTTTCAACGGAACTTCCATCGACGCT |
| | CGATTCCaacacgattaacatcgctaagaacgacttctctgacatcgaactggctgctatcccgttcaacactctggct |
| | gaccattacggtgagcgtttagctcgcgaacagttggcccttgagcatgagtcttacgagatgggtgaagcacgcttccgc |
| | aagatgtttgagcgtcaacttaaagctggtgaggttgcggataacgctgccgccaagcctctcatcactaccctactcccta |
| | agatgattgcacgcatcaacgactggtttgaggaagtgaaagctaagcgcggcaagcgcccgacagccttccagttcctg |
| | caagaaatcaagccggaagccgtagcgtacatcaccattaagaccactctggcttgcctaaccagtgctgacaatacaac |
| | cgttcaggctgtagcaagcgcaatcggtcgggccattgaggacgaggctcgcttcggtcgtatccgtgaccttgaagcta |
| | agcacttcaagaaaaacgttgaggaacaactcaacaagcgcgtagggcacgtctacaagaaagcatttatgcaagttgtc |
| | gaggctgacatgctctctaagggtctactcggtggcgaggcgtggtcttcgtggcataaggaagactctattcatgtagga |
| | gtacgctgcatcgagatgctcattgagtcaaccggaatggttagcttacaccgccaaaatgctggcgtagtaggtcaagac |
| | tctgagactatcgaactcgcacctgaatacgctgaggctatcgcaacccgtgcaggtgcgctggctggcatctctccgatg |
| | ttccaaccttgcgtagttcctcctaagccgtggactggcattactggtggtggctattgggctaacggtcgtcgtcctctggc |
| | gctggtgcgtactcacagtaagaaagcactgatgcgctacgaagacgtttacatgcctgaggtgtacaaagcgattaacat |
| | tgcgcaaaacaccgcatggaaaatcaacaagaaagtcctagcggtcgccaacgtaatcaccaagtggaagcattgtccg |
| | gtcgaggacatccctgcgattgagcgtgaagaactcccgatgaaaccggaagacatcgacatgaatcctgaggctctca |
| | ccgcgtggaaacgtgctgccgctgctgtgtaccgcaaggacaaggctcgcaagtctcgccgtatcagccttgagttcatg |
| | cttgagcaagccaataagtttgctaaccataaggccatctggttcccttacaacatggactggcgcggtcgtgtttacgctgt |
| | gtcaatgttcaacccgcaaggtaacgatatgaccaaaggactgcttacgctggcgaaaggtaaaccaatcggtaaggaa |
| | ggttactactggctgaaaatccacggtgcaaactgtgcgggtgtcgataaggttccgttccctgagcgcatcaagttcattg |
| | aggaaaaccacgagaacatcatggcttgcgctaagtctccactggagaacacttggtgggctgagcaagattctccgttct |
| | gcttccttgcgttctgctttgagtacgctggggtacagcaccacggcctgagctataactgctcccttccgctggcgtttgac |
| | gggtcttgctctggcatccagcacttctccgcgatgctccgagatgaggtaggtggtcgcgcggttaacttgcttcctagtg |
| | aaaccgttcaggacatctacgggattgttgctaagaaagtcaacgagattctacaagcagacgcaatcaatgggaccgat |
| | aacgaagtagttaccgtgaccgatgagaacactggtgaaatctctgagaaagtcaagctgggcactaaggcactggctg |
| | gtcaatggctggcttacggtgttactcgcagtgtgactaagcgttcagtcatgacgctggcttacgggtccaaagagttcgg |
| | cttccgtcaacaagtgctggaagataccattcagccagctattgattccggcaagggtctgatgttcactcagccgaatcag |
| | gctgctggatacatggctaagctgatttgggaatctgtgagcgtgacggtggtagctgcggttgaagcaatgaactggctt |
| | aagtctgctgctaagctgctggctgctgaggtcaaagataagaagactggagagattcttcgcaagcgttgcgctgtgcatt |
| | gggtaactcctgatggtttccctgtgtggcaggaatacaagaagcctattcagacgcgcttgaacctgatgttcctcggtca |
| | gttccgcttacagcctaccattaacaccaacaaagatagcgagattgatgcacacaaacaggagtctggtatcgctcctaa |
| | ctttgtacacagccaagacggtagccaccttcgtaagactgtagtgtgggcacacgagaagtacggaatcgaatcttttgc |
| | actgattcacgactccttcggtaccattccggctgacgctgcgaacctgttcaaagcagtgcgcgaaactatggttgacaca |
| | tatgagtcttgtgatgtactggctgatttctacgaccagttcgctgaccagttgcacgagtctcaattggacaaaatgccagc |
| | acttccggctaaaggtaacttgaacctccgtgacatcttagagtcggacttcgcgttcgcgtaa |
| 34 | MFKQASRLLSRSVAAASSKSVTTRAFSTELPSTLDSNTINIAKNDFSDIELAAIP |
| | FNTLADHYGERLAREQLALEHESYEMGEARFRKMFERQLKAGEVADNAAA |
| | KPLITTLLPKMIARINDWFEEVKAKRGKRPTAFQFLQEIKPEAVAYITIKTTLA |
| | CLTSADNTTVQAVASAIGRAIEDEARFGRIRDLEAKHFKKNVEEQLNKRVGH |
| | VYKKAFMQVVEADMLSKGLLGGEAWSSWHKEDSIHVGVRCIEMLIESTGM |
| | VSLHRQNAGVVGQDSETIELAPEYAEAIATRAGALAGISPMFQPCVVPPKPW |
| | TGITGGGYWANGRRPLALVRTHSKKALMRYEDVYMPEVYKAINIAQNTAW |
| | KINKKVLAVANVITKWKHCPVEDIPAIEREELPMKPEDIDMNPEALTAWKRA |
| | AAAVYRKDKARKSRRISLEFMLEQANKFANHKAIWFPYNMDWRGRVYAVS |
| | MFNPQGNDMTKGLLTLAKGKPIGKEGYYWLKIHGANCAGVDKVPFPERIKF |
| | IEENHENIMACAKSPLENTWWAEQDSPFCFLAFCFEYAGVQHHGLSYNCSLP |
| | LAFDGSCSGIQHFSAMLRDEVGGRAVNLLPSETVQDIYGIVAKKVNEILQAD |
| | AINGTDNEVVTVTDENTGEISEKVKLGTKALAGQWLAYGVTRSVTKRSVMT |
| | LAYGSKEFGFRQQVLEDTIQPAIDSGKGLMFTQPNQAAGYMAKLIWESVSVT |
| | VVAAVEAMNWLKSAAKLLAAEVKDKKTGEILRKRCAVHWVTPDGFPVWQ |
| | EYKKPIQTRLNLMFLGQFRLQPTINTNKDSEIDAHKQESGIAPNFVHSQDGSH |
| | LRKTVVWAHEKYGIESFALIHDSFGTIPADAANLFKAVRETMVDTYESCDVL |
| | ADFYDQFADQLHESQLDKMPALPAKGNLNLRDILESDFAFA |
| 35 | MFKQASRLLSRSVAAASSKSVTTRAFSTELPSTLDS |
| 36 | caagacctcactagtaaggaaggcacttgctgccggagttcaacaggcaaatataagaaaagaagtcctgttcacttcatc |
| | atctgtgggttgtactgcttgaaggttcttctgaggggtagaatttgaattccttctttgcttgtgagataaccatttccagaaac |
| | tcatatatagagagcgggtatcggtgaaaatggatcttaccaggagtggcattgaataggcaggctctgggatgtaatctca |
| | ctcaagaggtcatttgttggccccgccttcactagactagagttttaggataggttggggaacctatacgtcaagcccctac |
| | gaagattgagaaaaatcgatgcacataagccatccgaaaccagtattggaaagtgttcagtttcgttttccattctgaaatgtt |
| | catagtagtatagtatgttttccgttgggtcgacgccatgtgatcgctactaaagatagagtttccttggaaaaaccgaggcc |
| | agttgagatcagtctccctttctaggagcagagcttaaaaagatgggaaattccaatgaatttcgatcacaatcatgtggtaat |
| | aatgggtttgaatcagagagactcgatctggaaactcctcaatgattataacgtgaactcgttgaagagaaggagacaagc |
| | agaaatagacgctttttttgaaccatttgagagggcgcagcgtatccgtttcaataactggcagaacggaatagagttgttag |
| | atggggctgaatggaggaacggcgatatagttatccctggaggcggcggaccagtaatttcaagccccttggatcaattttt |
| | cattgatccattatttggtcttgatatgggtaacttttatttatcattcacaaatgaatccttgtctatggcggtaactgtcgttt |
| | tggtgccatctttatttggagttgttacgaaaaagggcgggggaaagtcagtgccaaatgcatggcaatccttggtagagcttatt |
| | tatgatttcgtgctgaacctggtaaacgaacaaataggtggaaatgttaaacaaaagtttttccctcgcatctcggtcactttta |
| | ctttttcgttatttcgtaatccccagggtatgataccctttagcttcacagtgacaagtcattttctcattactttggctctttca |
| | ttttccatttttataggcattacgatcgttggatttcaaagacatgggcttcatttttttagcttcttattaccagcgggagtccc |
| | actgccattagcaccttttttagtactccttgagctaatctctcattgttttcgtgcattaagctcaggaatacgtttatttgcta |
| | atatgatggccggtcatagttcagtaaagattttaagtgggttcgcttggactatgctatttctgaataatattttctatttcata |
| | ggagatcttggtcccttatttatTgtATtGgcTCTTacTggATtggaattaggtgtagctatattacaagctcatgtttctacgat |
| | ctcaatttgtatttacttgaatgatgctataaatctccatcaaaatgagtaatttcataattgaataaaaacgaggagccgaagat |
| | tttagggggcggga |
| 37 | GTCTGGCCCCAAATTCTAATTTCTACTGTTGTAGATTTCAATTATGAAATT |
| | ACTCAT |
| 38 | MNNGTNNFQNFIGISSLQKTLRNALIPTETTQQFIVKNGIIKEDELRGENRQIL |
| | KDIMDDYYRGFISETLSSIDDIDWTSLFEKMEIQLKNGDNKDTLIKEQTEYRK |
| | AIHKKFANDDRFKNMFSAKLISDILPEFVIHNNNYSASEKEEKTQVIKLFSRFA |
| | TSFKDYFKNRANCFSADDISSSSCHRIVNDNAEIFFSNALVYRRIVKSLSNDDI |
| | NKISGDMKDSLKEMSLEEIYSYEKYGEFITQEGISFYNDICGKVNSFMNLYCQ |
| | KNKENKNLYKLQKLHKQILCIADTSYEVPYKFESDEEVYQSVNGFLDNISSK |
| | HIVERLRKIGDNYNGYNLDKIYIVSKFYESVSQKTYRDWETINTALEIHYNNI |
| | LPGNGKSKADKVKKAVKNDLQKSITEINELVSNYKLCSDDNIKAETYIHEISH |
| | ILNNFEAQELKYNPEIHLVESELKASELKNVLDVIMNAFHWCSVFMTEELVD |
| | KDNNFYAELEEIYDEIYPVISLYNLVRNYVTQKPYSTKKIKLNFGIPTLADGW |
| | SKSKEYSNNAIILMRDNLYYLGIFNAKNKPDKKIIEGNTSENKGDYKKMIYNL |
| | LPGPNKMIPKVFLSSKTGVETYKPSAYILEGYKQNKHIKSSKDFDITFCHDLID |
| | YFKNCIAIHPEWKNFGFDFSDTSTYEDISGFYREVELQGYKIDWTYISEKDIDL |
| | LQEKGQLYLFQIYNKDFSKKSTGNDNLHTMYLKNLFSEENLKDIVLKLNGEA |
| | EIFFRKSSIKNPIIHKKGSILVNRTYEAEEKDQFGNIQIVRKNIPENIYQELYKYF |
| | NDKSDKELSDEAAKLKNVVGHHEAATNIVKDYRYTYDKYFLHMPITINFKA |
| | NKTGFINDRILQYIAKEKDLHVIGIDRGERNLIYVSVIDTCGNIVEQKSFNIVN |
| | GYDYQIKLKQQEGARQIARKEWKEIGKIKEIKEGYLSLVIHEISKMVIKYNAII |
| | AMEDLSYGFKKGRFKVERQVYQKFETMLINKLNYLVFKDISITENGGLLKGY |
| | QLTYIPDKLKNVGHQCGCIFYVPAAYTSKIDPTTGFVNIFKFKDLTVDAKREF |
| | IKKFDSIRYDSEKNLFCFTFDYNNFITQNTVMSKSSWSVYTYGVRIKRRFVNG |
| | RFSNESDTIDITKDMEKTLEMTDINWRDGHDLRQDIIDYEIVQHIFEIFRLTVQ |
| | MRNSLSELEDRDYDRLISPVLNENNIFYDSAKAGDALPKDADANGAYCIALK |
| | GLYEIKQITENWKEDGKFSRDKLKISNKDWFDFIQNKRYL |
| 39 | gaattcgaaaccatcttctcactctgacccccacatatcagatcccagatgcataggaaaagcggtatcaagaatagtagta |
| | taaagaaagatagtacagtactcaagtaaatgaattcgcctaaggatcgatggaaagatcaaggtccccgtgaaaaagta |
| | gatactagatcgatatgatactctcatctctggagtaacttcttccattatgctgatctctaggtccgttccatcatcatcgtaat |
| | agtatggtcccaggtgtccgagctatagatcaagatcatatCCagtcacatttctaccggtgcacttctcatgaaataattccc |
| | tttccaaggaaaggaaaacaagaactcgaatactcgtaatagcgatcccgatccacctacttttttctattctttgattcgaaac |
| | gtgctaaagcacaagccatttttatgcatggggcataagagtggacaatctatgttatcgaaggaagtaaataacaacactt |
| | cagcgtttaggtctaccttcagtaaaccaatagttttgcagcattggaatttgagttggccaggtaaggtcctctaaaaagaa |
| | aagaagaaactacttagaatagataaatgccattggttttctcgtactatacgatctttttttgttttgttttttggccatgattg |
| | tgctgctcctgtgaaggctagtgggaaagctcaccgttcgttgtgatgagtgggggccttgtatctgtattcggatcagctcctta |
| | acagagtttcctgcttgaaccctggctgggagctgggagaggtgtcccactacaggtgcaaataaaccatttgaccttaca |
| | ggggaaaggaaacaaaccactcaataatcggtagaaattcctcctactgaacagctttccttttctcgccttaactactacttc |
| | aaagcaaggcggaatatcacgggataggaatgaaagaacttcttactcaactttctagctatataaaaatagttagcaatatg |
| | aaacgagtaacttaagccctagtaaaaggctactctttgaatcccctctttaaggcatataaaattagtactcttcctgagctag |
| | cttaagcatatcttgagcgagtgagttgtatttccctccatcaagttctaagcgatcaaataaggtccttgctctcgagccaat |
| | gccaataccaatagagagggtctaaacgaaggattcaaa |
| 40 | GTCTGGCCCCAAATTCTAATTTCTACTGTTGTAGATGTCACATTTCTACCG |
| | GTGCAC |
| 41 | gaatttctctgacattccatgtttccgaaaCggatcctata |
| 42 | tgaacagtcactcacttttgacagttataCgattccagaa |
| 43 | AACCCCTTGGGGCCTCTAAACGGGTCTTGAGGGGTTTTTTGtagatagacctttttatt |
| | tttcgtcattcgatcacgaaaa |
| 44 | GGGCCCAAATTCAATTGTATATGAGCTCATATACAAGACCTCACTAGTAA |
| | GGAAGGCACTTGCTGCCGGAGTTCAACAGGCAAATATAAGAAAAGAAGT |
| | CCTGTTCACTTCATCATCTGTGGGTTGTACTGCTTGAAGGTTCTTCTGAGG |
| | GGTAGAATTTGAATTCCTTCTTTGCTTGTGAGATAACCATTTCCAGAAACT |
| | CATATATAGAGAGCGGGTATCGGTGAAAATGGATCTTACCAGGAGTGGC |
| | ATTGAATAGGCAGGCTCTGGGATGTAATCTCACTCAAGAGGTCATTTGTT |
| | GGCCCCGCCTTCACTAGACTAGAGTTTTAGGATAGGTTGGGGAACCTATA |
| | CGTCAAGCCCCTACGAAGATTGAGAAAAATCGATGCACATAAGCCATCC |
| | GAAACCAGTATTGGAAAGTGTTCAGTTTCGTTTTCCATTCTGAAATGTTCA |
| | TAGTAGTATAGTATGTTTTCCGTTGGGTCGACGCCATGTGATCGCTACTAA |
| | AGATAGAGTTTCCTTGGAAAAACCGAGGCCAGTTGAGATCAGTCTCCCTT |
| | TCTAGGAGCAGAGCTTAAAAAGATGGGAAATTCCAATGAATTTCGATCAC |
| | AATCATGTGGTAATAATGGGTTTGAATCAGAGAGACTCGATCTGGAAACT |
| | CCTCAATGATTATAACGTGAACTCGTTGAAGAGAAGGAGACAAGCAGAA |
| | ATAGACGCTTTTTTTGAACCATTTGAGAGGGCGCAGCGTATCCGTTTCAA |
| | TAACTGGCAGAACGGAATAGAGTTGTTAGATGGGGCTGAATGGAGGAAC |
| | GGCGATATAGTTATCCCTGGAGGCGGCGGACCAGTAATTTCAAGCCCCTT |
| | GGATCAATTTTTCATTGATCCATTATTTGGTCTTGATATGGGTAACTTTTA |
| | TTTATCATTCACAAATGAATCCTTGTCTATGGCGGTAACTGTCGTTTTGGT |
| | GCCATCTTTATTTGGAGTTGTTACGAAAAAGGGGGGGGGAAAGTCAGTGC |
| | CAAATGCATGGCAATCCTTGGTAGAGCTTATTTATGATTTCGTGCTGAAC |
| | CTGGTAAACGAACAAATAGGTGGAAATGTTAAACAAAAGTTTTTCCCTCG |
| | CATCTCGGTCACTTTTACTTTTTCGTTATTTCGTAATCCCCAGGGTATGAT |
| | ACCCTTTAGCTTCACAGTGACAAGTCATTTTCTCATTACTTTGGCTCTTTC |
| | ATTTTCCATTTTTATAGGCATTACGATCGTTGGATTTCAAAGACATGGGCT |
| | TCATTTTTTTAGCTTCTTATTACCAGCGGGAGTCCCACTGCCATTAGCACC |
| | TTTTTTAGTACTCCTTGAGCTAATCTCTCATTGTTTTCGTGCATTAAGCTCA |
| | GGAATACGTTTATTTGCTAATATGATGGCCGGTCATAGTTCAGTAAAGAT |
| | TTTAAGTGGGTTCGCTTGGACTATGCTATTTCTGAATAATATTTTCTATTT |
| | CATAGGAGATCTTGGTCCCTTATTTATTGTATTGGCTCTTACTGGATTGGA |
| | ATTAGGTGTAGCTATATTACAAGCTCATGTTTCTACGATCTCAATTTGTAT |
| | TTACTTGAATGATGCTATAAATCTCCATCAAAATGAGTAATTTCATAATTG |
| | AATAAAAACGAGGAGCCGAAGATTTTAGGGGGCGGGACAAACGCGGAA |
| | GTGTATTGCGTTACAAAAAATGACAACTAGCATTTGTTTTTTCATTTCATG |
| | TTCGAATTCGTTTTTCGTTGGAAAAACCAACGCCGACCCCAAACAAGTCT |
| | CTCCAATATAAGGAGAGCGGAGCTTAAAAATATTATTTTATTGTGCTATG |
| | GCAAATCTGGTCCGATGGCTCTTCTCCACTACCCGAGGGACTAACGGTCT |
| | TCCATATTTCATCTTCGGTGTCGTTGTAGGAGGCGCCCTGTTGTTTGCTTT |
| | GCTAAAGTATCAGGCCCCTCTGTACGACCCGGCTTTAATGGAAAAAATCA |
| | TAGATCATAATATAAAAGCCGGGCACCCTATAGAGGTTGACTATTCGTGG |
| | TGGGGCACCTCTATTCGTGTAGTCTTTCCTAAGTAAGAAAGACAGGACAG |
| | TGGTGGTTTGCTCATACTTTCATTACAAAACCATACTATGGAATTAGGGA |
| | TAACAGGGTAATAATCACAAGTGAGAACCACAGGTAGCAATAGGTATTA |
| | CAGAAATTTCCTCGAGTCTGCTTGAAAGCCTGCAGAGTCCAATTTTGAGT |
| | ATTTTCAGTTAGAATCTAGAGTCAGCCTATTCAGTTCTTAGCCCTTAAGGG |
| | TAAGGCAGGGGGTAATATGGATAGTCTCTGTCCCTGTATTCACATTCCAC |
| | CTTCAACAAAGTGTTGATTTCCCGTAAAGCTAACTGTAGTCCTTTAAGTA |
| | AGTAGATATCTTAGGCAAGTTAGCAATCTCGTTATATTACCAAGGCCTTC |
| | CCTTCTATTGTAGAAAGAGTTCTCAGCCATCTAATTGCAGTGCCAGTTGCC |
| | AGCTATCCAGTTTCATTTGAAGTTGCTGGGGGTCCAAACGAGCTAGTTGC |
| | TTTTATTCGTCCTATAAGTCCTTCCACAAGCGAGTCAATAGGGTGCTGGCT |
| | AGTTGTAGTTGTTGGCGTGCCTTTCCTTTCATCTTGAATATTAATAAATAT |
| | TTGGATAAATTACTTTAGAATAAGAAGTTCATGTTTTAATACGACTCACT |
| | ATAGTAAGTAATACGAATCCATACTAGGAAAATGAAAATGTGAGTCCTA |
| | GGCACTGGAATTGGTTCTCTTCTCCCTAATCCCTATAAGCCAGAAAGGGT |
| | AATAGGCTTCAGTGTAAGCATTTCCTTCAAGCAAGTCATCTCAAGTTTTA |
| | AATTCTAGAGAATAGCTCCGATCAACCCATTTTAGTTTGGTTCTGCAATTC |
| | ATTCGCATAAATGAAAAAAAAAGCGAGATGTGCACGAAAGAAGATCATA |
| | GTTCAGCTTTAAAATGGTGGTGTCCCTGTGTTAGTAAGTGGTTGAAATAG |
| | CTCATGGGAGTGTCTGCCCCATTCGATAATGGCATTTATGATCTAGTGGA |
| | GTGAGTGATTGTGTGGTGTTCAGTCTAAGGCTTTTTGAAAAGCGGATTTCT |
| | CCCTTCTCTCATCCATCGTCTTTGTTAAAGTGAATTTCTCTGACATTCCAT |
| | GTTTCCGAAAcGGATCCTATAAAGACCGGCCGTAAGAACCATCAACGA |
| | CATCATGTCCTTCCCGCACGAGGCCGAGTGGGGGCGGCGGCGGTCA |
| | GGTGCTCGGCGGTGTCCCCAGTCACCCCACCATCCCCAGCGCCACCA |
| | GCCACTCCACTCCGACCATGGGGGCCAGCCGAGCCCCGTAAGGGCG |
| | CGGACATCCTCGTGGAGGCGCTGGAGCGATGCGGCGTCAGCGACGT |
| | GTTCGCCTATCCAGGCGGCGCGTCCATGGAGATCCATCAAGCGCTGA |
| | CTCGTTCCCCAGTCATCACCAACCATCTCTTCCGTCATGAGCAAGGC |
| | GAGGCGTTCGCGGCGTCCGGGTATGCGCGTGCGTCCGGCCGTGTCG |
| | GGGTCTGCGTCGCCACCTCCGGCCCCGGGGCAACCAACCTCGTGTCC |
| | GCGCTCGCCGACGCGCTGCTCGACTCCGTCCCAATGGTCGCCATCAC |
| | TGGCCAAGTCCCCCGTCGTATGATCGGCACCGACGCCTTCCAAGAGA |
| | CTCCCATAGTCGAGGTCACCCGTTCCATCACCAAGCATAATTATCTTG |
| | TCCTTGATGTGGAGGACATCCCCCGTGTCATACAAGAAGCCTTCTTC |
| | CTCGCGTCCTCGGGCCGTCCTGGCCCAGTGCTGGTCGACATCCCCAA |
| | GGACATCCAACAACAAATGGCCGTGCCAGTCTGGGACACCTCGATGA |
| | ATCTACCAGGGTATATCGCACGTCTGCCCAAGCCACCCGCGACAGAA |
| | TTGCTTGAGCAAGTCTTGCGTCTGGTTGGCGAGTCACGACGTCCAAT |
| | TCTCTATGTCGGTGGTGGCTGCTCTGCATCTGGTGACGAATTGCGTT |
| | GGTTTGTTGAGCTGACTGGTATCCCAGTTACAACCACTCTGATGGGC |
| | CTCGGCAATTTCCCCAGTGACGACCCATTGTCCCTGCGTATGCTTGG |
| | GATGCATGGCACTGTGTATGCAAATTATGCCGTGGATAAGGCTGACC |
| | TGTTGCTTGCGTTTGGTGTGCGATTTGATGATCGTGTGACAGGGAAA |
| | ATTGAGGCTTTTGCAAGCAGGGCCAAGATTGTGCATATTGACATTGA |
| | TCCAGCAGAGATTGGAAAGAACAAGCAACCACATGTGTCAATTTGCG |
| | CAGATGTTAAACTTGCTTTACAAGGCTTGAATGCTCTGCTACAACAAA |
| | GCACAACAAAGACAAGTTCTGATTTTAGTGCATGGCATAATGAGTTG |
| | GACCAACAAAAGAGGGAGTTTCCTCTGGGGTATAAAACTTTTGGTGA |
| | AGAGATCCCACCACAATATGCCATTCAAGTGCTGGATGAGCTGACTA |
| | AAGGTGAGGCAATCATCGCTACTGGTGTTGGGCAACATCAAATGTGG |
| | GCGGCACAATATTATACCTATAAGCGACCACGACAATGGCTGTCTTC |
| | GGCTGGTCTGGGCGCAATGGGATTTGGGCTGCCTGCTGCAGCTGGT |
| | GCTTCTGTGGCTAACCCAGGTGTCACAGTTGTTGATATTGATGGGGA |
| | TGGTAGCTTCCTCATGAACATTCAAGAGCTGGCATTGATCCGTATTG |
| | AGAACCTCCCTGTGAAGGTGATGGTGTTGAACAACCAACATTTGGGT |
| | ATGGTGGTGCAATTGGAGGATAGGTTTTATAAGGCGAATAGGGCGCA |
| | TACATATTTGGGCAACCCAGAATGTGAGAGCGAGATATATCCAGATT |
| | TTGTGACTATTGCTAAGGGGTTCAATATTCCTGCAGTCCGTGTAACA |
| | AAGAAGAGTGAAGTCCGTGCCGCCATCAAGAAGATGCTTGAGACTCC |
| | AGGGCCATATTTGTTGGATATCATCGTCCCACATCAAGAGCATGTGC |
| | TGCCTATGATCCCAATTGGGGGCGCATTCAAGGACATGATCCTGGAT |
| | GGTGATGGCAGGACTGTGTATTAATGAGGTACCAAGCGATCGCAAACC |
| | TAGGAAAAGATCTAGACGAGGTGTAGCGCAGTCTGGTCAGCGCATCTGTT |
| | TTGGGTACAGAGGGCCATAGGTTCGAATCCTGTCACCTTGAGTCTGGCCC |
| | CAAATTCTAATTTCTACTGTTGTAGATTTCAATTATGAAATTACTCATGTC |
| | CCTTTCGTCCAGTGGTTAGGACATCGTCTTTTCATGTCGAAGACACGGGTT |
| | CGATTCCCGTAAGGGATAGTCTGGCCCCAAATTCTAATTTCTACTGTTGTA |
| | GATGTCACATTTCTACCGGTGCACATTCCAGCTTATTTGATACCCACTTCA |
| | AGTTTCTATCAAACCATGTCTTTTTCTTCGAACGTCAATCTCGTAGGAAAG |
| | ATCTAAAAAGCTTAAGCGGCCGCAAAAACCCCTTGGGGCCTCTAAACGG |
| | GTCTTGAGGGGTTTTTTGTAGATAGACCTTTTTATTTTTCGTCATTCGATC |
| | ACGAAAAGGCCGGCCAAACCTCGAGGTAATTAAAGCGGCCGAAATTAGC |
| | TAGCAAATAAGCATGCAATCACAAGTGAGAACCACAGGTAGCAATAGGT |
| | ATTACAGTAGGGATAACAGGGTAATGAATTCGAAACCATCTTCTCACTCT |
| | GACCCCCACATATCAGATCCCAGATGCATAGGAAAAGCGGTATCAAGAA |
| | TAGTAGTATAAAGAAAGATAGTACAGTACTCAAGTAAATGAATTCGCCTA |
| | AGGATCGATGGAAAGATCAAGGTCCCCGTGAAAAAGTAGATACTAGATC |
| | GATATGATACTCTCATCTCTGGAGTAACTTCTTCCATTATGCTGATCTCTA |
| | GGTCCGTTCCATCATCATCGTAATAGTATGGTCCCAGGTGTCCGAGCTAT |
| | AGATCAAGATCATATCCAGTCACATTTCTACCGGTGCACTTCTCATGAAA |
| | TAATTCCCTTTCCAAGGAAAGGAAAACAAGAACTCGAATACTCGTAATAG |
| | CGATCCCGATCCACCTACTTTTTTCTATTCTTTGATTCGAAACGTGCTAAA |
| | GCACAAGCCATTTTTATGCATGGGGCATAAGAGTGGACAATCTATGTTAT |
| | CGAAGGAAGTAAATAACAACACTTCAGCGTTTAGGTCTACCTTCAGTAAA |
| | CCAATAGTTTTGCAGCATTGGAATTTGAGTTGGCCAGGTAAGGTCCTCTA |
| | AAAAGAAAAGAAGAAACTACTTAGAATAGATAAATGCCATTGGTTTTCTC |
| | GTACTATACGATCTTTTTTTGTTTTGTTTTTTGGCCATGATTGTGCTGCTCC |
| | TGTGAAGGCTAGTGGGAAAGCTCACCGTTCGTTGTGATGAGTGGGGGCCT |
| | TGTATCTGTATTCGGATCAGCTCCTTAACAGAGTTTCCTGCTTGAACCCTG |
| | GCTGGGAGCTGGGAGAGGTGTCCCACTACAGGTGCAAATAAACCATTTG |
| | ACCTTACAGGGGAAAGGAAACAAACCACTCAATAATCGGTAGAAATTCC |
| | TCCTACTGAACAGCTTTCCTTTTCTCGCCTTAACTACTACTTCAAAGCAAG |
| | GCGGAATATCACGGGATAGGAATGAAAGAACTTCTTACTCAACTTTCTAG |
| | CTATATAAAAATAGTTAGCAATATGAAACGAGTAACTTAAGCCCTAGTAA |
| | AAGGCTACTCTTTGAATCCCCTCTTTAAGGCATATAAAATTAGTACTCTTC |
| | CTGAGCTAGCTTAAGCATATCTTGAGCGAGTGAGTTGTATTTCCCTCCATC |
| | AAGTTCTAAGCGATCAAATAAGGTCCTTGCTCTCGAGCCAATGCCAATAC |
| | CAATAGAGAGGGTCTAAACGAAGGATTCAAAGGCGCGCC |
| 45 | GGGCCCAAATTCAATTGTATATGAGCTCATATACAAGACCTCACTAGTAA |
| | GGAAGGCACTTGCTGCCGGAGTTCAACAGGCAAATATAAGAAAAGAAGT |
| | CCTGTTCACTTCATCATCTGTGGGTTGTACTGCTTGAAGGTTCTTCTGAGG |
| | GGTAGAATTTGAATTCCTTCTTTGCTTGTGAGATAACCATTTCCAGAAACT |
| | CATATATAGAGAGCGGGTATCGGTGAAAATGGATCTTACCAGGAGTGGC |
| | ATTGAATAGGCAGGCTCTGGGATGTAATCTCACTCAAGAGGTCATTTGTT |
| | GGCCCCGCCTTCACTAGACTAGAGTTTTAGGATAGGTTGGGGAACCTATA |
| | CGTCAAGCCCCTACGAAGATTGAGAAAAATCGATGCACATAAGCCATCC |
| | GAAACCAGTATTGGAAAGTGTTCAGTTTCGTTTTCCATTCTGAAATGTTCA |
| | TAGTAGTATAGTATGTTTTCCGTTGGGTCGACGCCATGTGATCGCTACTAA |
| | AGATAGAGTTTCCTTGGAAAAACCGAGGCCAGTTGAGATCAGTCTCCCTT |
| | TCTAGGAGCAGAGCTTAAAAAGATGGGAAATTCCAATGAATTTCGATCAC |
| | AATCATGTGGTAATAATGGGTTTGAATCAGAGAGACTCGATCTGGAAACT |
| | CCTCAATGATTATAACGTGAACTCGTTGAAGAGAAGGAGACAAGCAGAA |
| | ATAGACGCTTTTTTTGAACCATTTGAGAGGGCGCAGCGTATCCGTTTCAA |
| | TAACTGGCAGAACGGAATAGAGTTGTTAGATGGGGCTGAATGGAGGAAC |
| | GGCGATATAGTTATCCCTGGAGGCGGCGGACCAGTAATTTCAAGCCCCTT |
| | GGATCAATTTTTCATTGATCCATTATTTGGTCTTGATATGGGTAACTTTTA |
| | TTTATCATTCACAAATGAATCCTTGTCTATGGCGGTAACTGTCGTTTTGGT |
| | GCCATCTTTATTTGGAGTTGTTACGAAAAAGGGCGGGGGGAAAGTCAGTGC |
| | CAAATGCATGGCAATCCTTGGTAGAGCTTATTTATGATTTCGTGCTGAAC |
| | CTGGTAAACGAACAAATAGGTGGAAATGTTAAACAAAAGTTTTTCCCTCG |
| | CATCTCGGTCACTTTTACTTTTTCGTTATTTCGTAATCCCCAGGGTATGAT |
| | ACCCTTTAGCTTCACAGTGACAAGTCATTTTCTCATTACTTTGGCTCTTTC |
| | ATTTTCCATTTTTATAGGCATTACGATCGTTGGATTTCAAAGACATGGGCT |
| | TCATTTTTTTAGCTTCTTATTACCAGCGGGAGTCCCACTGCCATTAGCACC |
| | TTTTTTAGTACTCCTTGAGCTAATCTCTCATTGTTTTCGTGCATTAAGCTCA |
| | GGAATACGTTTATTTGCTAATATGATGGCCGGTCATAGTTCAGTAAAGAT |
| | TTTAAGTGGGTTCGCTTGGACTATGCTATTTCTGAATAATATTTTCTATTT |
| | CATAGGAGATCTTGGTCCCTTATTTATTGTATTGGCTCTTACTGGATTGGA |
| | ATTAGGTGTAGCTATATTACAAGCTCATGTTTCTACGATCTCAATTTGTAT |
| | TTACTTGAATGATGCTATAAATCTCCATCAAAATGAGTAATTTCATAATTG |
| | AATAAAAACGAGGAGCCGAAGATTTTAGGGGGCGGGACAAACGCGGAA |
| | GTGTATTGCGTTACAAAAAATGACAACTAGCATTTGTTTTTTCATTTCATG |
| | TTCGAATTCGTTTTTCGTTGGAAAAACCAACGCCGACCCCAAACAAGTCT |
| | CTCCAATATAAGGAGAGCGGAGCTTAAAAATATTATTTTATTGTGCTATG |
| | GCAAATCTGGTCCGATGGCTCTTCTCCACTACCCGAGGGACTAACGGTCT |
| | TCCATATTTCATCTTCGGTGTCGTTGTAGGAGGCGCCCTGTTGTTTGCTTT |
| | GCTAAAGTATCAGGCCCCTCTGTACGACCCGGCTTTAATGGAAAAAATCA |
| | TAGATCATAATATAAAAGCCGGGCACCCTATAGAGGTTGACTATTCGTGG |
| | TGGGGCACCTCTATTCGTGTAGTCTTTCCTAAGTAAGAAAGACAGGACAG |
| | TGGTGGTTTGCTCATACTTTCATTACAAAACCATACTATGGAATTAGGGA |
| | TAACAGGGTAATAATCACAAGTGAGAACCACAGGTAGCAATAGGTATTA |
| | CAGAAATTTCCTCGAGTCTGCTTGAAAGCCTGCAGAGTCCAATTTTGAGT |
| | ATTTTCAGTTAGAATCTAGAGTCAGCCTATTCAGTTCTTAGCCCTTAAGGG |
| | TAAGGCAGGGGGTAATATGGATAGTCTCTGTCCCTGTATTCACATTCCAC |
| | CTTCAACAAAGTGTTGATTTCCCGTAAAGCTAACTGTAGTCCTTTAAGTA |
| | AGTAGATATCTTAGGCAAGTTAGCAATCTCGTTATATTACCAAGGCCTTC |
| | CCTTCTATTGTAGAAAGAGTTCTCAGCCATCTAATTGCAGTGCCAGTTGCC |
| | AGCTATCCAGTTTCATTTGAAGTTGCTGGGGGTCCAAACGAGCTAGTTGC |
| | TTTTATTCGTCCTATAAGTCCTTCCACAAGCGAGTCAATAGGGTGCTGGCT |
| | AGTTGTAGTTGTTGGCGTGCCTTTCCTTTCATCTTGAATATTAATAAATAT |
| | TTGGATAAATTACTTTAGAATAAGAAGTTCATGTTTTAATACGACTCACT |
| | ATAGTAAGTAATACGAATCCATACTAGGAAAATGAAAATGTGAGTCCTA |
| | GGCACTGGAATTGGTTCTCTTCTCCCTAATCCCTATAAGCCAGAAAGGGT |
| | AATAGGCTTCAGTGTAAGCATTTCCTTCAAGCAAGTCATCTCAAGTTTTA |
| | AATTCTAGAGAATAGCTCCGATCAACCCATTTTAGTTTGGTTCTGCAATTC |
| | ATTCGCATAAATGAAAAAAAAAGCGAGATGTGCACGAAAGAAGATCATA |
| | GTTCAGCTTTAAAATGGTGGTGTCCCTGTGTTAGTAAGTGGTTGAAATAG |
| | CTCATGGGAGTGTCTGCCCCATTCGATAATGGCATTTATGATCTAGTGGA |
| | GTGAGTGATTGTGTGGTGTTCAGTCTAAGGCTTTTTGAAAAGCGGATTTCT |
| | CCCTTCTCTCATCCATCGTCTTTGTTAAAGTTGAACAGTCACTCACTTTTG |
| | ACAGTTATAcGATTCCAGAAAAGACCGGCCGTAAGAACCATCAACGAC |
| | ATCATGTCCTTCCCGCACGAGGCCGAGTGGGGGCGGCGGCGGTCAG |
| | GTGCTCGGCGGTGTCCCCAGTCACCCCACCATCCCCAGCGCCACCAG |
| | CCACTCCACTCCGACCATGGGGGCCAGCCGAGCCCCGTAAGGGCGC |
| | GGACATCCTCGTGGAGGCGCTGGAGCGATGCGGCGTCAGCGACGTG |
| | TTCGCCTATCCAGGCGGCGCGTCCATGGAGATCCATCAAGCGCTGAC |
| | TCGTTCCCCAGTCATCACCAACCATCTCTTCCGTCATGAGCAAGGCG |
| | AGGCGTTCGCGGCGTCCGGGTATGCGCGTGCGTCCGGCCGTGTCGG |
| | GGTCTGCGTCGCCACCTCCGGCCCCGGGGCAACCAACCTCGTGTCCG |
| | CGCTCGCCGACGCGCTGCTCGACTCCGTCCCAATGGTCGCCATCACT |
| | GGCCAAGTCCCCCGTCGTATGATCGGCACCGACGCCTTCCAAGAGAC |
| | TCCCATAGTCGAGGTCACCCGTTCCATCACCAAGCATAATTATCTTGT |
| | CCTTGATGTGGAGGACATCCCCCGTGTCATACAAGAAGCCTTCTTCC |
| | TCGCGTCCTCGGGCCGTCCTGGCCCAGTGCTGGTCGACATCCCCAAG |
| | GACATCCAACAACAAATGGCCGTGCCAGTCTGGGACACCTCGATGAA |
| | TCTACCAGGGTATATCGCACGTCTGCCCAAGCCACCCGCGACAGAAT |
| | TGCTTGAGCAAGTCTTGCGTCTGGTTGGCGAGTCACGACGTCCAATT |
| | CTCTATGTCGGTGGTGGCTGCTCTGCATCTGGTGACGAATTGCGTTG |
| | GTTTGTTGAGCTGACTGGTATCCCAGTTACAACCACTCTGATGGGCC |
| | TCGGCAATTTCCCCAGTGACGACCCATTGTCCCTGCGTATGCTTGGG |
| | ATGCATGGCACTGTGTATGCAAATTATGCCGTGGATAAGGCTGACCT |
| | GTTGCTTGCGTTTGGTGTGCGATTTGATGATCGTGTGACAGGGAAAA |
| | TTGAGGCTTTTGCAAGCAGGGCCAAGATTGTGCATATTGACATTGAT |
| | CCAGCAGAGATTGGAAAGAACAAGCAACCACATGTGTCAATTTGCGC |
| | AGATGTTAAACTTGCTTTACAAGGCTTGAATGCTCTGCTACAACAAA |
| | GCACAACAAAGACAAGTTCTGATTTTAGTGCATGGCATAATGAGTTG |
| | GACCAACAAAAGAGGGAGTTTCCTCTGGGGTATAAAACTTTTGGTGA |
| | AGAGATCCCACCACAATATGCCATTCAAGTGCTGGATGAGCTGACTA |
| | AAGGTGAGGCAATCATCGCTACTGGTGTTGGGCAACATCAAATGTGG |
| | GCGGCACAATATTATACCTATAAGCGACCACGACAATGGCTGTCTTC |
| | GGCTGGTCTGGGCGCAATGGGATTTGGGCTGCCTGCTGCAGCTGGT |
| | GCTTCTGTGGCTAACCCAGGTGTCACAGTTGTTGATATTGATGGGGA |
| | TGGTAGCTTCCTCATGAACATTCAAGAGCTGGCATTGATCCGTATTG |
| | AGAACCTCCCTGTGAAGGTGATGGTGTTGAACAACCAACATTTGGGT |
| | ATGGTGGTGCAATTGGAGGATAGGTTTTATAAGGCGAATAGGGCGCA |
| | TACATATTTGGGCAACCCAGAATGTGAGAGCGAGATATATCCAGATT |
| | TTGTGACTATTGCTAAGGGGTTCAATATTCCTGCAGTCCGTGTAACA |
| | AAGAAGAGTGAAGTCCGTGCCGCCATCAAGAAGATGCTTGAGACTCC |
| | AGGGCCATATTTGTTGGATATCATCGTCCCACATCAAGAGCATGTGC |
| | TGCCTATGATCCCAATTGGGGGCGCATTCAAGGACATGATCCTGGAT |
| | GGTGATGGCAGGACTGTGTATTAATGAGGTACCAAGCGATCGCAAACC |
| | TAGGAAAAGATCTAGACGAGGTGTAGCGCAGTCTGGTCAGCGCATCTGTT |
| | TTGGGTACAGAGGGCCATAGGTTCGAATCCTGTCACCTTGAGTCTGGCCC |
| | CAAATTCTAATTTCTACTGTTGTAGATTTCAATTATGAAATTACTCATGTC |
| | CCTTTCGTCCAGTGGTTAGGACATCGTCTTTTCATGTCGAAGACACGGGTT |
| | CGATTCCCGTAAGGGATAGTCTGGCCCCAAATTCTAATTTCTACTGTTGTA |
| | GATGTCACATTTCTACCGGTGCACATTCCAGCTTATTTGATACCCACTTCA |
| | AGTTTCTATCAAACCATGTCTTTTTCTTCGAACGTCAATCTCGTAGGAAAG |
| | ATCTAAAAAGCTTAAGCGGCCGCAAAAACCCCTTGGGGCCTCTAAACGG |
| | GTCTTGAGGGGTTTTTTGTAGATAGACCTTTTTATTTTTCGTCATTCGATC |
| | ACGAAAAGGCCGGCCAAACCTCGAGGTAATTAAAGCGGCCGAAATTAGC |
| | TAGCAAATAAGCATGCAATCACAAGTGAGAACCACAGGTAGCAATAGGT |
| | ATTACAGTAGGGATAACAGGGTAATGAATTCGAAACCATCTTCTCACTCT |
| | GACCCCCACATATCAGATCCCAGATGCATAGGAAAAGCGGTATCAAGAA |
| | TAGTAGTATAAAGAAAGATAGTACAGTACTCAAGTAAATGAATTCGCCTA |
| | AGGATCGATGGAAAGATCAAGGTCCCCGTGAAAAAGTAGATACTAGATC |
| | GATATGATACTCTCATCTCTGGAGTAACTTCTTCCATTATGCTGATCTCTA |
| | GGTCCGTTCCATCATCATCGTAATAGTATGGTCCCAGGTGTCCGAGCTAT |
| | AGATCAAGATCATATCCAGTCACATTTCTACCGGTGCACTTCTCATGAAA |
| | TAATTCCCTTTCCAAGGAAAGGAAAACAAGAACTCGAATACTCGTAATAG |
| | CGATCCCGATCCACCTACTTTTTTCTATTCTTTGATTCGAAACGTGCTAAA |
| | GCACAAGCCATTTTTATGCATGGGGCATAAGAGTGGACAATCTATGTTAT |
| | CGAAGGAAGTAAATAACAACACTTCAGCGTTTAGGTCTACCTTCAGTAAA |
| | CCAATAGTTTTGCAGCATTGGAATTTGAGTTGGCCAGGTAAGGTCCTCTA |
| | AAAAGAAAAGAAGAAACTACTTAGAATAGATAAATGCCATTGGTTTTCTC |
| | GTACTATACGATCTTTTTTTGTTTTGTTTTTTGGCCATGATTGTGCTGCTCC |
| | TGTGAAGGCTAGTGGGAAAGCTCACCGTTCGTTGTGATGAGTGGGGGCCT |
| | TGTATCTGTATTCGGATCAGCTCCTTAACAGAGTTTCCTGCTTGAACCCTG |
| | GCTGGGAGCTGGGAGAGGTGTCCCACTACAGGTGCAAATAAACCATTTG |
| | ACCTTACAGGGGAAAGGAAACAAACCACTCAATAATCGGTAGAAATTCC |
| | TCCTACTGAACAGCTTTCCTTTTCTCGCCTTAACTACTACTTCAAAGCAAG |
| | GCGGAATATCACGGGATAGGAATGAAAGAACTTCTTACTCAACTTTCTAG |
| | CTATATAAAAATAGTTAGCAATATGAAACGAGTAACTTAAGCCCTAGTAA |
| | AAGGCTACTCTTTGAATCCCCTCTTTAAGGCATATAAAATTAGTACTCTTC |
| | CTGAGCTAGCTTAAGCATATCTTGAGCGAGTGAGTTGTATTTCCCTCCATC |
| | AAGTTCTAAGCGATCAAATAAGGTCCTTGCTCTCGAGCCAATGCCAATAC |
| | CAATAGAGAGGGTCTAAACGAAGGATTCAAAGGCGCGCC |
| 46 | CAAACGCGGAAGTGTATTGCGTTACAAAAAATGACAACTAGCATTTGTTT |
| | TTTCATTTCATGTTCGAATTCGTTTTTCGTTGGAAAAACCAACGCCGACCC |
| | CAAACAAGTCTCTCCAATATAAGGAGAGCGGAGCTTAAAAATATTATTTT |
| | ATTGTGCTATGGCAAATCTGGTCCGATGGCTCTTCTCCACTACCCGAGGG |
| | ACTAACGGTCTTCCATATTTCATCTTCGGTGTCGTTGTAGGAGGCGCCCTG |
| | TTGTTTGCTTTGCTAAAGTATCAGGCCCCTCTGTACGACCCGGCTTTAATG |
| | GAAAAAATCATAGATCATAATATAAAAGCCGGGCACCCTATAGAGGTTG |
| | ACTATTCGTGGTGGGGCACCTCTATTCGTGTAGTCTTTCCTAAGTAAGAA |
| | AGACAGGACAGTGGTGGTTTGCTCATACTTTCATTACAAAACCATACTAT |
| | GGAAT |
| 47 | MANLVRWLFSTTRGTNGLPYFIFGVVVGGALLFALLKYQAPLYDPALMEKII |
| | DHNIKAGHPIEVDYSWWGTSIRVVFPK |
| 48 | agatctagacgaggtgtagcgcagtctggtcagcgcatctgttttgggtacagagggccataggttcgaatcctgtcacctt |
| | gaGTCTGGCCCCAAATTCTAATTTCTACTGTTGTAGATTTCAATTATGAAAT |
| | TACTCATgtccctttcgtccagtggttaggacatcgtcttttcatgtcgaagacacgggttcgattcccgtaagggata |
| | GTCTGGCCCCAAATTCTAATTTCTACTGTTGTAGATGTCACATTTCTACCG |
| | GTGCACattccagcttatttgatacccacttcaagtttctatcaaaccatgtctttttcttcgaacgtcaatctcgtaggaaa |
| | gatct |
| 49 | GTAGGGCTTTCTGAGGAGTAAGCCTAATTCCGTTAATGCAG |
| 50 | GAGAGACTTGTTTGGGGTCGGCGTTGG |
| 51 | AGTGCTCAGAATAATCCAGGTCGCTCGACG |
| 52 | ACCACAGGTAGCAATAGGTATTACAGTAGGGATAACAG |
| 53 | ATGACAAATATGGTTCGATGGCTCTTCTCCACTAGCAGGTTTACTGCTTTC |
| | TATTTGCACTTTTGTATTAAGTTTCCTTATATATACGATTTTTTATTATTTT |
| | CTATTTGTCTATTTTTCTTTTTAGTGCGTTTTATTTCGATTATTCTTCTCCCA |
| | ATTTGCAATCTTTTCGGAGCCTCCTTCATTATTACTCTTCCTCCAGAGATT |
| | CAGGATCCCCAAGCTCTAGCTCATTTAGCAGGGCTAAACTTCTATCTGAG |
| | CCTTTACGAGCAGGATCCTGGATGGGTTACGTTCATTCAGAACGAGCTTA |
| | ATCACAATACCCCTCTGGAGGACATACCTGGACGGCTTAAGCTCTTCCTA |
| | ATGGAAGAAAAGCTGTCTAGTATGCGACAAGATGTCATTCAGGAATTTGT |
| | GGCGCTTTATCAAAGAATAGGGCCTTATCTACCGATCGAGCCCTACTTGG |
| | TCGATGAAGCGCTTCGTTCCTATCTGGACCATATTCACGCAACTGATTCTT |
| | TCACTGTTCTCCAAGCGTCTTATCAAGATCTGCGGGAGAATGAGGGAGGA |
| | TCTGTTTTCTTTAGAGATGCTGTTTCCCACAACCGGGATCTCCTTGAGGCG |
| | GAAAGCTCCGCAAGGAGGTGCCTGGAAGTGGAACAGAGGATCCGATGGG |
| | AAGAAATCCCCAAGAGCAAGGCAAGTCTCGAAAGAGCTGAGCACGAGCA |
| | TGCTCTCGACTTGTTTAAGTCGGAGGATCTTAGAAGGGAATTAGAAAAAA |
| | AAAGAGCGGGGTAG |
| 54 | MTNMVRWLFSTSRFTAFYLHFCIKFPYIYDFLLFSICLFFFLVRFISIILLPICNLF |
| | GASFIITLPPEIQDPQALAHLAGLNFYLSLYEQDPGWVTFIQNELNHNTPLEDI |
| | PGRLKLFLMEEKLSSMRQDVIQEFVALYQRIGPYLPIEPYLVDEALRSYLDHI |
| | HATDSFTVLQASYQDLRENEGGSVFFRDAVSHNRDLLEAESSARRCLEVEQR |
| | IRWEEIPKSKASLERAEHEHALDLFKSEDLRRELEKKRAG |
| 55 | ATGCCTCAACTTGATAAATTAACTTATTTCTCACAATTCTTCTGGTTATGT |
| | CTTCTCCTCTTTACTTTTTATATTCTCTTATTTAATAATAATAATGGAATAC |
| | TTGGAATTAGTAGAATTCTCAAACTACGGAACCAACTGCTTTCGCACCGG |
| | GGGGGCGAGATCCGGAGCAAGGACCCTAAGAATCTGGAAGATATCTCGA |
| | GAAAAGGTTTTAGCACCGGTCTCTCATATATGTACTCCAGTTTATCCGAA |
| | GTATCCCAATGGTGTAAGACCGTCGACTATTTGGGAAATAAGATATCTTC |
| | TTCAATCTTTCTATACTATCTTAGGGGCGTCCTTTGCCCAATTTGCCTCTTA |
| | TTTTTTAAATTTCTTATTTCTTTCGCCTTCACCTGTGCACTTATTTATGTAT |
| | TCAAGGGGGGGGGTTTCGTGGCTATGGCTGCTACAAACGGAGCCTCTTCT |
| | TCCTTTCTGGAGAGCTCAGGTGAAATAGATGCACTGTTACAAACAACAAC |
| | AACAACACGAACACCCGAAAACGCGGATTACGAAGACGATAATACTTCT |
| | GTAAATCAAGAATTCCTCGAAGACGGACAAAGGGCCCGACAGGCTAAAC |
| | TACGGGAGTTAGAAAGACTCATTCTCCAGCAATATAAGGATTTCATCCGA |
| | CAGAAATATCCATGGATACCTAAAGGCGACATCCTTCTCCCGAGCATGAA |
| | GGGTGGAGTTGTCGAGGACGTAATGGAAAAATTAGAATTGGAAACGTAT |
| | TCCAGTAGCGACTTGACTGATTGGATCAACCATTTGCGGGCAAATCCGAA |
| | AACATTAAATTTTATCTTCAAGGATTTTGTCGCGTGA |
| 56 | MPQLDKLTYFSQFFWLCLLLFTFYILLFNNNNGILGISRILKLRNQLLSHRGGE |
| | IRSKDPKNLEDISRKGFSTGLSYMYSSLSEVSQWCKTVDYLGNKISSSIFLYYL |
| | RGVLCPICLLFFKFLISFAFTCALIYVFKGGGFVAMAATNGASSSFLESSGEID |
| | ALLQTTTTTRTPENADYEDDNTSVNQEFLEDGQRARQAKLRELERLILQQYK |
| | DFIRQKYPWIPKGDILLPSMKGGVVEDVMEKLELETYSSSDLTDWINHLRAN |
| | PKTLNFIFKDFVA |
| 57 | AATAAGATATCTTCTTCAATCTTTCTATACTATCTTAGGGGCGTCCTTTGC |
| | CCAATTTGCCTCTTATTTTTTAAATTTCTTATTTCTTTCGCCTTCACCTGTG |
| | CACTTATTTATGTATTCAAGGGGGGGGGTTTCGTGGCTATGGCTGCTACA |
| | AACGGAGCCTCTTCTTCCTTTCTGGAGAGCTCAGGTGAAATAGATGCACT |
| | GTTACAAACAACAACAACAACACGAACACCCGAAAACGCGGATTACGAA |
| | GACGATAATACTTCTGTAAATCAAGAATTCCTCGAAGACGGACAAAGGG |
| | CCCGACAGGCTAAACTACGGGAGTTAGAAAGACTCATTCTCCAGCAATAT |
| | AAGGATTTCATCCGACAGAAATATCCATGGATACCTAAAGGCGACATCCT |
| | TCTCCCGAGCATGAAGGGTGGAGTTGTCGAGGACGTAATGGAAAAATTA |
| | GAATTGGAAACGTATTCCAGTAGCGACTTGACTGATTGGATCAACCATTT |
| | GCGGGCAAATCCGAAAACATTAAATTTTATCTTCAAGGATTTTGTCGCGT |
| | GA |
| 58 | NKISSSIFLYYLRGVLCPICLLFFKFLISFAFTCALIYVFKGGGFVAMAATNGA |
| | SSSFLESSGEIDALLQTTTTTRTPENADYEDDNTSVNQEFLEDGQRARQAKLR |
| | ELERLILQQYKDFIRQKYPWIPKGDILLPSMKGGVVEDVMEKLELETYSSSDL |
| | TDWINHLRANPKTLNFIFKDFVA |
| 59 | CTGCAGTGCAGCGTGACCCGGTCGTGCCCCTCTCTAGAGATAATGAGCAT |
| | TGCATGTCTAAGTTATAAAAAATTACCACATATTTTTTTTGTCACACTTGT |
| | TTGAAGTGCAGTTTATCTATCTTTATACATATATTTAAACTTTACTCTACG |
| | AATAATATAATCTATAGTACTACAATAATATCAGTGTTTTAGAGAATCAT |
| | ATAAATGAACAGTTAGACATGGTCTAAAGGACAATTGAGTATTTTGACAA |
| | CAGGACTCTACAGTTTTATCTTTTTAGTGTGCATGTGTTCTCCTTTTTTTTT |
| | GCAAATAGCTTCACCTATATAATACTTCATCCATTTTATTAGTACATCCAT |
| | TTAGGGTTTAGGGTTAATGGTTTTTATAGACTAATTTTTTTAGTACATCTA |
| | TTTTATTCTATTTTAGCCTCTAAATTAAGAAAACTAAAACTCTATTTTAGT |
| | TTTTTTATTTAATAATTTAGATATAAAATAGAATAAAATAAAGTGACTAA |
| | AAATTAAACAAATACCCTTTAAGAAATTAAAAAAACTAAGGAAACATTTT |
| | TCTTGTTTCGAGTAGATAATGCCAGCCTGTTAAACGCCGTCGACGAGTCT |
| | AACGGACACCAACCAGCGAACCAGCAGCGTCGCGTCGGGCCAAGCGAAG |
| | CAGACGGCACGGCATCTCTGTCGCTGCCTCTGGACCCCTCTCGAGAGTTC |
| | CGCTCCACCGTTGGACTTGCTCCGCTGTCGGCATCCAGAAATTGCGTGGC |
| | GGAGCGGCAGACGTGAGCCGGCACGGCAGGCGGCCTCCTCCTCCTCTCAC |
| | GGCACCGGCAGCTACGGGGGATTCCTTTCCCACCGCTCCTTCGCTTTCCCT |
| | TCCTCGCCCGCCGTAATAAATAGACACCCCCTCCACACCCTCTTTCCCCAA |
| | CCTCGTGTTGTTCGGAGCGCACACACACACAACCAGATCTCCCCCAAATC |
| | CACCCGTCGGCACCTCCGCTTCAAGGTACGCCGCTCGTCCTCCCCCCCCCC |
| | CCCTCTCTACCTTCTCTAGATCGGCGTTCCGGTCCATGGTTAGGGCCCGGT |
| | AGTTCTACTTCTGTTCATGTTTGTGTTAGATCCGTGTTTGTGTTAGATCCGT |
| | GCTGCTAGCGTTCGTACACGGATGCGACCTGTACGTCAGACACGTTCTGA |
| | TTGCTAACTTGCCAGTGTTTCTCTTTGGGGAATCCTGGGATGGCTCTAGCC |
| | GTTCCGCAGACGGGATCGATTTCATGATTTTTTTTGTTTCGTTGCATAGGG |
| | TTTGGTTTGCCCTTTTCCTTTATTTCAATATATGCCGTGCACTTGTTTGTCG |
| | GGTCATCTTTTCATGCTTTTTTTTGTCTTGGTTGTGATGATGTGGTCTGGTT |
| | GGGCGGTCGTTCTAGATCGGAGTAGAATTAATTCTGTTTCAAACTACCTG |
| | GTGGATTTATTAATTTTGGATCTGTATGTGTGTGCCATACATATTCATAGT |
| | TACGAATTGAAGATGATGGATGGAAATATCGATCTAGGATAGGTATACAT |
| | GTTGATGCGGGTTTTACTGATGCATATACAGAGATGCTTTTTGTTCGCTTG |
| | GTTGTGATGATGTGGTGTGGTTGGGCGGTCGTTCATTCGTTCTAGATCGG |
| | AGTAGAATACTGTTTCAAACTACCTGGTGTATTTATTAATTTTGGAACTGT |
| | ATGTGTGTGTCATACATCTTCATAGTTACGAGTTTAAGATGGATGGAAAT |
| | ATCGATCTAGGATAGGTATACATGTTGATGTGGGTTTTACTGATGCATAT |
| | ACATGATGGCATATGCAGCATCTATTCATATGCTCTAACCTTGAGTACCT |
| | ATCTATTATAATAAACAAGTATGTTTTATAATTATTTTGATCTTGATATAC |
| | TTGGATGATGGCATATGCAGCAGCTATATGTGGATTTTTTTAGCCCTGCCT |
| | TCATACGCTATTTATTTGCTTGGTACTGTTTCTTTTGTCGATGCTCACCCTG |
| | TTGTTTGGTGTTACTTCTGCAGGTCGACTCTAGAGGATCCACCATGGCTAG |
| | TGAAGCTCGAAAAACAAAGAAAAAAATCAAAGGGATTCAGCAAGCCACT |
| | GCAGGAGTCTCACAAGACACTTCGGAAAATCCTAACAAAACAATAGTTC |
| | CTGCAGCATTACCACAGCTCACCCCTACCTTGGTGTCACTGCTGGAGGTG |
| | ATTGAACCCGAGGTGTTGTATGCAGGATATGATAGCTCTGTTCCAGATTC |
| | AGCATGGAGAATTATGACCACACTCAACATGTTAGGTGGGCGTCAAGTG |
| | ATTGCAGCAGTGAAATGGGCAAAGGCGATACCAGGCTTCAGAAACTTAC |
| | ACCTGGATGACCAAATGACCCTGCTACAGTACTCATGGATGTTTCTCATG |
| | GCATTTGCCCTGGGTTGGAGATCATACAGACAATCAAGTGGAAACCTGCT |
| | CTGCTTTGCTCCTGATCTGATTATTAATGAGCAGAGAATGTCTCTACCCTG |
| | CATGTATGACCAATGTAAACACATGCTGTTTGTCTCCTCTGAATTACAAA |
| | GATTGCAGGTATCCTATGAAGAGTATCTCTGTATGAAAACCTTACTGCTT |
| | CTCTCCTCAGTTCCTAAGGAAGGTCTGAAGAGCCAAGAGTTATTTGATGA |
| | GATTCGAATGACTTATATCAAAGAGCTAGGAAAAGCCATCGTCAAAAGG |
| | GAAGGGAACTCCAGTCAGAACTGGCAACGGTTTTACCAACTGACAAAGC |
| | TTCTGGACTCCATGCATGAGGTGGTTGAGAATCTCCTTACCTACTGCTTCC |
| | AGACATTTTTGGATAAGACCATGAGTATTGAATTCCCAGAGATGTTAGCT |
| | GAAATCATCACTAATCAGATACCAAAATATTCAAATGGAAATATCAAAA |
| | AGCTTCTGTTTCATCAAAAATCTACTAGCAAACCGGTAACGTTATACGAC |
| | GTCGCTGAATACGCCGGCGTTTCTCATCAAACCGTTTCTAGAGTGGTTAA |
| | CCAGGCTTCACATGTTAGCGCTAAAACCCGGGAAAAAGTTGAAGCTGCC |
| | ATGGCTGAGCTCAACTACATCCCGAACCGTGTTGCGCAGCAGCTGGCTGG |
| | TAAACAAAGCTTGCTGATCGGTGTCGCGACCTCGAGCTTGGCCCTGCACG |
| | CGCCGTCGCAAATTGTCGCGGCGATTAAATCTCGCGCCGATCAACTGGGT |
| | GCCAGCGTGGTGGTGTCGATGGTAGAACGAAGCGGCGTCGAAGCCTGTA |
| | AAGCGGCGGTGCACAATCTTCTCGCGCAACGCGTCAGTGGGCTGATCATT |
| | AACTATCCGCTGGATGACCAGGATGCCATTGCTGTGGAAGCTGCCTGCAC |
| | TAATGTTCCGGCGTTATTTCTTGATGTCTCTGACCAGACACCCATCAACAG |
| | TATTATTTTCTCCCATGAAGACGGTACGCGACTGGGCGTGGAGCATCTGG |
| | TCGCATTGGGTCACCAGCAAATCGCGCTGTTAGCGGGCCCATTAAGTTCT |
| | GTCTCGGCGCGTCTGCGTCTGGCTGGCTGGCATAAATATCTCACTCGCAA |
| | TCAAATTCAGCCGATAGCGGAACGGGAAGGCGACTGGAGTGCCATGTCC |
| | GGTTTTCAACAAACCATGCAAATGCTGAATGAGGGCATCGTTCCCACTGC |
| | GATGCTGGTTGCCAACGATCAGATGGCGCTGGGCGCAATGCGCGCCATTA |
| | CCGAGTCCGGGCTGCGCGTTGGTGCGGATATCTCGGTAGTGGGATACGAC |
| | GATACCGAAGACAGCTCATGTTATATCCCGCCGTTAACCACCATCAAACA |
| | GGATTTTCGCCTGCTGGGGCAAACCAGCGTGGACCGCTTGCTGCAACTCT |
| | CTCAGGGCCAGGCGGTGAAGGGCAATCAGCTGTTGCCCGTCTCACTGGTG |
| | AAAAGAAAAACCACTAGTGGATCGGAATTCGCTAACTTCAACCAGTCCG |
| | GAAACATCGCTGATTCTTCCTTGAGCTTCACTTTCACTAACTCTTCTAACG |
| | GACCTAACCTTATCACCACTCAGACCAACTCTCAGGCTCTTAGCCAGCCA |
| | ATCGCTAGCTCTAACGTGCACGACAACTTCATGAACAACGAGATCACTGC |
| | TAGCAAGATCGATGATGGTAACAATTCTAAGCCTCTTAGCCCAGGATGGA |
| | CTGATCAGACTGCTTACAACGCATTCGGTATCACTACCGGTATGTTCAAC |
| | ACCACTACCATGGACGATGTGTACAACTACCTCTTCGACGATGAGGATAC |
| | TCCACCTAACCCTAAGAAGGAGTGAACTAGAGTCCTGCTTTAATGAGATA |
| | TGCGAGACGCCTATGATCGCATGATATTTGCTTTCAATTCTGTTGTGCACG |
| | TTGTAAAAAACCTGAGCATGTGTAGCTCAGATCCTTACCGCCGGTTTCGG |
| | TTCATTCTAATGAATATATCACCCGTTACTATCGTATTTTTATGAATAATA |
| | TTCTCCGTTCAATTTACTGATTGTACCCTACTACTTATATGTACAATATTA |
| | AAATGAAAACAATATATTGTGCTGAATAGGTTTATAGCGACATCTATGAT |
| | AGAGCGCCACAATAACAAACAATTGCGTTTTATTATTACAAATCCAATTT |
| | TAAAAAAAGCGGCAGAACCGGTCAAACCTAAAAGACTGATTACATAAAT |
| | CTTATTCAAATTTCAAAAGTGCCCCAGGGGCTAGTATCTACGACACACCG |
| | AGCGGCGAACTTAATAACGCTCACTGAAGGGAACTCCGGTTCCCCGCCGG |
| | CGCGCATGGGTGAGATTCCTTGAAGTTGAGTATTGGCCGTCCGCTCTACC |
| | GAAAGTTACGGGCACCATTCAACCCGGTCCAGCACGGCGGCCGGGTAAC |
| | CGACTTGCTGCCCCGAGAATTATGCAGCATTTTTTTGGTGTATGTGGGCCC |
| | CAAATGAAGTGCAGGTCAAACCTTGACAGTGACGACAAATCGTTGGGCG |
| | GGTCCAGGGCGAATTTTGCGACAAC |
| 60 | GATCTAGTAACATAGATGACACCGCGCGCGATAATTTATCCTAGTTTGCG |
| | CGCTATATTTTGTTTTCTATCGCGTATTAAATGTATAATTGCGGGACTCTA |
| | ATCATAAAAACCCATCTCATAAATAACGTCATGCATTACATGTTAATTAT |
| | TACATGCTTAACGTAATTCAACAGAAATTATATGATAATCATCGCAAGAC |
| | CGGCAACAGGATTCAATCTTAAGAAACTTTATTGCCAAATGTTTGAACGA |
| | TCGGGGAAATTCGAGCTCGGTACCCTGCAGGTTATCACTTGTGCCCCAGT |
| | TTGCTAGGGAGGTCGCAGTATCTGGCCACAGCCACCTCGTGCTGCTCGAC |
| | GTATGTCTCTTTGTCGGCCTCCTTGATTCTTTCCAGTCTGTGGTCCACATA |
| | GTAGACGCCGGGCATCTTGAGGTTCTTAGCGGGTTTCTTGGATCTGTATGT |
| | GGTCTTGAAGTTGCAGATCAGGTGGCCCCCGCCCACGAGCTTCAGGGCCA |
| | TGTCGCTTCTGCCTTCCAGGCCGCCGTCAGCGGGGTACAGCATCTCGGTG |
| | TTGGCCTCCCAGCCGAGTGTTTTCTTCTGCATCACAGGGCCGTTGGATGG |
| | GAAGTTCACCCCTCTGATCTTGACGTTGTAGATGAGGCAGCCGTCCTGGA |
| | GGCTGGTGTCCTGGGTAGCGGTCAGCACGCCCCCGTCTTCGTATGTGGTG |
| | ACTCTCTCCCATGTGAAGCCCTCAGGGAAGGACTGCTTAAAGAAGTCGGG |
| | GATGCCCTGGGTGTGGTTGATGAAGGTTCTGCTGCCGTACATGAAGCTGG |
| | TAGCCAGGATGTCGAAGGCGAAGGGGAGAGGGCCGCCCTCGACCACCTT |
| | GATTCTCATGGTCTGGGTGCCCTCGTAGGGCTTGCCTTCGCCCTCGGATGT |
| | GCACTTGAAGTGGTGGTTGTTCACGGTGCCCTCCATGTACAGCTTCATGT |
| | GCATGTTCTCCTTAATCAGCTCGCTCATGGTGACTCGAGTCGACAAGCTT |
| | GCTAGCTGTAGTTGTAGAATGTAAAATGTAATGTTGTTGTTGTTTGTTGTT |
| | GTTGTTGGTAATTGTTGTAAAAATACGCGCGTCTAGCTTCAGCGTGTCCTC |
| | TCCAAATGAAATGAACTTCCTTATATAGAGGAAGGGTCTTGCGAAGATCG |
| | ATCCTCTAGTCTTTCAATTGTGAGCGCTCACAATTCTTTCTCTTCCCTTTCT |
| | TCTTTACTAGTCTTTCAATTGTGAGCGCTCACAATTCTTTCTCTTCCCTTTC |
| | TTCTTTCTAGTCTTTCAATTGTGAGCGCTCACAATTCTTTCTCTTCCCTTTC |
| | TTCTTTCTAGTCTTTCAATTGTGAGCGCTCACAATTCTTTCTCTTCCCTTTC |
| | TTCTTTCTAGTCTTTCAATTGTGAGCGCTCACAATTCTTTCTCTTCCCTTTC |
| | TTCTTTCTAGATTTCTTGAGCTCTCTAGTCTTTCAATTGTGAGCGCTCACA |
| | ATTCTTTCTCTTCCCTTTCTTCTTTCTAGCTCCACCGCGGTGGCGGCCGGCC |
| | GCTCTAGTGGATCGATCTTCGCAAGACCCTTCCTCTATATAAGGAAGTTC |
| | ATTTCATTTGGAGAGGACACGCTGAAGCTAGACGCGCGTATTTTTACAAC |
| | AATTACCAACAACAACAACAAACAACAACAACATTACATTTTACATTCTA |
| | CAACTACAGCTAGAGTCGAGTGGCCACCATGTTTAAACAAGCTTCTCGTCTC |
| | CTCTCCCGATCTGTCGCCGCCGCATCTTCCAAATCGGTGACGACTCGTGCCTT |
| | TTCAACGGAACTTCCATCGACGCTCGATTCCCCTCCTACTAAAAGATTTCG |
| | TATTCAAGCAAAAAACATATTTCTTACATATCCTCAGTGTTCTCTTTC |
| | AAAAGAAGAAGCTCTTGAGCAAATTCAAAGAATACAACTTTCATCTA |
| | ATAAAAAATATATTAAAATTGCCAGAGAGCTACACGAAGATGGGCAA |
| | CCTCATCTCCACGTCCTGCTTCAACTCGAAGGAAAAGTTCAGATCAC |
| | AAATATCAGATTATTCGACCTGGTATCCCCAACCAGGTCAGCACATTT |
| | CCATCCAAACATTCAGAGAGCTAAATCCAGCTCCGACGTCAAGTCCT |
| | ACGTAGACAAGGACGGAGACACAATTGAATGGGGAGAATTCCAGATC |
| | GACGGTAGAAGTGCTAGAGGAGGTCAACAGACAGCTAACGACTCATA |
| | TGCCAAGGCGTTAAACGCAACTTCTCTTGACCAAGCACTTCAAATATT |
| | GAAGGAAGAACAACCAAAGGATTACTTCCTTCAACATCACAATCTTTT |
| | GAACAATGCTCAAAAGATATTTCAGAGGCCACCTGATCCATGGACTC |
| | CACTATTTCCTCTGTCCTCATTCACAAACGTTCCTGAGGAAATGCAAG |
| | AATGGGCTGATGCATATTTCGGGGTTGATGCCGCTGCGCGGCCTTTA |
| | AGATATAATAGTATCATAGTAGAGGGTGATTCAAGAACAGGGAAGAC |
| | TATGTGGGCTAGATCTTTAGGGGCCCACAATTACATCACAGGGCACT |
| | TAGATTTTAGCCCTAGAACGTATTATGATGAAGTGGAATACAACGTC |
| | ATTGATGACGTAGATCCCACTTACTTAAAGATGAAACACTGGAAACA |
| | CCTTATTGGAGCACAAAAGGAGTGGCAGACAAACTTAAAGTATGGAA |
| | AACCACGTGTCATTAAAGGTGGTATCCCCTGCATTATATTATGCAATC |
| | CAGGACCTGAGAGCTCATACCAACAATTTCTTGAAAAACCAGAAAAT |
| | GAAGCCCTTAAGTCCTGGACATTACATAATTCAACCTTCTGCAAACTC |
| | CAAGGTCCGCTCTTTAATAACCAAGCAGCAGCATCCTCGCAAGGTGA |
| | CTCTACCCTGTAAAGGCAAACAATGAATCAACAACTCTCCTGGCGCACC |
| | ATCGTCGGCTACAGCCTCGGTGGGGAGTCCGCAAATCACCAGTCTCTCTC |
| | TACAAATCTATCTCTCTCTATTTTCTCCAGAATAATGTGTGAGTAGTTCCC |
| | AGATAAGGGAATTAGGGTTCTTATAGGGTTTCGCTCATGTGTTGAGCATA |
| | TAAGAAACCCTTAGTATGTATTTGTATTTGTAAAATACTTCTATCAATAAA |
| | ATTTCTAATTCCTAAAACCAAAATCCAGTGACC |
| 61 | ATGGTGGAGCACGACACTCTCGTCTACTCCAAGAATATCAAAGATACAGT |
| | CTCAGAAGACCAAAGGGCTATTGAGACTTTTCAACAAAGGGTAATATCG |
| | GGAAACCTCCTCGGATTCCATTGCCCAGCTATCTGTCACTTCATCAAAAG |
| | GACAGTAGAAAAGGAAGGTGGCACCTACAAATGCCATCATTGCGATAAA |
| | GGAAAGGCTATCGTTCAAGATGCCTCTGCCGACAGTGGTCCCAAAGATGG |
| | ACCCCCACCCACGAGGAGCATCGTGGAAAAAGAAGACGTTCCAACCACG |
| | TCTTCAAAGCAAGTGGATTGATGTGATAACATGGTGGAGCACGACACTCT |
| | CGTCTACTCCAAGAATATCAAAGATACAGTCTCAGAAGACCAAAGGGCT |
| | ATTGAGACTTTTCAACAAAGGGTAATATCGGGAAACCTCCTCGGATTCCA |
| | TTGCCCAGCTATCTGTCACTTCATCAAAAGGACAGTAGAAAAGGAAGGTG |
| | GCACCTACAAATGCCATCATTGCGATAAAGGAAAGGCTATCGTTCAAGAT |
| | GCCTCTGCCGACAGTGGTCCCAAAGATGGACCCCCACCCACGAGGAGCA |
| | TCGTGGAAAAAGAAGACGTTCCAACCACGTCTTCAAAGCAAGTGGATTG |
| | ATGTGATATCTCCACTGACGTAAGGGATGACGCACAATCCCACTATCCTT |
| | CGCAAGACCTTCCTCTATATAAGGAAGTTCATTTCATTTGGAGAGGACAC |
| | GCTGAAATCACCAGTCTCTCTCTACAAATCTATCTCTCTCGAGCTTTCGCA |
| | GATCCCGGGGGGCAATGAGATATGAAAAAGCCTGAACTCACCGCGACG |
| | TCTGTCGAGAAGTTTCTGATCGAAAAGTTCGACAGCGTCTCCGACCT |
| | GATGCAGCTCTCGGAGGGCGAAGAATCTCGTGCTTTCAGCTTCGATG |
| | TAGGAGGGCGTGGATATGTCCTGCGGGTAAATAGCTGCGCCGATGG |
| | TTTCTACAAAGATCGTTATGTTTATCGGCACTTTGCATCGGCCGCGCT |
| | CCCGATTCCGGAAGTGCTTGACATTGGGGAGTTTAGCGAGAGCCTGA |
| | CCTATTGCATCTCCCGCCGTGCACAGGGTGTCACGTTGCAAGACCTG |
| | CCTGAAACCGAACTGCCCGCTGTTCTACAACCGGTCGCGGAGGCTAT |
| | GGATGCGATCGCTGCGGCCGATCTTAGCCAGACGAGCGGGTTCGGC |
| | CCATTCGGACCGCAAGGAATCGGTCAATACACTACATGGCGTGATTT |
| | CATATGCGCGATTGCTGATCCCCATGTGTATCACTGGCAAACTGTGA |
| | TGGACGACACCGTCAGTGCGTCCGTCGCGCAGGCTCTCGATGAGCTG |
| | ATGCTTTGGGCCGAGGACTGCCCCGAAGTCCGGCACCTCGTGCACGC |
| | GGATTTCGGCTCCAACAATGTCCTGACGGACAATGGCCGCATAACAG |
| | CGGTCATTGACTGGAGCGAGGCGATGTTCGGGGATTCCCAATACGAG |
| | GTCGCCAACATCTTCTTCTGGAGGCCGTGGTTGGCTTGTATGGAGCA |
| | GCAGACGCGCTACTTCGAGCGGAGGCATCCGGAGCTTGCAGGATCG |
| | CCACGACTCCGGGCGTATATGCTCCGCATTGGTCTTGACCAACTCTA |
| | TCAGAGCTTGGTTGACGGCAATTTCGATGATGCAGCTTGGGCGCAGG |
| | GTCGATGCGACGCAATCGTCCGATCCGGAGCCGGGACTGTCGGGCG |
| | TACACAAATCGCCCGCAGAAGCGCGGCCGTCTGGACCGATGGCTGT |
| | GTAGAAGTACTCGCCGATAGTGGAAACCGACGCCCCAGCACTCGTCC |
| | GAGGGCAAAGAAATAGAGTAGATGCCGACCGGATCTGTCGATCGACAA |
| | GCTCGAGTTTCTCCATAATAATGTGTGAGTAGTTCCCAGATAAGGGAATT |
| | AGGGTTCCTATAGGGTTTCGCTCATGTGTTGAGCATATAAGAAACCCTTA |
| | GTATGTATTTGTATTTGTAAAATACTTCTATCAATAAAATTTCTAATTCCT |
| | AAAACCAAAATCCAGTACTAAAATCCAGATCCCCCGAATTA |
| 62 | AKAEEIVLQPIREISGAVQLPGSKSLSNRILLLSALSEGTTVVDNLLNSEDVHY |
| | MLEALKALGLSVEADKVAKRAVVVGCGGKFPVEKDAKEEVQLFLGNAGIA |
| | MRSLTAAVTAAGGNATYVLDGVPRMRERPIGDLVVGLKQLGADVDCFLGT |
| | ECPPVRVKGIGGLPGGKVKLSGSISSQYLSALLMAAPLALGDVEIEIIDKLISIP |
| | YVEMTLRLMERFGVKAEHSDSWDRFYIKGGQKYKSPGNAYVEGDASSASY |
| | FLAGAAITGGTVTVQGCGTTSLQGDVKFAEVLEMMGAKVTWTDTSVTVTG |
| | PPREPYGKKHLKAVDVNMNKMPDVAMTLAVVALFADGPTAIRDVASWRV |
| | KETERMVAIRTELTKLGASVEEGPDYCIITPPEKLNITAIDTYDDHRMAMAFS |
| | LAACADVPVTIRDPGCTRKTFPNYFDVLSTFVRN |
| 63 | GCGAAGGCGGAGGAGATCGTGCTCCAaCCCATCAGaGAGATtTCCGGGGC |
| | GGTTCAaCTGCCAGGGTCCAAGTCGCTCTCCAACAGaATCCTCCTCCTCTC |
| | CGCCCTCTCCGAGGGCACAACtGTGGTGGACAACTTGCTGAACAGTGAGG |
| | ATGTTCATTAtATGCTTGAGGCCCTGAAAGCCCTCGGGCTCTCTGTGGAAG |
| | CAGATAAAGTTGCAAAgAGAGCTGTAGTCGTTGGCTGTGGTGGCAAGTTT |
| | CCTGTTGAGAAGGATGCGAAAGAGGAAGTGCAACTCTTCTTGGGGAACG |
| | CTGGAAtTGCAATGCGAtCATTGACtGCtGCCGTGACTGCTGCTGGTGGAAA |
| | TGCAACTTATGTGCTTGATGGAGTGCCACGAATGAGaGAGcgtCCtATTGGT |
| | GAtTTGGTTGTCGGGTTGAAACAACTTGGTGCGGATGTUGACTGTTTCCTTG |
| | GCACTGAATGCCCACCTGTTCGTGTCAAGGGAATTGGAGGACTTCCTGGT |
| | GGCAAGGTTAAGCTCTCTGGTTCCATCAGCAGTCAaTAtTTGAGTGCCTTG |
| | CTGATGGCTGCTCCTTTGGCCCTTGGGGATGTGGAGATCGAAATCATTGA |
| | CAAACTAATCTCCATTCCTTAtGTTGAAATGACITTGAGATTGATGGAGCG |
| | TTTTGGTGTGAAGGCAGAGCATTCTGATAGTTGGGACAGATTCTATATTA |
| | AGGGAGGGCAaAAGTAtAAATCTCCTGGAAATGCCTATGTTGAAGGTGAT |
| | GCCTCAAGCGCGAGCTATTTCTTGGCTGGTGCTGCAATCACTGGAGGCAC |
| | TGTGACAGTTCAAGGTTGTGGTACaACCAGTTTGCAaGGTGATGTCAAATT |
| | TGCTGAGGTACTTGAGATGATGGGAGCAAAGGTTACATGGACTGACACC |
| | AGTGTAACaGTAACTGGTCCACCtagaGAGCCTTATGGGAAGAAACACTGA |
| | AAGCTGTTGATGTCAACATGAACAAAATGCCTGATGTTGCCATGACCCTT |
| | GCCGTTGTTGCACTCTTCGCTGATGGTCCAACTGCTATCAGAGATGTGGCT |
| | TCCTGGAGAGTAAAGGAAACtGAAAGaATGGTTGCAATTCGaACtGAGCTA |
| | ACAAAaCTtGGAGCATCGGTTGAAGAAGGTCCTGACTAtTGCATCATCACtC |
| | CACCIGAGAAGCTGAACATtACtGCAATCGACACtTAtGATGAcCAtAGaATG |
| | GCtATGGCCTTCTCCCTCGCTGCCTGCGCCGACGTGCCCGTGACtATCcgtGA |
| | CCCTGGTTGCACtagaAAGACaTTCCCCAACTATTCGACGTTCTAAGCACTT |
| | TCGTCAGaAACTGA |
| 64 | GAATTTCTCTGACATTCCATGTTTCCGAAACGGATCCTATAGCGAAGGCG |
| | GAGGAGATCGTGCTCCAaCCCATCAGaGAGATtTCCGGGGCGGTTCAaCTG |
| | CCAGGGTCCAAGTCGCTCTCCAACAGaATCCTCCTCCTCTCCGCCCTCTCC |
| | GAGGGCACAACtGTGGTGGACAACTTGCTGAACAGTGAGGATGTTCATAt |
| | ATGCTTGAGGCCCTGAAAGCCCTCGGGCTCTCTGTGGAAGCAGATAAAGT |
| | TGCAAAgAGAGCTGTAGTCGTTGGCTGTGGTGGCAAGTTTCCTGTTGAGA |
| | AGGATGCGAAAGAGGAAGTGCAACTCTTCTTGGGGAACGCTGGAAtTGCA |
| | ATGCGAtCATTGACtGCtGCCGTGACTGCTGCTGGTGGAAATGCAACTTATG |
| | TGCTTGATGGAGTGCCACGAATGAGaGAGcgtCCtATTGGTGAtTTGGTTGTC |
| | GGGTTGAAACAACTTGGTGCGGATGTUGACTGTTTCCTTGGCACTGAATGC |
| | CCACCTGTTCGTGTCAAGGGAATTGGAGGACTTCCTGGTGGCAAGGTTAA |
| | GCTCTCTGGTTCCATCAGCAGTCAaTATTGAGTGCCTTGCTGATGGCTGCT |
| | CCTTTGGCCCTTGGGGATGTGGAGATCGAAATCATTGACAAACTAATCTC |
| | CATTCCTTAtGTTGAAATGACITTGAGATTGATGGAGCGTTTTGGTGTGAA |
| | GGCAGAGCATTCTGATAGTTGGGACAGATTCTATATTAAGGGAGGGCAaA |
| | AGTAtAAATCTCCTGGAAATGCCTATGTTGAAGGTGATGCCTCAAGCGCG |
| | AGCTATTTCTTGGCTGGTGCTGCAATCACTGGAGGCACTGTGACAGTTCA |
| | AGGTTGTGGTACaACCAGTTTGCAaGGTGATGTCAAATTTGCTGAGGTACT |
| | TGAGATGATGGGAGCAAAGGTTACATGGACTGACACCAGTGTAACaGTAA |
| | CTGGTCCACCtagaGAGCCTTATGGGAAGAAACAtCTGAAAGCTGTTGATGT |
| | CAACATGAACAAAATGCCTGATGTTGCCATGACCCTTGCCGTTGTTGCAC |
| | TCTTCGCTGATGGTCCAACTGCTATCAGAGATGTGGCTTCCTGGAGAGTA |
| | AAGGAAACtGAAAGaATGGTTGCAATTCGaACtGAGCTAACAAAaCTtGGAG |
| | CATCGGTTGAAGAAGGTCCTGACTAtTGCATCATCACtCCACCtGAGAAGCT |
| | GAACATtACtGCAATCGACACtTAGATGAcCAtAGaATGGCtATGGCCTTCTC |
| | CCTCGCTGCCTGCGCCGACGTGCCCGTGACtATCcgtGACCCTGGTTGCACta |
| | gaAAGACaTTCCCCAACTATTCGACGTTCTAAGCACTTTCGTCAGaAACTG |
| | A |
| 65 | MDPIAKAEEIVLQPIREISGAVQLPGSKSLSNRILLLSALSEGTTVVDNLLNSE |
| | DVHYMLEALKALGLSVEADKVAKRAVVVGCGGKFPVEKDAKEEVQLFLGN |
| | AGIAMRSLTAAVTAAGGNATYVLDGVPRMRERPIGDLVVGLKQLGADVDC |
| | FLGTECPPVRVKGIGGLPGGKVKLSGSISSQYLSALLMAAPLALGDVEIEIIDK |
| | LISIPYVEMTLRLMERFGVKAEHSDSWDRFYIKGGQKYKSPGNAYVEGDASS |
| | ASYFLAGAAITGGTVTVQGCGTTSLQGDVKFAEVLEMMGAKVTWTDTSVT |
| | VTGPPREPYGKKHLKAVDVNMNKMPDVAMTLAVVALFADGPTAIRDVAS |
| | WRVKETERMVAIRTELTKLGASVEEGPDYCIITPPEKLNITAIDTYDDHRMA |
| | MAFSLAACADVPVTIRDPGCTRKTFPNYFDVLSTFVRN |
| 66 | TCTGCTTGAAAGCCTGCAGAGTCCAATTTTGAGTATTTTCAGTTAGAATCT |
| | AGAGTCAGCCTATTCAGTTCTTAGCCCTTAAGGGTAAGGCAGGGGGTAAT |
| | ATGGATAGTCTCTGTCCCTGTATTCACATTCCACCTTCAACAAAGTGTTGA |
| | TTTCCCGTAAAGCTAACTGTAGTCCTTTAAGTAAGTAGATATCTTAGGCA |
| | AGTTAGCAATCTCGTTATATTACCAAGGCCTTCCCTTCTATTGTAGAAAGA |
| | GTTCTCAGCCATCTAATTGCAGTGCCAGTTGCCAGCTATCCAGTTTCATTT |
| | GAAGTTGCTGGGGGTCCAAACGAGCTAGTTGCTTTTATTCGTCCTATAAG |
| | TCCTTCCACAAGCGAGTCAATAGGGTGCTGGCTAGTTGTAGTTGTTGGCG |
| | TGCCTTTCCTTTCATCTTGAATATTAATAAATATTTGGATAAATTACTTTA |
| | GAATAAGAAGTTCATGTTTTAAGTAATACGAATCCATACTAGGAAAATGA |
| | AAATGTGAGTCCTAGGCACTGGAATTGGTTCTCTTCTCCCTAATCCCTATA |
| | AGCCAGAAAGGGTAATAGGCTTCAGTGTAAGCATTTCCTTCAAGCAAGTC |
| | ATCTCAAGTTTTAAATTCTAGAGAATAGCTCCGATCAACCCATTTTAGTTT |
| | GGTTCTGCAATTCATTCGCATAAATGAAAAAAAAAGCGAGATGTGCACG |
| | AAAGAAGATCATAGTTCAGCTTTAAAATGGTGGTGTCCCTGTGTTAGTAA |
| | GTGGTTGAAATAGCTCATGGGAGTGTCTGCCCCATTCGATAATGGCATTT |
| | ATGATCTAGTGGAGTGAGTGATTGTGTGGTGTTCAGTCTAAGGCTTTTTG |
| | AAAAGCGGATTTCTCCCTTCTCTCATCCATCGTCTTTGTTAAAGT |
| 67 | TGAAAAAGGGAAAGCATGCAAAAGGGAGAGGTACCAAAGATCTAAAAA |
| | GCTTAAGCGGCCGCAAATAGATAGACCTTTTTATTTTTCGTCATTCGATCACG |
| | AAAAGGCCGGCCAAACCTCGAGGACCACAGGTAGCAATAGGTATTACAGT |
| | AGGGATAACAGGGTAATGAAAGACAGGACAGTGGTGGTTTGCTCATA |
| | CTTTCATTACAAAACCATACTATGGAATTCTGGTGGACCAGGCAAAT |
| | ATCCCACCCCTACAAGGTGGGAGGGCGGCCGGGAACCCGTGGTGGC |
| | GGTTCCTCCCGTGATGATCGACTAATGGATAAGTCGCAATGATCGAC |
| | TAATGGATAAGTCGCATTGAGATATTGGTTCGAATCCAATTCATTACG |
| | AGCTCTTCTGGAGAGCATTATCTCTAAGAGAATAAAATGAAAAACCCA |
| | GCTAGGAGAAAAAAACAGTAAGTTAACAAGATAG |
| 68 | ACGCCATGTGATCGCTACTAAAGATAGAGTTTCCTTGGAAAAACCGAGGC |
| | CAGTTGAGATCAGTCTCCCTTTCTAGGAGCAGAGCTTAAAAAGATGGGAA |
| | ATTCCAATGAATTTCGATCACAATCATGTGGTAATAATGGGTTTGAATCA |
| | GAGAGACTCGATCTGGAAACTCCTCAATGATTATAACGTGAACTCGTTGA |
| | AGAGAAGGAGACAAGCAGAAATAGACGCTTTTTTTGAACCATTTGAGAG |
| | GGCGCAGCGTATCCGTTTCAATAACTGGCAGAACGGAATAGAGTTGTTAG |
| | ATGGGGCTGAATGGAGGAACGGCGATATAGTTATCCCTGGAGGCGGCGG |
| | ACCAGTAATTTCAAGCCCCTTGGATCAATTTTTCATTGATCCATTATTTGG |
| | TCTTGATATGGGTAACTTTTATTTATCATTCACAAATGAATCCTTGTCTAT |
| | GGCGGTAACTGTCGTTTTGGTGCCATCTTTATTTGGAGTTGTTACGAAAAA |
| | GGGCGGGGGAAAGTCAGTGCCAAATGCATGGCAATCCTTGGTAGAGCTT |
| | ATTTATGATTTCGTGCTGAACCTGGTAAACGAACAAATAGGTGGAAATGT |
| | TAAACAAAAGTTTTTCCCTCGCATCTCGGTCACTTTTACTTTTTCGTTATTT |
| | CGTAATCCCCAGGGTATGATACCCTTTAGCTTCACAGTGACAAGTCATTTT |
| | CTCATTACTTTGGCTCTTTCATTTTCCATTTTTATAGGCATTACGATCGTTG |
| | GATTTCAAAGACATGGGCTTCATTTTTTTAGCTTCTTATTACCAGCGGGAG |
| | TCCCACTGCCATTAGCACCTTTTTTAGTACTCCTTGAGCTAATCTCTCATT |
| | GTTTTCGTGCATTAAGCTCAGGAATACGTTTATTTGCTAATATGATGGCCG |
| | GTCATAGTTCAGTAAAGATTTTAAGTGGGTTCGCTTGGACTATGCTATTTC |
| | TGAATAATATTTTCTATTTCATAGGAGATCTTGGTCCCTTATTTATAGTTC |
| | TAGCATTAACCGGTTTTGAATTAGGTGTAGCTATATTACAAGCTCATGTT |
| | TCTACGATCTCAATTTGTATTTACTTGAATGATGCTATAAATCTCCATCAA |
| | AATGAGTAATTTCATAATTGAATAAAAACGAGGAGCCGAAGATTTTAGG |
| | GGGCGGGA |
| 69 | AAACCATCTTCTCACTCTGACCCCCACATATCAGATCCCAGATGCATAGG |
| | AAAAGCGGTATCAAGAATAGTAGTATAAAGAAAGATAGTACAGTACTCA |
| | AGTAAATGAATTCGCCTAAGGATCGATGGAAAGATCAAGGTCCCCGTGA |
| | AAAAGTAGATACTAGATCGATATGATACTCTCATCTCTGGAGTAACTTCT |
| | TCCATTATGCTGATCTCTAGGTCCGTTCCATCATCATCGTAATAGTATGGT |
| | CCCAGGTGTCCGAGCTATAGATCAAGATCATATTTAGTCACATTTCTACC |
| | GGTGCACTTCTCATGAAATAATTCCCTTTCCAAGGAAAGGAAAACAAGAA |
| | CTCGAATACTCGTAATAGCGATCCCGATCCACCTACTTTTTTCTATTCTTT |
| | GATTCGAAACGTGCTAAAGCACAAGCCATTTTTATGCATGGGGCATAAGA |
| | GTGGACAATCTATGTTATCGAAGGAAGTAAATAACAACACTTCAGCGTTT |
| | AGGTCTACCTTCAGTAAACCAATAGTTTTGCAGCATTGGAATTTGAGTTG |
| | GCCAGGTAAGGTCCTCTAAAAAGAAAAGAAGAAACTACTTAGAATAGAT |
| | AAATGCCATTGGTTTTCTCGTACTATACGATCTTTTTTTGTTTTGTTTTTTG |
| | GCCATGATTGTGCTGCTCCTGTGAAGGCTAGTGGGAAAGCTCACCGTTCG |
| | TTGTGATGAGTGGGGGCCTTGTATCTGTATTCGGATCAGCTCCTTAACAG |
| | AGTTTCCTGCTTGAACCCTGGCTGGGAGCTGGGAGAGGTGTCCCACTACA |
| | GGTGCAAATAAACCATTTGACCTTACAGGGGAAAGGAAACAAACCACTC |
| | AATAATCGGTAGAAATTCCTCCTACTGAACAGCTTTCCTTTTCTCGCCTTA |
| | ACTACTACTTCAAAGCAAGGCGGAATATCACGGGATAGGAATGAAAGAA |
| | CTTCTTACTCAACTTTCTAGCTATATAAAAATAGTTAGCAATATGAAACG |
| | AGTAACTTAAGCCCTAGTAAAAGGCTACTCTTTGAATCCCCTCTTTAAGG |
| | CATATAAAATTAGTACTCTTCCTGAGCTAGCTTAAGCATATCTTGAGCGA |
| | GTGAGTTGTATTTCCCTCCATCAAGTTCTAAGCGATCAAATAAGGTCCTTG |
| | CTCTCGAGCCAATGCCAATACCAATAGAGAGGGTCTAAACGAAGGATTC |
| | AAA |
| 70 | CAAACGCGGAAGTGTATTGCGTTACAAAAAATGACAACTAGCATTTGTTT |
| | TTTCATTTCATGTTCGAATTCGTTTTTCGTTGGAAAAACCAACGCCGACCC |
| | CAAACAAGTCTCTCCAATATAAGGAGAGCGGAGCTTAAAAATATTATTTT |
| | ATTGTGCTATGGCAAATCTGGTCCGATGGCTCTTCTCCACTACCCGAG |
| | GGACTAACGGTCTTCCATATTTCATCTTCGGTGTCGTTGTAGGAGGC |
| | GCCCTGTTGTTTGCTTTGCTAAAGTATCAGGCCCCTCTGTACGACCC |
| | GGCTTTAATGGAAAAAATCATAGATCATAATATAAAAGCCGGGCACC |
| | CTATAGAGGTTGACTATTCGTGGTGGGGCACCTCTATTCGTGTAGTC |
| | TTTCCTAAGTAAGAAAGACAGGACAGTGGTGGTTTGCTCATACTTTCATT |
| | ACAAAACCATACTATGGAATTCTGGTGGACCAGGCAAATATCCCACCCCT |
| | ACAAGGTGGGAGGGCGGCCGGGAACCCGTGGTGGCGGTTCCTCCCGTGA |
| | TGATCGACTAATGGATAAGTCGCAATGATCGACTAATGGATAAGTCGCAT |
| | TGAGATATTGGTTCGAATCCAATTCATTACGAGCTCTTCTGGAGAGCATT |
| | ATCTCTAAGAGAATAAAATGAAAAACCCAGCTAGGAGAAAAAAACAGTA |
| | AGTTAACAAGATAG |
| 71 | TGTACTCCGATGACGTGGCTTAGCATATTAACATATCTATTGGAGTATTG |
| | GAGTATTATATATATTAGTACAACTTTCATAAGGGCCATCCGTTATAATAT |
| | TACCGGATGGCCCGAAAAAAATGGGCACCCAATCAAAACGTGACACGTG |
| | GAAGGGGACTGTTGAATGATGTGACGTTTTTGAGCGGGAAACTTCCTGAA |
| | G |
| 72 | MSSSLLTDLVNLDLSESTDKVIAEYIWVGGTGMDVRSKARTLSGPVDDPSKL |
| | PKWNFDGSGTGQATGDDSEVILHPQAIFRDPFRKGKNILVMCDCYAPNGEPI |
| | PTNNRYNAARIFSHPDVKAEEPWYGIEQEYTLLQKHINWPLGWPLGGYPGPQ |
| | GPYYCAAGADKSYGRDIVDAHYKACLFAGINISGINAEVMPGQWEFQIGPVV |
| | GVSAGDHVWVARYILERITEIAGVVVSFDPKPIPGDWNGAGAHTNYSTKSM |
| | RSNGGYEVIKKAIKKLGMRHREHIAAYGDGNERRLTGRHETADINNFVWGV |
| | ANRGASVRVGRDTEKDGKGYFEDRRPASNMDPYLVTAMIAETTILWEPSHG |
| | HGHGQSNGK |
| 73 | TCGTCGTCCCTGCTCACTGACCTCGTTAACCTCGACCTGTCGGAGAGCACt |
| | GACAAGGTCATCGCCGAGTAtATATGGGTTGGTGGTACTGGGATGGATGT |
| | GAGaAGCAAAGCCAGAACaTTGTCTGGACCTGTTGATGACCCAAGCAAGC |
| | TaCCAAAGTGGAACTTTGATGGCTCCgGCACtGGTCAaGCTACtGGTGACGA |
| | CAGTGAAGTtATCCTCCAtCCTCAAGCCATCTTCcgtGACCCATTCAGaAAGG |
| | GGAAGAACATCCTGGTCATGTGTGACTGTTATGCGCCtAATGGCGAGCCaA |
| | TTCCtACtAACAACCGaTAtAATGCAGCAAGaATCTTCAGTCATCCTGATGTC |
| | AAGGCTGAAGAaCCtTGGTATGGGATTGAGCAaGAGTAtACCCTTCTTCAaA |
| | AGCAtATCAACTGGCCTCTTGGCTGGCCACTAGGTGGCTATCCAGGCCCTC |
| | AaGGTCCaTAtTAtTGTGCGGCGGGAGCCGATAAATCGTAtGGGCGtGACAT |
| | CGTTGATGCCCATTAtAAGGCCTGCCTGTTTGCCGGCATCAACATCAGCGG |
| | GATCAACGCAGAAGTCATGCCtGGGCAaTGGGAGTTCCAaATTGGCCCTGT |
| | CGTTGGCGTaTCCGCAGGGGATCATGTCTGGGTGGCACGUTAtATTCTTGAG |
| | AGaATCACTGAGATTGCTGGCGTCGTCGTGTCCTTCGACCCCAAGCCCATT |
| | CCtGGAGACTGGAATGGGCCGGTGCTCAtACCAAtTAtAGCACCAAGTCGA |
| | TGAGaAGCAATGGCGGCTACGAGGTGATCAAGAAAGCGATCAAGAAGCTC |
| | GGtATGCGCCAtCGTGAGCAtATCGCCGCCTAtGGCGACGGCAACGAGCGtag |
| | aCTCACtGGtCGtCAtGAGACtGCCGACATCAACAACTTCGTCTGGGGCGTAG |
| | CGAACCGtGGCGCGTCGGTGCGTGTCGGtCGaGACACCGAGAAGGACGGC |
| | AAAGGTTACTTCGAGGACAGacgaCCaGCGTCCAACATGGACCCaTAtCTGG |
| | TGACCGCCATGATCGCCGAGACtACCATCCTCTGGGAGCCCAGtCAtGGtCA |
| | tGGaCAtGGCCAATCCAACGGCAAGTGA |
| 74 | GAATTTCTCTGACATTCCATGTTTCCGAAACGGATCCTATATCGTCGTCCC |
| | TGCTCACTGACCTCGTTAACCTCGACCTGTCGGAGAGCACIGACAAGGTC |
| | ATCGCCGAGTAtATATGGGTTGGTGGTACTGGGATGGATGTGAGaAGCAA |
| | AGCCAGAACaTTGTCTGGACCTGTTGATGACCCAAGCAAGCTaCCAAAGT |
| | GGAACTTTGATGGCTCCgGCACtGGTCAaGCTACtGGTGACGACAGTGAAG |
| | TtATCCTCCAtCCTCAAGCCATCTTCcgtGACCCATTCAGaAAGGGGAAGAAC |
| | ATCCTGGTCATGTGTGACTGTTATGCGCCLAATGGCGAGCCaATTCCtACtA |
| | ACAACCGaTAtAATGCAGCAAGaATCTTCAGTCATCCTGATGTCAAGGCTG |
| | AAGAaCCtTGGTATGGGATTGAGCAaGAGTAtACCCTTCTTCAaAAGCAtATC |
| | AACTGGCCTCTTGGCTGGCCACTAGGTGGCTATCCAGGCCCTCAaGGTCCa |
| | TAtTAtTGTGCGGCGGGAGCCGATAAATCGTAtGGGCGtGACATCGTTGATG |
| | CCCAtTAtAAGGCCTGCCTGTTTGCCGGCATCAACATCAGCGGGATCAACG |
| | CAGAAGTCATGCCtGGGCAaTGGGAGTTCCAaATTGGCCCTGTCGTTGGCG |
| | TaTCCGCAGGGGATCATGTCTGGGTGGCACGUTAtATTCTTGAGAGaATCAC |
| | TGAGATTGCTGGCGTCGTCGTGTCCTTCGACCCCAAGCCCATTCCtGGAGA |
| | CTGGAATGGtGCCGGTGCTCAtACCAAtTAtAGCACCAAGTCGATGAGaAGC |
| | AATGGCGGCTACGAGGTGATCAAGAAAGCGATCAAGAAGCTcGGtATGCG |
| | CCAtCGTGAGCAtATCGCCGCCTAtGGCGACGGCAACGAGCGtagaCTCACtG |
| | GtCGtCAtGAGACtGCCGACATCAACAACTTCGTCTGGGGCGTAGCGAACCG |
| | tGGCGCGTCGGTGCGTGTCGGtCGaGACACCGAGAAGGACGGCAAAGGTT |
| | ACTTCGAGGACAGacgaCCaGCGTCCAACATGGACCCaTAtCTGGTGACCGC |
| | CATGATCGCCGAGACtACCATCCTCTGGGAGCCCAGICAtGGtCAtGGaCAtG |
| | GCCAATCCAACGGCAAGTGA |
| 75 | MDPISSSLLTDLVNLDLSESTDKVIAEYIWVGGTGMDVRSKARTLSGPVDDP |
| | SKLPKWNFDGSGTGQATGDDSEVILHPQAIFRDPFRKGKNILVMCDCYAPNG |
| | EPIPTNNRYNAARIFSHPDVKAEEPWYGIEQEYTLLQKHINWPLGWPLGGYP |
| | GPQGPYYCAAGADKSYGRDIVDAHYKACLFAGINISGINAEVMPGQWEFQIG |
| | PVVGVSAGDHVWVARYILERITEIAGVVVSFDPKPIPGDWNGAGAHTNYSTK |
| | SMRSNGGYEVIKKAIKKLGMRHREHIAAYGDGNERRLTGRHETADINNFVW |
| | GVANRGASVRVGRDTEKDGKGYFEDRRPASNMDPYLVTAMIAETTILWEPS |
| | HGHGHGQSNGK |
| 76 | MVSKGEELFTGVVPILVELDGDVNGHKFSVSGEGEGDATYGKLTLKFICTTG |
| | KLPVPWPTLVTTLTYGVQCFSRYPDHMKQHDFFKSAMPEGYVQERTIFFKD |
| | DGNYKTRAEVKFEGDTLVNRIELKGIDFKEDGNILGHKLEYNYNSHNVYIMA |
| | DKQKNGIKVNFKIRHNIEDGSVQLADHYQQNTPIGDGPVLLPDNHYLSTQSA |
| | LSKDPNEKRDQMVLLEFVTAAGITLGMDELYK |
| 77 | PVAT |
| 78 | KTGRKNHQRHHVLPARGRVGAAAVRCSAVSPVTPPSPAPPATPLRPWGPAE |
| | PRKGADILVEALERCGVSDVFAYPGGASMEIHQALTRSPVITNHLFRHEQGE |
| | AFAASGYARASGRVGVCVATSGPGATNLVSALADALLDSVPMVAITGQVPR |
| | RMIGTDAFQETPIVEVTRSITKHNYLVLDVEDIPRVIQEAFFLASSGRPGPVLV |
| | DIPKDIQQQMAVPVWDTSMNLPGYIARLPKPPATELLEQVLRLVGESRRPILY |
| | VGGGCSASGDELRWFVELTGIPVTTTLMGLGNFPSDDPLSLRMLGMHGTVY |
| | ANYAVDKADLLLAFGVRFDDRVTGKIEAFASRAKIVHIDIDPAEIGKNKQPH |
| | VSICADVKLALQGLNALLQQSTTKTSSDFSAWHNELDQQKREFPLGYKTFGE |
| | EIPPQYAIQVLDELTKGEAIIATGVGQHQMWAAQYYTYKRPRQWLSSAGLG |
| | AMGFGLPAAAGASVANPGVTVVDIDGDGSFLMNIQELALIRIENLPVKVMVL |
| | NNQHLGMVVQLEDRFYKANRAHTYLGNPECESEIYPDFVTIAKGFNIPAVRV |
| | TKKSEVRAAIKKMLETPGPYLLDIIVPHQEHVLPMIPIGGAFKDMILDGDGRT |
| | VYPVATMVSKGEELFTGVVPILVELDGDVNGHKFSVSGEGEGDATYGKL |
| | TLKFICTTGKLPVPWPTLVTTLTYGVQCFSRYPDHMKQHDFFKSAMPE |
| | GYVQERTIFFKDDGNYKTRAEVKFEGDTLVNRIELKGIDFKEDGNILGH |
| | KLEYNYNSHNVYIMADKQKNGIKVNFKIRHNIEDGSVQLADHYQQNTPI |
| | GDGPVLLPDNHYLSTQSALSKDPNEKRDQMVLLEFVTAAGITLGMDELY |
| | K |
| 79 | AAGACCGGCCGTAAGAACCATCAACGACATCATGTCCTTCCCGCACGAG |
| | GCCGAGTGGGGGCGGCGGCGGTCAGGTGCTCGGCGGTGTCCCCAGTCAC |
| | CCCACCATCCCCAGCGCCACCAGCCACTCCACTCCGACCATGGGGGCCAG |
| | CCGAGCCCCGTAAGGGCGCGGACATCCTCGTGGAGGCGCTGGAGCGATG |
| | CGGCGTCAGCGACGTGTTCGCCTATCCAGGCGGCGCGTCCATGGAGATCC |
| | ATCAAGCGCTGACTCGTTCCCCAGTCATCACCAACCATCTCTTCCGTCATG |
| | AGCAAGGCGAGGCGTTCGCGGCGTCCGGGTATGCGCGTGCGTCCGGCCG |
| | TGTCGGGGTCTGCGTCGCCACCTCCGGCCCCGGGGCAACCAACCTCGTGT |
| | CCGCGCTCGCCGACGCGCTGCTCGACTCCGTCCCAATGGTCGCCATCACT |
| | GGCCAAGTCCCCCGTCGTATGATCGGCACCGACGCCTTCCAAGAGACTCC |
| | CATAGTCGAGGTCACCCGTTCCATCACCAAGCATAATTATCTTGTCCTTGA |
| | TGTGGAGGACATCCCCCGTGTCATACAAGAAGCCTTCTTCCTCGCGTCCT |
| | CGGGCCGTCCTGGCCCAGTGCTGGTCGACATCCCCAAGGACATCCAACAA |
| | CAAATGGCCGTGCCAGTCTGGGACACCTCGATGAATCTACCAGGGTATAT |
| | CGCACGTCTGCCCAAGCCACCCGCGACAGAATTGCTTGAGCAAGTCTTGC |
| | GTCTGGTTGGCGAGTCACGACGTCCAATTCTCTATGTCGGTGGTGGCTGC |
| | TCTGCATCTGGTGACGAATTGCGTTGGTTTGTTGAGCTGACTGGTATCCCA |
| | GTTACAACCACTCTGATGGGCCTCGGCAATTTCCCCAGTGACGACCCATT |
| | GTCCCTGCGTATGCTTGGGATGCATGGCACTGTGTATGCAAATTATGCCG |
| | TGGATAAGGCTGACCTGTTGCTTGCGTTTGGTGTGCGATTTGATGATCGTG |
| | TGACAGGGAAAATTGAGGCTTTTGCAAGCAGGGCCAAGATTGTGCATATT |
| | GACATTGATCCAGCAGAGATTGGAAAGAACAAGCAACCACATGTGTCAA |
| | TTTGCGCAGATGTTAAACTTGCTTTACAAGGCTTGAATGCTCTGCTACAAC |
| | AAAGCACAACAAAGACAAGTTCTGATTTTAGTGCATGGCATAATGAGTTG |
| | GACCAACAAAAGAGGGAGTTTCCTCTGGGGTATAAAACTTTTGGTGAAG |
| | AGATCCCACCACAATATGCCATTCAAGTGCTGGATGAGCTGACTAAAGGT |
| | GAGGCAATCATCGCTACTGGTGTTGGGCAACATCAAATGTGGGCGGCAC |
| | AATATTATACCTATAAGCGACCACGACAATGGCTGTCTTCGGCTGGTCTG |
| | GGCGCAATGGGATTTGGGCTGCCTGCTGCAGCTGGTGCTTCTGTGGCTAA |
| | CCCAGGTGTCACAGTTGTTGATATTGATGGGGATGGTAGCTTCCTCATGA |
| | ACATTCAAGAGCTGGCATTGATCCGTATTGAGAACCTCCCTGTGAAGGTG |
| | ATGGTGTTGAACAACCAACATTTGGGTATGGTGGTGCAATTGGAGGATAG |
| | GTTTTATAAGGCGAATAGGGCGCATACATATTTGGGCAACCCAGAATGTG |
| | AGAGCGAGATATATCCAGATTTTGTGACTATTGCTAAGGGGTTCAATATT |
| | CCTGCAGTCCGTGTAACAAAGAAGAGTGAAGTCCGTGCCGCCATCAAGA |
| | AGATGCTTGAGACTCCAGGGCCATATTTGTTGGATATCATCGTCCCACAT |
| | CAAGAGCATGTGCTGCCTATGATCCCAATTGGGGGCGCATTCAAGGACAT |
| | GATCCTGGATGGTGATGGCAGGACTGTGTATCCAGTTGCTACTATGGTGA |
| | GTAAGGGAGAGGAGCTGTTCACCGGGGTGGTGCCTATCCTGGTCGAGCTG |
| | GATGGTGATGTAAACGGTCATAAATTCAGTGTGTCCGGTGAAGGTGAAG |
| | GTGATGCCACCTATGGTAAGCTGACCCTTAAGTTCATCTGTACCACCGGA |
| | AAGCTGCCTGTGCCTTGGCCTACCCTCGTGACCACCCTGACATATGGAGT |
| | GCAATGTTTCAGTCGTTATCCTGATCATATGAAGCAACATGATTTCTTTAA |
| | ATCCGCCATGCCTGAAGGTTATGTCCAAGAGCGTACCATATTCTTTAAAG |
| | ATGATGGTAACTATAAGACCCGTGCCGAGGTGAAGTTCGAGGGTGATAC |
| | CCTGGTGAACCGTATTGAGCTTAAGGGTATCGATTTCAAGGAGGATGGAA |
| | ACATCCTGGGGCATAAGCTGGAGTATAACTATAACAGTCATAACGTCTAT |
| | ATCATGGCCGATAAGCAAAAGAACGGTATCAAGGTGAACTTCAAGATCC |
| | GTCATAATATCGAAGATGGAAGTGTGCAACTCGCCGATCATTATCAACAA |
| | AACACCCCTATCGGTGATGGTCCTGTGCTGCTGCCTGATAACCATTATCTG |
| | AGTACCCAATCCGCCCTGAGTAAAGATCCTAACGAGAAGCGTGATCAAA |
| | TGGTACTGCTTGAGTTCGTTACCGCCGCCGGGATCACTCTCGGTATGGAT |
| | GAGCTGTATAAGTAA |
| 80 | GAATTTCTCTGTGAACAGTCACTCACTTTTGACAGTTATACGATTCCAGA |
| | AGATGATCCAGAATTGGATCCTATA |
| 81 | GAATTTCTCTGTGAACAGTCACTCACTTTTGACAGTTATACGATTCCAGA |
| | AGATGATCCAGAATTGGATCCTATAAAGACCGGCCGTAAGAACCATCAA |
| | CGACATCATGTCCTTCCCGCACGAGGCCGAGTGGGGGCGGCGGCGGTCA |
| | GGTGCTCGGCGGTGTCCCCAGTCACCCCACCATCCCCAGCGCCACCAGCC |
| | ACTCCACTCCGACCATGGGGGCCAGCCGAGCCCCGTAAGGGCGCGGACA |
| | TCCTCGTGGAGGCGCTGGAGCGATGCGGCGTCAGCGACGTGTTCGCCTAT |
| | CCAGGCGGCGCGTCCATGGAGATCCATCAAGCGCTGACTCGTTCCCCAGT |
| | CATCACCAACCATCTCTTCCGTCATGAGCAAGGCGAGGCGTTCGCGGCGT |
| | CCGGGTATGCGCGTGCGTCCGGCCGTGTCGGGGTCTGCGTCGCCACCTCC |
| | GGCCCCGGGGCAACCAACCTCGTGTCCGCGCTCGCCGACGCGCTGCTCGA |
| | CTCCGTCCCAATGGTCGCCATCACTGGCCAAGTCCCCCGTCGTATGATCG |
| | GCACCGACGCCTTCCAAGAGACTCCCATAGTCGAGGTCACCCGTTCCATC |
| | ACCAAGCATAATTATCTTGTCCTTGATGTGGAGGACATCCCCCGTGTCAT |
| | ACAAGAAGCCTTCTTCCTCGCGTCCTCGGGCCGTCCTGGCCCAGTGCTGG |
| | TCGACATCCCCAAGGACATCCAACAACAAATGGCCGTGCCAGTCTGGGA |
| | CACCTCGATGAATCTACCAGGGTATATCGCACGTCTGCCCAAGCCACCCG |
| | CGACAGAATTGCTTGAGCAAGTCTTGCGTCTGGTTGGCGAGTCACGACGT |
| | CCAATTCTCTATGTCGGTGGTGGCTGCTCTGCATCTGGTGACGAATTGCGT |
| | TGGTTTGTTGAGCTGACTGGTATCCCAGTTACAACCACTCTGATGGGCCTC |
| | GGCAATTTCCCCAGTGACGACCCATTGTCCCTGCGTATGCTTGGGATGCA |
| | TGGCACTGTGTATGCAAATTATGCCGTGGATAAGGCTGACCTGTTGCTTG |
| | CGTTTGGTGTGCGATTTGATGATCGTGTGACAGGGAAAATTGAGGCTTTT |
| | GCAAGCAGGGCCAAGATTGTGCATATTGACATTGATCCAGCAGAGATTG |
| | GAAAGAACAAGCAACCACATGTGTCAATTTGCGCAGATGTTAAACTTGCT |
| | TTACAAGGCTTGAATGCTCTGCTACAACAAAGCACAACAAAGACAAGTTC |
| | TGATTTTAGTGCATGGCATAATGAGTTGGACCAACAAAAGAGGGAGTTTC |
| | CTCTGGGGTATAAAACTTTTGGTGAAGAGATCCCACCACAATATGCCATT |
| | CAAGTGCTGGATGAGCTGACTAAAGGTGAGGCAATCATCGCTACTGGTGT |
| | TGGGCAACATCAAATGTGGGCGGCACAATATTATACCTATAAGCGACCAC |
| | GACAATGGCTGTCTTCGGCTGGTCTGGGCGCAATGGGATTTGGGCTGCCT |
| | GCTGCAGCTGGTGCTTCTGTGGCTAACCCAGGTGTCACAGTTGTTGATATT |
| | GATGGGGATGGTAGCTTCCTCATGAACATTCAAGAGCTGGCATTGATCCG |
| | TATTGAGAACCTCCCTGTGAAGGTGATGGTGTTGAACAACCAACATTTGG |
| | GTATGGTGGTGCAATTGGAGGATAGGTTTTATAAGGCGAATAGGGCGCAT |
| | ACATATTTGGGCAACCCAGAATGTGAGAGCGAGATATATCCAGATTTTGT |
| | GACTATTGCTAAGGGGTTCAATATTCCTGCAGTCCGTGTAACAAAGAAGA |
| | GTGAAGTCCGTGCCGCCATCAAGAAGATGCTTGAGACTCCAGGGCCATAT |
| | TTGTTGGATATCATCGTCCCACATCAAGAGCATGTGCTGCCTATGATCCC |
| | AATTGGGGGCGCATTCAAGGACATGATCCTGGATGGTGATGGCAGGACT |
| | GTGTATCCAGTTGCTACTATGGTGAGTAAGGGAGAGGAGCTGTTCACCGG |
| | GGTGGTGCCTATCCTGGTCGAGCTGGATGGTGATGTAAACGGTCATAAAT |
| | TCAGTGTGTCCGGTGAAGGTGAAGGTGATGCCACCTATGGTAAGCTGACC |
| | CTTAAGTTCATCTGTACCACCGGAAAGCTGCCTGTGCCTTGGCCTACCCTC |
| | GTGACCACCCTGACATATGGAGTGCAATGTTTCAGTCGTTATCCTGATCA |
| | TATGAAGCAACATGATTTCTTTAAATCCGCCATGCCTGAAGGTTATGTCC |
| | AAGAGCGTACCATATTCTTTAAAGATGATGGTAACTATAAGACCCGTGCC |
| | GAGGTGAAGTTCGAGGGTGATACCCTGGTGAACCGTATTGAGCTTAAGG |
| | GTATCGATTTCAAGGAGGATGGAAACATCCTGGGGCATAAGCTGGAGTA |
| | TAACTATAACAGTCATAACGTCTATATCATGGCCGATAAGCAAAAGAACG |
| | GTATCAAGGTGAACTTCAAGATCCGTCATAATATCGAAGATGGAAGTGTG |
| | CAACTCGCCGATCATTATCAACAAAACACCCCTATCGGTGATGGTCCTGT |
| | GCTGCTGCCTGATAACCATTATCTGAGTACCCAATCCGCCCTGAGTAAAG |
| | ATCCTAACGAGAAGCGTGATCAAATGGTACTGCTTGAGTTCGTTACCGCC |
| | GCCGGGATCACTCTCGGTATGGATGAGCTGTATAAGTAA |
| 82 | MIPEDDPELDPIKTGRKNHQRHHVLPARGRVGAAAVRCSAVSPVTPPSPAPP |
| | ATPLRPWGPAEPRKGADILVEALERCGVSDVFAYPGGASMEIHQALTRSPVIT |
| | NHLFRHEQGEAFAASGYARASGRVGVCVATSGPGATNLVSALADALLDSVP |
| | MVAITGQVPRRMIGTDAFQETPIVEVTRSITKHNYLVLDVEDIPRVIQEAFFLA |
| | SSGRPGPVLVDIPKDIQQQMAVPVWDTSMNLPGYIARLPKPPATELLEQVLR |
| | LVGESRRPILYVGGGCSASGDELRWFVELTGIPVTTTLMGLGNFPSDDPLSLR |
| | MLGMHGTVYANYAVDKADLLLAFGVRFDDRVTGKIEAFASRAKIVHIDIDP |
| | AEIGKNKQPHVSICADVKLALQGLNALLQQSTTKTSSDFSAWHNELDQQKR |
| | EFPLGYKTFGEEIPPQYAIQVLDELTKGEAIIATGVGQHQMWAAQYYTYKRP |
| | RQWLSSAGLGAMGFGLPAAAGASVANPGVTVVDIDGDGSFLMNIQELALIRI |
| | ENLPVKVMVLNNQHLGMVVQLEDRFYKANRAHTYLGNPECESEIYPDFVTI |
| | AKGFNIPAVRVTKKSEVRAAIKKMLETPGPYLLDIIVPHQEHVLPMIPIGGAF |
| | KDMILDGDGRTVYPVATMVSKGEELFTGVVPILVELDGDVNGHKFSVSG |
| | EGEGDATYGKLTLKFICTTGKLPVPWPTLVTTLTYGVQCFSRYPDHMK |
| | QHDFFKSAMPEGYVQERTIFFKDDGNYKTRAEVKFEGDTLVNRIELKGI |
| | DFKEDGNILGHKLEYNYNSHNVYIMADKQKNGIKVNFKIRHNIEDGSVQ |
| | LADHYQQNTPIGDGPVLLPDNHYLSTQSALSKDPNEKRDQMVLLEFVTA |
| | AGITLGMDELYK |
| 83 | TAAGGTACCAAAATAATACGACTCACTATAGACCTAGGAAAAGATCTAGAA |
| | GCGGGGTAGAGGAATTGGTCAACTCATCAGGCTCATGACCTGAAGACTG |
| | CAGGTTCGAATCCTGTCCCCGCCTGTCTGGCCCCAAATTCTAATTTCTACT |
| | GTTGTAGATGTATCGATCATATATTCTAGGCGAGGTGTAGCGCAGTCTGG |
| | TCAGCGCATCTGTTTTGGGTACAGAGGGCCATAGGTTCGAATCCTGTCAC |
| | CTTGAGTCTGGCCCCAAATTCTAATTTCTACTGTTGTAGATAACTGAAATG |
| | GGCTGATCTAGGTCAGGATAGCTCAGTTGGTAGAGCAGAGGATTGAAAA |
| | TCCTCGTGTCACCAGTTCAAATCTGGTTCCTGGCAGTCTGGCCCCAAATTC |
| | TAATTTCTACTGTTGTAGATTGTAAAGGAGTACAAGGTGCTGGAGAGATG |
| | GCCGAGTGGTTCAAGGCGTAGCATTGGAACTGCTATGTAGGCTTTTGTTT |
| | ACCGAGGGTTCGAATCCCTCTCTTTCCGGTCTGGCCCCAAATTCTAATTTC |
| | TACTGTTGTAGATTCAACACCAGCTTCTCACCTGGGGTGTATAGCTCAGTT |
| | GGTAGAGCATTGGGCTTTTAACCTAATGGTCGCAGGTTCAAGTCCTGCTA |
| | TACCCAAACCCCTTGGGGCCTCTAAACGGGTCTTGAGGGGTTTTTTGGTACCA |
| | ACCCCTTGGGGCCTCTAAACGGGTCTTGAGGGGTTTTTTGATAGACCTTTTT |
| | ATTTTTCGTCATTCGATCACGAAAACAGGGATTCTGGAACGGCCAAG |
| | AATCCCAGCGGTTGTTCGGGTCGAAAAACCGAGAACAAGACATGCCA |
| | CAAAGTGGCAGATGAAGGCAGGGGGGAGAGCCTAGTCCTCAACCTC |
| | TTCTTCCCCAAAAGGTAGTTATGAACGTGCCAAACTTATTGGATTTAT |
| | TCTTGGAATGCTCATAACCACCTTTACTCTTTTTTTCATTCTTTACTCA |
| | GAGGAAGCCATGCCGTTTGGAGAAGAGCACCAAGTGGGGGGAGTGT |
| | GGAGTCCCCGAAAGAGGAGCTTTCTAAAGGCAAGAGAAAAGCTCCG |
| | ATGGAGCCCTTGGAGCTACAGGGACCACCAACCCTTCGCAGCTTGGA |
| | CGATTTGATTCTTGTGCCACTCAGCCC |
| 84 | TGAGGTATTATGGGATTTATGTTTATACCAAAGCCTCTACTTTAGATATTT |
| | ACGCTGATGAAGGAAAGCCTTGTTAAAGACTACGACTTTGGACTTCCTTT |
| | TTCGTACTCTTCTTTCATCCTCCTTACCAGAACGCTTTTCCTGGTTCAGAA |
| | GAATAAGATACTTAAATTGTGGCTTACATATGGAAAATGCCTTCGTCTTG |
| | AAGCTACCAATGCACATCACGGTGAAATCATTGTCAATTAATTGTTAGAT |
| | TCCCCATATCACCCATAGCCACCCAAATCTTTTTTTTGCCTCAAACTCTGC |
| | TGCCCTATCACTCCTCGATAAGTGTACTTACTCAGGTTGTGCACTTGGCTG |
| | AGCGAGATCTTCCCCTTTGGCATGAGAACCGCCTACCTACGCTAACGTAA |
| | CTAATGCAGATGGCGGAAGTTAGTAGTTCTCCTATGCCAACATGAACACA |
| | AACGAAATCGTCGAATTTCGTATAGAAAGAAAAGGAACCACTTCTATTCT |
| | CTTTTCTTAAGTATAAGAGGGATAAGTACTTATTCCTGGCCGGCGGTATC |
| | ATTGCTGCTCCCCGCCTAATGCGGATCATTGTGCAATGCTTATGTGAAATC |
| | TCAATCCAAAACTTATTCGTTTCGTTGGAAAAACCAACGCCGATCTAAAA |
| | CTAAGTCTTCCTTTCAAAAGTGAGCGAGCAGAGCTGAAAAAGATGGAGTT |
| | ACCTGGAGATGAGATTTCTTTCTACGGATATGAAGGATAGAAATATGCTA |
| | TTTGCTGCTATTACAACGAATCAACCAATTCGTAGTAAGTGTTCCCGTCTT |
| | CCCGATCTACATGATTTTTTCCCAACCAACATCTCTCAGAACTTTGCTATA |
| | ACGCCAAACTTGGATATAACGCCAACGCCTGAGCGAATTGCCGGCGTCAC |
| | AATAGTTTTACAAATAGAAGAGTATTTGGGCCAAAATGAGTCCGAACAG |
| | GGAGCAGTCAATTTAGCTAGAACAGTATTGGGAGCCCGCCACCGAAATG |
| | GCGAAACTTGGCAGGGCATATTAGAGGATATTCGGGCGGGTGGTGGTAT |
| | GGATAATTTTATCCAGAATCTGCCTGGTGCCTACCCGGAAACCCCATTGG |
| | ATCAATTTGCCATTATCCCAATAATTGATCTTCATGTGGGCAACTTTTATT |
| | TATCATTTACAAATGAAGTCTTGTATATGCTGCTCACTGTCGTTTTGGTCG |
| | TTTTTCTTTTTTTTGTTGTTACGAAAAAGGGAGGTGGAAAGTCAGTGCCAA |
| | ATGCATGGCAATCCTTGGTCGAGCTTATTTATGATTTCGTGCTGAACCTGG |
| | TAAACGAACAAATAGGTGGTCTTTCCGGAAATGTGAAACAAAAGTTTTTC |
| | CCTCGCATCTCGGTCACTTTTACTTTTTCGTTATTTCGTAATCCCCAGGGT |
| | ATGATACCCTTTAGCTTCACAGTGACAAGTCATTTTCTCATTACTTTGGCT |
| | CTTTCATTTTCCATTTTTATAGGCATTACGATCGTTGGATTTCAAAGACAT |
| | GGGCTTCATTTTTTTAGCTTCTTATTACCTGCGGGAGTCCCACTGCCGTTA |
| | GCACCTTTCTTAGTACTCCTTGAGCTAATCTCTTATTGTTTTCGTGCATTAA |
| | GCTTAGGAATACGTTTATTTGCTAATATGATGGCCGGTCATAGTTTAGTA |
| | AAGATTTTAAGTGGGTTTGCTTGGACTATGCTATTTCTGAATAATATTTTC |
| | TATTTCATAGGAGATCTTGGTCCCTTATTTATAGTTCTAGCATTAACCGGT |
| | TTTGAATTAGGTGTAGCTATATCACAAGCTCATGTTTCTACGATCTCAATT |
| | TGTATTTACTTGAATGATGCTACAAATCTCCATCAAAATGAGTCATTTCAT |
| | AATTGAAAGAAGGCTACCGAAAGAACAAGCAACGGATTTCGCGCTCATC |
| | CCTTGCTAAAGCGCATACGTTTTCTTGCTCCTTGGTACTTGTCTGATCAAT |
| | CACACTGTTCATTAGTTCACTTGATTTTTCGTTCGATGTCTTGAACCGCTT |
| | ACTAACCTGA |
| 85 | TAGGGATAACAGGGTAATTGAGTGGAGCAATATATTTAATAACCTTTGTA |
| | GTGATCTCAGTTCATGGAGATCACGAGGCCACTGATACCCGAAGTGGTTA |
| | GGTGGGATAGAAGAGTGGGTATGTGGGCTTCTATCAAAAGAGACCGCTA |
| | TCGCTACGCTCATATACGTCAAGGAAAGGAAGGGAGCGAAAAGATACGT |
| | TAATGTTTCCACCTACTCTCTTTT |
| 86 | AATCCAATCCCACAAAGGATTGTTCATGGAACTACTATCGAAATTATTCG |
| | GACCATATTTCCAAGTGTCATTCTTTTGTTCATTGCTATACCATCGTTTGG |
| | TCTTGCGTTGACAGTACTAATAATGGCATTTGTTGTCTTATTCGGAGTTCA |
| | AGGAATAGCCTTTCATCTTGGGAATGAGAATGTCGCGGATCTCAATGTTC |
| | TCGTCATGACCAATGCTCCTAACGGGGGTGACTTTCCCATAGACCAACCT |
| | CCCGTTGGCGGACTACCAGACCTCCAGCAGGAAATGGCGCACGCCGCCC |
| | ATCCCGAAGACATAATTGCGGAGTTGACTTCCAAGGTTGAAAGAATATTT |
| | CACGAAGTAGGCACTCCTCTTCCTTTAGAGGAAGGTGAATCTGCGCGATC |
| | GTTCACTGAAAATCATGTATTGTGGAATAGTGAGGGAGGAGATACTCTTC |
| | AACAAATCTTCTCCGATTATACGGAAGCCGGGGACGCAAGCGAATTCGTC |
| | GCAATAGCCACCAATCTGGCGAAGCGGTTTCGCCGCGCCGAGCTCGGTGA |
| | ACCGGATTCTCCCGACCCCGATGCGCCTTTGGAAGAGCAGGAGGCTCATT |
| | CCACGGGALTCCCCTGAAAGCACAAAAGCAAATGATGGGGCGAACGAGG |
| | CAGGCCCGTCTTCTTCGCGGAAGCGTAAAAGGTGGGATGATGATTCGGGG |
| | TCCGAAGATGATGATTCGGGGGGCCCCGGGGAATCTCCAGATCCAGTGG |
| | AAATCGTTTACGAAGGGGATGCACAAGGGTACAACTGGGAGGGGAATTA |
| | GGTTGGCCGCCAACTTCGCCTGCCTTTCTATCTGAGTTCTTCCCTCTTGAT |
| | GCTTTCGAACGACTCCTAAATTTCACAAAATCCTTTTTTTCTTATTTGAAA |
| | TCCAAATCGAAATGCCTCAACTTGATAAATTAACTTATTTCTCACAATTCTTCTG |
| | GTTATGTCTTCTCCTCTTTACTTTTTATATTCTCTTATTTAATAATAATAATGGAAT |
| | ACTTGGAATTAGTAGAATTCTCAAACTACGGAACCAACTGCTTTCGCACCGGGG |
| | GGGCGAGATCCGGAGCAAGGACCCTAAGAATCTGGAAGATATCTCGAGAAAA |
| | GGTTTTAGCACCGGTCTCTCATATATGTACTCCAGTTTATCCGAAGTATCCCAAT |
| | GGTGTAAGACCGTCGACTATTTGGGAAATAAGATATCTTCTTCAATCTTTCTATA |
| | CTATCTTAGGGGCGTCCTTTGCCCAATTTGCCTCTTATTTTTTAAATTTCTTATTT |
| | CTTTCGCCTTCACCTGTGCACTTATTTATGTATTCAAGGGGGGGGGTTTCGTGG |
| | CTATGGCTGCTACAAACGGAGCCTCTTCTTCCTTTCTGGAGAGCTCAGGTGAA |
| | ATAGATGCACTGTTACAAACAACAACAACAACACGAACACCCGAAAACGCGGA |
| | TTACGAAGACGATAATACTTCTGTAAATCAAGAATTCCTCGAAGACGGACAAAG |
| | GGCCCGACAGGCTAAACTACGGGAGTTAGAAAGACTCATTCTCCAGCAATATA |
| | AGGATTTCATCCGACAGAAATATCCATGGATACCTAAAGGCGACATCCTTCTCC |
| | CGAGCATGAAGGGTGGAGTTGTCGAGGACGTAATGGAAAAATTAGAATTGGAA |
| | ACGTATTCCAGTAGCGACTTGACTGATTGGATCAACCATTTGCGGGCAAATCC |
| | GAAAACATTAAATTTTATCTTCAAGGATTTTGTCGCGTGAGTGGAGCAATATAT |
| | TTAATAACCTTTGTAGTGATCTCAGTTCATGGAGATCACGAGGCCACTGA |
| | TACCCGAAGTGGTTAGGTGGGATAGAAGAGTGGGTATGTGGGCTTCTATC |
| | AAAAGAGACCGCTATCGCTACGCTCATATACGTCAAGGAAAGGAAGGGA |
| | GCGAAAAGATACGTTAATGTTTCCACCTACTCTCTTTT |
| 87 | AAATGAGATAAGGAGCAGTAAGGAGGATAAAAGAAGTAGAGTTAGGTTC |
| | TTGTGTGTAATCGATATGTCTCCCGGGAGAAAACCCTCTATCAACACAAC |
| | TATCCGAACACCTATAGCTAGCTTGCTTTAGCTCCAACTACTGCTAGCTTT |
| | ACCTGCAACGCAACTATCTTCCGAAAAACGAGTTGTTCGTTGCCCGACGC |
| | GGCTCTGGCTCTGCCTGCCTGGCACTAAAGCATCTATAGATTGAGGATTT |
| | GCCTATGTACCATCATCCGTCCTCTAAGTAATATGTATTCTTCCTCTAAGG |
| | TTCCCCCTCTAAGGAGGGTTGTGTAGTATTTAGCTTACAAGGATTGACTC |
| | GACACCATCGATGCGTAGTCTATCAATCAATTTAATGGGACTTATTTATAT |
| | GCCTGTTACGAGGGGAATCTGCCTGTTATTCAATCTATCTCTTCAATCTGC |
| | TTACTGGTTTCCTTCTCGGCCAATGGAACATCTATTGCTTCCTAATCTGGA |
| | ACTGAACTAAACAGAAATCTTGGTCAAGTATTGGGAAAAGGAAAGAGGC |
| | CCTTTTCTTTTTCAAAACTAAGAAGGAGATGTGCATAGCGTTGTAAAGAT |
| | CGGCAGAAGAGGAAGGGTGCAGTTAAGTTACAACATCTTCTGTTTCCAAG |
| | TCGATAGGCTTCTTTTGATAAAGGAAACTATTCCTCTGCTCGCTATACTCA |
| | AACCCTTGCCATTTCCTCGATTCCCGTTGAGAAAACAATCGATTGACCATT |
| | TTAGGACCTATTCTTCAGAATATAGAGGTATCGAAGATCTAGAAACGATG |
| | CGATGACTCCAGTTCGATCCTTTTCCGATGAAATGAATGAATTACCACGC |
| | TTTCCGCCGGCTGATAAAGAACTTTTTGATGATCGATTGCTTCACCCATGA |
| | TCTGATGATCTTGCTATAACATAACGATACTGCATTTCCCCTCTTCCTTGT |
| | CTCTACTTAACTTACTTGTTCATTGCTCGACTCGAGGGAGGGGCTCTATAT |
| | AGCTGTTTTGCCTTCTTTCTTGGACTCTCAGGTGAGCGCTTAGGCCAAGCC |
| | ACAATTACCAAGGCATGATTGAATCCGCCAAAGAGAAGGGAATAGATGA |
| | TCTCACTGGAAAAAGTTGCGTAACCCCCCCCCCCCCCGTTTTTTTGGCATG |
| | ATTTCAAGATTTTGACCTGATTTATGAGAAAAGAACTTCGT |
| 88 | ATGAGATTTCTTTCTACGGATATGAAGGATAGAAATATGCTATTTGCTGC |
| | TATTACAACGAATCAACCAATTCGTAGTAAGTGTTCCCGTCTTCCCGATCT |
| | ACATGATTTTTTCCCAACCAACATCTCTCAGAACTTTGCTATAACGCCAAA |
| | CTTGGATATAACGCCAACGCCTGAGCGAATTGCCGGCGTCACAATAGTTT |
| | TACAAATAGAAGAGTATTTGGGCCAAAATGAGTCCGAACAGGGAGCAGT |
| | CAATTTAGCTAGAACAGTATTGGGAGCCCGCCACCGAAATGGCGAAACTT |
| | GGCAGGGCATATTAGAGGATATTCGGGCGGGTGGTGGTATGGATAATTTT |
| | ATCCAGAATCTGCCTGGTGCCTACCCGGAAACCCCATTGGATCAATTTGC |
| | CATTATCCCAATAATTGATCTTCATGTGGGCAACTTTTATTTATCATTTAC |
| | AAATGAAGTCTTGTATATGCTGCTCACTGTCGTTTTGGTCGTTTTTCTTTTT |
| | TTTGTTGTTACGAAAAAGGGAGGTGGAAAGTCAGTGCCAAATGCATGGC |
| | AATCCTTGGTCGAGCTTATTTATGATTTCGTGCTGAACCTGGTAAACGAA |
| | CAAATAGGTGGTCTTTCCGGAAATGTGAAACAAAAGTTTTTCCCTCGCAT |
| | CTCGGTCACTTTTACTTTTTCGTTATTTCGTAATCCCCAGGGTATGATACC |
| | CTTTAGCTTCACAGTGACAAGTCATTTTCTCATTACTTTGGCTCTTTCATTT |
| | TCCATTTTTATAGGCATTACGATCGTTGGATTTCAAAGACATGGGCTTCAT |
| | TTTTTTAGCTTCTTATTACCTGCGGGAGTCCCACTGCCGTTAGCACCTTTC |
| | TTAGTACTCCTTGAGCTAATCTCTTATTGTTTTCGTGCATTAAGCTTAGGA |
| | ATACGTTTATTTGCTAATATGATGGCCGGTCATAGTTTAGTAAAGATTTTA |
| | AGTGGGTTTGCTTGGACTATGCTATTTCTGAATAATATTTTCTATTTCATA |
| | GGAGATCTTGGTCCCTTATTTATAGTTCTAGCATTAACCGGTTTTGAATTA |
| | GGTGTAGCTATATCACAAGCTCATGTTTCTACGATCTCAATTTGTATTTAC |
| | TTGAATGATGCTACAAATCTCCATCAAAATGAGTCATTTCATAATTGATTT |
| | CATAATTGAATAAAAACGAGGAGCCGAAGATTTTAGGGGGGGGACAAA |
| | CGCGGAAGTGTATTGCGTTACAAAAAATGACAACTAGCATTTGTTTTTTC |
| | ATTTCATGTTCGAATTCGTTTTTCGTTGGAAAAACCAACGCCGACCCCAA |
| | ACAAGTCTCTCCAATATAAGGAGAGCGGAGCTTAAAAATATTATTTTATT |
| | GTGCTATGGCAAATCTGGTCCGATGGCTCTTCTCCACTACCCGAGGG |
| | ACTAACGGTCTTCCATATTTCATCTTCGGTGTCGTTGTAGGAGGCGC |
| | CCTGTTGTTTGCTTTGCTAAAGTATCAGGCCCCTCTGTACGACCCGG |
| | CTTTAATGGAAAAAATCATAGATCATAATATAAAAGCCGGGCACCCT |
| | ATAGAGGTTGACTATTCGTGGTGGGGCACCTCTATTCGTGTAGTCTT |
| | TCCTAAGTAAATAAAAACGAGGAGCCGAAGATTCCTGA |
| 89 | AGGGAAATTCATCCCAACCAATAGCAGTGA |
| 90 | CATCGGACCAGATTTGCCATAGCACA |
| 91 | CTGAGGAGTAAGCCTAATTCCGTTAATGCAG |
| 92 | CTTATATTGGAGAGACTTGTTTGGGGTCGGC |
| 93 | CGCGTTAGCGCATTCGGAACCA |
| 94 | TCGAGGTAATTAAAGCGGCCGAAATTAGC |
| 95 | CTCAGAATAATCCAGGTCGCTCGACG |
| 96 | CACAGGTAGCAATAGGTATTACAGTAGGGATAACAG |
| 97 | ATGCACATAAGCCATCCGAAACCAGTATTG |
| 98 | GAATGGAGGAACGGCGATATAG |
| 99 | CTTCTTATTACCAGCGGGAGTCC |
| 100 | GAAGGTAGACCTAAACGCTGAAG |
| 101 | GGGAAAGGAAACAAACCACTC |
| 102 | TCCCGTGATATTCCGCCTTG |
| 103 | GATAGAAGAGTACCAACCAAAGAAGCAAAAGCAAGACCTCACTAGTAA |
| | GGAAGGCACTTGCTGCCGGAGTTCAACAGGCAAATATAAGAAAAGAA |
| | GTCCTGTTCACTTCATCATCTGTGGGTTGTACTGCTTGAAGGTTCTTC |
| | TGAGGGGTAGAATTTGAATTCCTTCTTTGCTTGTGAGATAACCATTTC |
| | CAGAAACTCATATATAGAGAGCGGGTATCGGTGAAAATGGATCTTAC |
| | CAGGAGTGGCATTGAATAGGCAGGCTCTGGGATGTAATCTCACTCAA |
| | GAGGTCATTTGTTGGCCCCGCCTTCACTAGACTAGAGTTTTAGGATA |
| | GGTTGGGGAACCTATACGTCAAGCCCCTACGAAGATTGAGAAAAATC |
| | GATGCACATAAGCCATCCGAAACCAGTATTGGAAAGTGTTCAGTTTC |
| | GTTTTCCATTCTGAAATGTTCATAGTAGTATAGTATGTTTTCCGTTGG |
| | GTCGACGCCATGTGATCGCTACTAAAGATAGAGTTTCCTTGGAAAAA |
| | CCGAGGCCAGTTGAGATCAGTCTCCCTTTCTAGGAGCAGAGCTTAAA |
| | AAGATGGGAAATTCCAATGAATTTCGATCACAATCATGTGGTAATAA |
| | TGGGTTTGAATCAGAGAGACTCGATCTGGAAACTCCTCAATGATTAT |
| | AACGTGAACTCGTTGAAGAGAAGGAGACAAGCAGAAATAGACGCTTT |
| | TTTTGAACCATTTGAGAGGGCGCAGCGTATCCGTTTCAATAACTGGC |
| | AGAACGGAATAGAGTTGTTAGATGGGGCTGAATGGAGGAACGGCGA |
| | TATAGTTATCCCTGGAGGCGGCGGACCAGTAATTTCAAGCCCCTTGG |
| | ATCAATTTTTCATTGATCCATTATTTGGTCTTGATATGGGTAACTTTT |
| | ATTTATCATTCACAAATGAATCCTTGTCTATGGCGGTAACTGTCGTTT |
| | TGGTGCCATCTTTATTTGGAGTTGTTACGAAAAAGGGGGGGGGAAAG |
| | TCAGTGCCAAATGCATGGCAATCCTTGGTAGAGCTTATTTATGATTTC |
| | GTGCTGAACCTGGTAAACGAACAAATAGGTGGAAATGTTAAACAAAA |
| | GTTTTTCCCTCGCATCTCGGTCACTTTTACTTTTTCGTTATTTCGTAAT |
| | CCCCAGGGTATGATACCCTTTAGCTTCACAGTGACAAGTCATTTTCTC |
| | ATTACTTTGGCTCTTTCATTTTCCATTTTTATAGGCATTACGATCGTT |
| | GGATTTCAAAGACATGGGCTTCATTTTTTTAGCTTCTTATTACCAGCG |
| | GGAGTCCCACTGCCATTAGCACCTTTTTTAGTACTCCTTGAGCTAATC |
| | TCTCATTGTTTTCGTGCATTAAGCTCAGGAATACGTTTATTTGCTAAT |
| | ATGATGGCCGGTCATAGTTCAGTAAAGATTTTAAGTGGGTTCGCTTG |
| | GACTATGCTATTTCTGAATAATATTTTCTATTTCATAGGAGATCTTGG |
| | TCCCTTATTTATTGTATTGGCTCTTACTGGATTGGAATTAGGTGTAGC |
| | TATATTACAAGCTCATGTTTCTACGATCTCAATTTGTATTTACTTGAA |
| | TGATGCTATAAATCTCCATCAAAATGAGTAATTTCATAATTGAATAAA |
| | AACGAGGAGCCGAAGATTTTAGGGGGCGGGACAAACGCGGAAGTGTAT |
| | TGCGTTACAAAAAATGACAACTAGCATTTGTTTTTTCATTTCATGTTCGAATTCGT |
| | TTTTCGTTGGAAAAACCAAC |
| 104 | GGTAATGAATTCGAAACCATCTTCTCACTCTGACCCCCACATATCAGAT |
| | CCCAGATGCATAGGAAAAGCGGTATCAAGAATAGTAGTATAAAGAAA |
| | GATAGTACAGTACTCAAGTAAATGAATTCGCCTAAGGATCGATGGAA |
| | AGATCAAGGTCCCCGTGAAAAAGTAGATACTAGATCGATATGATACT |
| | CTCATCTCTGGAGTAACTTCTTCCATTATGCTGATCTCTAGGTCCGTT |
| | CCATCATCATCGTAATAGTATGGTCCCAGGTGTCCGAGCTATAGATC |
| | AAGATCATATCCAGTCACATTTCTACCGGTGCACTTCTCATGAAATAA |
| | TTCCCTTTCCAAGGAAAGGAAAACAAGAACTCGAATACTCGTAATAG |
| | CGATCCCGATCCACCTACTTTTTTCTATTCTTTGATTCGAAACGTGCT |
| | AAAGCACAAGCCATTTTTATGCATGGGGCATAAGAGTGGACAATCTA |
| | TGTTATCGAAGGAAGTAAATAACAACACTTCAGCGTTTAGGTCTACC |
| | TTCAGTAAACCAATAGTTTTGCAGCATTGGAATTTGAGTTGGCCAGG |
| | TAAGGTCCTCTAAAAAGAAAAGAAGAAACTACTTAGAATAGATAAAT |
| | GCCATTGGTTTTCTCGTACTATACGATCTTTTTTTGTTTTGTTTTTTGG |
| | CCATGATTGTGCTGCTCCTGTGAAGGCTAGTGGGAAAGCTCACCGTT |
| | CGTTGTGATGAGTGGGGGCCTTGTATCTGTATTCGGATCAGCTCCTT |
| | AACAGAGTTTCCTGCTTGAACCCTGGCTGGGAGCTGGGAGAGGTGTC |
| | CCACTACAGGTGCAAATAAACCATTTGACCTTACAGGGGAAAGGAAA |
| | CAAACCACTCAATAATCGGTAGAAATTCCTCCTACTGAACAGCTTTCC |
| | TTTTCTCGCCTTAACTACTACTTCAAAGCAAGGCGGAATATCACGGG |
| | ATAGGAATGAAAGAACTTCTTACTCAACTTTCTAGCTATATAAAAATA |
| | GTTAGCAATATGAAACGAGTAACTTAAGCCCTAGTAAAAGGCTACTC |
| | TTTGAATCCCCTCTTTAAGGCATATAAAATTAGTACTCTTCCTGAGCT |
| | AGCTTAAGCATATCTTGAGCGAGTGAGTTGTATTTCCCTCCATCAAGT |
| | TCTAAGCGATCAAATAAGGTCCTTGCTCTCGAGCCAATGCCAATACC |
| | AATAGAGAGGGTCTAAACGAAGGATTCAAAGGAGGTTACCGGTTAAGG |
| | GCAAATGCAATCTCTAAGCCAGTTGGAGAAGAAAGGTAATCAAAGAAAG |
| | TTTCAGTTGGACTTACCCTGCCAACCCTACCCTAATTTCATACCCTGCGGT |
| | TTTCGTTCCTCTATTGATATTTCGTTCCTCTCCTTA |
| 105 | GGCCAACTCAAATTCCAATGCTG |
| 106 | GCACTTGCTGCCGGAGTTCAACAGGCAAATATAAGAAAAGAAGTCCTGTT |
| | CACTTCATCATCTGTGGGTTGTACTGCTTGAAGGTTCTTCTGAGGGGTAGA |
| | ATTTGAATTCCTTCTTTGCTTGTGAGATAACCATTTCCAGAAACTCATATA |
| | TAGAGAGCGGGTATCGGTGAAAATGGATCTTACCAGGAGTGGCATTGAA |
| | TAGGCAGGCTCTGGGATGTAATCTCACTCAAGAAGTCATTTGTTGGCCCC |
| | GCCTTCACTAGACTAGAGTTTTAGGATAGGTTGGGGAACCTATACGTCAA |
| | GCCCCTACGAAGATTGAGAAAAATCGATGCACATAAGCCATCCGAAACC |
| | AGTATTGGAAAGTGTTCAGTTTCGTTTTCCATTCTGAAATGTTCATAGTNN |
| | TATAGTATGTTTTCCGTTGGGTCGACGCCATGTGATCGCTACTAAAGAT |
| | AGAGTTTCCTTGGAAAAACCGAGGCCAGTTGAGATCAGTCTCCCTTT |
| | CTAGGAGCAGAGCTTAAAAAGATGGGAAATTCCAATGAATTTCGATC |
| | ACAATCATGTGGTAATAATGGGTTTGAATCAGAGAGACTCGATCTGG |
| | AAACTCCTCAATGATTATAACGTGAACTCGTTGAAGAGAAGGAGACA |
| | AGCAGAAATAGACGCTTTTTTTGAACCATTTGAGAGGGCGCAGCGTA |
| | TCCGTTTCAATAACTGGCAGAACGGAATAGAGTTGTTAGATGGGGCT |
| | GAATGGAGGAACGGCGATATAGTTATCCCTGGAGGCGGCGGACCAG |
| | TAATTTCAAGCCCCTTGG |
| 107 | CCATTATTTGGTCTTGATATGGGTAACTTTTATTTATCATTCACAAAT |
| | GAATCCTTGTCTATGGCGGTAACTGTCGTTTTGGTGCCATCTTTATTT |
| | GGAGTTGTTACGAAAAAGGGCGGGGGAAAGTCAGTGCCAAATGCAT |
| | GGCAATCCTTGGTAGAGCTTATTTATGATTTCGTGCTGAACCTGGTA |
| | AACGAACAAATAGGTGGAAATGTTAAACAAAAGTTTTTCCCTCGCAT |
| | CTCGGTCACTTTTACTTTTTCGTTATTTCGTAATCCCCAGGGTATGAT |
| | ACCCTTTAGCTTCACAGTGACAAGTCATTTTCTCATTACTTTGGCTCT |
| | TTCATTTTCCATTTTTATAGGCATTACGATCGTTGGATTTCAAAGACA |
| | TGGGCTTCATTTTTTTAGCTTCTTATTACCAGCGGGAGTCCCACTGCC |
| | ATTAGCACCTTTTTTAGTACTCCTTGAGCTAATCTCTCATTGTTTTCG |
| | TGCATTAAGCTCAGGAATACGTTTATTTGCTAATATGATGGCCGGTC |
| | ATAGTTCAGTAAAGATTTTAAGTGGGTTCGCTTGGACTATGCTATTTC |
| | TGAATAATATTTTCTATTTCATAGGAGATCTTGGTCCCTTATTTATAG |
| | TTCTAGCATTAACCGGTCTGGAATTAGGTGTAGCTATATTACAAGCTC |
| | ATGTTTCTACGATCTCAATTTGTATTTACTTGAATGATGCTATAAATC |
| | TCCATCAAAATGAGTAATTTCATAATTGAATAAAAACGAGGAGCCGA |
| | AGATTTTAGGGGGCGGGACAAACGCGGAAGTGTATTGCGTTACAAAAAATG |
| | ACAACTAGCATTTGTTTTTTCATTTCATGTTCGAATT |
| 108 | AGCAATAGGTATTACAGTAGGGATAACAGGGTAATGAATTCGAACCATCTTC |
| | TCACTCTGACCCCCACATATCAGATCCCAGATGCATAGAAAAAGCGG |
| | TATCAAGAATAGTAGTATAAAGAAAGATAGTACAGTACTCAAGTAAA |
| | TGAATTCGCCTAAGGATCGATGGAAAGATCAAGGTCCCCGTGAAAAA |
| | GTAGATACTAGATCGATATGATACTCTCATCTCTGGAGTAACTTCTTC |
| | CATTATGCTGATCTCTAGGTCCGTTCCATCATCATCGTAATAGTATGG |
| | TCCCAGGTGTCCGAGCTATAGATCAAGATCATATCCAGTCACATTTCT |
| | ACCGGTGCACTTCTCATGAAATAATTCCCTTTCCAAGGAAAGGAAAA |
| | CAAGAACTCGAATACTCGTAATAGCGATCCCGATCCACCTACTTTTTT |
| | CTATTCTTTGATTCGAAACGTGCTAAAGCACAAGCCATTTTTATGCAT |
| | GGGGCATAAGAGTGGACAATCTATGTTATCGAAGGAAGTAAATAACA |
| | ACACTTCAGCGTTTAGGTCTACCTTCAGTAAACCAATAGTTTTGCAGC |
| | ATTGGAATTTGAGTTGGCCAGGTAAGGTCCTCTAAAAAGAAAAGAAG |
| | AAACTACTTAGAATAGATAAATGCCATTGGTTTTCTCGTACTATACGA |
| | TCTTTTTTTGTTTTGTTTTTTGGCCATGATTGTGCTGCTCCTGTGAAG |
| | GCTAGTGGGAAAGCTCACCGTTCGTTGTGATGAGTGGGGGCCTTGTA |
| | TCTGTATTCGGATCAGCTCCTTAACAGAGTTTCCTGCTTGAACCCTGG |
| | CTGGGNNCTGGGAGAGGTGTCCCACTACAGNTGCAAATAAACCATTT |
| | GACCTTACAGGGGAAAGGAAACAAACCACTCAATAATCGGTAGAAAT |
| | TCCTCCTACTGAACAGCTTTCCTTTTCTCGCCTTAACTACTACTTCAA |
| | AGCAAGGCGNANATCACGGGATAGGAATGAAANNACTTCTTACTCAC |
| | TTTCTAGCTATATAAAAATAGTTAGCANATGAAACGAGTAACTTAAGC |
| | CCTAGTAAAAGGCTACTCTTTGAATCCCCTCTTTAAGGCATATAAAAT |
| | TAGTACTCTTCCTGAGCTAGCTTAAGCATATCTTGAGCGAGTGAGTT |
| | GTATTTCCCTCCATCAAGTTCTAAGCGATCGAATAAGGTCCTTGCTCT |
| | CGAGCCAATGCCAATACCAATAGAGAGGGTCTAAACGAAGGATTCAA |
| | AGGAGGTTACCGGTTAAGGGCAAATGCAATCTCTAAGCCAGTTGGAGAA |
| | GAAAGGTAATCAAAGAAAGTTTCAGTTGGACTTACCCTGCCAACCCTACC |
| | CTAATTTCATACCCTGCGGTTTTCGT |
| 109 | TGGATCAATTTTTCATTGATCCATTATTTGGTCTTGATATGGGTAACTTTT |
| | ATTTATCATTCACAAATGAATCNTTGTCTATGGCGGTAACTGCCGTTTTGG |
| | TGCCATCTTTATTTGGAGTTGTTACGAAAAAGGGGGGGGGAAAGTCAGTG |
| | CCAAATGCATGGCAATCCTTGGTAGAGCTTATTTATGATTTCGTGCTGAA |
| | CCTGGTAAACGAACAAATAGGTGGAAATGTTAAACAAAAGTTTTTCC |
| | CTCGCATCTCGGTCACTTTTACTTTTTCGTTATTTCGTAATCCCCAGG |
| | GTATGATACCCTTTAGCTTCACAGTGACAAGTCATTTTCTCATTACTT |
| | TGGCTCTTTCATTTTCCATTTTTATAGGCATTACGATCGTTGGATTTC |
| | AAAGACATGGGCTTCATTTTTTTAGCTTCTTATTACCAGCGGGAGTCC |
| | CACTGCCATTAGCACCTTTTTTAGTACTCCTTGAGCTAATCTCTCATT |
| | GTTTTCGTGCATTAAGCTCAGGAATACGTTTATTTGCTAATATGATGG |
| | CCGGTCATAGTTCAGTAAAGATTTTAAGTGGGTTCGCTTGGACTATG |
| | CTATTTCTGAATAATATTTTCTATTTCATAGGAGATCTTGGTCCCTTA |
| | TTTATAGTTCTAGCATTAACCGGTCTGGAATTAGGTGTAGCTATATTA |
| | CAAGCTCATGTTTCTACGATCTCAATTTGTATTTACTTGAATGATGCT |
| | ATAAATCTCCATCAAAATGAGTAATTTCATAATTGAATAAAAACGAGG |
| | AGCCGAAGATTTTAGGGGGCGGGACAAACTCGGAAGTGTATTGCGTTACA |
| | AAAAATGACAACTAGCATTTGTTTTTTCATTTCATGTTCGAATTC |
| 110 | CAATAGGTATTACAGTAGGGATAACAGGGTAATGAATTCGAAACCATCTTCT |
| | CACTCTGACCCCCACATATCAGATCCCAGATGCATAGGAAAAGCGGT |
| | ATCAAGAATAGTAGTATAAAGAAAGATAGTACAGTACTCAAGTAAAT |
| | GAATTCGCCTAAGGATCGATGGAAAGATCAAGGTCCCCGTGAAAAAG |
| | TAGATACTAGATCGATATGATACTCTCATCTCTGGAGTAACTTCTTCC |
| | ATTATGCTGATCTCTAGGTCCGTTCCATCATCATCGTAATAGTATGGT |
| | CCCAGGTGCCCGAGCTATAGATCAAGATCATATCCAGTCACATTTCT |
| | ACCGGTGCACTTCTCATGAAATAATTCCCTTTCCAAGGAAAGGAAAA |
| | CAAGAACTCGAATACTCGTAATAGCGATCCCGATCCACCTACTTTTTT |
| | CTATTCTTTGATTCGAAACGTGCTAAAGCACAAGCCATTTTTATGCAT |
| | GGGGCATAAGAGTGGACAATCTATGTTATCGAAGGAAGTAAATAACA |
| | ACACTTCAGCGTTTAGGTCTACCTTTCAGTAAACCAATAGTTTTGCAG |
| | CATTGGAATTTGAGTTGGCCAGGTAAGGTCCTCTAAAAAGAAAAGAA |
| | GAAACTACTTAGAATAGATAAATGCCATTGGTTTTCTCGTACTATACG |
| | ATCTTTTTTTGTTTTGTTTTTTGGCCATGATTGTGCTGCTCCTGTGAA |
| | GGCTAGTGGGAAAGCTCACCGTTCGTTGTGGTGAGTGGGGGCCTTGT |
| | ATCTGTATTCGGATCAGCTCCTTAACAGAGTTTCCTGCTTGAACCCTG |
| | GCTGGGAGCTGGGAGAGGTGTCCCACTACAGGTGCAAATAAACCATT |
| | TGACCTTACAGGGGAAAGGAAACAAACCACTCAATAATCGGTAGAAA |
| | TTCCTCCTACTGAACAGCTTTCCTTTTCTCGCCTTAACTACTACTTCA |
| | GAGCAAGGCGGAATATCACGGGATAGGAATGAAAGAACTTCTTACTC |
| | AACTTTCTAGCTATATAAAAATAGTTAGCAATATGAAACGAGTAACTT |
| | AAGCCCTAGTAAAAGGCTACTCTTTGAATCCCCTCTTTAAGGCATATA |
| | AAATTAGTACTCTTCCTGAGCTAGCTTAAGCATATCTTGAGCGAGTGAG |
| | TTGTATTTCCCTCCATCAAGTTCTAAGCGATCAAATAAGGTCCTTGCTCTC |
| | GAGCCAATGCCAATACCAATAGAGAGGGTCTAAACGAAGGATTCAAAGG |
| | GGGTTACCGGTTAAGGGCAAATGCAATCTCTAAGCCAGTTGGAGAAGAA |
| | AGGTAGTCAAAGAAAGTTTCAGTTGGACTTACCCTGCCAACCCTACCCTA |
| | ATTTCATACCCTGCGGTTTTCGTTCCTCTAT |
| 111 | MFKQASRLLSRSVAAASSKSVTTRAFSTELPSTLDSPPTKRFRIQAKNIFLTYP |
| | QCSLSKEEALEQIQRIQLSSNKKYIKIARELHEDGQPHLHVLLQLEGKVQITNI |
| | RLFDLVSPTRSAHFHPNIQRAKSSSDVKSYVDKDGDTIEWGEFQIDGRSARG |
| | GQQTANDSYAKALNATSLDQALQILKEEQPKDYFLQHHNLLNNAQKIFQRPP |
| | DPWTPLFPLSSFTNVPEEMQEWADAYFGVDAAARPLRYNSIIVEGDSRTGKT |
| | MWARSLGAHNYITGHLDFSPRTYYDEVEYNVIDDVDPTYLKMKHWKHLIG |
| | AQKEWQTNLKYGKPRVIKGGIPCIILCNPGPESSYQQFLEKPENEALKSWTLH |
| | NSTFCKLQGPLFNNQAAASSQGDSTL |

1.-152. (canceled)

153. A method for transforming a mitochondrion of a cell, the method comprising:
a) introducing a first polynucleotide encoding a first polypeptide into the mitochondrion, wherein the cell is a plant cell or an algal cell, wherein the first polypeptide is a variant of a naturally occurring polypeptide, wherein the naturally occurring polypeptide comprises an enzyme activity that is inhibited by an herbicide, wherein the variant of the naturally occurring polypeptide comprises an enzyme activity that is resistant to the herbicide;
b) growing the cell under conditions wherein the first polypeptide is expressed;
c) growing the cell in a medium wherein the herbicide is present at an effective concentration; and
d) selecting a transformed cell comprising a transformed mitochondrion, wherein the transformed mitochondrion comprises the first polynucleotide.

154. The method of claim 153, wherein the enzyme activity of the naturally occurring polypeptide comprises an acetolactate synthase (ALS) activity, 5-enol-pyruvyl-shikimate-3-phosphate synthase (EPSPS) activity, glutamine synthetase (GS) activity, or any combination thereof.

155. The method of claim 154, wherein the enzyme activity of the naturally occurring polypeptide is acetolactate synthase activity and the herbicide is an inhibitor of acetolactate synthase.

156. The method of claim 155, wherein the first polypeptide comprises an amino acid sequence having at least 70% sequence identity to SEQ ID NO: 24.

157. The method of claim 156, wherein the first polypeptide comprises the amino acid sequence of SEQ ID NO: 24.

158. The method of claim 156, further comprising introducing into the mitochondrion a donor DNA comprising a cytoplasmic male sterility (CMS) coding region.

159. A method for transforming a mitochondrion of a cell, the method comprising:
a) introducing into the mitochondrion, wherein the cell is a plant cell or an algal cell:
i) a first polynucleotide encoding a first polypeptide, wherein the first polypeptide is a variant of a naturally occurring polypeptide, wherein the naturally occurring polypeptide comprises an enzyme activity that is inhibited by an herbicide, wherein the variant of the naturally occurring polypeptide comprises an enzyme activity that is resistant to the herbicide, and
ii) a second polynucleotide encoding a selectable marker, wherein the selectable marker enables the cell to grow in the presence of a selective agent, wherein the second polynucleotide does not encode the first polypeptide;
b) growing the cell under conditions wherein the selectable marker is expressed;
c) growing the cell in a medium comprising the selective agent, wherein the selective agent is present at an effective concentration; and
d) selecting a transformed cell comprising a transformed mitochondrion, wherein the transformed mitochondrion comprises the first polynucleotide, and further wherein the transformed cell comprising the transformed mitochondrion is capable of growing in a medium comprising the herbicide when the herbicide is present at an effective concentration.

160. The method of claim 159, wherein the selective agent is toxic to the cell.

161. The method of claim 160, wherein the selectable marker is a phosphite dehydrogenase enzyme or a biologically active fragment thereof, and wherein the selective agent is a phosphite.

162. The method of claim 159, wherein the enzyme activity of the naturally occurring polypeptide comprises acetolactate synthase (ALS) activity, 5-enol-pyruvyl-shikimate-3-phosphate synthase (EPSPS) activity, glutamine synthetase (GS) activity or any combination thereof.

163. The method of claim 162, wherein the enzyme activity of the naturally occurring polypeptide is acetolactate synthase activity and the herbicide is an inhibitor of acetolactate synthase.

164. The method of claim 163, wherein the first polypeptide comprises an amino acid sequence having at least 70% sequence identity to SEQ ID NO: 24.

165. The method of claim 163, wherein the first polypeptide comprises the amino acid sequence of SEQ ID NO: 24.

166. The method of claim 164, wherein the method further comprises:
introducing into the mitochondrion an mALS-SS polynucleotide encoding a regulatory subunit of an acetolactate synthase or a biologically active fragment thereof, and
growing the cell under conditions wherein the regulatory subunit of the acetolactate synthase or the biologically active fragment thereof is expressed.

167. The method of claim 164, wherein the method further comprises,
introducing into a nucleus of the cell a nMTS-ALS-SS polynucleotide encoding a modified regulatory subunit of an acetolactate synthase or a modified biologically active fragment thereof, wherein the modified regulatory subunit of the acetolactate synthase or the modified biologically active fragment thereof comprises a regulatory subunit of the acetolactate synthase or a biologically active fragment thereof operably linked to a mitochondrial targeting peptide, and
growing the cell under conditions wherein the modified regulatory subunit of the acetolactate synthase or the modified biologically active fragment thereof is expressed.

168. The method of claim 167, wherein the modified regulatory subunit of the acetolactate synthase or the modified biologically active fragment thereof comprises an amino acid sequence having at least 70% sequence identity to SEQ ID NO: 21.

169. The method of claim 168, wherein the modified regulatory subunit of the acetolactate synthase or the modified biologically active fragment thereof comprises the amino acid sequence of SEQ ID NO: 21.

170. The method of claim 164, further comprising introducing into the mitochondrion a donor DNA comprising a cytoplasmic male sterility (CMS) coding region.

171. A method comprising growing a plurality of plants in a presence of an herbicide that is an inhibitor of a plant enzyme, wherein at least one plant of the plurality of plants comprises a mitochondrion comprising a heterologous polynucleotide that encodes a variant of the plant enzyme, wherein the variant of the plant enzyme has an enzyme activity resistant to the herbicide, wherein the presence of the herbicide is sufficient to selectively promote growth of the at least one plant of the plurality of plants, resulting in an increased growth of the at least one plant of the plurality of plants relative to plants lacking the heterologous polynucleotide.

172. The cell of claim 171, wherein the plant enzyme is acetolactate synthase and the variant of the of the plant enzyme comprises an amino acid sequence having at least about 80% sequence identity to SEQ ID NO: 24.


SEQUENCE LISTING (111 sequences)

SEQ ID NO: 1 - protein, 9 aa, synthetic construct
LAGLIDADG

SEQ ID NO: 2 - protein, 36 aa, synthetic construct
DEVDFQGPCNDSSDPLVVAASIIGILHLILWILDRL

SEQ ID NO: 3 - protein, 4 aa, synthetic construct
DEVD

SEQ ID NO: 4 - RNA, 27 nt, Triticum aestivum
  source 1..27: mol_type = genomic RNA
acttttgacagttatacgattccagaa

SEQ ID NO: 5 - RNA, 27 nt, Triticum aestivum
  source 1..27: mol_type = genomic RNA
tgggctgtaccttcctcaggtgtcaaa

SEQ ID NO: 6 - RNA, 27 nt, Triticum aestivum
  source 1..27: mol_type = genomic RNA
gctgtacctggtcgttcaaatcttacc

SEQ ID NO: 7 - protein, 644 aa, Oryza sativa
MATTAAAAAAALSAAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 8 - DNA, 54 bp, Oryza sativa
  source 1..54: mol_type = genomic DNA
atggctacgaccgccgcggccgcggccgccgccctgtccgccgccgcgacggcc

SEQ ID NO: 9 - DNA, 168 bp, Arabidopsis thaliana
  source 1..168: mol_type = genomic DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatg

SEQ ID NO: 10 - DNA, 2049 bp, synthetic construct
  source 1..2049: mol_type = other DNA
atggccgccaagatccgcatcgtgatgaaatcttttatgagccaagctaacaaagttgaaggggttattccatacgcgcagaaggttggattgcctgaatcacgatccttgtataccgtgctacgatcgcctcacatcgacaagaagtcgagggagcagttctcgatgaagaccggccgtaagaaccaccagcgacaccacgtccttcccgctcgaggccgggtgggggcggcggcggtcaggtgctcggcggtgtccccggtcaccccgccgtccccggcgccgccggccacgccgctccggccgtgggggccggccgagccccgcaagggcgcggacatcctcgtggaggcgctggagcggtgcggcgtcagcgacgtgttcgcctacccgggcggcgcgtccatggagatccaccaggcgctgacgcgctccccggtcatcaccaaccacctcttccgccacgagcagggcgaggcgttcgcggcgtccgggtacgcgcgcgcgtccggccgcgtcggggtctgcgtcgccacctccggccccggggcaaccaacctcgtgtccgcgctcgccgacgcgctgctcgactccgtcccgatggtcgccatcacgggccaggtcccccgccgcatgatcggcaccgacgccttccaggagacgcccatagtcgaggtcacccgctccatcaccaagcacaattaccttgtccttgatgtggaggacatcccccgcgtcatacaggaagccttcttcctcgcgtcctcgggccgtcctggcccggtgctggtcgacatccccaaggacatccagcagcagatggccgtgccggtctgggacacctcgatgaatctaccagggtacatcgcacgcctgcccaagccacccgcgacagaattgcttgagcaggtcttgcgtctggttggcgagtcacggcgcccgattctctatgtcggtggtggctgctctgcatctggtgacgaattgcgctggtttgttgagctgactggtatcccagttacaaccactctgatgggcctcggcaatttccccagtgacgacccgttgtccctgcgcatgcttgggatgcatggcacggtgtacgcaaattatgccgtggataaggctgacctgttgcttgcgtttggtgtgcggtttgatgatcgtgtgacagggaaaattgaggcttttgcaagcagggccaagattgtgcacattgacattgatccagcagagattggaaagaacaagcaaccacatgtgtcaatttgcgcagatgttaagcttgctttacagggcttgaatgctctgctacaacagagcacaacaaagacaagttctgattttagtgcatggcacaatgagttggaccagcagaagagggagtttcctctggggtacaaaacttttggtgaagagatcccaccgcaatatgccattcaggtgctggatgagctgacgaaaggtgaggcaatcatcgctactggtgttgggcagcaccagatgtgggcggcacaatattacacctacaagcggccacggcagtggctgtcttcggctggtctgggcgcaatgggatttgggctgcctgctgcagctggtgcttctgtggctaacccaggtgtcacagttgttgatattgatggggatggtagcttcctcatgaacattcaggagctggcattgatccgcattgagaacctccctgtgaaggtgatggtgttgaacaaccaacatttgggtatggtggtgcaattggaggataggttttacaaggcgaatagggcgcatacatacttgggcaacccggaatgtgagagcgagatatatccagattttgtgactattgctaaggggttcaatattcctgcagtccgtgtaacaaagaagagtgaagtccgtgccgccatcaagaagatgctcgagactccagggccatacttgttggatatcatcgtcccgcaccaggagcatgtgctgcctatgatcccaattgggggcgcattcaaggacatgatcctggatggtgatggcaggactgtgtattaa

SEQ ID NO: 11 - protein, 682 aa, synthetic construct
MAAKIRIVMKSFMSQANKVEGVIPYAQKVGLPESRSLYTVLRSPHIDKKSREQFSMKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 12 - DNA, 2015 bp, synthetic construct
  source 1..2015: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcc

SEQ ID NO: 13 - DNA, 253 bp, Rhizobium radiobacter
  source 1..253: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 14 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 15 - DNA, 781 bp, synthetic construct
  source 1..781: mol_type = other DNA
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 16 - DNA, 209 bp, synthetic construct
  source 1..209: mol_type = other DNA
gatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 17 - protein, 476 aa, Oryza sativa
MATATAASRLAVAGAEPARARRHRPTTVAVCGGARPRSRPAAVVAAAGAAAPSPATGGVAPVPPSPRGSIIKRHTLSVFVGDESGMINRIAGVFARRGYNIESLAVGLNKDKALFTIVVSGTEKILKQVVEQLNKLVNVIQVDDLSKEPQVERELMLIKLNVEPDKRPEVMGLVDIFRAKVVDLSDHTLTIEVTGDPGKIVAVQRNLSKFGIKEIARTGKIALRREKMGESAPFWRFSAASYPDLEVAMPSKSHVNTAMKTANQNSEESSQGDVYPVESYENFTTNQILDAHWGVMADGDPTGLCSHSLSILVNDFPGVLNVVTGVFSRRGYNIQSLAVGPAEKEGTSRITTVVPGTDESIAKLVHQLYKLIDVYEVQDLTHLPFAARELMIIKIAVNTTARRAILDIADIFRAKTVDVSDHTVTLQLTGDLDKMVALQRMLEPYGICEVARTGRVALRRESGVDSKYLRGFSLPL

SEQ ID NO: 18 - protein, 47 aa, Oryza sativa
MATATAASRLAVAGAEPARARRHRPTTVAVCGGARPRSRPAAVVAAA

SEQ ID NO: 19 - DNA, 108 bp, Arabidopsis thaliana
  source 1..108: mol_type = genomic DNA
atgtttaaacaagcttctcgtctcctctcccgatctgtcgccgccgcatcttccaaatcggtgacgactcgtgccttttcaacggaacttccatcgacgctcgattcc

SEQ ID NO: 20 - DNA, 1398 bp, synthetic construct
  source 1..1398: mol_type = other DNA
atgtttaaacaagcttctcgtctcctctcccgatctgtcgccgccgcatcttccaaatcggtgacgactcgtgccttttcaacggaacttccatcgacgctcgattccggcgccgccgctccctctccggccaccggcggcgtcgcgccggttccgccaagccccaggggctcgattataaagcgtcatacactatcagtttttgttggagatgaaagtgggatgattaatcgaattgctggagtttttgctagaagaggatacaacattgagtcattggcagttgggctgaacaaggacaaggcattatttacaatagtagtatctggaactgagaaaatactgaaacaggttgtagagcaactaaacaaacttgttaatgttatacaggttgacgatctgtcgaaggaaccacaagttgaaagagagctaatgcttataaaactaaatgttgagccagataagcgaccggaggtgatgggtttggttgacatcttcagagcaaaggtggttgacctttctgaccacacactaactattgaggtaactggagatcctggaaaaattgttgctgtacagaggaacctaagcaaatttggaatcaaagaaattgccagaactggcaagatagctttgcggcgtgagaaaatgggagaaagtgctcctttttggcggttctctgcagcttcctatcctgatcttgaagtggcaatgccatcaaaatctcatgtcaacactgcgatgaaaacagctaaccagaattctgaagaatcttcacaaggtgatgtctatccagtggagtcatatgaaaacttcacaacaaatcaaattcttgatgctcattggggtgtcatggctgacggtgatccaacagggctttgttcacattctttgtccattttggtgaatgatttccctggagttctcaatgttgtaacaggtgttttctcccgaagaggctacaatattcagagtctggctgttggcccagctgaaaaagaaggcacttctcggatcactactgttgtccctggaactgatgagtctattgccaagctagtacaccaactgtataagctcattgatgtttatgaggtccaggatcttactcatttaccatttgctgctagagagttaatgatcataaagattgctgtaaacaccacagcccgcagggctatcctagatattgctgatattttccgggccaaaactgtggatgtatcagatcacaccgtaactcttcagcttactggagaccttgataaaatggtcgcattacaaaggatgctcgagccctatggcatttgtgaggttgcacgaactggcagggttgcgttgcgccgtgagtcaggagtcgattccaaatacctccgtgggttttcccttcctctgtag

SEQ ID NO: 21 - protein, 465 aa, synthetic construct
MFKQASRLLSRSVAAASSKSVTTRAFSTELPSTLDSGAAAPSPATGGVAPVPPSPRGSIIKRHTLSVFVGDESGMINRIAGVFARRGYNIESLAVGLNKDKALFTIVVSGTEKILKQVVEQLNKLVNVIQVDDLSKEPQVERELMLIKLNVEPDKRPEVMGLVDIFRAKVVDLSDHTLTIEVTGDPGKIVAVQRNLSKFGIKEIARTGKIALRREKMGESAPFWRFSAASYPDLEVAMPSKSHVNTAMKTANQNSEESSQGDVYPVESYENFTTNQILDAHWGVMADGDPTGLCSHSLSILVNDFPGVLNVVTGVFSRRGYNIQSLAVGPAEKEGTSRITTVVPGTDESIAKLVHQLYKLIDVYEVQDLTHLPFAARELMIIKIAVNTTARRAILDIADIFRAKTVDVSDHTVTLQLTGDLDKMVALQRMLEPYGICEVARTGRVALRRESGVDSKYLRGFSLPL

SEQ ID NO: 22 - DNA, 1245 bp, Oryza sativa
  source 1..1245: mol_type = genomic DNA
tagctagcatactcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaag

SEQ ID NO: 23 - DNA, 618 bp, synthetic construct
  source 1..618: mol_type = other DNA
gagtttgaatcaaatcttcacttgtttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcggga

SEQ ID NO: 24 - protein, 631 aa, synthetic construct
MAATAKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVY

SEQ ID NO: 25 - DNA, 1896 bp, synthetic construct
  source 1..1896: mol_type = other DNA
atggctgcgactgccaagaccggccgtaagaaccatcaacgacatcatgtccttcccgctcgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaagcttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgctcgagactccagggccatatttgttggatatcatcgtcccacatcaagagcatgtgctgcctatgatcccaattgggggcgcattcaaggacatgatcctggatggtgatggcaggactgtgtattaa

SEQ ID NO: 26 - DNA, 18 bp, unidentified
  source 1..18: mol_type = unassigned DNA
taatacgactcactatag

SEQ ID NO: 27 - DNA, 919 bp, synthetic construct
  source 1..919: mol_type = other DNA
tctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagt

SEQ ID NO: 28 - DNA, 41 bp, unidentified
  source 1..41: mol_type = unassigned DNA
aaccccttggggcctctaaacgggtcttgaggggttttttg

SEQ ID NO: 29 - DNA, 774 bp, synthetic construct
  source 1..774: mol_type = other DNA
aaccccttggggcctctaaacgggtcttgaggggttttttgtagatagacctttttatttttcgtcattcgatcacgaaaacagggattctggaacggccaagaatcccagcggttgttcgggtcgaaaaaccgagaacaagacatgccacaaagtggcagatgaaggcaggggggagagcctagtcctcaacctcttcttccccaaaaggtagttatgaacgtgccaaacttattggatttattcttggaatgctcataaccacctttactctttttttcattctttactcagaggaagccatgccgtttggagaagagcaccaagtggggggagtgtggagtccccgaaagaggagctttctaaaggcaagagaaaagctccgatggagcccttggagctacagggaccaccaacccttcgcagcttggacgatttgattcttgtgccactcagccctgaggagggcgcctgctcgacccagtcaggtactactccgccgccggccccgagtaactctgcgggggtcggtgcagccctttcttctattccggagtgcataaacaaggatcctcaaaaagcgaaatcatttcgttaatggcatttcagaaatgagtcataggcgcctgtacaatgacagaatagagagtcctttttttccagaatgaatcattctattcaaatctcacaagttctctttacgcgtcttctaggggcattgttgaacgcaatctgcaggaacaagaaatgattctttcttattttgaaacagaa

SEQ ID NO: 30 - DNA, 744 bp, synthetic construct
  source 1..744: mol_type = other DNA
acttttgacagttatacgattccagaagtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 31 - DNA, 899 bp, Oryza sativa
  source 1..899: mol_type = genomic DNA
ggggtcatcccattggccagactgagcatcaagccagccaagaagtaaaagctgagaaggagtgactcgcatgagtcaacacttactactcaggtccggtagagcaatctcaaattatcatatagaaatgttaatgttatgatttcggtattgatcaaaaggtgctgggaccttagggcatacattagtgccatgccctattgcggaacggtcgtatcctggtaacctagcccccgtaagagctctacctaatcgtcggggtagaaggctgtgcttattctcggcaaatagctaagtcgacaccccgagggagcaactcaactcttcgtagatcaaaacaagtgttcactggaaagtggatcaaagaaaaaaacttcttcgtttcgttggaaaaaccgacgccaatatcatattgactctctctcgtccaataagagtttccgagagttactttattcaaattctctcctttccaaagctccacaaggcaggcaaaaagagtaataggacaacaagcaatcttgtctttcatttatttggagttctttctttgttgagatggaaatcgacgttcttttgaaaagggctaggtagtttgcacgcaggcaaaacttcttcatgaaaggtaataaatagacttttttttcatgggtttcttaatgactagtcgttcgtttgaagccttaagaaaccggcagttttttttccgaatgaccttatttcgagaatcaactaaccgacaaatccgtagcccaggtgattcgctgcctccctctcgccaaaatgggatgaatcttctcatgcagcttttttcttgttcagggcgcagcgaagccaatttccatcaaggcaagggggtaaataagggggaagaggagttgtcacgatagaaaagagaa

SEQ ID NO: 32 - DNA, 182 bp, Oryza sativa
  source 1..182: mol_type = genomic DNA
tagacggatgagactgatcacacctgatcagtgatcaattctggcacaatgaatttacgagttattttacacaatgaatttacaagcagatgagtttgcaacggtagacctatctcctgaaaagagttcagtaaacaagggaacgaagcgaccgataacgtcccctcggggaggagtgtttt

SEQ ID NO: 33 - DNA, 2757 bp, synthetic construct
  source 1..2757: mol_type = other DNA
atgtttaaacaagcttctcgtctcctctcccgatctgtcgccgccgcatcttccaaatcggtgacgactcgtgccttttcaacggaacttccatcgacgctcgattccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 34 - protein, 918 aa, synthetic construct
MFKQASRLLSRSVAAASSKSVTTRAFSTELPSTLDSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 35 - protein, 36 aa, Arabidopsis thaliana
MFKQASRLLSRSVAAASSKSVTTRAFSTELPSTLDS

SEQ ID NO: 36 - DNA, 1615 bp, synthetic construct
  source 1..1615: mol_type = other DNA
caagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcggga

SEQ ID NO: 37 - DNA, 57 bp, synthetic construct
  source 1..57: mol_type = other DNA
gtctggccccaaattctaatttctactgttgtagatttcaattatgaaattactcat

SEQ ID NO: 38 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 39 - DNA, 1210 bp, synthetic construct
  source 1..1210: mol_type = other DNA
gaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaa

SEQ ID NO: 40 - DNA, 57 bp, synthetic construct
  source 1..57: mol_type = other DNA
gtctggccccaaattctaatttctactgttgtagatgtcacatttctaccggtgcac

SEQ ID NO: 41 - DNA, 41 bp, Oryza sativa
  source 1..41: mol_type = genomic DNA
gaatttctctgacattccatgtttccgaaacggatcctata

SEQ ID NO: 42 - DNA, 40 bp, Oryza sativa
  source 1..40: mol_type = genomic DNA
tgaacagtcactcacttttgacagttatacgattccagaa

SEQ ID NO: 43 - DNA, 81 bp, synthetic construct
  source 1..81: mol_type = other DNA
aaccccttggggcctctaaacgggtcttgaggggttttttgtagatagacctttttatttttcgtcattcgatcacgaaaa

SEQ ID NO: 44 - DNA, 6837 bp, synthetic construct
  source 1..6837: mol_type = other DNA
gggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagtgaatttctctgacattccatgtttccgaaacggatcctataaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaatgaggtaccaagcgatcgcaaacctaggaaaagatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcgcc

SEQ ID NO: 45 - DNA, 6836 bp, synthetic construct
  source 1..6836: mol_type = other DNA
gggcccaaattcaattgtatatgagctcatatacaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattagggataacagggtaataatcacaagtgagaaccacaggtagcaataggtattacagaaatttcctcgagtctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaatacgactcactatagtaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagttgaacagtcactcacttttgacagttatacgattccagaaaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtattaatgaggtaccaagcgatcgcaaacctaggaaaagatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatctaaaaagcttaagcggccgcaaaaaccccttggggcctctaaacgggtcttgaggggttttttgtagatagacctttttatttttcgtcattcgatcacgaaaaggccggccaaacctcgaggtaattaaagcggccgaaattagctagcaaataagcatgcaatcacaagtgagaaccacaggtagcaataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggcgcgcc

SEQ ID NO: 46 - DNA, 457 bp, Oryza sativa
  source 1..457: mol_type = genomic DNA
caaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaat

SEQ ID NO: 47 - protein, 79 aa, Oryza sativa
MANLVRWLFSTTRGTNGLPYFIFGVVVGGALLFALLKYQAPLYDPALMEKIIDHNIKAGHPIEVDYSWWGTSIRVVFPK

SEQ ID NO: 48 - DNA, 352 bp, synthetic construct
  source 1..352: mol_type = other DNA
agatctagacgaggtgtagcgcagtctggtcagcgcatctgttttgggtacagagggccataggttcgaatcctgtcaccttgagtctggccccaaattctaatttctactgttgtagatttcaattatgaaattactcatgtccctttcgtccagtggttaggacatcgtcttttcatgtcgaagacacgggttcgattcccgtaagggatagtctggccccaaattctaatttctactgttgtagatgtcacatttctaccggtgcacattccagcttatttgatacccacttcaagtttctatcaaaccatgtctttttcttcgaacgtcaatctcgtaggaaagatct

SEQ ID NO: 49 - DNA, 41 bp, Oryza sativa
  source 1..41: mol_type = genomic DNA
gtagggctttctgaggagtaagcctaattccgttaatgcag

SEQ ID NO: 50 - DNA, 27 bp, Oryza sativa
  source 1..27: mol_type = genomic DNA
gagagacttgtttggggtcggcgttgg

SEQ ID NO: 51 - DNA, 30 bp, Oryza sativa
  source 1..30: mol_type = genomic DNA
agtgctcagaataatccaggtcgctcgacg

SEQ ID NO: 52 - DNA, 38 bp, synthetic construct
  source 1..38: mol_type = other DNA
accacaggtagcaataggtattacagtagggataacag

SEQ ID NO: 53 - DNA, 771 bp, Triticum aestivum
  source 1..771: mol_type = genomic DNA
atgacaaatatggttcgatggctcttctccactagcaggtttactgctttctatttgcacttttgtattaagtttccttatatatacgattttttattattttctatttgtctatttttctttttagtgcgttttatttcgattattcttctcccaatttgcaatcttttcggagcctccttcattattactcttcctccagagattcaggatccccaagctctagctcatttagcagggctaaacttctatctgagcctttacgagcaggatcctggatgggttacgttcattcagaacgagcttaatcacaatacccctctggaggacatacctggacggcttaagctcttcctaatggaagaaaagctgtctagtatgcgacaagatgtcattcaggaatttgtggcgctttatcaaagaatagggccttatctaccgatcgagccctacttggtcgatgaagcgcttcgttcctatctggaccatattcacgcaactgattctttcactgttctccaagcgtcttatcaagatctgcgggagaatgagggaggatctgttttctttagagatgctgtttcccacaaccgggatctccttgaggcggaaagctccgcaaggaggtgcctggaagtggaacagaggatccgatgggaagaaatccccaagagcaaggcaagtctcgaaagagctgagcacgagcatgctctcgacttgtttaagtcggaggatcttagaagggaattagaaaaaaaaagagcggggtag

SEQ ID NO: 54 - protein, 256 aa, Triticum aestivum
MTNMVRWLFSTSRFTAFYLHFCIKFPYIYDFLLFSICLFFFLVRFISIILLPICNLFGASFIITLPPEIQDPQALAHLAGLNFYLSLYEQDPGWVTFIQNELNHNTPLEDIPGRLKLFLMEEKLSSMRQDVIQEFVALYQRIGPYLPIEPYLVDEALRSYLDHIHATDSFTVLQASYQDLRENEGGSVFFRDAVSHNRDLLEAESSARRCLEVEQRIRWEEIPKSKASLERAEHEHALDLFKSEDLRRELEKKRAG

SEQ ID NO: 55 - DNA, 840 bp, Triticum aestivum
  source 1..840: mol_type = genomic DNA
atgcctcaacttgataaattaacttatttctcacaattcttctggttatgtcttctcctctttactttttatattctcttatttaataataataatggaatacttggaattagtagaattctcaaactacggaaccaactgctttcgcaccgggggggcgagatccggagcaaggaccctaagaatctggaagatatctcgagaaaaggttttagcaccggtctctcatatatgtactccagtttatccgaagtatcccaatggtgtaagaccgtcgactatttgggaaataagatatcttcttcaatctttctatactatcttaggggcgtcctttgcccaatttgcctcttattttttaaatttcttatttctttcgccttcacctgtgcacttatttatgtattcaagggggggggtttcgtggctatggctgctacaaacggagcctcttcttcctttctggagagctcaggtgaaatagatgcactgttacaaacaacaacaacaacacgaacacccgaaaacgcggattacgaagacgataatacttctgtaaatcaagaattcctcgaagacggacaaagggcccgacaggctaaactacgggagttagaaagactcattctccagcaatataaggatttcatccgacagaaatatccatggatacctaaaggcgacatccttctcccgagcatgaagggtggagttgtcgaggacgtaatggaaaaattagaattggaaacgtattccagtagcgacttgactgattggatcaaccatttgcgggcaaatccgaaaacattaaattttatcttcaaggattttgtcgcgtga

SEQ ID NO: 56 - protein, 279 aa, Triticum aestivum
MPQLDKLTYFSQFFWLCLLLFTFYILLFNNNNGILGISRILKLRNQLLSHRGGEIRSKDPKNLEDISRKGFSTGLSYMYSSLSEVSQWCKTVDYLGNKISSSIFLYYLRGVLCPICLLFFKFLISFAFTCALIYVFKGGGFVAMAATNGASSSFLESSGEIDALLQTTTTTRTPENADYEDDNTSVNQEFLEDGQRARQAKLRELERLILQQYKDFIRQKYPWIPKGDILLPSMKGGVVEDVMEKLELETYSSSDLTDWINHLRANPKTLNFIFKDFVA

SEQ ID NO: 57 - DNA, 552 bp, Triticum timopheevii
  source 1..552: mol_type = genomic DNA
aataagatatcttcttcaatctttctatactatcttaggggcgtcctttgcccaatttgcctcttattttttaaatttcttatttctttcgccttcacctgtgcacttatttatgtattcaagggggggggtttcgtggctatggctgctacaaacggagcctcttcttcctttctggagagctcaggtgaaatagatgcactgttacaaacaacaacaacaacacgaacacccgaaaacgcggattacgaagacgataatacttctgtaaatcaagaattcctcgaagacggacaaagggcccgacaggctaaactacgggagttagaaagactcattctccagcaatataaggatttcatccgacagaaatatccatggatacctaaaggcgacatccttctcccgagcatgaagggtggagttgtcgaggacgtaatggaaaaattagaattggaaacgtattccagtagcgacttgactgattggatcaaccatttgcgggcaaatccgaaaacattaaattttatcttcaaggattttgtcgcgtga

SEQ ID NO: 58 - protein, 183 aa, Triticum timopheevii
NKISSSIFLYYLRGVLCPICLLFFKFLISFAFTCALIYVFKGGGFVAMAATNGASSSFLESSGEIDALLQTTTTTRTPENADYEDDNTSVNQEFLEDGQRARQAKLRELERLILQQYKDFIRQKYPWIPKGDILLPSMKGGVVEDVMEKLELETYSSSDLTDWINHLRANPKTLNFIFKDFVA

SEQ ID NO: 59 - DNA, 4945 bp, synthetic construct
  source 1..4945: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatggctagtgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatctactagcaaaccggtaacgttatacgacgtcgctgaatacgccggcgtttctcatcaaaccgtttctagagtggttaaccaggcttcacatgttagcgctaaaacccgggaaaaagttgaagctgccatggctgagctcaactacatcccgaaccgtgttgcgcagcagctggctggtaaacaaagcttgctgatcggtgtcgcgacctcgagcttggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccactagtggatcggaattcgctaacttcaaccagtccggaaacatcgctgattcttccttgagcttcactttcactaactcttctaacggacctaaccttatcaccactcagaccaactctcaggctcttagccagccaatcgctagctctaacgtgcacgacaacttcatgaacaacgagatcactgctagcaagatcgatgatggtaacaattctaagcctcttagcccaggatggactgatcagactgcttacaacgcattcggtatcactaccggtatgttcaacaccactaccatggacgatgtgtacaactacctcttcgacgatgaggatactccacctaaccctaagaaggagtgaactagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaacttaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaac

SEQ ID NO: 60 - DNA, 3149 bp, synthetic construct
  source 1..3149: mol_type = other DNA
gatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtaccctgcaggttatcacttgtgccccagtttgctagggaggtcgcagtatctggccacagccacctcgtgctgctcgacgtatgtctctttgtcggcctccttgattctttccagtctgtggtccacatagtagacgccgggcatcttgaggttcttagcgggtttcttggatctgtatgtggtcttgaagttgcagatcaggtggcccccgcccacgagcttcagggccatgtcgcttctgccttccaggccgccgtcagcggggtacagcatctcggtgttggcctcccagccgagtgttttcttctgcatcacagggccgttggatgggaagttcacccctctgatcttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgccctgggtgtggttgatgaaggttctgctgccgtacatgaagctggtagccaggatgtcgaaggcgaaggggagagggccgccctcgaccaccttgattctcatggtctgggtgccctcgtagggcttgccttcgccctcggatgtgcacttgaagtggtggttgttcacggtgccctccatgtacagcttcatgtgcatgttctccttaatcagctcgctcatggtgactcgagtcgacaagcttgctagctgtagttgtagaatgtaaaatgtaatgttgttgttgtttgttgttgttgttggtaattgttgtaaaaatacgcgcgtctagcttcagcgtgtcctctccaaatgaaatgaacttccttatatagaggaagggtcttgcgaagatcgatcctctagtctttcaattgtgagcgctcacaattctttctcttccctttcttctttactagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagatttcttgagctctctagtctttcaattgtgagcgctcacaattctttctcttccctttcttctttctagctccaccgcggtggcggccggccgctctagtggatcgatcttcgcaagacccttcctctatataaggaagttcatttcatttggagaggacacgctgaagctagacgcgcgtatttttacaacaattaccaacaacaacaacaaacaacaacaacattacattttacattctacaactacagctagagtcgagtggccaccatgtttaaacaagcttctcgtctcctctcccgatctgtcgccgccgcatcttccaaatcggtgacgactcgtgccttttcaacggaacttccatcgacgctcgattcccctcctactaaaagatttcgtattcaagcaaaaaacatatttcttacatatcctcagtgttctctttcaaaagaagaagctcttgagcaaattcaaagaatacaactttcatctaataaaaaatatattaaaattgccagagagctacacgaagatgggcaacctcatctccacgtcctgcttcaactcgaaggaaaagttcagatcacaaatatcagattattcgacctggtatccccaaccaggtcagcacatttccatccaaacattcagagagctaaatccagctccgacgtcaagtcctacgtagacaaggacggagacacaattgaatggggagaattccagatcgacggtagaagtgctagaggaggtcaacagacagctaacgactcatatgccaaggcgttaaacgcaacttctcttgaccaagcacttcaaatattgaaggaagaacaaccaaaggattacttccttcaacatcacaatcttttgaacaatgctcaaaagatatttcagaggccacctgatccatggactccactatttcctctgtcctcattcacaaacgttcctgaggaaatgcaagaatgggctgatgcatatttcggggttgatgccgctgcgcggcctttaagatataatagtatcatagtagagggtgattcaagaacagggaagactatgtgggctagatctttaggggcccacaattacatcacagggcacttagattttagccctagaacgtattatgatgaagtggaatacaacgtcattgatgacgtagatcccacttacttaaagatgaaacactggaaacaccttattggagcacaaaaggagtggcagacaaacttaaagtatggaaaaccacgtgtcattaaaggtggtatcccctgcattatattatgcaatccaggacctgagagctcataccaacaatttcttgaaaaaccagaaaatgaagcccttaagtcctggacattacataattcaaccttctgcaaactccaaggtccgctctttaataaccaagcagcagcatcctcgcaaggtgactctaccctgtaaaggcaaacaatgaatcaacaactctcctggcgcaccatcgtcggctacagcctcggtggggagtccgcaaatcaccagtctctctctacaaatctatctctctctattttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacc

SEQ ID NO: 61 - DNA, 2066 bp, synthetic construct
  source 1..2066: mol_type = other DNA
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 62 - protein, 445 aa, synthetic construct
AKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 63 - DNA, 1338 bp, synthetic construct
  source 1..1338: mol_type = other DNA
gcgaaggcggaggagatcgtgctccaacccatcagagagatttccggggcggttcaactgccagggtccaagtcgctctccaacagaatcctcctcctctccgccctctccgagggcacaactgtggtggacaacttgctgaacagtgaggatgttcattatatgcttgaggccctgaaagccctcgggctctctgtggaagcagataaagttgcaaagagagctgtagtcgttggctgtggtggcaagtttcctgttgagaaggatgcgaaagaggaagtgcaactcttcttggggaacgctggaattgcaatgcgatcattgactgctgccgtgactgctgctggtggaaatgcaacttatgtgcttgatggagtgccacgaatgagagagcgtcctattggtgatttggttgtcgggttgaaacaacttggtgcggatgttgactgtttccttggcactgaatgcccacctgttcgtgtcaagggaattggaggacttcctggtggcaaggttaagctctctggttccatcagcagtcaatatttgagtgccttgctgatggctgctcctttggcccttggggatgtggagatcgaaatcattgacaaactaatctccattccttatgttgaaatgactttgagattgatggagcgttttggtgtgaaggcagagcattctgatagttgggacagattctatattaagggagggcaaaagtataaatctcctggaaatgcctatgttgaaggtgatgcctcaagcgcgagctatttcttggctggtgctgcaatcactggaggcactgtgacagttcaaggttgtggtacaaccagtttgcaaggtgatgtcaaatttgctgaggtacttgagatgatgggagcaaaggttacatggactgacaccagtgtaacagtaactggtccacctagagagccttatgggaagaaacatctgaaagctgttgatgtcaacatgaacaaaatgcctgatgttgccatgacccttgccgttgttgcactcttcgctgatggtccaactgctatcagagatgtggcttcctggagagtaaaggaaactgaaagaatggttgcaattcgaactgagctaacaaaacttggagcatcggttgaagaaggtcctgactattgcatcatcactccacctgagaagctgaacattactgcaatcgacacttatgatgaccatagaatggctatggccttctccctcgctgcctgcgccgacgtgcccgtgactatccgtgaccctggttgcactagaaagacattccccaactatttcgacgttctaagcactttcgtcagaaactga

SEQ ID NO: 64 - DNA, 1379 bp, synthetic construct
  source 1..1379: mol_type = other DNA
gaatttctctgacattccatgtttccgaaacggatcctatagcgaaggcggaggagatcgtgctccaacccatcagagagatttccggggcggttcaactgccagggtccaagtcgctctccaacagaatcctcctcctctccgccctctccgagggcacaactgtggtggacaacttgctgaacagtgaggatgttcattatatgcttgaggccctgaaagccctcgggctctctgtggaagcagataaagttgcaaagagagctgtagtcgttggctgtggtggcaagtttcctgttgagaaggatgcgaaagaggaagtgcaactcttcttggggaacgctggaattgcaatgcgatcattgactgctgccgtgactgctgctggtggaaatgcaacttatgtgcttgatggagtgccacgaatgagagagcgtcctattggtgatttggttgtcgggttgaaacaacttggtgcggatgttgactgtttccttggcactgaatgcccacctgttcgtgtcaagggaattggaggacttcctggtggcaaggttaagctctctggttccatcagcagtcaatatttgagtgccttgctgatggctgctcctttggcccttggggatgtggagatcgaaatcattgacaaactaatctccattccttatgttgaaatgactttgagattgatggagcgttttggtgtgaaggcagagcattctgatagttgggacagattctatattaagggagggcaaaagtataaatctcctggaaatgcctatgttgaaggtgatgcctcaagcgcgagctatttcttggctggtgctgcaatcactggaggcactgtgacagttcaaggttgtggtacaaccagtttgcaaggtgatgtcaaatttgctgaggtacttgagatgatgggagcaaaggttacatggactgacaccagtgtaacagtaactggtccacctagagagccttatgggaagaaacatctgaaagctgttgatgtcaacatgaacaaaatgcctgatgttgccatgacccttgccgttgttgcactcttcgctgatggtccaactgctatcagagatgtggcttcctggagagtaaaggaaactgaaagaatggttgcaattcgaactgagctaacaaaacttggagcatcggttgaagaaggtcctgactattgcatcatcactccacctgagaagctgaacattactgcaatcgacacttatgatgaccatagaatggctatggccttctccctcgctgcctgcgccgacgtgcccgtgactatccgtgaccctggttgcactagaaagacattccccaactatttcgacgttctaagcactttcgtcagaaactga

SEQ ID NO: 65 - protein, 449 aa, synthetic construct
MDPIAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 66 - DNA, 901 bp, Oryza sativa
  source 1..901: mol_type = genomic DNA
tctgcttgaaagcctgcagagtccaattttgagtattttcagttagaatctagagtcagcctattcagttcttagcccttaagggtaaggcagggggtaatatggatagtctctgtccctgtattcacattccaccttcaacaaagtgttgatttcccgtaaagctaactgtagtcctttaagtaagtagatatcttaggcaagttagcaatctcgttatattaccaaggccttcccttctattgtagaaagagttctcagccatctaattgcagtgccagttgccagctatccagtttcatttgaagttgctgggggtccaaacgagctagttgcttttattcgtcctataagtccttccacaagcgagtcaatagggtgctggctagttgtagttgttggcgtgcctttcctttcatcttgaatattaataaatatttggataaattactttagaataagaagttcatgttttaagtaatacgaatccatactaggaaaatgaaaatgtgagtcctaggcactggaattggttctcttctccctaatccctataagccagaaagggtaataggcttcagtgtaagcatttccttcaagcaagtcatctcaagttttaaattctagagaatagctccgatcaacccattttagtttggttctgcaattcattcgcataaatgaaaaaaaaagcgagatgtgcacgaaagaagatcatagttcagctttaaaatggtggtgtccctgtgttagtaagtggttgaaatagctcatgggagtgtctgccccattcgataatggcatttatgatctagtggagtgagtgattgtgtggtgttcagtctaaggctttttgaaaagcggatttctcccttctctcatccatcgtctttgttaaagt

SEQ ID NO: 67 - DNA, 468 bp, synthetic construct
  source 1..468: mol_type = other DNA
tgaaaaagggaaagcatgcaaaagggagaggtaccaaagatctaaaaagcttaagcggccgcaaatagatagacctttttatttttcgtcattcgatcacgaaaaggccggccaaacctcgaggaccacaggtagcaataggtattacagtagggataacagggtaatgaaagacaggacagtggtggtttgctcatactttcattacaaaaccatactatggaattctggtggaccaggcaaatatcccacccctacaaggtgggagggcggccgggaacccgtggtggcggttcctcccgtgatgatcgactaatggataagtcgcaatgatcgactaatggataagtcgcattgagatattggttcgaatccaattcattacgagctcttctggagagcattatctctaagagaataaaatgaaaaacccagctaggagaaaaaaacagtaagttaacaagatag

SEQ ID NO: 68 - DNA, 1168 bp, synthetic construct
  source 1..1168: mol_type = other DNA
acgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttatagttctagcattaaccggttttgaattaggtgtagctatattacaagctcatgtttctacgatctcaatttgtatttacttgaatgatgctataaatctccatcaaaatgagtaatttcataattgaataaaaacgaggagccgaagattttagggggcggga

SEQ ID NO: 69 - DNA, 1203 bp, Oryza sativa
  source 1..1203: mol_type = genomic DNA
aaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatttagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaa

SEQ ID NO: 70 - DNA, 699 bp, Oryza sativa
  source 1..699: mol_type = genomic DNA
caaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaagaaagacaggacagtggtggtttgctcatactttcattacaaaaccatactatggaattctggtggaccaggcaaatatcccacccctacaaggtgggagggcggccgggaacccgtggtggcggttcctcccgtgatgatcgactaatggataagtcgcaatgatcgactaatggataagtcgcattgagatattggttcgaatccaattcattacgagctcttctggagagcattatctctaagagaataaaatgaaaaacccagctaggagaaaaaaacagtaagttaacaagatag

SEQ ID NO: 71 - DNA, 201 bp, Beet curly top virus
  source 1..201: mol_type = genomic DNA
tgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaag

SEQ ID NO: 72 - protein, 370 aa, synthetic construct
MSSSLLTDLVNLDLSESTDKVIAEYIWVGGTGMDVRSKARTLSGPVDDPSKLPKWNFDGSGTGQATGDDSEVILHPQAIFRDPFRKGKNILVMCDCYAPNGEPIPTNNRYNAARIFSHPDVKAEEPWYGIEQEYTLLQKHINWPLGWPLGGYPGPQGPYYCAAGADKSYGRDIVDAHYKACLFAGINISGINAEVMPGQWEFQIGPVVGVSAGDHVWVARYILERITEIAGVVVSFDPKPIPGDWNGAGAHTNYSTKSMRSNGGYEVIKKAIKKLGMRHREHIAAYGDGNERRLTGRHETADINNFVWGVANRGASVRVGRDTEKDGKGYFEDRRPASNMDPYLVTAMIAETTILWEPSHGHGHGQSNGK

SEQ ID NO: 73 - DNA, 1110 bp, synthetic construct
  source 1..1110: mol_type = other DNA
tcgtcgtccctgctcactgacctcgttaacctcgacctgtcggagagcactgacaaggtcatcgccgagtatatatgggttggtggtactgggatggatgtgagaagcaaagccagaacattgtctggacctgttgatgacccaagcaagctaccaaagtggaactttgatggctccggcactggtcaagctactggtgacgacagtgaagttatcctccatcctcaagccatcttccgtgacccattcagaaaggggaagaacatcctggtcatgtgtgactgttatgcgcctaatggcgagccaattcctactaacaaccgatataatgcagcaagaatcttcagtcatcctgatgtcaaggctgaagaaccttggtatgggattgagcaagagtatacccttcttcaaaagcatatcaactggcctcttggctggccactaggtggctatccaggccctcaaggtccatattattgtgcggcgggagccgataaatcgtatgggcgtgacatcgttgatgcccattataaggcctgcctgtttgccggcatcaacatcagcgggatcaacgcagaagtcatgcctgggcaatgggagttccaaattggccctgtcgttggcgtatccgcaggggatcatgtctgggtggcacgttatattcttgagagaatcactgagattgctggcgtcgtcgtgtccttcgaccccaagcccattcctggagactggaatggtgccggtgctcataccaattatagcaccaagtcgatgagaagcaatggcggctacgaggtgatcaagaaagcgatcaagaagctcggtatgcgccatcgtgagcatatcgccgcctatggcgacggcaacgagcgtagactcactggtcgtcatgagactgccgacatcaacaacttcgtctggggcgtagcgaaccgtggcgcgtcggtgcgtgtcggtcgagacaccgagaaggacggcaaaggttacttcgaggacagacgaccagcgtccaacatggacccatatctggtgaccgccatgatcgccgagactaccatcctctgggagcccagtcatggtcatggacatggccaatccaacggcaagtga

SEQ ID NO: 74 - DNA, 1151 bp, synthetic construct
  source 1..1151: mol_type = other DNA
gaatttctctgacattccatgtttccgaaacggatcctatatcgtcgtccctgctcactgacctcgttaacctcgacctgtcggagagcactgacaaggtcatcgccgagtatatatgggttggtggtactgggatggatgtgagaagcaaagccagaacattgtctggacctgttgatgacccaagcaagctaccaaagtggaactttgatggctccggcactggtcaagctactggtgacgacagtgaagttatcctccatcctcaagccatcttccgtgacccattcagaaaggggaagaacatcctggtcatgtgtgactgttatgcgcctaatggcgagccaattcctactaacaaccgatataatgcagcaagaatcttcagtcatcctgatgtcaaggctgaagaaccttggtatgggattgagcaagagtatacccttcttcaaaagcatatcaactggcctcttggctggccactaggtggctatccaggccctcaaggtccatattattgtgcggcgggagccgataaatcgtatgggcgtgacatcgttgatgcccattataaggcctgcctgtttgccggcatcaacatcagcgggatcaacgcagaagtcatgcctgggcaatgggagttccaaattggccctgtcgttggcgtatccgcaggggatcatgtctgggtggcacgttatattcttgagagaatcactgagattgctggcgtcgtcgtgtccttcgaccccaagcccattcctggagactggaatggtgccggtgctcataccaattatagcaccaagtcgatgagaagcaatggcggctacgaggtgatcaagaaagcgatcaagaagctcggtatgcgccatcgtgagcatatcgccgcctatggcgacggcaacgagcgtagactcactggtcgtcatgagactgccgacatcaacaacttcgtctggggcgtagcgaaccgtggcgcgtcggtgcgtgtcggtcgagacaccgagaaggacggcaaaggttacttcgaggacagacgaccagcgtccaacatggacccatatctggtgaccgccatgatcgccgagactaccatcctctgggagcccagtcatggtcatggacatggccaatccaacggcaagtga

SEQ ID NO: 75 - protein, 373 aa, synthetic construct
MDPISSSLLTDLVNLDLSESTDKVIAEYIWVGGTGMDVRSKARTLSGPVDDPSKLPKWNFDGSGTGQATGDDSEVILHPQAIFRDPFRKGKNILVMCDCYAPNGEPIPTNNRYNAARIFSHPDVKAEEPWYGIEQEYTLLQKHINWPLGWPLGGYPGPQGPYYCAAGADKSYGRDIVDAHYKACLFAGINISGINAEVMPGQWEFQIGPVVGVSAGDHVWVARYILERITEIAGVVVSFDPKPIPGDWNGAGAHTNYSTKSMRSNGGYEVIKKAIKKLGMRHREHIAAYGDGNERRLTGRHETADINNFVWGVANRGASVRVGRDTEKDGKGYFEDRRPASNMDPYLVTAMIAETTILWEPSHGHGHGQSNGK

SEQ ID NO: 76 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 77 - protein, 4 aa, synthetic construct
PVAT

SEQ ID NO: 78 - protein, 869 aa, synthetic construct
KTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVYPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 79 - DNA, 2610 bp, synthetic construct
  source 1..2610: mol_type = other DNA
aagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtatccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 80 - DNA, 75 bp, synthetic construct
  source 1..75: mol_type = other DNA
gaatttctctgtgaacagtcactcacttttgacagttatacgattccagaagatgatccagaattggatcctata

SEQ ID NO: 81 - DNA, 2685 bp, synthetic construct
  source 1..2685: mol_type = other DNA
gaatttctctgtgaacagtcactcacttttgacagttatacgattccagaagatgatccagaattggatcctataaagaccggccgtaagaaccatcaacgacatcatgtccttcccgcacgaggccgagtgggggcggcggcggtcaggtgctcggcggtgtccccagtcaccccaccatccccagcgccaccagccactccactccgaccatgggggccagccgagccccgtaagggcgcggacatcctcgtggaggcgctggagcgatgcggcgtcagcgacgtgttcgcctatccaggcggcgcgtccatggagatccatcaagcgctgactcgttccccagtcatcaccaaccatctcttccgtcatgagcaaggcgaggcgttcgcggcgtccgggtatgcgcgtgcgtccggccgtgtcggggtctgcgtcgccacctccggccccggggcaaccaacctcgtgtccgcgctcgccgacgcgctgctcgactccgtcccaatggtcgccatcactggccaagtcccccgtcgtatgatcggcaccgacgccttccaagagactcccatagtcgaggtcacccgttccatcaccaagcataattatcttgtccttgatgtggaggacatcccccgtgtcatacaagaagccttcttcctcgcgtcctcgggccgtcctggcccagtgctggtcgacatccccaaggacatccaacaacaaatggccgtgccagtctgggacacctcgatgaatctaccagggtatatcgcacgtctgcccaagccacccgcgacagaattgcttgagcaagtcttgcgtctggttggcgagtcacgacgtccaattctctatgtcggtggtggctgctctgcatctggtgacgaattgcgttggtttgttgagctgactggtatcccagttacaaccactctgatgggcctcggcaatttccccagtgacgacccattgtccctgcgtatgcttgggatgcatggcactgtgtatgcaaattatgccgtggataaggctgacctgttgcttgcgtttggtgtgcgatttgatgatcgtgtgacagggaaaattgaggcttttgcaagcagggccaagattgtgcatattgacattgatccagcagagattggaaagaacaagcaaccacatgtgtcaatttgcgcagatgttaaacttgctttacaaggcttgaatgctctgctacaacaaagcacaacaaagacaagttctgattttagtgcatggcataatgagttggaccaacaaaagagggagtttcctctggggtataaaacttttggtgaagagatcccaccacaatatgccattcaagtgctggatgagctgactaaaggtgaggcaatcatcgctactggtgttgggcaacatcaaatgtgggcggcacaatattatacctataagcgaccacgacaatggctgtcttcggctggtctgggcgcaatgggatttgggctgcctgctgcagctggtgcttctgtggctaacccaggtgtcacagttgttgatattgatggggatggtagcttcctcatgaacattcaagagctggcattgatccgtattgagaacctccctgtgaaggtgatggtgttgaacaaccaacatttgggtatggtggtgcaattggaggataggttttataaggcgaatagggcgcatacatatttgggcaacccagaatgtgagagcgagatatatccagattttgtgactattgctaaggggttcaatattcctgcagtccgtgtaacaaagaagagtgaagtccgtgccgccatcaagaagatgcttgagactccagggccatatttgttggatatcatcgtcccacatcaagagcatgtgctgcctatgatcccaattgggggcgcattcaaggacatgatcctggatggtgatggcaggactgtgtatccagttgctactatggtgagtaagggagaggagctgttcaccggggtggtgcctatcctggtcgagctggatggtgatgtaaacggtcataaattcagtgtgtccggtgaaggtgaaggtgatgccacctatggtaagctgacccttaagttcatctgtaccaccggaaagctgcctgtgccttggcctaccctcgtgaccaccctgacatatggagtgcaatgtttcagtcgttatcctgatcatatgaagcaacatgatttctttaaatccgccatgcctgaaggttatgtccaagagcgtaccatattctttaaagatgatggtaactataagacccgtgccgaggtgaagttcgagggtgataccctggtgaaccgtattgagcttaagggtatcgatttcaaggaggatggaaacatcctggggcataagctggagtataactataacagtcataacgtctatatcatggccgataagcaaaagaacggtatcaaggtgaacttcaagatccgtcataatatcgaagatggaagtgtgcaactcgccgatcattatcaacaaaacacccctatcggtgatggtcctgtgctgctgcctgataaccattatctgagtacccaatccgccctgagtaaagatcctaacgagaagcgtgatcaaatggtactgcttgagttcgttaccgccgccgggatcactctcggtatggatgagctgtataagtaa

SEQ ID NO: 82 - protein, 881 aa, synthetic construct
MIPEDDPELDPIKTGRKNHQRHHVLPARGRVGAAAVRCSAVSPVTPPSPAPPATPLRPWGPAEPRKGADILVEALERCGVSDVFAYPGGASMEIHQALTRSPVITNHLFRHEQGEAFAASGYARASGRVGVCVATSGPGATNLVSALADALLDSVPMVAITGQVPRRMIGTDAFQETPIVEVTRSITKHNYLVLDVEDIPRVIQEAFFLASSGRPGPVLVDIPKDIQQQMAVPVWDTSMNLPGYIARLPKPPATELLEQVLRLVGESRRPILYVGGGCSASGDELRWFVELTGIPVTTTLMGLGNFPSDDPLSLRMLGMHGTVYANYAVDKADLLLAFGVRFDDRVTGKIEAFASRAKIVHIDIDPAEIGKNKQPHVSICADVKLALQGLNALLQQSTTKTSSDFSAWHNELDQQKREFPLGYKTFGEEIPPQYAIQVLDELTKGEAIIATGVGQHQMWAAQYYTYKRPRQWLSSAGLGAMGFGLPAAAGASVANPGVTVVDIDGDGSFLMNIQELALIRIENLPVKVMVLNNQHLGMVVQLEDRFYKANRAHTYLGNPECESEIYPDFVTIAKGFNIPAVRVTKKSEVRAAIKKMLETPGPYLLDIIVPHQEHVLPMIPIGGAFKDMILDGDGRTVYPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDQMVLLEFVTAAGITLGMDELYK

SEQ ID NO: 83 - DNA, 1162 bp, synthetic construct
  source 1..1162: mol_type = other DNA
taaggtaccaaaataatacgactcactatagacctaggaaaagatctagaagcggggtagaggaattggtcaactcatcaggctcatgacctgaagactgcaggttcgaatcctgtccccgcctgtctggccccaaattctaatttctactgttgtagatgtatcgatcatatattctaggcgaggtgtagcgcagtctggtcagcgcatctgttttgggtacagagggccataggttcgaatcctgtcaccttgagtctggccccaaattctaatttctactgttgtagataactgaaatgggctgatctaggtcaggatagctcagttggtagagcagaggattgaaaatcctcgtgtcaccagttcaaatctggttcctggcagtctggccccaaattctaatttctactgttgtagattgtaaaggagtacaaggtgctggagagatggccgagtggttcaaggcgtagcattggaactgctatgtaggcttttgtttaccgagggttcgaatccctctctttccggtctggccccaaattctaatttctactgttgtagattcaacaccagcttctcacctggggtgtatagctcagttggtagagcattgggcttttaacctaatggtcgcaggttcaagtcctgctatacccaaaccccttggggcctctaaacgggtcttgaggggttttttggtaccaaccccttggggcctctaaacgggtcttgaggggttttttgatagacctttttatttttcgtcattcgatcacgaaaacagggattctggaacggccaagaatcccagcggttgttcgggtcgaaaaaccgagaacaagacatgccacaaagtggcagatgaaggcaggggggagagcctagtcctcaacctcttcttccccaaaaggtagttatgaacgtgccaaacttattggatttattcttggaatgctcataaccacctttactctttttttcattctttactcagaggaagccatgccgtttggagaagagcaccaagtggggggagtgtggagtccccgaaagaggagctttctaaaggcaagagaaaagctccgatggagcccttggagctacagggaccaccaacccttcgcagcttggacgatttgattcttgtgccactcagccc

SEQ ID NO: 84 - DNA, 2029 bp, synthetic construct
  source 1..2029: mol_type = other DNA
tgaggtattatgggatttatgtttataccaaagcctctactttagatatttacgctgatgaaggaaagccttgttaaagactacgactttggacttcctttttcgtactcttctttcatcctccttaccagaacgcttttcctggttcagaagaataagatacttaaattgtggcttacatatggaaaatgccttcgtcttgaagctaccaatgcacatcacggtgaaatcattgtcaattaattgttagattccccatatcacccatagccacccaaatcttttttttgcctcaaactctgctgccctatcactcctcgataagtgtacttactcaggttgtgcacttggctgagcgagatcttcccctttggcatgagaaccgcctacctacgctaacgtaactaatgcagatggcggaagttagtagttctcctatgccaacatgaacacaaacgaaatcgtcgaatttcgtatagaaagaaaaggaaccacttctattctcttttcttaagtataagagggataagtacttattcctggccggcggtatcattgctgctccccgcctaatgcggatcattgtgcaatgcttatgtgaaatctcaatccaaaacttattcgtttcgttggaaaaaccaacgccgatctaaaactaagtcttcctttcaaaagtgagcgagcagagctgaaaaagatggagttacctggagatgagatttctttctacggatatgaaggatagaaatatgctatttgctgctattacaacgaatcaaccaattcgtagtaagtgttcccgtcttcccgatctacatgattttttcccaaccaacatctctcagaactttgctataacgccaaacttggatataacgccaacgcctgagcgaattgccggcgtcacaatagttttacaaatagaagagtatttgggccaaaatgagtccgaacagggagcagtcaatttagctagaacagtattgggagcccgccaccgaaatggcgaaacttggcagggcatattagaggatattcgggcgggtggtggtatggataattttatccagaatctgcctggtgcctacccggaaaccccattggatcaatttgccattatcccaataattgatcttcatgtgggcaacttttatttatcatttacaaatgaagtcttgtatatgctgctcactgtcgttttggtcgtttttcttttttttgttgttacgaaaaagggaggtggaaagtcagtgccaaatgcatggcaatccttggtcgagcttatttatgatttcgtgctgaacctggtaaacgaacaaataggtggtctttccggaaatgtgaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattacctgcgggagtcccactgccgttagcacctttcttagtactccttgagctaatctcttattgttttcgtgcattaagcttaggaatacgtttatttgctaatatgatggccggtcatagtttagtaaagattttaagtgggtttgcttggactatgctatttctgaataatattttctatttcataggagatcttggtcccttatttatagttctagcattaaccggttttgaattaggtgtagctatatcacaagctcatgtttctacgatctcaatttgtatttacttgaatgatgctacaaatctccatcaaaatgagtcatttcataattgaaagaaggctaccgaaagaacaagcaacggatttcgcgctcatcccttgctaaagcgcatacgttttcttgctccttggtacttgtctgatcaatcacactgttcattagttcacttgatttttcgttcgatgtcttgaaccgcttactaacctga

SEQ ID NO: 85 - DNA, 222 bp, synthetic construct
  source 1..222: mol_type = other DNA
tagggataacagggtaattgagtggagcaatatatttaataacctttgtagtgatctcagttcatggagatcacgaggccactgatacccgaagtggttaggtgggatagaagagtgggtatgtgggcttctatcaaaagagaccgctatcgctacgctcatatacgtcaaggaaaggaagggagcgaaaagatacgttaatgtttccacctactctctttt

SEQ ID NO: 86 - DNA, 1952 bp, synthetic construct
  source 1..1952: mol_type = other DNA
aatccaatcccacaaaggattgttcatggaactactatcgaaattattcggaccatatttccaagtgtcattcttttgttcattgctataccatcgtttggtcttgcgttgacagtactaataatggcatttgttgtcttattcggagttcaaggaatagcctttcatcttgggaatgagaatgtcgcggatctcaatgttctcgtcatgaccaatgctcctaacgggggtgactttcccatagaccaacctcccgttggcggactaccagacctccagcaggaaatggcgcacgccgcccatcccgaagacataattgcggagttgacttccaaggttgaaagaatatttcacgaagtaggcactcctcttcctttagaggaaggtgaatctgcgcgatcgttcactgaaaatcatgtattgtggaatagtgagggaggagatactcttcaacaaatcttctccgattatacggaagccggggacgcaagcgaattcgtcgcaatagccaccaatctggcgaagcggtttcgccgcgccgagctcggtgaaccggattctcccgaccccgatgcgcctttggaagagcaggaggctcattccacgggattcccctgaaagcacaaaagcaaatgatggggcgaacgaggcaggcccgtcttcttcgcggaagcgtaaaaggtgggatgatgattcggggtccgaagatgatgattcggggggccccggggaatctccagatccagtggaaatcgtttacgaaggggatgcacaagggtacaactgggaggggaattaggttggccgccaacttcgcctgcctttctatctgagttcttccctcttgatgctttcgaacgactcctaaatttcacaaaatcctttttttcttatttgaaatccaaatcgaaatgcctcaacttgataaattaacttatttctcacaattcttctggttatgtcttctcctctttactttttatattctcttatttaataataataatggaatacttggaattagtagaattctcaaactacggaaccaactgctttcgcaccgggggggcgagatccggagcaaggaccctaagaatctggaagatatctcgagaaaaggttttagcaccggtctctcatatatgtactccagtttatccgaagtatcccaatggtgtaagaccgtcgactatttgggaaataagatatcttcttcaatctttctatactatcttaggggcgtcctttgcccaatttgcctcttattttttaaatttcttatttctttcgccttcacctgtgcacttatttatgtattcaagggggggggtttcgtggctatggctgctacaaacggagcctcttcttcctttctggagagctcaggtgaaatagatgcactgttacaaacaacaacaacaacacgaacacccgaaaacgcggattacgaagacgataatacttctgtaaatcaagaattcctcgaagacggacaaagggcccgacaggctaaactacgggagttagaaagactcattctccagcaatataaggatttcatccgacagaaatatccatggatacctaaaggcgacatccttctcccgagcatgaagggtggagttgtcgaggacgtaatggaaaaattagaattggaaacgtattccagtagcgacttgactgattggatcaaccatttgcgggcaaatccgaaaacattaaattttatcttcaaggattttgtcgcgtgagtggagcaatatatttaataacctttgtagtgatctcagttcatggagatcacgaggccactgatacccgaagtggttaggtgggatagaagagtgggtatgtgggcttctatcaaaagagaccgctatcgctacgctcatatacgtcaaggaaaggaagggagcgaaaagatacgttaatgtttccacctactctctttt

SEQ ID NO: 87 - DNA, 1200 bp, Triticum aestivum
  source 1..1200: mol_type = genomic DNA
aaatgagataaggagcagtaaggaggataaaagaagtagagttaggttcttgtgtgtaatcgatatgtctcccgggagaaaaccctctatcaacacaactatccgaacacctatagctagcttgctttagctccaactactgctagctttacctgcaacgcaactatcttccgaaaaacgagttgttcgttgcccgacgcggctctggctctgcctgcctggcactaaagcatctatagattgaggatttgcctatgtaccatcatccgtcctctaagtaatatgtattcttcctctaaggttccccctctaaggagggttgtgtagtatttagcttacaaggattgactcgacaccatcgatgcgtagtctatcaatcaatttaatgggacttatttatatgcctgttacgaggggaatctgcctgttattcaatctatctcttcaatctgcttactggtttccttctcggccaatggaacatctattgcttcctaatctggaactgaactaaacagaaatcttggtcaagtattgggaaaaggaaagaggcccttttctttttcaaaactaagaaggagatgtgcatagcgttgtaaagatcggcagaagaggaagggtgcagttaagttacaacatcttctgtttccaagtcgataggcttcttttgataaaggaaactattcctctgctcgctatactcaaacccttgccatttcctcgattcccgttgagaaaacaatcgattgaccattttaggacctattcttcagaatatagaggtatcgaagatctagaaacgatgcgatgactccagttcgatccttttccgatgaaatgaatgaattaccacgctttccgccggctgataaagaactttttgatgatcgattgcttcacccatgatctgatgatcttgctataacataacgatactgcatttcccctcttccttgtctctacttaacttacttgttcattgctcgactcgagggaggggctctatatagctgttttgccttctttcttggactctcaggtgagcgcttaggccaagccacaattaccaaggcatgattgaatccgccaaagagaagggaatagatgatctcactggaaaaagttgcgtaaccccccccccccccgtttttttggcatgatttcaagattttgacctgatttatgagaaaagaacttcgt

SEQ ID NO: 88 - DNA, 1636 bp, synthetic construct
  source 1..1636: mol_type = other DNA
atgagatttctttctacggatatgaaggatagaaatatgctatttgctgctattacaacgaatcaaccaattcgtagtaagtgttcccgtcttcccgatctacatgattttttcccaaccaacatctctcagaactttgctataacgccaaacttggatataacgccaacgcctgagcgaattgccggcgtcacaatagttttacaaatagaagagtatttgggccaaaatgagtccgaacagggagcagtcaatttagctagaacagtattgggagcccgccaccgaaatggcgaaacttggcagggcatattagaggatattcgggcgggtggtggtatggataattttatccagaatctgcctggtgcctacccggaaaccccattggatcaatttgccattatcccaataattgatcttcatgtgggcaacttttatttatcatttacaaatgaagtcttgtatatgctgctcactgtcgttttggtcgtttttcttttttttgttgttacgaaaaagggaggtggaaagtcagtgccaaatgcatggcaatccttggtcgagcttatttatgatttcgtgctgaacctggtaaacgaacaaataggtggtctttccggaaatgtgaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattacctgcgggagtcccactgccgttagcacctttcttagtactccttgagctaatctcttattgttttcgtgcattaagcttaggaatacgtttatttgctaatatgatggccggtcatagtttagtaaagattttaagtgggtttgcttggactatgctatttctgaataatattttctatttcataggagatcttggtcccttatttatagttctagcattaaccggttttgaattaggtgtagctatatcacaagctcatgtttctacgatctcaatttgtatttacttgaatgatgctacaaatctccatcaaaatgagtcatttcataattgatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaacgccgaccccaaacaagtctctccaatataaggagagcggagcttaaaaatattattttattgtgctatggcaaatctggtccgatggctcttctccactacccgagggactaacggtcttccatatttcatcttcggtgtcgttgtaggaggcgccctgttgtttgctttgctaaagtatcaggcccctctgtacgacccggctttaatggaaaaaatcatagatcataatataaaagccgggcaccctatagaggttgactattcgtggtggggcacctctattcgtgtagtctttcctaagtaaataaaaacgaggagccgaagattcctga

SEQ ID NO: 89 - DNA, 30 bp, synthetic construct
  source 1..30: mol_type = other DNA
agggaaattcatcccaaccaatagcagtga

SEQ ID NO: 90 - DNA, 26 bp, synthetic construct
  source 1..26: mol_type = other DNA
catcggaccagatttgccatagcaca

SEQ ID NO: 91 - DNA, 31 bp, synthetic construct
  source 1..31: mol_type = other DNA
ctgaggagtaagcctaattccgttaatgcag

SEQ ID NO: 92 - DNA, 31 bp, synthetic construct
  source 1..31: mol_type = other DNA
cttatattggagagacttgtttggggtcggc

SEQ ID NO: 93 - DNA, 22 bp, synthetic construct
  source 1..22: mol_type = other DNA
cgcgttagcgcattcggaacca

SEQ ID NO: 94 - DNA, 29 bp, synthetic construct
  source 1..29: mol_type = other DNA
tcgaggtaattaaagcggccgaaattagc

SEQ ID NO: 95 - DNA, 26 bp, synthetic construct
  source 1..26: mol_type = other DNA
ctcagaataatccaggtcgctcgacg

SEQ ID NO: 96 - DNA, 36 bp, synthetic construct
  source 1..36: mol_type = other DNA
cacaggtagcaataggtattacagtagggataacag

SEQ ID NO: 97 - DNA, 30 bp, synthetic construct
  source 1..30: mol_type = other DNA
atgcacataagccatccgaaaccagtattg

SEQ ID NO: 98 - DNA, 22 bp, synthetic construct
  source 1..22: mol_type = other DNA
gaatggaggaacggcgatatag

SEQ ID NO: 99 - DNA, 23 bp, synthetic construct
  source 1..23: mol_type = other DNA
cttcttattaccagcgggagtcc

SEQ ID NO: 100 - DNA, 23 bp, synthetic construct
  source 1..23: mol_type = other DNA
gaaggtagacctaaacgctgaag

SEQ ID NO: 101 - DNA, 21 bp, synthetic construct
  source 1..21: mol_type = other DNA
gggaaaggaaacaaaccactc

SEQ ID NO: 102 - DNA, 20 bp, synthetic construct
  source 1..20: mol_type = other DNA
tcccgtgatattccgccttg

SEQ ID NO: 103 - DNA, 1739 bp, synthetic construct
  source 1..1739: mol_type = other DNA
gatagaagagtaccaaccaaagaagcaaaagcaagacctcactagtaaggaaggcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaggtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtagtatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttggatcaatttttcattgatccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttattgtattggctcttactggattggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaattcgtttttcgttggaaaaaccaac

SEQ ID NO: 104 - DNA, 1370 bp, synthetic construct
  source 1..1370: mol_type = other DNA
ggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcaaagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaaggaggttaccggttaagggcaaatgcaatctctaagccagttggagaagaaaggtaatcaaagaaagtttcagttggacttaccctgccaaccctaccctaatttcataccctgcggttttcgttcctctattgatatttcgttcctctcctta

SEQ ID NO: 105 - DNA, 23 bp, synthetic construct
  source 1..23: mol_type = other DNA
ggccaactcaaattccaatgctg

SEQ ID NO: 106 - DNA, 796 bp, synthetic construct
  source 1..796: mol_type = other DNA
  misc_difference 400: note = a, c, t, g, unknown or other
  misc_difference 401: note = a, c, t, g, unknown or other
gcacttgctgccggagttcaacaggcaaatataagaaaagaagtcctgttcacttcatcatctgtgggttgtactgcttgaaggttcttctgaggggtagaatttgaattccttctttgcttgtgagataaccatttccagaaactcatatatagagagcgggtatcggtgaaaatggatcttaccaggagtggcattgaataggcaggctctgggatgtaatctcactcaagaagtcatttgttggccccgccttcactagactagagttttaggataggttggggaacctatacgtcaagcccctacgaagattgagaaaaatcgatgcacataagccatccgaaaccagtattggaaagtgttcagtttcgttttccattctgaaatgttcatagtnntatagtatgttttccgttgggtcgacgccatgtgatcgctactaaagatagagtttccttggaaaaaccgaggccagttgagatcagtctccctttctaggagcagagcttaaaaagatgggaaattccaatgaatttcgatcacaatcatgtggtaataatgggtttgaatcagagagactcgatctggaaactcctcaatgattataacgtgaactcgttgaagagaaggagacaagcagaaatagacgctttttttgaaccatttgagagggcgcagcgtatccgtttcaataactggcagaacggaatagagttgttagatggggctgaatggaggaacggcgatatagttatccctggaggcggcggaccagtaatttcaagccccttgg

SEQ ID NO: 107 - DNA, 850 bp, synthetic construct
  source 1..850: mol_type = other DNA
ccattatttggtcttgatatgggtaacttttatttatcattcacaaatgaatccttgtctatggcggtaactgtcgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttatagttctagcattaaccggtctggaattaggtgtagctatattacaagctcatgtttctacgatctcaatttgtatttacttgaatgatgctataaatctccatcaaaatgagtaatttcataattgaataaaaacgaggagccgaagattttagggggcgggacaaacgcggaagtgtattgcgttacaaaaaatgacaactagcatttgttttttcatttcatgttcgaatt

SEQ ID NO: 108 - DNA, 1365 bp, synthetic construct
  source 1..1365: mol_type = other DNA
  misc_difference 819: note = a, c, t, g, unknown or other
  misc_difference 820: note = a, c, t, g, unknown or other
  misc_difference 844: note = a, c, t, g, unknown or other
  misc_difference 965: note = a, c, t, g, unknown or other
  misc_difference 967: note = a, c, t, g, unknown or other
  misc_difference 988: note = a, c, t, g, unknown or other
  misc_difference 989: note = a, c, t, g, unknown or other
  misc_difference 1030: note = a, c, t, g, unknown or other
agcaataggtattacagtagggataacagggtaatgaattcgaaccatcttctcactctgacccccacatatcagatcccagatgcatagaaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgtccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctaccttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtgatgagtgggggccttgtatctgtattcggatcagctccttaacagagtttcctgcttgaaccctggctgggnnctgggagaggtgtcccactacagntgcaaataaaccatttgaccttacaggggaaaggaaacaaaccactcaataatcggtagaaattcctcctactgaacagctttccttttctcgccttaactactacttcaaagcaaggcgnanatcacgggataggaatgaaannacttcttactcactttctagctatataaaaatagttagcanatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcgaataaggtccttgctctcgagccaatgccaataccaatagagagggtctaaacgaaggattcaaaggaggttaccggttaagggcaaatgcaatctctaagccagttggagaagaaaggtaatcaaagaaagtttcagttggacttaccctgccaaccctaccctaatttcataccctgcggttttcgt

SEQ ID NO: 109 - DNA, 871 bp, synthetic construct
  source 1..871: mol_type = other DNA
  misc_difference 74: note = a, c, t, g, unknown or other
tggatcaatttttcattgatccattatttggtcttgatatgggtaacttttatttatcattcacaaatgaatcnttgtctatggcggtaactgccgttttggtgccatctttatttggagttgttacgaaaaagggcgggggaaagtcagtgccaaatgcatggcaatccttggtagagcttatttatgatttcgtgctgaacctggtaaacgaacaaataggtggaaatgttaaacaaaagtttttccctcgcatctcggtcacttttactttttcgttatttcgtaatccccagggtatgataccctttagcttcacagtgacaagtcattttctcattactttggctctttcattttccatttttataggcattacgatcgttggatttcaaagacatgggcttcatttttttagcttcttattaccagcgggagtcccactgccattagcaccttttttagtactccttgagctaatctctcattgttttcgtgcattaagctcaggaatacgtttatttgctaatatgatggccggtcatagttcagtaaagattttaagtgggttcgcttggactatgctatttctgaataatattttctatttcataggagatcttggtcccttatttatagttctagcattaaccggtctggaattaggtgtagctatattacaagctcatgtttctacgatctcaatttgtatttacttgaatgatgctataaatctccatcaaaatgagtaatttcataattgaataaaaacgaggagccgaagattttagggggcgggacaaactcggaagtgtattgcgttacaaaaaatgacaactagcatttgttttttcatttcatgttcgaattc

SEQ ID NO: 110 - DNA, 1376 bp, synthetic construct
  source 1..1376: mol_type = other DNA
caataggtattacagtagggataacagggtaatgaattcgaaaccatcttctcactctgacccccacatatcagatcccagatgcataggaaaagcggtatcaagaatagtagtataaagaaagatagtacagtactcaagtaaatgaattcgcctaaggatcgatggaaagatcaaggtccccgtgaaaaagtagatactagatcgatatgatactctcatctctggagtaacttcttccattatgctgatctctaggtccgttccatcatcatcgtaatagtatggtcccaggtgcccgagctatagatcaagatcatatccagtcacatttctaccggtgcacttctcatgaaataattccctttccaaggaaaggaaaacaagaactcgaatactcgtaatagcgatcccgatccacctacttttttctattctttgattcgaaacgtgctaaagcacaagccatttttatgcatggggcataagagtggacaatctatgttatcgaaggaagtaaataacaacacttcagcgtttaggtctacctttcagtaaaccaatagttttgcagcattggaatttgagttggccaggtaaggtcctctaaaaagaaaagaagaaactacttagaatagataaatgccattggttttctcgtactatacgatctttttttgttttgttttttggccatgattgtgctgctcctgtgaaggctagtgggaaagctcaccgttcgttgtggtgagtgggggccttgtatctgtattcggatcagctccttaacagagtttcctgcttgaaccctggctgggagctgggagaggtgtcccactacaggtgcaaataaaccatttgaccttacaggggaaaggaaacaaaccactcaataatcggtagaaattcctcctactgaacagctttccttttctcgccttaactactacttcagagcaaggcggaatatcacgggataggaatgaaagaacttcttactcaactttctagctatataaaaatagttagcaatatgaaacgagtaacttaagccctagtaaaaggctactctttgaatcccctctttaaggcatataaaattagtactcttcctgagctagcttaagcatatcttgagcgagtgagttgtatttccctccatcaagttctaagcgatcaaataaggtccttgctctcgagccaatgccaataccaatagagagggtctaaacgaaggattcaaagggggttaccggttaagggcaaatgcaatctctaagccagttggagaagaaaggtagtcaaagaaagtttcagttggacttaccctgccaaccctaccctaatttcataccctgcggttttcgttcctctat

SEQ ID NO: 111 - protein, 393 aa, synthetic construct
MFKQASRLLSRSVAAASSKSVTTRAFSTELPSTLDSPPTKRFRIQAKNIFLTYPQCSLSKEEALEQIQRIQLSSNKKYIKIARELHEDGQPHLHVLLQLEGKVQITNIRLFDLVSPTRSAHFHPNIQRAKSSSDVKSYVDKDGDTIEWGEFQIDGRSARGGQQTANDSYAKALNATSLDQALQILKEEQPKDYFLQHHNLLNNAQKIFQRPPDPWTPLFPLSSFTNVPEEMQEWADAYFGVDAAARPLRYNSIIVEGDSRTGKTMWARSLGAHNYITGHLDFSPRTYYDEVEYNVIDDVDPTYLKMKHWKHLIGAQKEWQTNLKYGKPRVIKGGIPCIILCNPGPESSYQQFLEKPENEALKSWTLHNSTFCKLQGPLFNNQAAASSQGDSTL